(19) United States
(12) Patent Application Publication
(10) Pub. No.: US 2007/0060558 A1
(43) Pub. Date: Mar. 15, 2007

(54) Hybrid molecules QA where Q is an aminoquinoline and A is an antibiotic residue, the synthesis and uses thereof as antibacterial agents

(75) Inventors: Muriel Sanchez, Balma (FR); Bernard Meunier, Castanet-Tolosan (FR)

Correspondence Address: LE-NHUNG MCLELAND, 9679-C MAIN STREET, FAIRFAX, VA, 22031-3766, US

(73) Assignees: PALUMED S.A., Labege Cedex (FR); Centre National de la Recherche Scientifique (CNRS), Paris (FR)

(21) Appl. No.: 11/516,692

(22) Filed: Sep. 7, 2006

Related U.S. Application Data
(63) Continuation-in-part of application No. 11/019,450, filed on Dec. 23, 2004, now abandoned.
(63) Continuation-in-part of application No. PCT/FR05/01937, filed on Jul. 26, 2005.

(30) Foreign Application Priority Data
Jul. 30, 2004 (FR) 0408441

(51) Int. Cl. A61K 31/545; A61K 31/43; A61K 31/4709; A61K 31/496

(52) U.S. Cl. 514/192; 514/202; 514/210.02; 514/210.09; 514/210.06; 514/253.08; 514/312; 540/224; 540/313; 540/200; 540/350; 544/363; 546/156

(57) ABSTRACT

The invention concerns an aminoquinoline-antibiotic hybrid compound of general formula (I): Q—(Y₁)ₚ—(U)ₚ—(Y₂)ₚ-A: wherein Q represents an aminoquinoline, (Y₁)ₚ(U)ₚ—(Y₂)_{p″} is an optional spacer and A is an antibiotic residue. The invention enables the antibiotic residue activity to be unexpectedly enhanced.

An object of the invention is hybrid molecules “QA” containing an aminoquinoline moiety (Q) which is covalently linked to an antibiotic residue (A). The invention also relates to their synthesis and their uses as antibacterial agents.

### STATE OF THE ART

Over the last 50 years, the introduction of penicillin followed by many other antimicrobial agents has represented one of the greatest successes of modern medicine in the treatment of bacterial infections (Greenwood, D. et al. in Antimicrobial Chemotherapy, Greenwood, D., Ed.; Oxford University Press: New York, United States, 2000). The appearance and the propagation of bacterial strains which are resistant to practically all the anti-microbial agents currently available are becoming a serious problem for public health (World Health Organization. Résistance aux antimicrobiens: une menace pour le monde. Médicaments essentiels: Le Point, 2000, 28 and 29, 1-35. Accessible on www.who.int).

The problem of bacterial resistance is also analyzed by Coates, A.; et al. in Nature Rev. Drug Discov. 2002, 1, 895-910, entitled: “The Future Challenges Facing the Development of New antimicrobial Drugs”.

The aminoquinolines (Q) are known molecules.

Moreover, it has been demonstrated by Malléa et al. in the literature that the aminoquinolines (Q), as a mixture with various classes of antibiotics, inhibited the active efflux of the antibiotics (vide Malléa, M.; et al. Alkylaminoquinolines inhibit the bacterial antibiotic efflux pump in multidrug-resistant clinical isolates. Biochem. J. 2003, 376, 801-805). This publication is considered by the inventors to be the most similar document to the invention. Various documents show that specific antibiotics can be coupled by specific covalent bonds to aromatic compounds defined by a general formula to improve the antibiotic properties. However, these documents disclose the aromatic part that is coupled to the antibiotic very generally and do not show specific activity of an aminoquinoline substituent.

### AIMS OF THE INVENTION

A main aim of the present invention is to solve the novel technical problem which consists of providing a solution which makes it possible to find novel antibiotic molecules less prone to bacterial resistance.

A further main aim of the invention is to find novel antibiotic molecules that are more effective than current antibiotics.

A further aim of the invention is to find novel antibiotic molecules that can be active on bacterial strains that are resistant to certain current antibiotics.

Yet another main aim of the present invention is to solve these novel technical problems by providing novel antibiotic molecules, the manufacture of which is relatively easy according to an inexpensive manufacturing procedure which gives good industrial yields.

The present invention solves, for the first time, the whole of these technical problems in a satisfactory, safe and reliable manner, which can be used on an industrial scale, in particular on a pharmaceutical scale.

### SUMMARY OF THE INVENTION

The innovative character of the present invention concerns the preparation and the evaluation of the hybrid molecules “QA”. According to the invention, the aminoquinoline part (Q) of these novel molecules has been covalently fixed to an antibiotic residue (A).

These hybrid molecules QA are generally named “antibioquines” or particularly “peniciquines”, “cephaloquines”, “quinoloquines” “nitoimidaquines”, “streptogramiquines”, “diaminopyrimiquines”. “vancomyquines” or “oxazoquines” where the A moiety is an antibiotic residue, respectively, a penicillin, cephalosporin, quinolone, nitroimidazole, pristinamycin, diamnopyrimidine, vancomycin or oxazolidinone moiety.

In an unexpected and non-obvious way it has been discovered according to the invention that the covalent anchoring of an aminoquinoline onto an antibiotic did not lead to a loss of the antibiotic activity, but on the contrary, led to a synergistic effect increasing the antibiotic activity, wherein constituting the basis of the present invention. None of the disclosures of the prior art known by the inventors shows, nor obviously suggests, that the aminoquinoline type compounds makes it possible to obtain a synergistic effect increasing the antibiotic activity when they are covalently coupled with an antibiotic. A person skilled in the art would rather expect a risk of loss of activity in covalently binding an antibiotic residue to an aminoquinoline.

In particular, aminoquinolines make it possible to combine an inhibitory effect on the efflux pumps of certain resistant bacteria and the antibacterial effect of the antibiotic.

Unexpectedly, the hybrid molecule QA has much greater antibacterial activity than one or other of the components A or Q taken separately.

Another particularly unexpected effect of the invention resides in the fact that it has been surprisingly discovered that the antibiotic activity was preserved in the case of a covalent bond with an aminoquinoline for various classes of antibiotics. Thus, this unexpected improvement in the activity is not limited to a particular type of antibiotic.

This constitutes a particularly significant technical improvement of the invention insofar as the actual tendency for an antibiotic treatment is no longer the use of broad spectrum antibiotics. In fact, broad spectrum antibiotics currently strongly participate in the selection of resistant organisms, and, moreover, they bear within them an inherent danger of deep modifications of the flora with a development of secondary complications which are sometimes dangerous. Hence, the use of antibiotics should tend to the use of an antibiotic which is as selective as possible on the germ in question, for as short a time period as possible.

By virtue of the fact that the invention is not limited to a particular class of antibiotics, it will in contrast thus be possible to modify the various families of antibiotics without reducing their effectiveness.

The invention will therefore make it possible to have a panel of molecules at one's disposal which are active on resistant strains and which will be able to be used as a function of their specific activity.

It will be possible for the person skilled in the art to assess the major significance of the present invention, which covalently links an aminoquinoline type moiety (Q) to a residue (A) representing an antibiotic residue, linked to each other via a covalent bond which is represented by —(Y₁)ₚ—(U)_{p′}—(U₂)_{p″}—, a covalent bond which can be direct or indirect by the use of a spacer arm.

### DETAILED DESCRIPTION OF THE INVENTION

The invention relates essentially to novel hybrid antibiotic molecules which are represented by the general formula (I):
Q—(Y₁)ₚ—(U)_{p′}—(Y₂)_{p″}-A (I)
in which

Q represents an aminoquinoline-type molecule;

A represents an antibiotic residue;

which are linked together via a covalent bond which is represented by —(Y₁)ₚ—(U)ₚ—(Y₂)_{p″}—, a covalent bond which can be direct or indirect by the use of a spacer arm.

The antibiotic residue A is covalently linked either directly to the aminoquinoline, or to the spacer arm and can be linked notably to Q, Y₁, U, or Y₂, in particular as defined below, in any fixing site, notably by reaction with one of the reactive functions of the compounds A.

The present invention also relates to their method of preparation, their various uses, to pharmaceutical compositions containing them, as well as to a method of therapeutic treatment. These novel molecules can also be used as an antibacterial agent.

According to a first aspect, the present invention provides a hybrid aminoquinoline-antibiotic compound, wherein it has the following general formula (I):
Q—(Y₁)ₚ—(U)_{p′}—(Y₂)_{p″}-A (I)
in which:

Q represents an aminoquinoline having the following formula (IIa), (IIb), (IIIa), (IIIb), (IIIc) or (IIId):

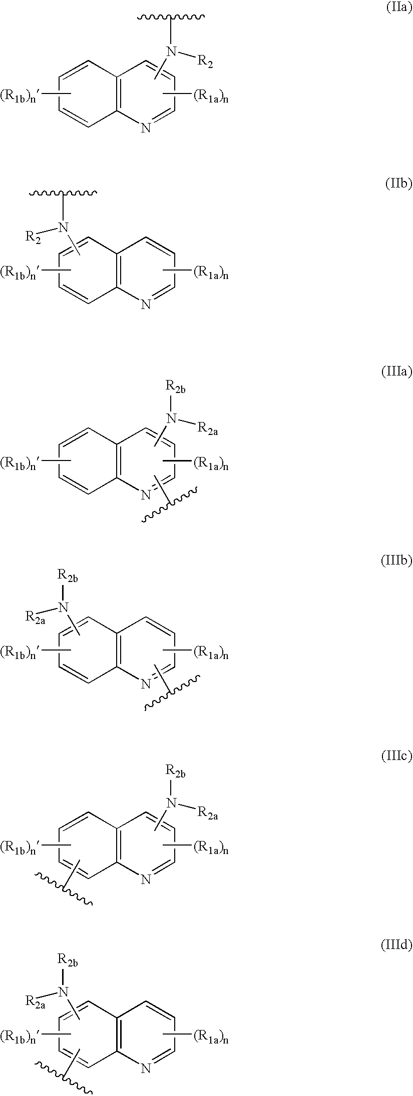

In the above formulae:

the sign [symbol] indicates the anchoring site of the other fragment, e.g. either Y₁, or U, or Y₂, or A;

n and n′ represent, independently of each other, 0, 1, 2 or 3;

R₁ₐ and R_{1b} (generally R₁) represent one or more substituents which are identical or different, occupying any position and representing a substituent which is selected from the group consisting of halogen, hydroxy, trifluoromethyl, trifluoromethoxy, carboxy, amine, sulfate, sulfonate, phosphate, phosphonate, nitro, cyano, aryl, heteroaryl such as those defined herein after or alkyl, alkylamino, alkoxy, alkylthio, alkylsulfonyl, alkylsulfamoyl, alkylsulfonylamino, alkylcarbamoyl, dialkylcarbamoyl, alkylcarbonyloxy, alkoxycarbonyl, alkylcarbonylamino, the said alkyl groups comprising 1, 2 3, 4, 5 or 6 carbon atoms, which are linear, branched or cyclic, saturated or unsaturated, containing if need be one or more amine, amide, thioamide, sulfonyl, sulfonamide, carboxy, thiocarboxy, carbonyl, thiocarbonyl, hydroxyimine, ether or thioether functions and themselves being able to bear 1 to 4 substituents, which are identical or different, and which are selected from halogen, hydroxy, trifluoromethyl, trifluoromethoxy, carboxy, carbonyl, amine, nitro, urea, aryl, or heteroaryl such as defined herein after,

R₂ₐ and R_{2b} (generally R₂) being substituents which are identical or different, being able if need be to form a cyclic structure together or with Y₁, Y₂, U or A and representing a hydrogen atom or a linear, branched or cyclic C1, C2, C3, C4, C5 or C6 alkyl substituent containing if need be one or more amine, amide, thioamide, sulfonyl, urea, thiourea, carbamate, oxime, sulfonamide, carboxy, thiocarboxy, carbonyl, thiocarbonyl, ether or thioether functions and being able to bear 1 to 4 substituents, which are identical or different, and which are selected from halogen, hydroxy, trifluoromethyl, trifluoromethoxy, methoxy, carboxy, amine, nitro, aryl, or heteroaryl such as those defined herein after,

p, p′, p″ are, independently of each other, 0 or 1,

Y₁ and Y₂, which are identical or different, and can be linked by a single or multiple bond to Q, U or A, and represent a saturated or unsaturated, linear, branched or cyclic C1, C2, C3, C4, C5 or C6 alkyl chain, containing if need be one or more amine, amide, thioamide, sulfonyl, sulfonamide, oxo, carboxy, thiocarboxy, carbonyl, thiocarbonyl, urea, thiourea, carbamate, oxime, ether or thioether function, or an aryl or heteroaryl group, such as defined herein after, wherein the alkyl chain can additionally bear 1 to 4 substituents, which are identical or different, and which are selected from the group consisting of halogen, hydroxy, trifluoromethyl, trifluoromethoxy, methoxy, carboxy, carbonyl, amine, nitro, oxime, aryl or heteroaryl such as defined herein after, or selected from among substituents of the type alkyl, alkylamino, dialkylamino, alkoxy, alkylthio, alkylsulfonyl, alkylsulfonamino, alkylsulfamoyl, alkylureido, alkylcarbamoyloxy, alkoxycarbonylamino, alkylcarbamoyl, dialkylcarbamoyl, alkylcarbonylamino, alkylcarbonyl, alkylcarbonyloxy, alkoxycarbonyl, alkoxyimine, the said alkyl groups comprising from 1 to 6 linear, branched or cyclic carbon atoms which can themselves contain one or more amine, amide, thioamide, sulfonyl, sulfonamide, carboxy, thiocarboxy, carbonyl, thiocarbonyl, oxime, ether or thioether function, or an aryl or heteroaryl group such as those defined herein after, wherein the C1, C2, C3, C4, C5 or C6 chain may form a cyclic structure with R₂ including N from the aminoquinoline part and/or the function U and Y₁ and Y₂ may be linked together or to Q, U or A by a single or multiple bond,

U, which can be linked by a single or multiple bond to Q, Y₁, Y₂ or A, is an amine, amide, thioamide, sulfonyl, sulfonamide, carboxy, thiocarboxy, carbonyl, urea, thiourea, carbamate, ether, thioether, thiocarbonyl, sulfonate, oxime, oxyamine, alkylamine (NR), alkoxyimine (C═N—OR) or alkoxyiminocarbonyl (C(O)—C═N—OR) function with R representing a hydrogen atom or a C1, C2, C3, C4, C5 or C6 alkyl substituent, which is linear, branched or cyclic, containing if need be one or more amine, amide, thioamide, sulfonyl, sulfonamide, carboxy, thiocarboxy, carbonyl, thiocarbonyl, ether or thioether functions,

A represents an antibiotic residue.

It is understood that the aryl or heteroaryl groups are preferably an aromatic ring having 5 to 6 members comprising 1 to 4 heteroatoms selected from nitrogen, sulfur and oxygen and that the aryl or heteroaryl groups can themselves bear one or more substituents selected from the group: halogen, hydroxy, trifluoromethyl, trifluoromethoxy, carboxy, amine, nitro or cyano.

By heterocycle the following is preferably understood: a saturated or unsaturated ring having 5 to 6 members comprising 1 to 4 heteroatoms selected from nitrogen, sulfur and oxygen and that can itself bear one or more substituents selected from the group: halogen, hydroxy, oxo, trifluoromethyl, trifluoromethoxy, methoxy, carboxy, amine, nitro or cyano.

In the definition of the compounds having the formula (I) above and in the following, the term ‘halogen’ is understood as meaning a fluorine, chlorine, bromine or iodine atom. In the definition of the compounds having the formula (I) above and in the following, the term ‘antibiotic residue’ is understood as meaning constituted by part A of the hybrid molecules, a chemical entity that has come from an antibiotic, from a modification of an antibiotic or an antibiotic precursor.

Certain compounds are described ‘accidentally’ in the prior art, therefore the invention does not cover:
1) When A is 1-cyclopropyl-6-fluoro-4-oxo-1,4-dihydro-quinoline-3-carboxylic acid or 1-cyclopropyl-6,8-difluoro-4-oxo-1,4-dihydro-quinoline-3-carboxylic acid, and when the link —(Y₁)ₚ—(U)_{p′}—(Y₂)_{p″}— between A and Q is a piperazine, then Q is other than 7-chloro-4-aminoquinoline; i.e. compounds having the formulae:

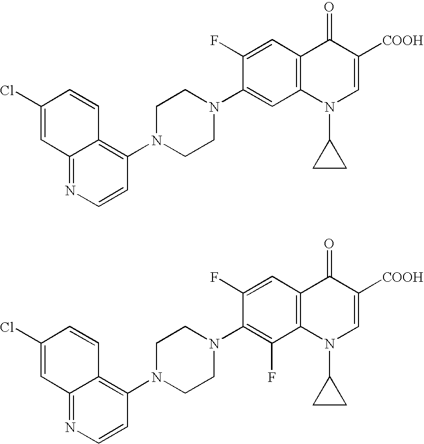

2) When A is (4S,5R,6S)-6-[(R)-1-hydroxyethyl]-4-methyl-7-oxo-1-aza-bicyclo[3.2.0]hept-2-ene-2-carboxylic acid and when the link —(Y₁)ₚ—(U)_{p′}—(Y₂)_{p″}— between A and Q is 3-thioazetidine, then the quinoline part of the substituent Q can not be attached to the link by the 2 position, i.e. for example the compound having the formula:

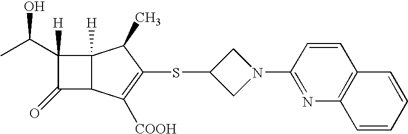

3) When A is a β-lactam having the formula 3-chloro-azetidin-2-one substituted at the 4 position, and when in the link —(Y₁)ₚ—(U)_{p′}—(Y₂)_{p″}—, p, p′, and p″ equal 0, thus forming a direct covalent bond between the nitrogen N1 of A and the extracyclic nitrogen of a 2-aminoquinoline, then Q is other than 2-amino-4-methylquinoline, i.e. for example, compounds having the formula:

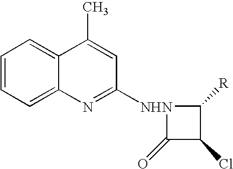

4) When A is a cephalosporin, and when the link —(Y₁)ₚ—(U)_{p′}—(Y₂)_{p″}— is located in the 3 position of the cephalosporin and this link contains an amide function, then Q is other than a 6,7-dihydroxy-4-dimethylaminoquinolin-3-yl, i.e. for example, the compound having the formula:

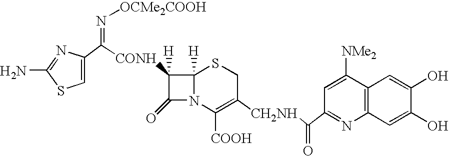

5) When A is a penicillin, and the link —(Y₁)ₚ—(U)_{p′}—(Y₂)_{p″}— contains an amide function, and when Q is a 4-aminoquinoline linked by the 3 position, then the amine function of the 4-aminoquinoline can not be a free amine, i.e. for example, compounds having the formula:

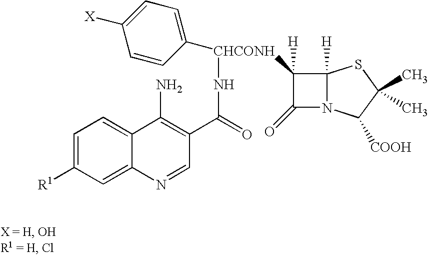

6) When A is a penicillin or a cephalosporin substituted in the 3 position by the link —(Y₁)ₚ—(U)_{p′}—(Y₂)_{p″}—, and the link —(Y₁)ₚ—(U)_{p′}—(Y₂)_{p″}— contains an amide, thioamide, urea or thiourea function then Q is other than a 3-aminoquinoline or a 6-aminoquinoline, i.e. for example, compounds having the following formula: 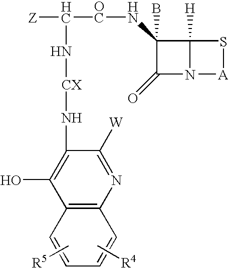
A forms a penicillin or a cephalosporin
i.e. A=—CMe₂CH(COOH)— or —CH₂—CE=C(COOH)—
E=halogen, alkoxy, methyl, CH₂OH, OCOCH₃, OCONH₂,
or E= 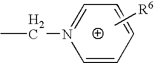
B=H, OMe
R⁶═H, CONH₂
W═H, OH, alkyl
X═O, S
Z=phenyl, alkoxyphenyl, cyclohexen-1-yl, cyclohexa-1,4-dienyl, thienyl
R⁴,R⁵=alkyl, alkoxy, halogen, dialkylamino

7) When A is a penicillin, and the link —(Y₁)ₚ—(U)_{p′}—(Y₂)_{p″}— contains an amide function, then Q is other than 4-hydroxy-6-acetylamino-quinolin-3-yl, i.e. for example, the compound having the formula:

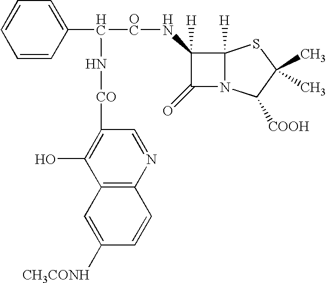

8) When A is (6R,7R)-7-[2-(2-amino-thiazol-4-yl)-2(Z)-methoxyimino-acetylamino]-8-oxo-5-thia-1-aza-bicyclo[4.2.0]oct-2-ene carboxylic acid, and the link —(Y₁)ₚ—(U)_{p′}—(Y₂)_{p″} is a methylene link then Q is other than 5-aminoquinolin-1-yl, i.e. the compound having the formula:

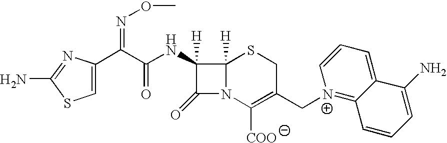

9) When A is (5S)-4-{5-(acetylamino-methyl)-2-oxo-oxazolidin-3-yl}-2-fluoro-phenyl, and the link —(Y₁)ₚ—(U)_{p′}—(Y₂)_{p″} is a 4-piperazin-1-yl link including R₂ and N of the aminoquinoline then Q is other than quinolin-4-yl, i.e. the compound having the formula:

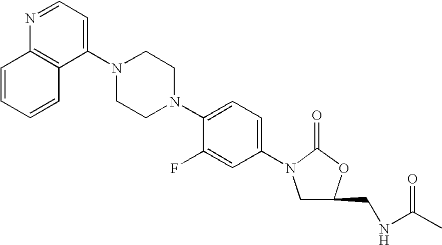

10) When A is a diaminopyrimidine and the link —(Y₁)ₚ—(U)_{p′}—(Y₂)_{p″}— is a methylene link, then Q is other than the following quinolines: 2-morpholino-4-methyl-quinolin-7-yl, 4-methyl-8-aminoquinolin-6-yl, 4-methyl-5-aminoquinolin-6-yl, 2-dimethylamino-4-methylquinolin-6-yl, 2-dimethylamino-4,8-dimethylquinolin-6-yl, 2-morpholino-4,8-dimethylquinolin-6-yl, 2-methyl-4-dimethylamino-8-methoxyquinolin-6-yl, i.e. for example compounds having the formula:

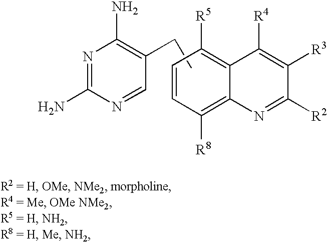

11) When A is 2-methyl-5-nitro-imidazol-1-yl linked directly to the extracyclic nitrogen atom of the aminoquinoline Q (p=p′=p″=0), then Q is other than the following quinolines: 7-chloro-quinolin-4-ylamino, 2-methyl-8-hydroxyquinolin-4-ylamino, 2-methyl-3-n-propyl-8-hydroxy-quinolin-4-ylamino, 2-methyl-5-nitro-8-hydroxyquinolin-4-ylamino, i.e. compounds having the formulae:

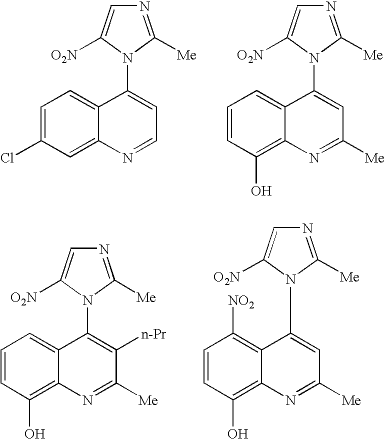

12) When A is 2-methyl-5-nitro-imidazol-1-yl, and the link —(Y₁)ₚ—(U)_{p′}—(Y₂)_{p″}— is 2-ethyl-(1-cyclohexan-4-yl)-amine, then Q is other than a 7-chloro-quinolin-4-ylamino, i.e. the compound having the formula:

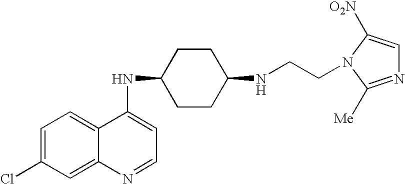

In addition the compounds of formula (I) of the present invention are as follows:

U is not a carbonyl when said antibiotic A is an oxazolidinone of formula (XIVa):

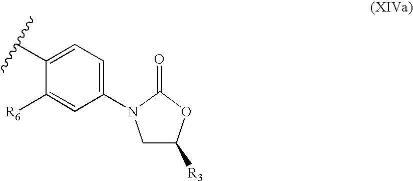

Q being not a 2-aminoquinoline when A is a carbapenem,

Q being not a 3-aminoquinoline when A is a nitroimidazole or an oxazolidinone,

Q being not a 6-aminoquinoline when A is a macrolide or a quinolone,

Q being not an aminoquinolinium when A is a cephalosporin of formula (VIIIb), (VIIId) or (IXb),

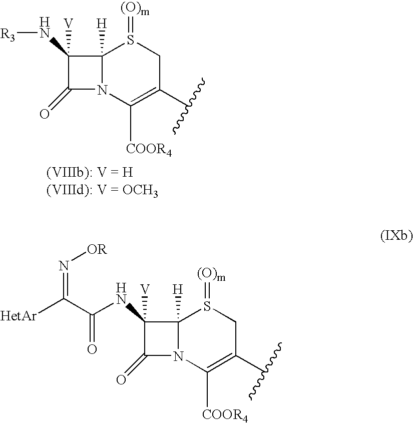

when A is oxazolidinone, the link —(Y₁)ₚ—(U)_{p′}—(Y₂)_{p″} is not a direct link (p=p′=p″=0), not a carbonyl (p=p″=0, p′=1), and not a C1-alkyl group (p′=p″=0, p=1), and

According to the preferred compounds of the invention, the Q part of the hybrid molecules having the formula (I) represents either an aminoquinoline having the formula (IIa) or (IIb), in which the antibiotic part is fixed onto the amine function, or an aminoquinoline having the formula (IIIa), (IIIb) (IIIc) or (IIId) wherein the antibiotic is directly fixed onto the quinoline nucleus.

According to one embodiment, the hybrid molecules containing an aminoquinoline having the formula (IIa) or (IIb) were prepared from haloquinolines and amine derivatives also containing a reactive function for fixing the antibiotic or from the reactive amine function of an aminoquinoline.

According to another embodiment, the quinoline precursors of the hybrid molecules containing an aminoquinoline of type (IIIa), (IIIb) (IIIc) or (IIId) are aminoquinolines which also possess a reactive function such as halogen, haloalkyl, hydroxy, amine, hydroxyalkyl, sulfonamide or carboxy.

According to the invention which covers compounds having the formula (I), A represents an antibiotic residue. This residue can advantageously be selected from the large families of antibiotics which are known to the person skilled in the art, such as, for example, β-lactams, quinolones, oxazolidinones, derivatives of fosfomycin, nitroimidazoles, nitrofurans, sulfamides, streptogramins, synergistins, lincosamides, tetracyclines, derivatives of chloramphenicol, derivatives of fusidic acid, diaminopyrimidines, aminosides, macrolides, polypeptides, glycopeptides, rifamycins or lipodepsipeptides. In the following embodiments of compounds having the formula (I) covered by the invention, some examples of formulae of the antibiotic A are given as non-limiting examples.

Aminoquinoline-β-Lactam Hybrid Molecules

According to an advantageous embodiment of the compounds having the formula (I) according to the invention, A can be selected from the family of β-lactams which contains, amongst others: penams (or penicillins) having the formula (IV), oxapenams having the formula (V), penems having the formula (VI), carbapenems having the formula (VII), cephems (or cephalosporins) having the formula (VIIIa), (VIIIb), (IXa) or (IXb), cephamycins having the formula (VIIIc) or (VIIId), oxacephems having the formula (Xa) or (Xb), carbacephems having the formula (XIa) or (XIb) and monobactams having the formula (XII), as follows: 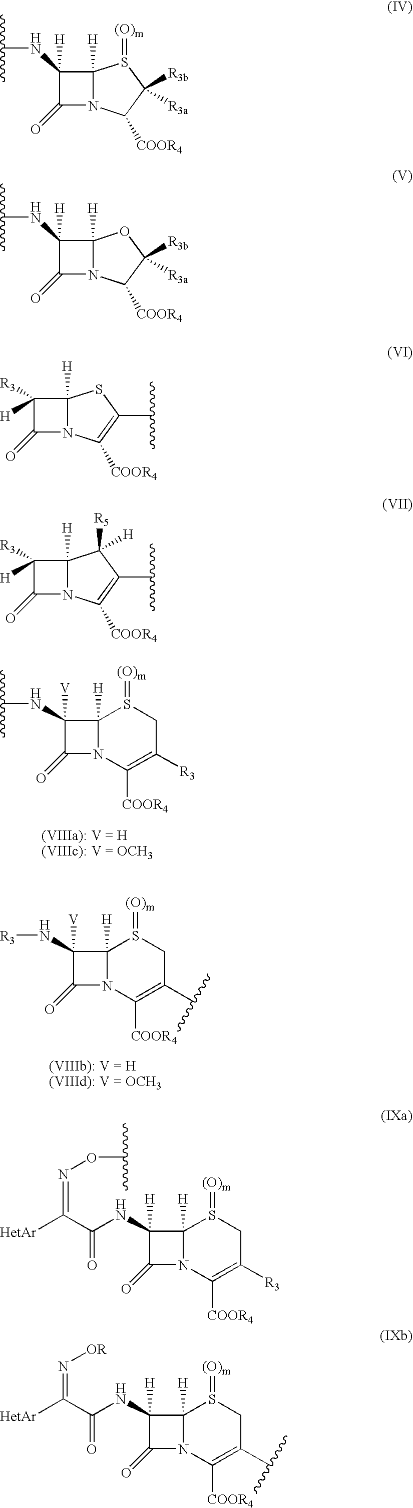 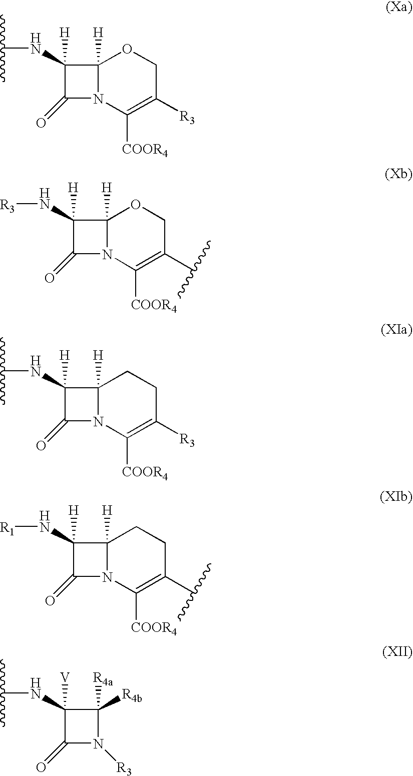
in which

R₁ is as defined above,

R₃ₐ and R_{3b} (generally R₃) represent substituents which are identical or different and which are selected from the group consisting of halogen, hydroxy, trifluoromethyl, trifluoromethoxy, carboxy, aldehyde, amine, sulfate, sulfonate, phosphate, phosphonate, nitro, cyano, aryl or heteroaryl such as previously described, or alkyl, alkylamino, dilkylamino, alkoxy, alkylthio, alkylsulfonyl, alkylsulfonylamino, alkylsulfamoyl, alkylureido, alkylcarbamoyloxy, alkyloxycarbonylamino, alkylcarbamoyl, dialkylcarbamoyl, alkylcarbonylamino, alkylcarbonyl, alkylcarbonyloxy, alkoxycarbonyl, alkoxyimine, the said alkyl groups comprising 1, 2, 3, 4, 5 or 6 carbon atoms, which are saturated or unsaturated, linear, branched or cyclic, containing if need be one or more amine, amide, thioamide, sulfonyl, sulfonamide, oxo, carboxy, thiocarboxy, carbonyl, thiocarbonyl, urea, thiourea, carbamate, oxime, ether or thioether functions and themselves being able to bear 1 to 4 substituents, which are identical or different, and which are selected from halogen, hydroxy, trifluoromethyl, methyl, trifluoromethoxy, methoxy, carboxy, carbonyl, amine, nitro, urea, aryl, or heteroaryl or heterocycle such as previously described,

R₄ₐ and R_{4b} (generally R₄) which are identical or different, being able if need be to form, together, a cyclic structure or a multiple bond, represent a hydrogen atom or a saturated or unsaturated, linear, branched or cyclic C1, C2, C3, C4, C5 or C6 alkyl substituent, containing if need be one or more amine, amide, thioamide, sulfonyl, sulfonamide, carboxy, thiocarboxy, carbonyl, thiocarbonyl, oxime, urea, carbamate, ether or thioether functions and being able to bear 1 to 4 substituents, which are identical or different, and which are selected from halogen, hydroxy, trifluoromethyl, trifluoromethoxy, methoxy, carboxy, amine, nitro, aryl, or heteroaryl such as previously described,

R₅ is a hydrogen atom or a saturated or unsaturated, linear, branched or cyclic C1, C2, C3, C4, C5 or C6 alkyl substituent,

V represents a methoxy group or a hydrogen atom.

“HetAr” represents a heteroaryl such as defined before.

The β-lactams having the formulae (IV), (V), (VIb), (VIIIa), (VIIIc), (Xa), (XIa) and (XII) can be, for example, coupled to a quinoline moiety by making use of their amine function.

The coupling reaction with the carbapenems having the formula (VIIb) can be carried out, for example, from a carbonyl or hydroxy function.

A reactive function of hydroxy, halogen, or alkene type can be advantageously used for fixing cephalosporins, cephamycins, oxacephems and carbacephems having the respective formulae (VIIIb), (VIIId), (IXa), (IXb), (Xb) and (XIb).

Aminoquinoline-Quinolone Hybrid Molecules

In another family of compounds according to the invention, A represents a quinolone moiety such as the one described by the following formula (XIIIa) or (XIIIb), 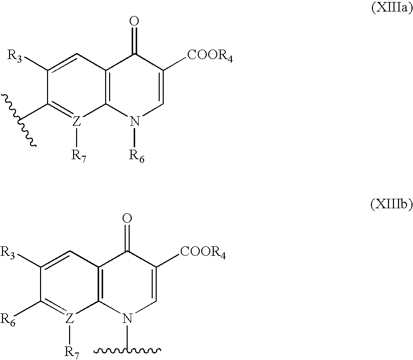
in which

R₃ and R₄ are as defined above,

R₆ and R₇ are substituents which are identical or different, being able if need be to form, together, a cyclic structure and representing a hydrogen atom or a substituent which is selected from the group consisting of halogen, hydroxy, heterocycle, aryl or heteroaryl such as described previously, or an alkyl, alkoxy or alkylamine substituent, the said alkyl groups comprising 1, 2, 3, 4, 5 or 6 carbon atoms, which are saturated or unsaturated, linear, branched or cyclic, containing if need be one or more amine, amide, thioamide, sulfonyl, sulfonamide, carboxy, thiocarboxy, carbonyl, thiocarbonyl, ether or thioether functions and being able to bear 1 to 4 substituents, which are identical or different, and which are selected from halogen, hydroxy, trifluoromethyl, trifluoromethoxy, carboxy, carbonyl, amine, nitro, aryl, or heteroaryl such as described previously,

Z is a nitrogen or carbon atom.

A reactive function of amine or halogen type of the quinolones known to the person skilled in the art can advantageously be used for the coupling reaction with a quinoline type derivative.

Aminoquinoline Oxazolidinone Hybrid Molecules

In another embodiment of the compounds according to the invention, A represents an oxazolidinone residue such as those described by the following formula (XIVa), (XIVb) or (XIVc), 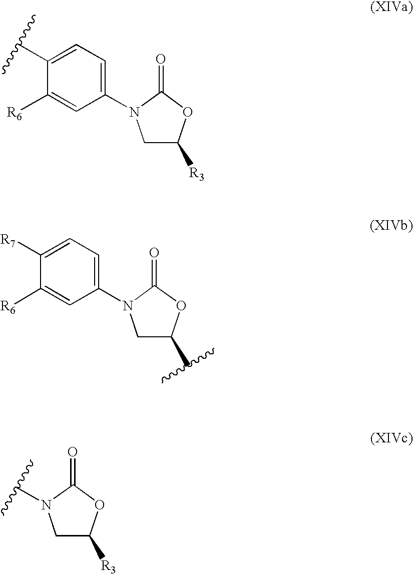
in which R₃, R₆ and R₇ are as defined above.

Such hybrid molecules can advantageously be prepared either by making use of an amine, hydroxy or halogen type reactive function of an oxazolidinone or by synthesis of the oxazolidinone ring from an aminoquinoline comprising a protected amine function and from (R)-glycidyl butyrate according to methods known to the person skilled in the art.

Aminoquinoline-Fosfomycin Hybrid Molecules

In another embodiment of the compounds according to the invention, A represents a derivative of fosfomycin such as the one described by the formula (XV) as follows, 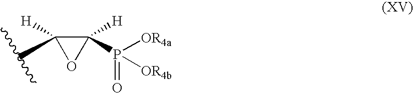
in which R₄ₐ and R_{4b}, which are identical or different, being able if need be to form, together, a cyclic structure are as defined above.

The synthesis of hybrid molecules derived from fosfomycin can be, for example, carried out by epoxidation of an alkene type precursor before or after fixing onto the aminoquinoline.

Aminoquinoline-Nitroimidazole or Aminoquinoline-Nitrofuran Hybrid Molecules

In another family of compounds according to the invention, A represents a nitroimidazole residue such as those described by the formulae (XVIa) or (XVIb) or a nitrofuran residue such as the one described by the formula (XVII), as follows, 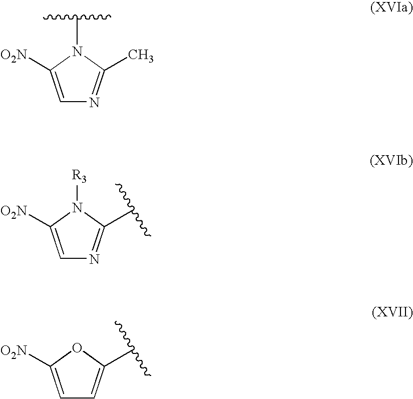
in which R₃ is as defined above.

A reactive function of hydroxy, epoxy, amine or halogen type can be, for example, used in the coupling reaction of the nitroimidazole or nitrofuran derivatives having the formula (XVI) or (XVII) with a quinoline moiety.

Aminoquinoline-Sulfamide Hybrid Molecules

In another embodiment of the compounds according to the invention, A represents a sulfamide residue such as the one described by the following formula (XVIII),

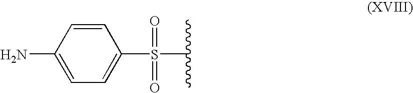

This residue can, for example, be fixed onto a quinoline from a sulfonamide or sulfonic acid type reactive function.

Aminoquinoline-Streptogramin or -Synergistin Hybrid Molecules

In another family of compounds according to the invention, A represents a streptogramin or a synergistin residue such as those described by the formulae (XIXa), (XIXb), (XIXc), (XXa) or (XXb), as follows, 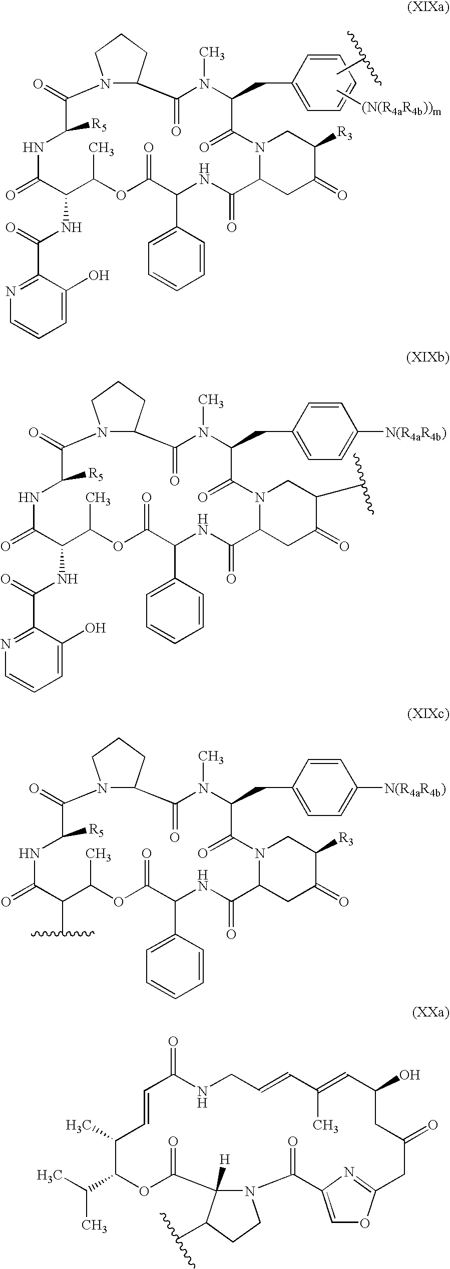 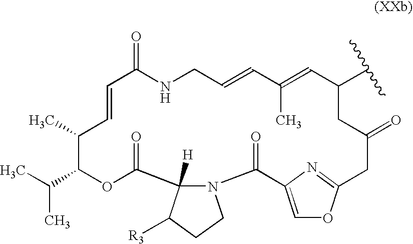
in which R₃, R₄ₐ, R_{4b}, R₅ and m are as defined above.

The synthesis of hybrid molecules incorporating a streptogramin or synergistin derivative can be carried out, for example, from precursors of pristinamycin or virginiamycin type.

Aminoquinoline-Lincosamide Hybrid Molecules

In another embodiment of the compounds according to the invention, A represents a lincosamide residue such as the one described by the formula (XXI) as follows,

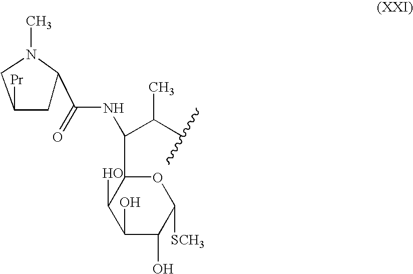

Lincosamides possess a hydroxy function or a halogen atom which can be used, for example, for grafting them onto an aminoquinoline.

Aminoquinoline-Tetracycline Hybrid Molecules

In another embodiment of the compounds according to the invention, A represents a tetracycline residue such as those described by the formulae (XXIIa), (XXIIb) and (XXIIc) as follows, 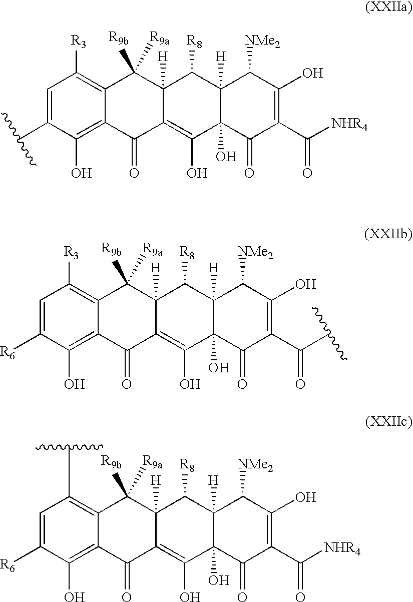
in which

R₃, R₄ and R₆ are as defined above,

R₈ and R₉ₐ, R_{9b}, which are identical or different, represent a hydrogen atom or a substituent which is selected from the group: hydroxy or methyl,

The coupling reaction with the tetracyclines having the formula (XXIIa), (XXIIb) or (XXIIc) can be carried out, for example, from their amide function or by modification of an aromatic CH moiety.

Aminoquinoline-Chloramphenicol Hybrid Molecules

In another family of compounds according to the invention, A represents a derivative of chloramphenicol such as those described by the formulae (XXIIIa) or (XXIIIb), as follows, 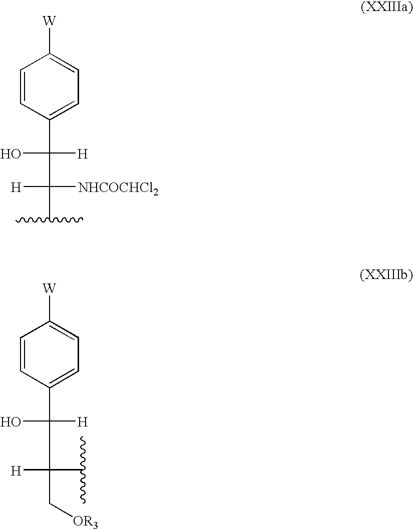
in which

R₃ is as defined above,

W represents an NO₂ or SO₂R₅ substituent, R₅ being as defined above.

For example, a reactive function of hydroxy or halogen type can be used for fixing the chloramphenicol derivatives according to the modes (XXIIIa) and (XXIIIb).

Aminoquinoline-Fusidic Acid Hybrid Molecules

In another embodiment of the compounds according to the invention, A represents a derivative of fusidic acid such as those described by the following formulae (XXIVa), (XXIVb) or (XXIVc),

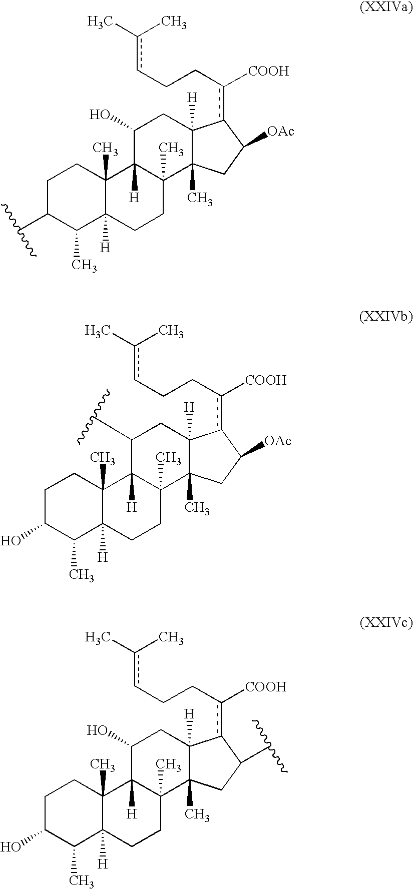

The fusidic acid derivatives having the formulae (XXIVa), (XXIVb) and (XXIVc) as defined above can be grafted onto an aminoquinoline, for example from an hydroxy function.

Aminoquinoline-Diaminopyrimidine Hybrid Molecules

In another family of compounds according to the invention, A represents a diaminopyrimidine residue such as those described by the formula (XXV) as follows,

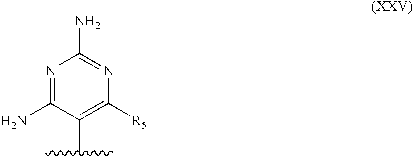

in which R₅ is as defined above.

Hybrid molecules incorporating a diaminopyrimidine residue can be prepared in particular by making use of a hydroxy or halogen type reactive function of a known diaminopyrimidine or by cyclization with guanidine of a precursor of acrylonitrile type.

Aminoquinoline-Aminoside Hybrid Molecules

In another family of compounds according to the invention, A represents an aminoside residue which is formed by the union of a genin moiety from the group of aminocyclitols with one or more oses at least one of which is an aminosugar, which are linked together via glycosidic bridges. Many aminosides with diverse chemical structures exist that can be coupled to an aminoquinoline by making use of one of their amino or hydroxy type reactive functions.

Aminoquinoline-Macrolide Hybrid Molecules

In another embodiment of the compounds according to the invention, A represents a macrolide residue:

having 14 atoms such as those described the formulae (XXVIa), (XXVIb), (XXVIc) and (XXVId),

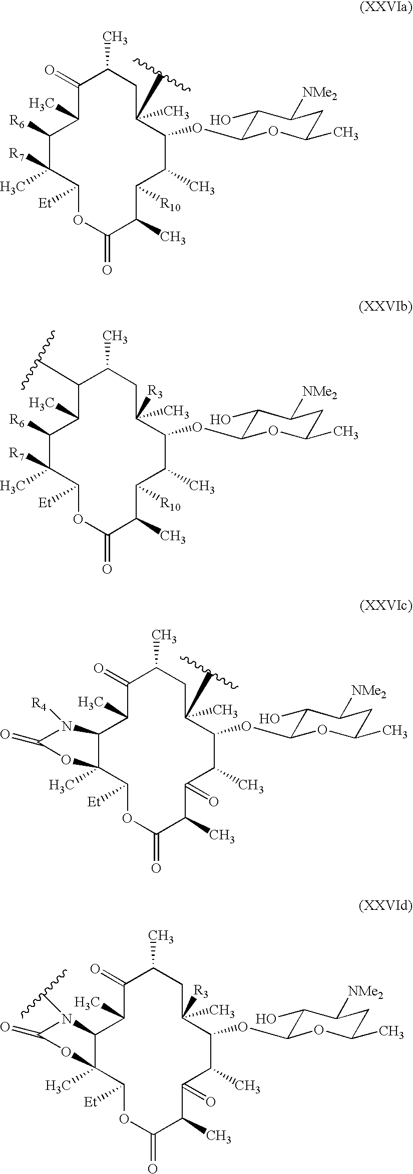

having 15 atoms such as those described the following formulae (XXVIIa), (XXVIIb), (XXVIIc) and (XXVIId), 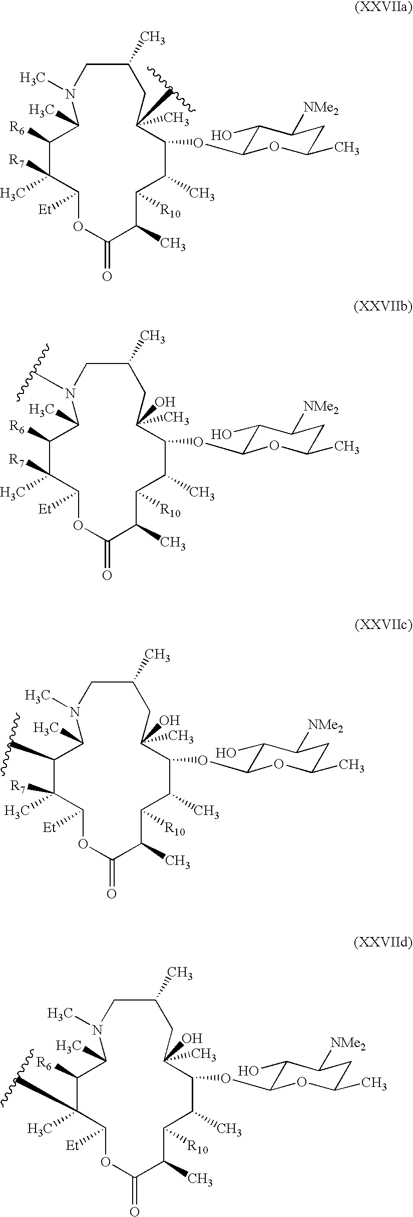 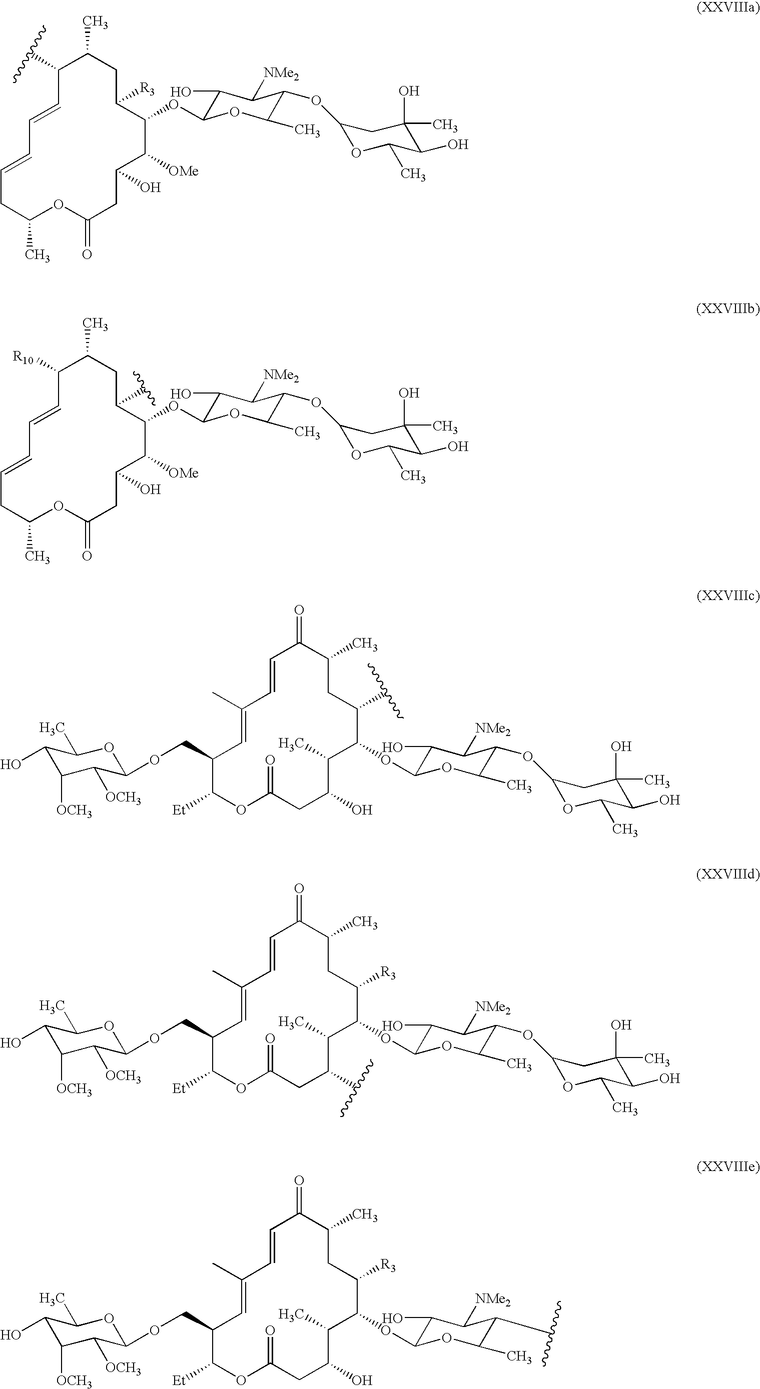
in which

R₃, R₄, R₆ and R₇ are as defined above,

R₁₀ is an oxygen atom linked via a double bond of carbonyl type to the macrocycle or a hydroxy group or an osidic derivative linked via a glycosidic bridge to the macrocycle and being able to bear 1 to 6 substituents, which are identical or different, and which are selected from hydroxy, alkyl, alkylamino, dialkylamino or alkoxy, the said alkyl groups comprising 1 to 6 carbon atoms which are linear, branched or cyclic, saturated or unsaturated, and may bear a carboxy substituent.

Advantageously, the reactive functions of the macrolides of hydroxy, amino or carbonyl type can be used for the coupling reaction with the aminoquinolines.

Aminoquinoline-Polypeptide Hybrid Molecules

In another family of compounds according to the invention, A represents a polypeptide residue such as derivatives of polymyxins or of bacitracin linking various peptidic structures. These residues can be grafted onto an aminoquinoline notably via one of their free amino functions.

Aminoquinoline-Glycopeptide Hybrid Molecules

In another embodiment of the compounds according to the invention, A represents a glycopeptide residue such as:

the derivatives of vancomycin described by the formulae (XXIXa), (XXIXb), (XXIXc), (XXIXd), (XXIXe) and (XXIXf) as follows,

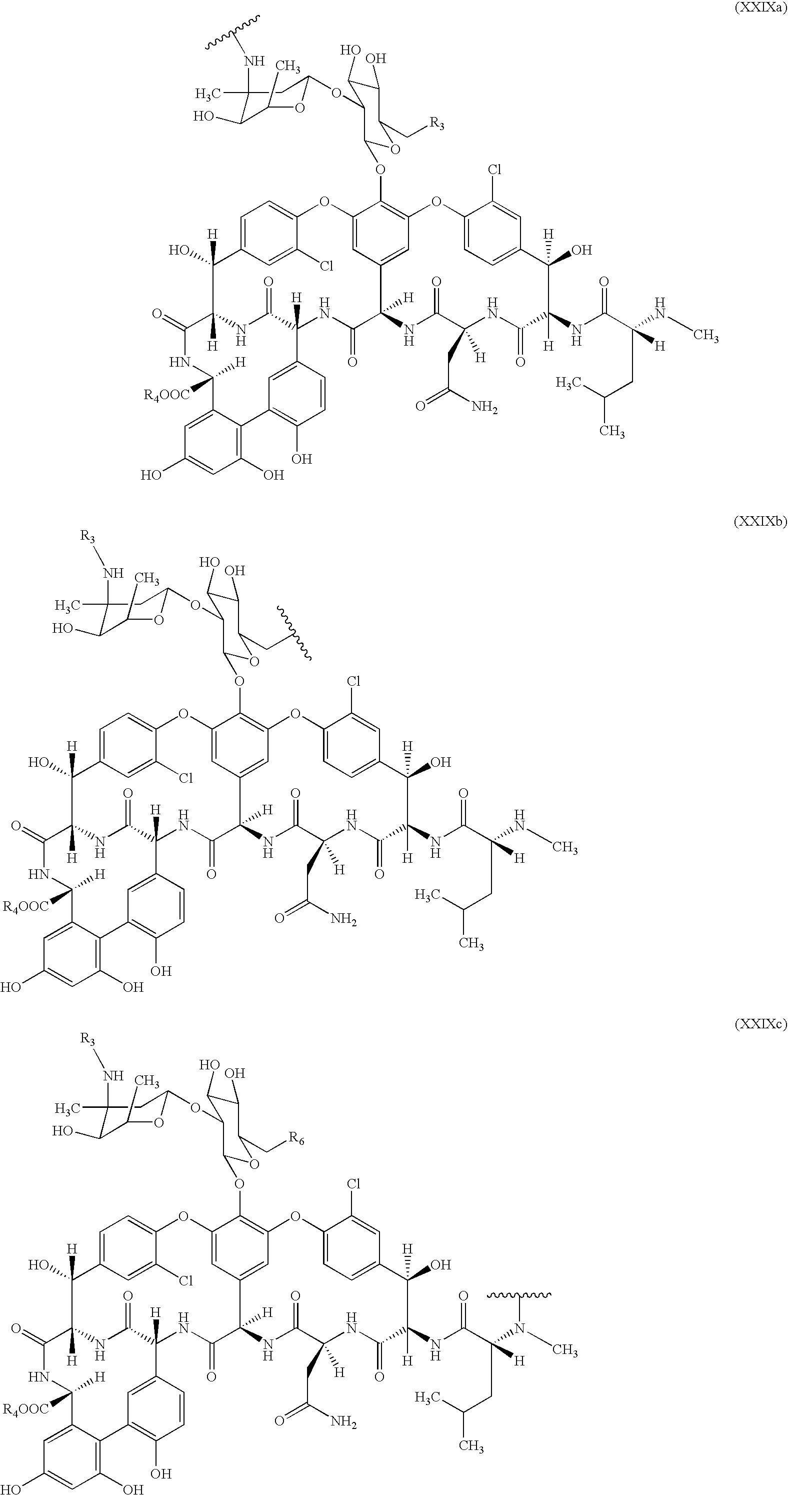

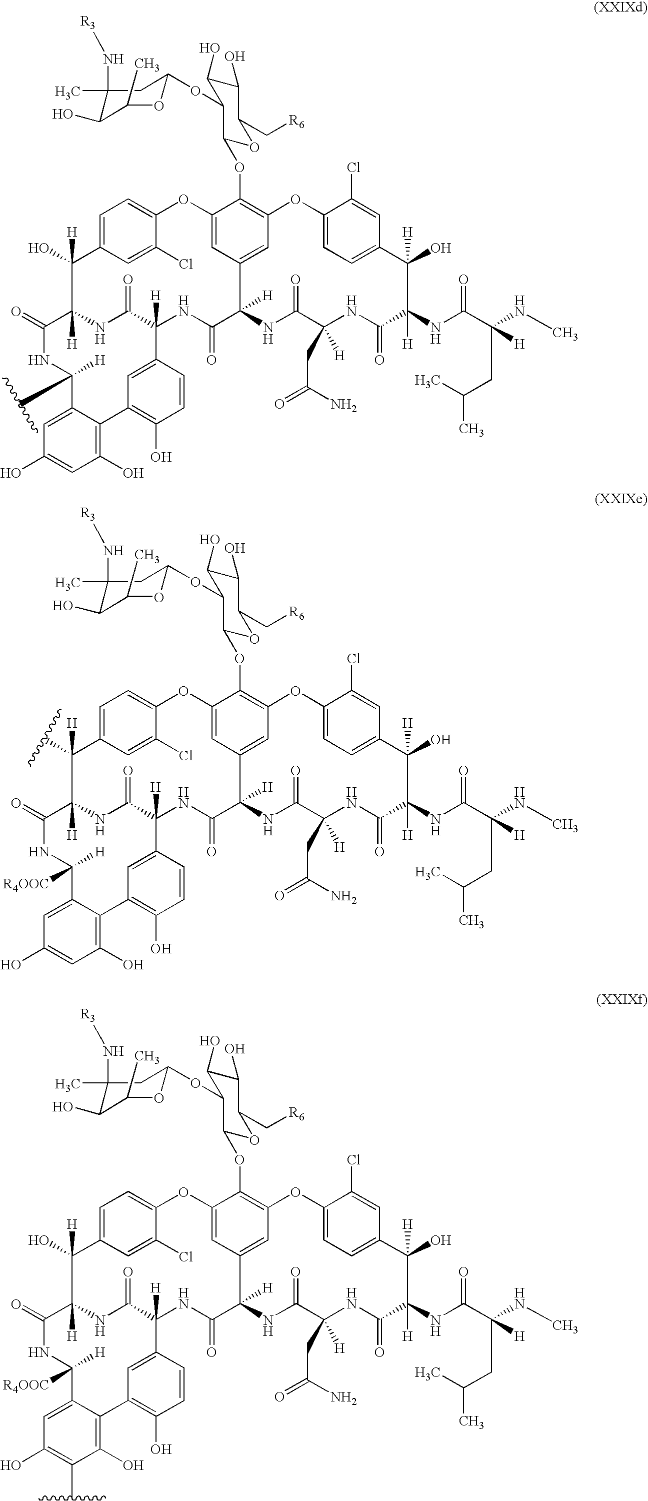

or the derivatives of teicoplanin described by the formula (XXXa) or (XXXb), as follows, 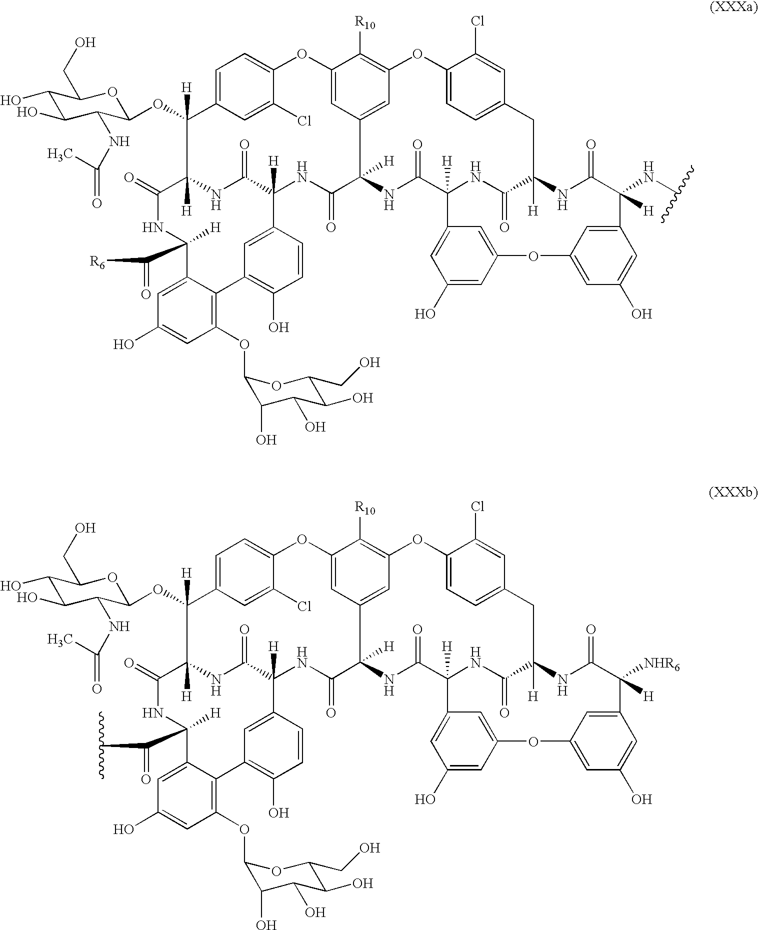
in which R₃, R₄ and R₆ are as defined above.

The derivatives of vancomycin and of teicoplanin can be, for example, fixed onto an aminoquinoline moiety from one of their amino, carboxy, amide, hydroxy type reactive functions, or by modification of a CH aromatic moiety.

Aminoquinoline-Rifamycin Hybrid Molecules

In another family of compounds according to the invention, A represents a rifamycin residue such as those described by the formulae (XXXIa) and (XXXIb), as follows, 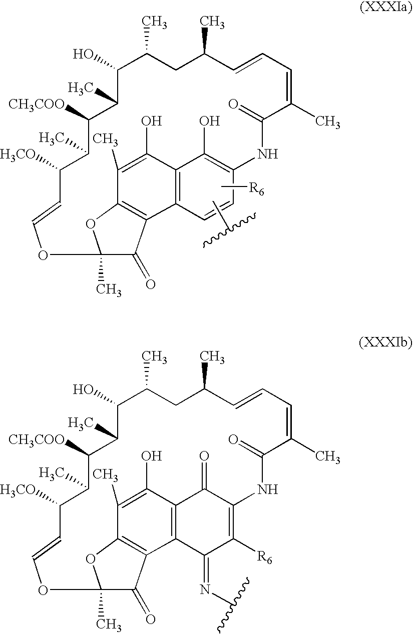
in which Rᵣ occupy any position and may form a cyclic structure with Y₁, Y₂ or U which are as defined above.

The preparation of an aminoquinoline-rifamycin hybrid molecule can be carried out, for example, from one of rifamycin's reactive functions of amino, halogen, hydroxy or aldehyde type.

Aminoquinoline-Lipodepsipeptide Hybrid Molecules

In another embodiment of the compounds according to the invention, A represents a lipodepsipeptide residue such as the derivatives of daptomycin described by the following formula (XXXII),

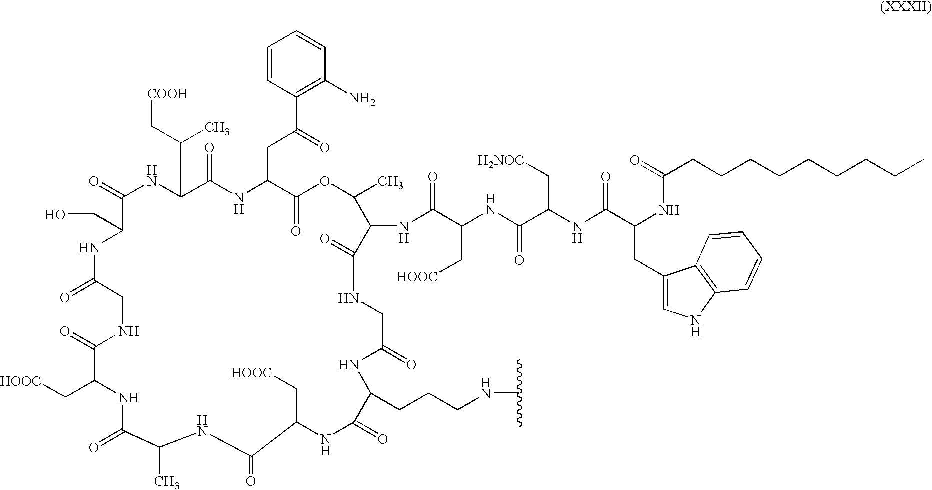

The lipodepsipeptides can be grafted onto a quinoline, for example from one of their amino, hydroxy or carboxy type reactive functions.

The formulae (IV) to (XXXII) give examples of sites for grafting an aminoquinoline onto a residue A, but other anchoring sites have been envisaged on the compounds A. It is understood that the invention covers the hybrid molecules aminoquinoline—A which are linked via any anchoring site.

The invention also covers any hybrid molecule having the formula (I) which covalently links an aminoquinoline to an antibiotic residue A other than those described by the formulae (IV) to (XXXII).

When the link —(Y₁)ₚ—(U)_{p′}—(Y₂)_{p″} bears one or more asymmetric centers the invention covers mixtures of stereoisomers in all proportions as well as pure stereoisomers.

The compounds of the invention can also exist in the forms of salts of the addition with an acid, salts of the addition with a base or zwitterions as well as prodrugs or salts of prodrugs. The invention also covers these different forms and their mixtures.

Advantageously, the compounds having the formula (I) are those having the Q substituent representing a substituent having the formula (IIa) or (IIIa) defined previously.

Advantageously, the compounds having the formula (I) are those having the Q substituent representing a substituent having the formula (IIb) defined previously.

Advantageously, the compounds having the formula (I) are those having the A substituent representing a substituent having the formula (IV) defined previously.

Advantageously, the compounds having the formula (I) are those having the A substituent representing a substituent having the formula (VIIIa), (IXa) or (IXb) defined previously.

Advantageously, the compounds having the formula (I) are those having the A substituent representing a substituent having the formula (XIIIa) or (XIIIb) defined previously.

Advantageously, the compounds having the formula (I) are those having the A substituent representing a substituent having the formula (XIVa) or (XIVb) defined previously.

Advantageously, the compounds having the formula (I) are those having the A substituent representing a substituent having the formula (XVIa) defined previously.

Advantageously, the compounds having the formula (I) are those having the A substituent representing a substituent having the formula (XIXb) defined previously.

Advantageously, the compounds having the formula (I) are those having the A substituent representing a substituent having the formula (XXV) defined previously.

Advantageously, the compounds having the formula (I) are those having the A substituent representing a substituent having the formula (XXVIb), (XXVIc) or (XXVId) defined previously.

Advantageously, the compounds having the formula (I) are those having the A substituent representing a substituent having the formula (XXIXa) defined previously.

According to another preferred mode of preparation the aminoquinolines are of the 4-aminoquinoline, 2-aminoquinoline or 8-aminoquinoline type. Their synthesis can be carried out from commercially available synthons, which gives these compounds a rather interesting advantage in addition to their activity.

In the hybrid molecules having the formula (I) that conform to the invention, aminoquinolines having the formula (IIa) and (IIIa) in which the amino substituent is in the 4 position with respect to the endocyclic nitrogen atom (this is thus 4-aminoquinolines) or in the 2 position with respect to the endocyclic nitrogen atom (this is thus 2-aminoquinolines) are more especially preferred, or even aminoquinolines having the formula (IIb) in which the amino substituent is in the 8 position (8-aminoquinolines).

These 4-aminoquinolines, 2-aminoquinolines and 8-aminoquinolines have the following formulae (XXXIIIa), (XXXIIIb), (XXXIIIc), (XXXIIId) and (XXXIIIe), 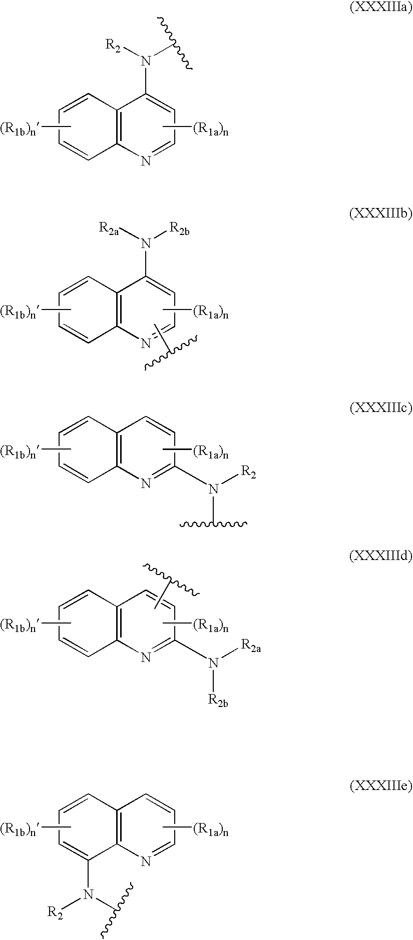
in which R₁ₐ, R_{1b}, R₂, n and n′ are as defined above. According to a preferred disposition of the invention, R₁ advantageously represents only one substituent, this substituent being a halogen atom or a hydroxy, methyl, methoxy, trifluoromethyl, trifluoromethoxy, carboxy, cyano, amine or nitro group occupying any position. According to another preferred disposition, in the formulae (XXXIIIa), (XXXIIIb) and (XXXIIIe), R₂ advantageously represents a hydrogen atom or a methyl group or forms a cyclic structure with Y₁ and eventually with U including N of the aminoquinoline (preferably a piperidine or a piperazine). In the formulae (XXXIIIb) and (XXXIIId) R₂ₐ and R_{2b} advantageously represent identical or different substituents that can form a cyclic structure together, these substituents preferably being a hydrogen atom or a methyl, cyclopropyl or 2-(diethylamino)ethyl group, or a heterocycle when R₂, and R_{2b} form a cyclic structure together (preferably aziridin-1-yl, morpholin-4-yl, piperidin-1-yl, piperazin-1-yl, or 4-methylpiperazin-1-yl).

Advantageously, the compounds having the formula (I) are those having the —(Y₁)ₚ—(U)_{p′}—(Y₂)_{p″}— substituents representing a group in which p=p′=p″=0, wherein the link between Q and A is direct.

Advantageously, the compounds having the formula (I) are those having the —(Y₁)ₚ—(U)_{p′}—(Y₂)_{p″}—substituents representing a group in which p′=1 and p=p″=0, U being as defined previously and advantageously representing a carbonyl group.

Advantageously, the compounds having the formula (I) are those having the —(Y₁)ₚ—(U)_{p′}—(Y₂)_{p″} substituents representing a group in which p′=1 and p=p″=0, U being as defined previously and advantageously representing a thioether group.

Advantageously, the compounds having the formula (I) are those having the —(Y₁)ₚ—(U)_{p′}—(Y₂)_{p″} substituents representing a group in which p′=1 and p=p″=0, U being as defined previously and advantageously representing an alkoxyaminocarbonyl group (preferably hydroxyiminocarbonyl or methoxyiminocarbonyl).

Advantageously, the compounds having the formula (I) are those having the —(Y₁)ₚ—(U)_{p′}—(Y₂)_{p″} substituents representing a group in which p′=1 and p=p″=0, Y₁ being as defined previously and advantageously representing a linear or branched C1, C2, C3, C4, C5, or C6 alkyl chain and being able to form a cyclic structure with A or R₂ including the N of the aminoquinoline.

Advantageously, the compounds having the formula (I) are those having the —(Y₁)ₚ—(U)_{p′}—(Y₂)_{p″}—substituents representing a group in which p=1 and p′=p″=0, Y₁ being as defined previously and advantageously representing a C1, C2, C3, C4, C5, or C6 alkyl chain substituted by fluorine atoms.

Advantageously, the compounds having the formula (I) are those having the —(Y₁)ₚ—(U)_{p′}—(Y₂)_{p″}—substituents representing a group in which p=1 and p′=p″=0, Y₁ being as defined previously and advantageously representing a C1, C2, C3, C4, C5, or C6 alkyl chain containing an amine or ether function.

Advantageously, the compounds having the formula (I) are those having the —(Y₁)ₚ—(U)_{p′}—(Y₂)_{p″}—substituents representing a group in which p=p′=1 and p″=0, U being as defined previously and advantageously representing a carbonyl group and Y₁ being as defined previously and advantageously representing a linear or branched C1, C2, C3, C4, C5, or C6 alkyl chain and being able to form a cyclic structure with R₂ including the N of the aminoquinoline.

Advantageously, the compounds having the formula (I) are those having the —(Y₁)ₚ—(U)_{p′}—(Y₂)_{p″}—substituents representing a group in which p=p′=1 and p″=0, U being as defined previously and advantageously representing an amine group and Y₁ being as defined previously and advantageously representing a linear or branched C1, C2, C3, C4, C5, or C6 alkyl chain and being able to contain an amine, ether, amide or urea function and being able to form a cyclic structure with U and/or R₂ including the N of the aminoquinoline.

Advantageously, the compounds having the formula (I) are those having the —(Y₁)ₚ—(U)_{p′}—(Y₂)_{p″} substituents representing a group in which p=p′=1 and p″=0, U being as defined previously and advantageously representing a thioether function and Y₁ being as defined previously and advantageously representing a linear or branched C1, C2, C3, C4, C5, or C6 alkyl chain and being able to be substituted by fluorine atoms.

Advantageously, the compounds having the formula (I) are those having the —(Y₁)ₚ—(U)_{p′}—(Y₂)_{p″}—substituents representing a group in which p=p′=1 and p″=0, U being as defined previously and advantageously representing an ether function and Y₁ being as defined previously and advantageously representing a C1, C2, C3, C4, C5, or C6 alkyl chain.

Advantageously, the compounds having the formula (I) are those having the —(Y₁)ₚ—(U)_{p′}—(Y₂)_{p″}—substituents representing a group in which p=p′=1 and p″=0, U being as defined previously and advantageously representing a carbamate function and Y₁ being as defined previously and advantageously representing a linear or branched, saturated or unsaturated C1, C2, C3, C4, C5, or C6 alkyl chain and being able to contain an ether and/or aryl group.

Advantageously, the compounds having the formula (I) are those having the —(Y₁)ₚ—(U)_{p′}—(Y₂)_{p″} substituents representing a group in which p=p′=1 and p″=0, U being as defined previously and advantageously representing an amide function and Y₂ being as defined previously and advantageously representing a linear or branched C1, C2, C3, C4, C5, or C6 alkyl chain being able to contain an amine or thioether function.

Advantageously, the compounds having the formula (I) are those having the —(Y₁)ₚ—(U)_{p′}—(Y₂)_{p″} substituents representing a group in which p=p′=p″=1, U being as defined previously and advantageously representing an amine function and Y₁ and Y₂ being as defined previously and advantageously representing a linear or branched C1, C2, C3, C4, C5, or C6 alkyl chain, being able to be substituted by fluorine atoms or a hydroxy group and being able to form a cyclic structure with U and/or R₂ including the N of the aminoquinoline.

Advantageously, the compounds having the formula (I) are those having the —(Y₁)ₚ—(U)_{p′}—(Y₂)_{p″}—substituents representing a group in which p=p′=p″=1, U being as defined previously and advantageously representing an ether function and Y₁ and Y₂ being as defined previously and advantageously representing a linear or branched C1, C2, C3, C4, C5, or C6 alkyl chain being able to contain an aryl group.

Advantageously, the compounds having the formula (I) are those having the —(Y₁)ₚ—(U)_{p′}—(Y₂)_{p″} substituents representing a group in which p=p′=p″=1, U being as defined previously and advantageously representing a thioether function and Y₁ and Y₂ being as defined previously and advantageously representing a linear or branched C1, C2, C3, C4, C5, or C6 alkyl chain.

Advantageously, the compounds having the formula (I) are those having the —(Y₁)ₚ—(U)_{p′}—(Y₂)_{p″}—substituents representing a group in which p=p′=p″=1, U being as defined previously and advantageously representing an amide function and Y₁ and Y₂ being as defined previously and advantageously representing a linear or branched C1, C2, C3, C4, C5, or C6 alkyl chain being able to be substituted by fluorine atoms.

Advantageously, the compounds having the formula (I) are those having the —(Y₁)ₚ—(U)_{p′}—(Y₂)_{p″}—substituents representing a group in which p=p′=p″=1, U being as defined previously and advantageously representing a carbamate function and Y₁ and Y₂ being as defined previously and advantageously representing a linear or branched C1, C2, C3, C4, C5, or C6 alkyl chain being able to be substituted by fluorine atoms.

Advantageously, the compounds having the formula (I) are those having the —(Y₁)ₚ—(U)_{p′}—(Y₂)_{p″} substituents representing a group in which p=p′=p″=1, U being as defined previously and advantageously representing an urea function and Y₁ and Y₂ being as defined previously and advantageously representing a linear or branched C1, C2, C3, C4, C5, or C6 alkyl chain being able to be substituted by fluorine atoms.

Advantageously, according to the invention, A represents a cephalosporin, a penicillin, a quinolone, a nitroimidazole, a streptogramin, a diaminopyrimidine, a macrolide, a glycopeptide or an oxazolidinone.

The preferred aminoquinolines Q are covalently linked to an antibiotic residue A to form hybrid molecules, notably the hybrid molecules that follow.

Aminoquinoline-β-Lactam Hybrid Molecules

Notably, an aim of the invention is hybrid molecules which correspond to the coupling product comprising a 4-aminoquinoline having the formula (XXXIIIa) or an 8-aminoquinoline having the formula (XXXIIIe) and a residue A from the family of penicillins having the formula (IV). Such molecules are of the structure (XXXIVa), (XXXIVb) or (XXXIVc) in which R₁ₐ, R_{1b}, R₂, R₃ at R_{3b}, R₄, Y₁, Y₂, U, p, p′, p″, m, n and n′ are as defined above.

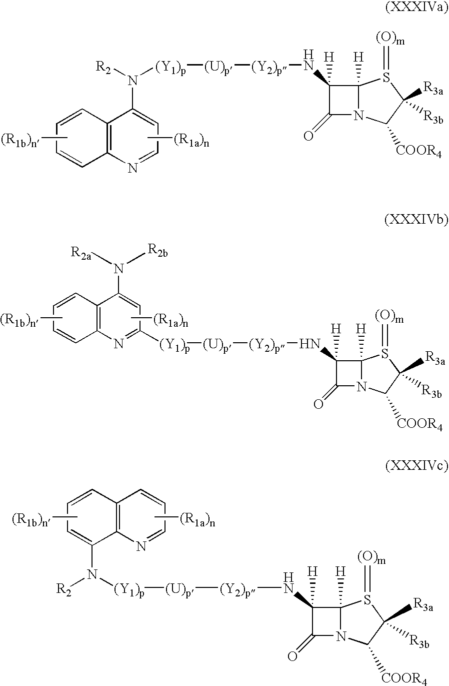

Other preferred hybrid molecules correspond to the coupling product comprising a 4 aminoquinoline having the formula (XXXIIIa) or (XXXIIIb), or an 8-aminoquinoline having the formula (XXXIIIe) and a residue A from the cephalosporin family having the formula (VIIIa). These important hybrid molecules are of the structure (XXXVa) or (XXXVb) or (XXXVc) in which R₁, R₂, R₃, R₄, Y₁, Y₂, U, p, p′, p″, m, n and n′ are as defined above.

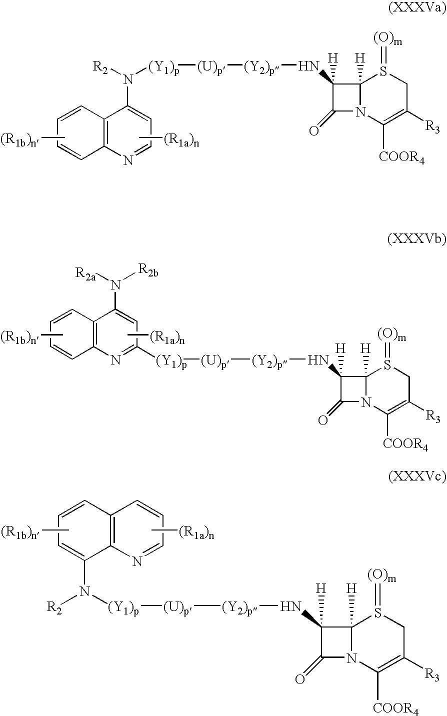

Other types of preferred hybrid molecules from the family of aminoquinoline-cephalosporin hybrid molecules are composed of a 4-aminoquinoline having the formula (XXXIIIa) or (XXXIIIb) and of cephalosporins having the formula (IXa) or (IXb). These hybrid molecules have the structure (XXXVd), (XXXVe), (XXXVf), or (XXXVg) in which R, R₁, R₂, R₃, R₄, HetAr, Y₁, Y₂, U, p, p′, p″, m, n and n′ are as defined above.

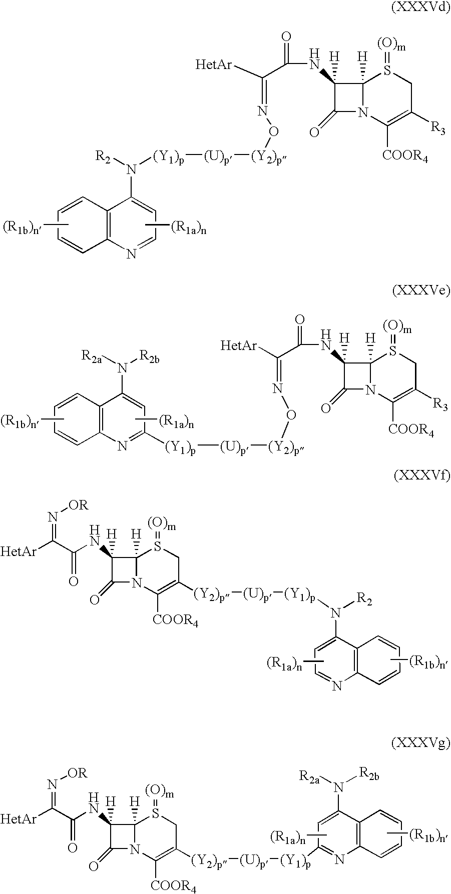

According to a preferred disposition, in the hybrid molecules of aminoquinoline-penicillin or aminoquinoline-cephalosporin type having the formula (XXXIVa), (XXXIVb), (XXXIVc), (XXXVa), (XXXVb), (XXXVc), (XXXVd), (XXXVe), (XXXVf), or (XXXVg), R₁ and R₂ advantageously represent the preferred aminoquinoline substituents (XXXIIIa), (XXXIIIb) and (XXXIIIe) defined previously and R₄ is a hydrogen atom or a moiety that is easily hydrolyzable in vivo in the area of prodrug molecules (such as 2,2-dimethyl-propionyloxymethyl).

In the aminoquinoline-penicillin hybrid molecules having the formula (XXXIVa) or (XXXIVb), according to a preferred disposition R₃ₐ and R_{3b} advantageously represent two identical substituents of alkyl type (such as two methyl substituents).

In the aminoquinoline-cephalosporin type hybrid molecules having the formulae (XXXVa), (XXXVb), (XXXVc), (XXXVd) or (XXXVe), R₃ advantageously represents a halogen or a saturated or unsaturated C1, C2, C3, C4, C5, or C6 alkyl chain possibly containing a carboxy or ether function (such as a methyl, vinyl, acetoxymethyl or methoxymethyl group) and being able to bear a heteroaryl or heterocycle substituent (such as pyridinium-1-ylmethyl, 1-methyl-1H-tetrazol-5-ylsulfanylmethyl or 6-hydroxy-2-methyl-5-oxo-2,5-dihydro-[1,2,4]triazin-3-ylsulfanylmethyl).

In the aminoquinoline-cephalosporin type hybrid molecules having the formula (XXXVf), or (XXXVg), R advantageously represents a hydrogen atom or a C1, C2, C3, C4, C5, or C6 alkyl substituent (preferably methyl) and “HetAr” represents a heteroaryl as defined previously and is advantageously selected from the group of 2-amino-thiazol-4-yl, 2-amino-5-chloro-thiazol-4-yl or 5-amino-[1,2,4]-thiadiazol-3-yl.

In the aminoquinoline-penicillin or aminoquinoline-cephalosporin type hybrid molecules having the formulae (XXXIVa), (XXXIVb), (XXXIVc), (XXXVa), (XXXVb), (XXXVc), (XXXVd), (XXXVe), (XXXVf), or (XXXVg) the following is preferred as (Y₁)ₚ—(U)_{p′}—(Y₂)_{p″} group: a group in which p, p′ et p″ are, independently of each other, 0 or 1, U being as defined above and advantageously representing a carbonyl, amide, thioether or alkoxyiminocarbonyl function and Y₁ and Y₂ being as defined above and advantageously representing a linear or branched, cyclic or acyclic C1, C2, C3, C4, C5, or C6 alkyl chain, possibly being able to contain an amine or thioether function and being able to be substituted by fluorine atoms.

The following are particularly preferred:

the compounds having the formula (XXXIVa), (XXXIVb), (XXXIVc), (XXXVa), (XXXVb), (XXXVc) according to the invention, which comprise as (Y₁)ₚ—(U)_{p′}—(Y₂)_{p″} group a carbonyl moiety (p′=1, p=p″=0), alkoxyiminocarbonyl (p′=1, p=p″=0) (preferably hydroxyiminocarbonyl or methoxylaminocarbonyl), or C1, C2, C3, C4, C5, or C6 alkylcarbonyl (p=p′=1, p″=0) (preferably acetyl, 3-propionyl, 2-propionyl, 2-methyl-2-propionyl, 4-butyryl, 3-methyl-3-butyryl or piperidine-4-carbonyl (which include R₂ and the N of the aminoquinoline)),

the compounds having the formula (XXXVd) according to the invention, which comprise as (Y₁)ₚ—(U)_{p′}—(Y₂)_{p″} group a C1, C2, C3, C4, C5, ou C6 alkyl moiety (p=1, p′=p″=0) (preferably 2-ethyl, 3-propyl, 2-propyl, 2-methyl-2-propyl, 2,2-difluoro-3-propyl, or 4-piperidin-1-yl),

the compounds having the formula (XXXVe) according to the invention, which comprise as (Y₁)ₚ—(U)_{p′}—(Y₂)_{p″} group an alkylcarbamoyl moiety (p=0, p′=p″=1) (preferably 2-ethylcarbamoyl, 3-propylcarbamoyl, 2-propylcarbamoyl, 1-carbonylpiperidin-4-yl),

the compounds having the formula (XXXVf) according to the invention, which comprise as (Y₁)ₚ—(U)_{p′}—(Y₂)_{p″} group an alkylamine moiety (p=p′=1, p″=0) (preferably methylamino, 2-ethylamino, 3-propylamino, 2-propylamino, 2,2-difluoro-3-propylamino, 4-piperidin-1-yl, 4-piperazin-1-yl or piperidin-4-ylamino (which include R₂ and the N of the aminoquinoline)), dialkylamine (p=p′=p″=1) (preferably methylamino-2-ethyl, methylamino-3-propyl, methylamino-2-propyl, methylamino-2,2-difluoro-3-propyl, 4-piperidin-1-ylmethyl, 4-methylpiperazin-1-yl or 4-methylaminopiperidin-1-yl (which include R₂ and the N of the aminoquinoline)), alkylsulfanyl (p=p′=1, p″=0) (preferably methylsulfanyl, 2-ethylsulfanyl, 3-propylsulfanyl, 2-propylsulfanyl, 2,2-difluoro-3-propylsulfanyl, or piperidin-4-ylsulfanyl (which include R₂ and the N of the aminoquinoline)) or dialkylsulfanyl (p=p′=p″=1) (preferably methylsulfanyl-2-ethyl, methylsulfanyl-3-propyl, methylsulfanyl-2-propyl, methylsulfanyl-2,2-difluoro-3-propyl, 4-methylsulfanylpiperidin-1-yl (which include R₂ and the N of the aminoquinoline)),

the compounds having the formula (XXXVg) according to the invention, which comprise as (Y₁)ₚ—(U)_{p′}—(Y₂)_{p″} group a thioether moiety (p′=1, p=p″=0), alkylsulfanyl (p′=p″=1, p=0) (preferably methylsulfanyl), alkylaminoalkylcarbamoyl (p=0, p′=p″=1) (preferably methylamino-2-ethylcarbamoyl, methylamino-3-propylcarbamoyl, methylamino-2-propylcarbamoyl, 4-methylpiperazine-1-carbonyl, 4 methylaminopiperidine-1-carbonyl, 1-methylpiperidin-4-ylcarbamoyl) or alkylsulfanylalkylcarbamoyl (p=0, p′=p″=1) (preferably methylsulfanyl-2-ethylcarbamoyl, methylsulfanyl-3-propylcarbamoyl, methylsulfanyl-2-propylcarbamoyl, 4-methylsulfanylpiperidine-1-carbonyl).

Aminoquinoline-Quinolone Hybrid Molecules

Another type of preferred compounds is wherein it relates to the aminoquinoline-quinolone hybrid molecules having the formula (XXXVIa) or (XXXVIb) in which R₁, R₂, R₄, R₆, R₇, Y₁, Y₂, U, Z, p, p′, p″, n and n′ are as defined above.

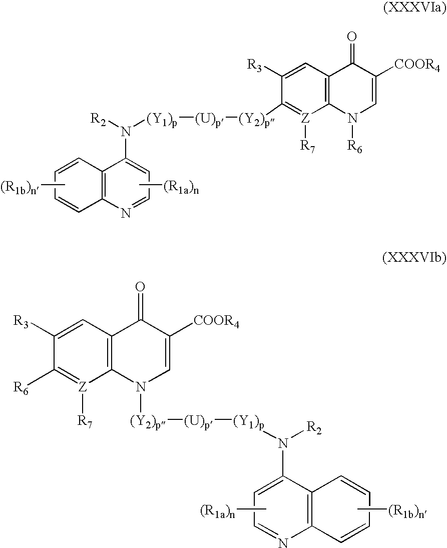

In the hybrid molecules of aminoquinoline-quinolone type having the formulae (XXXVIa) and (XXXVIb), according to a preferred disposition, Z is a carbon atom, R₁ and R₂ advantageously represent the preferred aminoquinoline substituents having the formula (XXXIIIa) previously defined, R₃ is a hydrogen or fluorine atom and R₄ is a hydrogen atom.

In the hybrid molecules of aminoquinoline-quinolone type having the formulae (XXXVIa),

according to a preferred disposition, R₆ is a linear, branched or cyclic C1, C2, C3, C4, C5, or C6 alkyl chain (preferably an ethyl or cyclopropyl substituent) or forms a cyclic structure with R₇ and R₇ is a hydrogen or halogen atom, a methoxy moiety or forms a cyclic structure with R₆ such as a 3-methyl-3,4-dihydro-2H-[1,4]oxazine;

as (Y₁)ₚ—(U)_{p′}—(Y₂)_{p″} group, the following group is preferred, in which p=p′=p″=0, Q being directly linked to A, or a group in which p=p′=1 and p″=0, U being as defined above and advantageously representing an amine function and Y₁ being as defined above and advantageously representing a C1, C2, C3, C4, C5, or C6 alkyl chain and that can form a cyclic structure with U or R₂ (including the N of the aminoquinoline) and possibly containing an amine function. In particular the compounds having the formula (XXXVIa) according to the invention are preferred, notably those whose link (Y₁)ₚ—(U)_{p′}—(Y₂)_{p″} is absent or which comprise a 2-ethylamino, 4-ethyl-piperazin-1-yl or 4-piperazin-1-yl (including R₂ and the N of the aminoquinoline) as (Y₁)ₚ—(U)_{p′}—(Y₂)_{p″} group.

In the hybrid molecules of aminoquinoline-quinolone type having the formula (XXXVIb),

according to a preferred disposition, R₆ is a heterocycle preferably containing 1 or 2 heteroatoms (such as piperazin-1-yl, N-methylpiperazin-1-yl, 3-methylpiperazin-1-yl or 3-amino-pyrrolidin-1-yl);

as (Y₁)ₚ—(U)_{p′}—(Y₂)_{p″} group the following group is preferred, in which p=p′=p″=0, Q being directly linked to A, and the exocyclic nitrogen atom of the aminoquinoline corresponds to the endocyclic nitrogen atom of the quinolone, or a group in which p=1 and p′=p″=0, Y₁ being as defined above and advantageously representing a C1, C2, C3, C4, C5, or C6 alkyl chain and that can form a cyclic structure with R₂. In particular the compounds having the formula (XXXVIb) according to the invention are preferred, notably those whose link (Y₁)ₚ—(U)_{p′}—(Y₂)_{p″} is absent or which comprise a 2-ethyl or 4-piperidin-1-yl (including R₂ and the N of the aminoquinoline) as (Y₁)ₚ—(U)_{p′}—(Y₂)_{p″} group.

Aminoquinoline-Nitroimidazole Hybrid Molecules

In the aminoquinoline-nitroimidazole hybrid molecules, the compounds having the formula (XXXVII) are more especially preferred, in which R₁, R₂, R₃, R₄, Y₁, Y₂, U, p, p′, p″, m, n and n′ are as defined above.

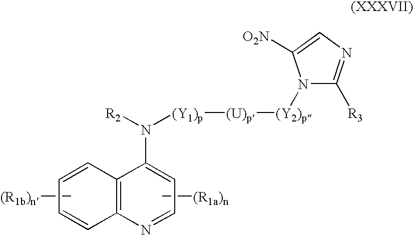

According to a preferred disposition in aminoquinoline-nitroimidazole hybrid molecules having the formula (XXXVII), R₁ and R₂ advantageously represent the substituents of the preferred aminoquinolines (XXXIIIa), R₃ is a methyl group and as (Y₁)ₚ—(U)_{p′}—(Y₂)_{p″} group a group in which p=1 and p′=p″=0 is preferred, Y₁ being as defined above and advantageously representing a C1, C2, C3, C4, C5, or C6 alkyl chain or a group in which p=p′=p″=1, U being as defined above and advantageously representing an amine function, Y₁ being as defined above and advantageously representing a C1, C2, C3, C4, C5, or C6 alkyl chain and that can form a cyclic structure with R₂ including the N of the aminoquinoline and Y₂ being as defined above and advantageously representing a C1, C2, C3, C4, C5, or C6 alkyl chain bearing a hydroxy function. In particular the compounds having the formula (XXXVII) according to the invention are preferred, notably those that comprise a 2-ethyl, 3-propyl, 2-propyl, 1-2-ethylamino)-propan-2-ol, 1-(3-propylamino)-propan-2-ol, 1-(2-propylamino)-propan-2-ol, or 1-(4-piperazin-1-yl)propan-2-ol moiety as the (Y₁)ₚ—(U)_{p′}—(Y₂)_{p″} group.

Aminoquinoline-Streptogramin Hybrid Molecules

Another type of preferred compounds is wherein it relates to aminoquinoline-streptogramin hybrid molecules having the formula (XXXVIII) in which R₁, R₂, R₄ₐ, R_{4b}, R₅, Y₁, Y₂, U, p, p′, p″, n and n′ are as defined above.

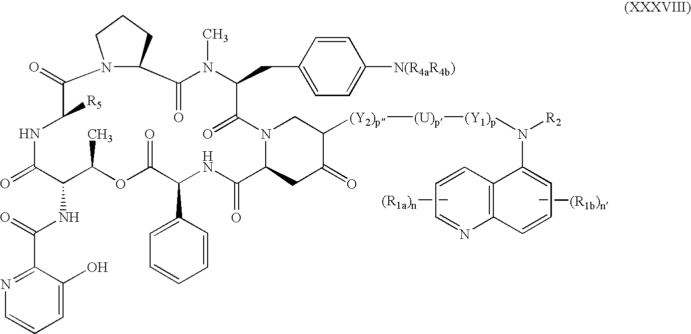

In the hybrid molecules of aminoquinoline-streptogramin type having the formula (XXXVIII),

according to a preferred disposition R₁ and R₂ advantageously represent the preferred substituents of aminoquinolines (XXXIIIa) defined previously, R₄ₐ, R_{4b} and R₅ are C1, C2, C3, C4, C5, or C6 alkyl chains (preferably R₄ₐ and R_{4b} are methyl substituent and R₅ an ethyl substituent);

as (Y₁)ₚ—(U)_{p′}—(Y₂)_{p″} group the following is preferred: a group in which p=p′=p″=1, U being as defined above and advantageously representing a thioether function and Y₁ and Y₂ being as defined above and advantageously representing a C1, C2, C3, C4, C5, or C6 alkyl chain. In particular the compounds having the formula (XXXVIII) according to the invention are preferred, that comprise a 1-2-ethylamino)methylsulfanyl, 1-(2-propylamino)-methylsulfanyl, 1-(3-propylamino)methylsulfanyl, or 1-piperidin-4-ylsulfanylmethyl moiety as the (Y₁)ₚ—(U)_{p′}—(Y₂)_{p″} group.

Aminoquinoline-Diaminopyrimidine Hybrid Molecules

In the aminoquinoline-diaminopyrimidine hybrid molecules, the compounds having the formula (XXXIX) are more especially preferred, in which R₁, R₂, R₄, R₅, Y₁, Y₂, U, p, p′, p″, m, n and n′ are as defined above.

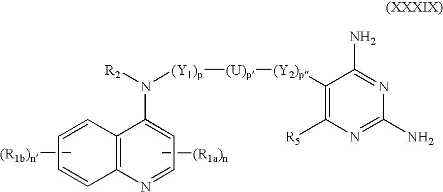

According to a preferred disposition in aminoquinoline-diaminopyrimidine hybrid molecules having the formula (XXXIX), R₁ and R₂ advantageously represent the substituents of the preferred aminoquinolines (XXXIIIa) defined previously, R₅ is a hydrogen atom and as (Y₁)ₚ—(U)_{p′}—(Y₂)_{p″} group a group in which p=p′=p″=1 is preferred, U being as defined above and advantageously representing an ether function and Y₁ being as defined above and advantageously representing a C1, C2, C3, C4, C5, or C6 alkyl chain, and Y₂ being as defined above and advantageously representing a C1, C2, C3, C4, C5, or C6 alkyl chain containing an aryl group as defined previously that can itself bear 1 to 4 identical or different substituents. In particular the compounds having the formula (XXXIX) according to the invention are preferred, that comprise a 4-(2-ethoxy)-benzyl, 4-(2-ethoxy)-3-methoxy-benzyl, 4-(2-ethoxy)-3,5-dimethoxy-benzyl or 3-(2-ethoxy)-4,5-dimethoxy-benzyl moiety as the (Y₁)ₚ—(U)_{p′}—(Y₂)_{p″} group.

Aminoquinoline-Macrolide Hybrid Molecules

Another type of preferred compounds is wherein it relates to aminoquinoline-macrolide hybrid molecules having the formula (XLa), (XLb) or (XLc) in which R₁, R₂, R₅, R₆, R₇, R₁₀, Y₁, Y₂, U, p, p′, p″, n and n′ are as defined above.

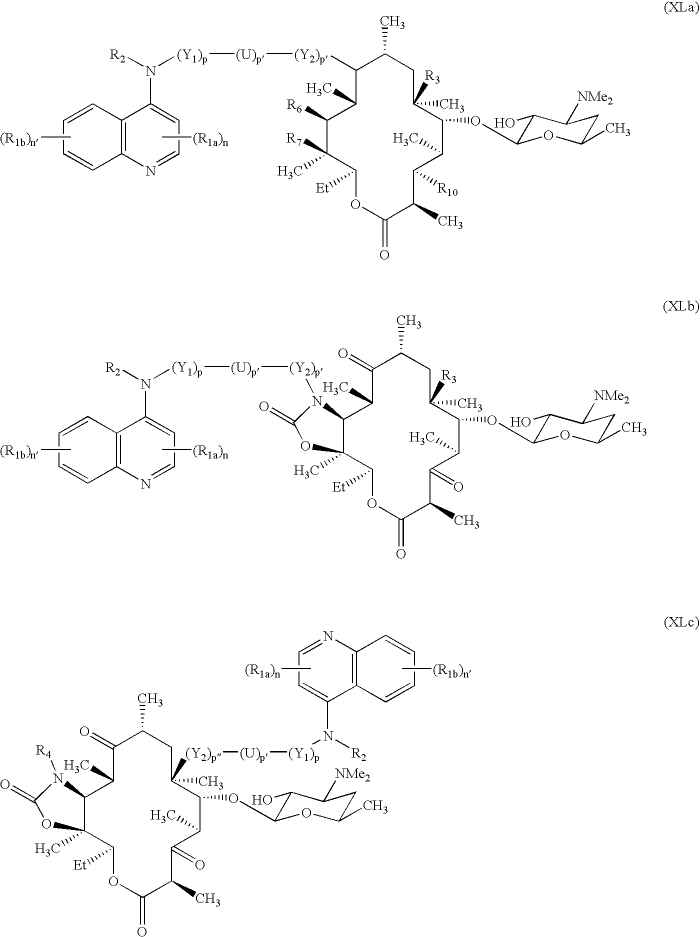

In the hybrid molecules of aminoquinoline-macrolide type having the formulae (XLa), (XLb) and (XLc), according to a preferred disposition, R₁ and R₂ advantageously represent the preferred aminoquinoline substituents (XXXIIIa), R₃ is a hydroxy or methoxy moiety, R₄ is a hydrogen atom, R₆ and R₇ are hydroxy moieties, R₁₀ is an oxygen atom linked by a carbonyl type double bond to the macrocycle or an osidic derivative linked by a glycosidic bridge to the macrocycle and that can bear 1 to 6 substituents (preferably a L-cladinose derivative).

In the aminoquinoline-macrolide type hybrid molecules having the formula (XLa), the following is preferred as (Y₁)ₚ—(U)_{p′}—(Y₂)_{p″} group: a group in which p=p′=1 and p″=0, U being as defined above and advantageously representing an oxyamine function linked by a double bond to A (thus forming an oxime function) and Y₁ being as defined above and advantageously representing a C1, C2, C3, C4, C5, or C6 alkyl chain, that can contain an ether function. In particular the compounds having the formula (XLa) according to the invention are preferred, that comprise a 0-2-ethyl-oxime, 0-3-propyl-oxime, 0-2-propyl-oxime, 04-butyl-oxime or O[2-(2-ethoxy)-ethyl]-oxime moiety as the (Y₁)ₚ—(U)_{p′}—(Y₂)_{p″} group.

In the aminoquinoline-macrolide having the formula (XLb), the following is preferred as (Y₁)ₚ—(U)_{p′}—(Y₂)_{p″} group: a group in which p=1 and p′=p″=0, Y₁ being as defined previously and advantageously representing a C1, C2, C3, C4, C5, or C6 alkyl chain that can contain an ether function. In particular the compounds having the formula (XLb) according to the invention are preferred, that comprise a 2-ethyl, 3-propyl, 2-propyl, 4-butyl or 2-(2-ethoxy)-ethyl moiety as the (Y₁)ₚ—(U)_{p′}—(Y₂)_{p″} group.

In the aminoquinoline-macrolide type hybrid molecules having the formula (XLc), the following is preferred as (Y₁)ₚ—(U)_{p′}—(Y₂)_{p″} group: a group in which p=p′=1 and p″=0, U being as defined above and advantageously representing an ether or carbamate function and Y₁ being as defined above and advantageously representing a saturated or unsaturated C1, C2, C3, C4, C5, or C6 alkyl chain, that can contain an ether function and/or aryl group. In particular the compounds having the formula (XLc) according to the invention are preferred, that comprise a 2-ethoxy, 3-propoxy, 2-propoxy, 2-ethoxy-2-ethoxy, 3-allyloxy, 2-ethylcarbamoyloxy, 3-propylcarbamoyloxy, 4-butylcarbamoyloxy, 4-(2-ethoxy)-benzylcarbamoyloxy moiety as the (Y₁)ₚ—(U)_{p′}—(Y₂)_{p″} group.

Aminoquinoline-Glycopeptide Hybrid Molecules

In the aminoquinoline-glycopeptide hybrid molecules, the compounds having the formulae (XLIa) ou (XLIb) are more especially preferred, in which R₁, R₂, Y₁, Y₂, U, p, p′, p″, n and n′ are as defined above.

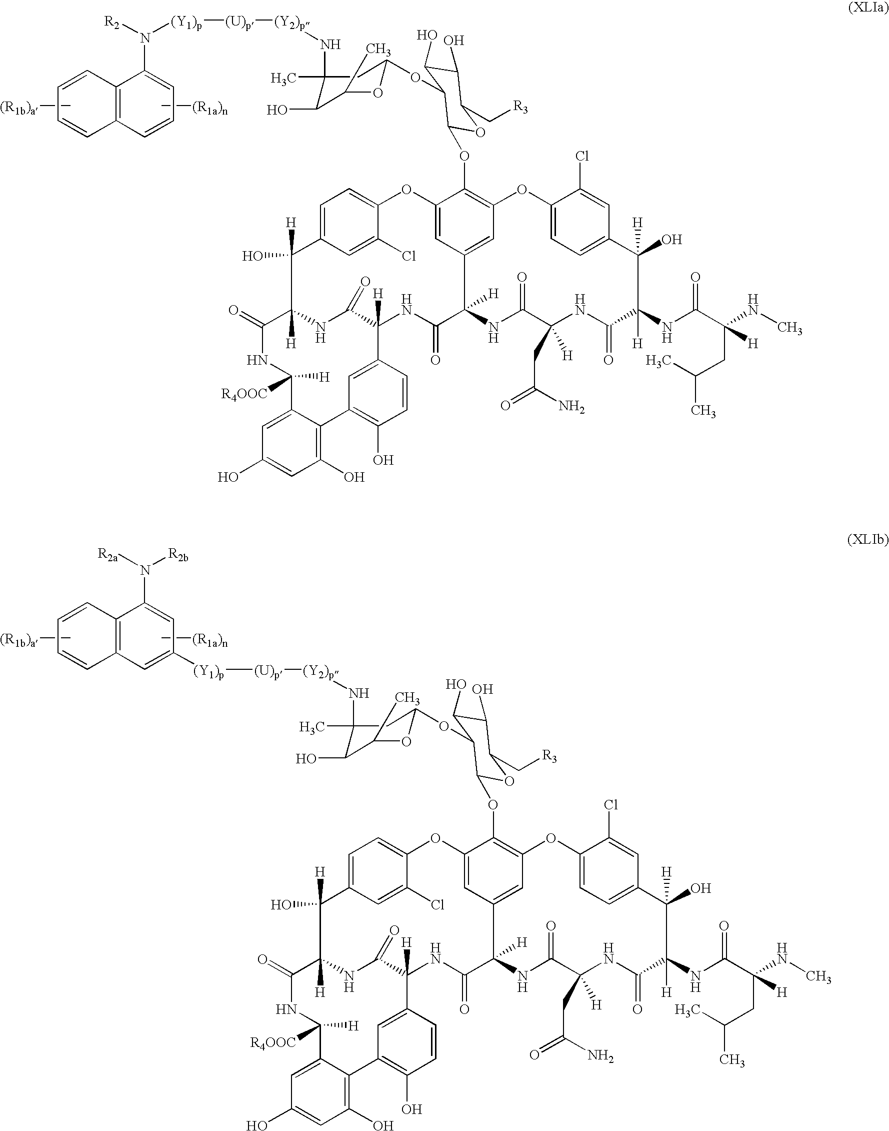

In the hybrid molecules of aminoquinoline-glycopeptide type having the formula (XLIa) or (XLIb), according to a preferred disposition, R₁ and R₂ advantageously represent the preferred aminoquinoline substituents (XXXIIIa) and (XXXIIIb), R₄ is a hydrogen atom and R₃ is a hydroxy moiety.

In the aminoquinoline-glycopeptide hybrid molecules having the formula (XLIa), as (Y₁)ₚ—(U)_{p′}—(Y₂)_{p″} group a group in which p=1 and p′=p″=0 is preferred, Y₁ being as defined above and advantageously representing a C1, C2, C3, C4, C5, or C6 alkyl chain that can form a cyclic structure with the A residue and R₂ (including the N of the aminoquinoline) and that can be substituted by fluorine atoms or a group in which p=p′=p″=1, U being as defined above and advantageously representing an ether or amine function, Y₁ being as defined above and advantageously representing a C1, C2, C3, C4, C5, or C6 alkyl chain that can form a cyclic structure with U and R₂ (including the N of the aminoquinoline), Y₂ being as defined above and advantageously representing a C1, C2, C3, C4, C5, or C6 alkyl chain that can contain an aryl moiety as previously defined, that can itself bear 1 to 4 identical or different substituents. In particular the compounds having the formula (XLIa) according to the invention are preferred, that comprise a 2-ethyl, 3-propyl, 4-butyl 2,2-difluoro-propyl, 4-piperazin-1-yl, 4-piperazin-1-ylmethyl or 4-(2-ethoxy)-benzyl moiety as the (Y₁)ₚ—(U)_{p′}—(Y₂)_{p″} group.

In the aminoquinoline-glycopeptide hybrid molecules having the formula (XLb), the following is preferred as (Y₁)ₚ—(U)_{p′}—(Y₂)_{p″} group: a group in which p=1 and p′=p″=0, Y₁ being as defined above and advantageously representing a C1, C2, C3, C4, C5, or C6 alkyl chain or a group in which p=0 and p′=1, U being as defined above and advantageously representing an amide function, Y₂ being as defined above and advantageously representing a C1, C2, C3, C4, C5, or C6 alkyl chain. In particular the compounds having the formula (XLIb) according to the invention are preferred, that comprise a methyl, ethylcarbamoyl, propylcarbamoyl or butylcarbamoyl moiety as the (Y₁)ₚ—(U)_{p′}—(Y₂)_{p″} group.

Aminoquinoline-Oxazolidinone Hybrid Molecules

Other types of preferred hybrid molecules are composed of a 4-aminoquinoline having the formula (XXXIIIa) or a 2-aminoquinoline having the formula (XXXIIIc) and of an oxazolidinone having the formula (XIVa) or (XIVb). These aminoquinoline-oxazolidinone hybrid molecules have the formula (XLIIa), (XLIIb) or (XLIIc) in which R₁, R₂, R₆, R₇, Y₁, Y₂, U, p, p′, p″, m, n and n′ are as defined above.

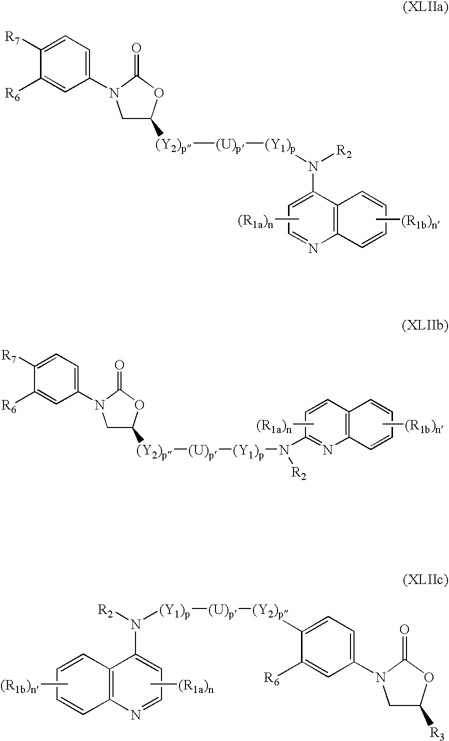

In the aminoquinoline-oxazolidinone hybrid molecules having the formula (XLIIa), (XLIIb), or (XLIIc), according to a preferred disposition, R₁ and R₂ advantageously represent the preferred substituents of the aminoquinolines (XXXIIIa) and (XXXIIIc), R₆ is a hydrogen or fluorine atom, R₇ is a 5 to 6 membered heterocycle comprising 1 to 4 heteroatoms chosen from among nitrogen, sulfur and oxygen (preferably morpholin-4-yl or piperazin-1-yl) and R₃ is advantageously a C1, C2, C3, C4, C5, or C6 alkyl chain that can contain an amide (such as an acetylaminomethyl chain), carbamate (such as a methoxycarbonylaminomethyl chain) or ether function and that can be substituted by a heterocycle (such as a [1,2,3]-triazol-1-ylmethyl or isoxazol-3-ylmethyl chain).

In the aminoquinoline-oxazolidinone type hybrid molecules having the formula (XLIIa), the following is preferred as (Y₁)ₚ—(U)_{p′}—(Y₂)_{p″} group: a group in which p=p′=p″=1, U being as defined above and advantageously representing an amide or carbamate function and Y₁ and Y₂ being as defined above and advantageously representing a C1, C2, C3, C4, C5, or C6 alkyl chain that can form a cyclic structure with U and/or R₂ including the N of the aminoquinoline. In particular the compounds having the formula (XLIIa) according to the invention are preferred, that comprise a (methylcarbamoyl)methyl, 2-(methylcarbamoyl)-ethyl, 1-(methylcarbamoyl)-ethyl, 1-(1-methyl)-1-(methylcarbamoyl)ethyl, 3-(methylcarbamoyl)-propyl, 2-(methylcarbamoyl)-propyl, 2-(2-methyl)-2-(methylcarbamoyl)-propyl, 4-(methylcarbamoyl)-piperidin-1-yl or 2-ethylcarbamoyloxymethyl, 2-(1-methyl)-ethylcarbamoyloxymethyl, 3-propylcarbamoyloxymethyl, 2-propylcarbamoyloxymethyl, 4-piperazine-1-carbonyloxymethyl moiety as the (Y₁)ₚ—(U)_{p′}—(Y₂)_{p″} group.

In the aminoquinoline-oxazolidinone type hybrid molecules having the formula (XLIIb), the following is preferred as (Y)ₚ—(U)_{p′}—(Y₂)_{p″} group: a group in which p=p′=p″=1, U being as defined above and advantageously representing a carbamate function and Y₁ and Y₂ being as defined above and advantageously representing a C1, C2, C3, C4, C5, or C6 alkyl chain that can form a cyclic structure with U and/or R₂ including the N of the aminoquinoline. In particular the compounds having the formula (XLIIb) according to the invention are preferred, that comprise a 2-ethylcarbamoyloxymethyl, 2-(1-methyl)-ethylcarbamoyloxymethyl, 3-propylcarbamoyloxymethyl, 2-propylcarbamoyloxymethyl, 4-piperazine-1-carbonyloxymethyl moiety as the (Y₁)ₚ—(U)_{p′}—(Y₂)_{p″} group.

In the aminoquinoline-oxazolidinone hybrid molecules having the formula (XLIIc) the following group is preferred as (Y₁)ₚ—(U)_{p′}—(Y₂)_{p″} group, in which p=p′=p″=0, Q being directly linked to A, or a group in which p=p′=1 and p″=0, U being as defined above and advantageously representing an amine function and Y₁ being as defined above and advantageously representing a linear, branched or cyclic C1, C2, C3, C4, C5 or C6 alkyl chain that can form a cyclic structure with U and/or R₂ including the N of the aminoquinoline and possibly containing an amine, amide, urea or carbamate function. In particular the compounds having the formula (XLIIc) according to the invention are preferred, that comprise either a direct link between Q and A or a 2-ethylamino, 2-(1-methyl)ethylamino, 3-propylamino, 2-propylamino, 3-(2-methyl)propylamino, 2,2-difluoro-3-propylamino, 4-piperazin-1-yl, 4(2-ethyl)-piperazin-1-yl moiety, or 4-(2-acetyl)-piperazin-1-yl, 4-(3-propionyl)-piperazin-1-yl, 4-2-propionyl)-piperazin-1-yl, 4(2-methyl-3-propionyl)-piperazin-1-yl, or 4-(2-ethylcarbamoyl)-piperazin-1-yl, 4-(3-propylcarbamoyl)-piperazin-1-yl, 4-(2-propylcarbamoyl)-piperazin-1-yl, 4-[3-2-methyl)propylcarbamoyl)-piperazin-1-yl, 4-[3-(2,2-difluoro)propylcarbamoyl]-piperazin-1-yl or 4-2-ethoxycarbonyl)-piperazin-1-yl, 4-3-propoxycarbonyl) -piperazin-1-yl, 4-[2-2-methyl)propoxycarbonyl)-piperazin-1-yl as the (Y₁)ₚ—(U)_{p′}—(Y₂)_{p″} group.

The invention also covers methods of synthesis of the molecules having the formula (I) defined above.

These methods comprise the reaction of reactive derivatives or precursors of aminoquinolines Q and of reactive derivatives or precursors having antibiotic activity A, so as to form, between these derivatives, a coupling arm-(Y₁)ₚ—(U)_{p′}—(Y₂)_{p″} as defined with respect to formula (I).

Various synthetic routes will be easily accessible to the person skilled in the art in proceeding according to classical techniques.

Advantageously, the method of preparing a compound Q—(Y₁)ₚ—(U)_{p′}—(Y₂)_{p″}-A, as defined above comprises:

a) either fixing the (Y₁)ₚ—(U)_{p′}—(Y₂)_{p″} group onto an aminoquinoline Q, and then reacting this intermediate compound with A, notably an antibiotic;

b) or fixing the (Y₁)ₚ—(U)_{p′}—(Y₂)_{p″} group with A, notably an antibiotic, and then coupling this intermediate with an aminoquinoline Q;

c) or fixing an amino-(Y₁)ₚ—(U)_{p′}—(Y₂)_{p″} group onto a corresponding quinoline making it possible to obtain an intermediate compound Q—(Y₁)ₚ—(U)_{p′}—(Y₂)_{p″}—, and then grafting this intermediate compound onto A, notably onto an antibiotic A.

Aminoquinoline-β-Lactam Hybrid Molecules

It is advantageous to prepare hybrid molecules having a 4-aminoquinoline having the formula (XXXIIIa) as derivative Q and a penicillin having the formula (IV) as residue A as follows:

a-1) a reaction can be performed between a compound having the formula (XLIII): 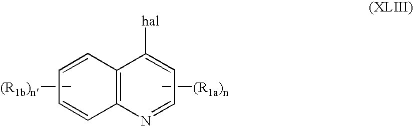
in which R₁ₐ and R_{1b}, n and n′ are as defined above and “hal” represents a halogen atom, with a derivative having the formula (XLIV):
R₂NH —(Y₁)ₚ—(U)_{p′} (XLIV)
wherein R₂, Y₁, p and p′ are as defined above and U represents a carboxy or carboxyalkyl group (preferably U═COOH), which leads to a 4-aminoquinoline having the formula (XLV): 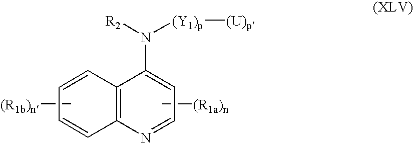
in which R₁ₐ, R_{1b}, R₂, Y₁, n, n′, p and p′ are as defined above,

b-1) the coupling of the 4-aminoquinoline having the formula (XLV) is then carried out in the presence of an activator of the U function, with a precursor of the antibiotic residue A having the formula (XLVI), if need be as an addition salt with an acid (such as p toluenesulfonic acid) in which R₃ₐ, R_{3b}, R₄, and m are as defined above, 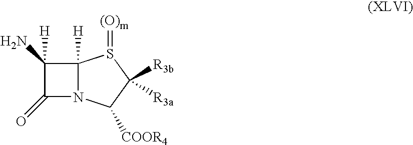
which leads to the hybrid molecules having the formula (XXXIVa) in which p″=0.

Step a-1) is advantageously carried out in molten phenol, at a temperature of 120° C. to 150° C. under stirring for 24 hours. After cooling to room temperature, the product is obtained after various washings and/or extractions and, if need be, recrystallization by dissolution in carbonate-containing water and then precipitation by adding hydrochloric acid.

Step b-1) is advantageously carried out in a solvent such as an amide (preferably dimethylformamide) in the presence of an activator of the U function (PyBOP® or the dicyclohexylcarbodiimide/hydroxybenzotriazole system, for example) at room temperature.

It is also advantageous for the preparation of the hybrid molecules having an 8-aminoquinoline having the formula (XXXIIIe) as derivative Q and as residue A a penicillin having the formula (IV) to proceed in the following manner:

a-2) the coupling reaction is carried out between a reactive derivative of 8-aminoquinoline having the formula (XLVII) wherein R₁ₐ, R_{1b}, R₂, Y₁, n, n′, p and p′ are as defined above and U represents a carboxy or carboxyalkyl group (preferably U═COOH): 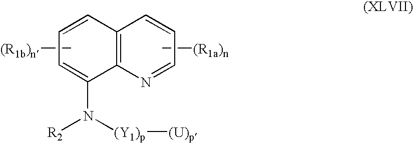
and a precursor of antibiotic residue A having the formula (XLVI). This coupling reaction leads to the hybrid molecules having the formula (XXXIVc) in which p″=0.

Step a-2) is advantageously carried out according to the conditions described for step b-1) in the presence of an activator of the U function (PyBOP® or the dicyclohexylcarbodiimide/hydroxybenzotriazole system, for example).

In another method, in order to prepare hybrid molecules having a cephalosporin having the formula (VIIIa) as residue A and an aminoquinoline having the formula (XXXIIIa) as derivative Q:

a-3) the coupling of the reactive derivative of aminoquinoline having the formula (XLV) is carried out, in the presence of an activator of the U function, with a cephalosporin having the formula (XLVIII), if need be as an addition salt with an acid (such as t toluenesulfonic acid) in which R₃ and R₄ are as defined above and m=0. 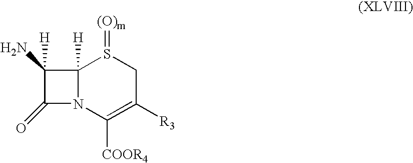
which produces of a mixture of isomers of Δ² and Δ³ cephems having the formula (XLIX): 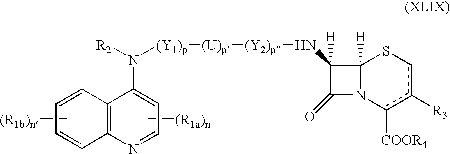
wherein R₁ₐ, R_{1b}, R₂, R₃, R₄, Y₁, U, n, n′, p and p′ are as defined above and p″=0,

b-3) an oxidation of the mixture of Δ²/Δ³ isomers having the formula (XLIX) is then carried out, which leads to hybrid molecules of formula (XXXVa) in which R₁ₐ, R_{1b}, R₂, R₃, R₄, Y₁, U, m, n, n′, p and p′ are as defined above, p″=0 and m=1. This oxidation is followed if need be by an acid hydrolysis of the ester function COOR₄ for the synthesis of the hybrid molecules having the formula (XXXVa) in which R₄═H and m=1. The latter molecules can then be obtained as a salt by reaction with a pharmacologically acceptable acid,

c-3) the compounds having the formula (XXXVa) in which p″=0 and m=1 are subsequently reduced in order to obtain the aminoquinoline-cephalosporin hybrid molecules having the formula (XXXVa) wherein R₁ₐ, R_{1b}, R₂, R₃, R₄, Y₁, U, n, n′, p and p′ are as defined above and m=p″=0. In the case in which R₄ is a protecting group, the deprotection can be carried out by acid hydrolysis. This step is followed if need be by a protonation with a pharmacologically acceptable acid, in order to obtain the product as a salt.

Step a-3) is advantageously carried out according to the conditions described for step b-1) in the presence of an activator of the U function (PyBOP® or the dicyclohexylcarbodiimide/hydroxybenzotriazole system for example).

Step b-3) is advantageously carried out in a halogenated solvent (dichloromethane for example) at 0° C. by slowly adding a solution of the oxidizing agent (for example 3-chloroperoxybenzoic acid).

Step c-3) is advantageously carried out at low temperature (−20° C.) in an amide solvent (dimethylformamide for example) under an inert atmosphere and in the presence of a reducing agent such as trichlorophosphine.

Another advantageous method to prepare in one step (instead of steps a3, b3, and c3) hybrid molecules of formula (XXXVa) wherein R₁ₐ, R_{1b}, R₂, R₃, R₄, Y₁, U, m, n, n′, p and p′ are as defined above and p″=0 is:

a′-3) to carry out the coupling reaction between the reactive aminoquinoline of formula (XLV) in which U represents a carboxy group and a cephalosporin having the formula (XLVIII) using an halogenated phosphorus compound (preferably phosphorus oxychloride) as the activator of the U function and in the presence of a base (preferably 2,4,6-collidine). If needed, this step may be followed by a deprotection reaction by an acidic hydrolysis and formation of a salt with a pharmacologically acceptable acid or base. Step a′-3) is advantageously carried out at low temperature (−30° C.) under an inert atmosphere and in a dried organic solvent (such as anhydrous tetrahydrofuran).

When a deprotection step is necessary to obtain hybrid molecules of formula (XXXVa) in which R₄═H, it is advantageously carried out in a halogenated solvent, under an inert atmosphere, in the presence of a compound used for trapping the carbocation released (anisole for example). The hydrolysis can be carried out by adding an acid (such as trifluoroacetic acid) at 0° C. followed by stirring at room temperature.

Hybrid molecules of formula (XXXVa) can be converted to pharmacologically acceptable salts such as acid salts by addition of an organic or inorganic acid (e.g. hydrochloric acid) or base salts such as alkali metal salt by addition of a base and an alkali metal salt.

In another method, the preparation of hybrid molecules containing a cephalosporin having the formula (VIIIa) as residue A and an aminoquinoline having the formula (XXXIIIb) as derivative Q can be done as follows:

a-4) halogenation of a hydroxyquinoline having the formula (L) where R₁ₐ, R_{1b}, n, n′ and p′ are as defined above and U represents a carboxylic ester. 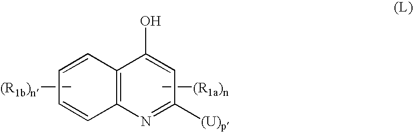
to obtain a halogenated quinoline having the formula (LI): 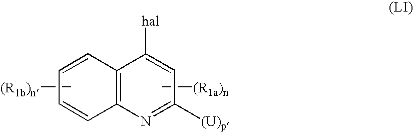
in which “hal” represents a halogen atom,

b4) reacting the halogenated quinoline having the formula (LI) where R₁ₐ, R_{1b}, n, n′ and p′ are as defined above and U represents a carboxylic ester with an amine having the formula (LII) where R₂ₐ and R_{2b} are as defined above: 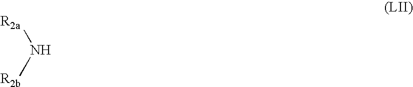
which produces an aminoquinoline having the formula (LIII)

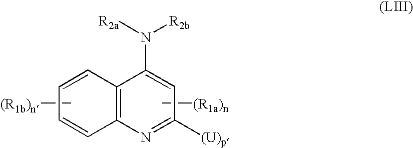

c-4) saponification of the aminoquinoline having the formula (LIII) where R₁ₐ, R_{1b}, n, n′ and p′ are as defined above and U represents a carboxylic ester to obtain the aminoquinoline having the formula (LIII) where U is a carboxylic acid,

d-4) coupling the reactive aminoquinoline derivative having the formula (LIII) in the presence of an activator of function U, with a cephalosporin having the formula (XLVIII), if need be in the form of an addition salt with an acid (such as p-toluene sulfonic acid) in which R₃ and R₄ are as defined above and m=0, which produces a mixture of isomers and Δ² and Δ³ cephems having the formula (LIV): 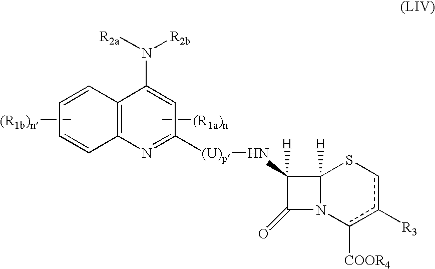
where R₁ₐ, R_{1b}, R₂, R₃, R₄, Y₁, U, n, n′, p and p′ are as defined above.

e-4) oxidation of the mixture of Δ²/Δ³ isomers having the formula (LIV) which leads to hybrid molecules of formula (XXXVb) where R₁ₐ, R_{1b}, R₂, R₃, R₄, Y₁, U, m, n, n′, p and p′ are as defined above, p=p″=0 and m=1.

f4) the compounds having the formula (XXXVb) in which m=1 are reduced in order to obtain the aminoquinoline-cephalosporin hybrid molecules having the formula (XXXVb) in which R₁ₐ, R_{1b}, R₂, R₃, R₄, Y₁, U, n, n′, p and p′ are as defined above and p=p′=m=0. In the case in which R₄ is a protecting group, the deprotection can be carried out by acid hydrolysis. This step is followed if need be by a protonation with a pharmacologically acceptable acid, in order to obtain the product as a salt.

Step a4) is advantageously carried out with a chlorinating agent (such as trichlorooxyphosphine) at reflux.

Step b4) is advantageously carried out at reflux in an excess of amine having the formula (L).

Step c<) is advantageously carried out in a mixture of alcoholic solvent (for example ethanol) and a mineral base in aqueous solution (such as an aqueous sodium hydroxide solution).

Step d<) is advantageously carried out according to the conditions described for step b-1) in the presence of an activator of the U function (PyBOP®, for example).

Step c 1) is advantageously carried out according to the conditions described for step b-3).

Step f4) is advantageously carried out according to the conditions described for step c-3).

In another method, to prepare hybrid molecules of formula (XXXVd) in which p′=p″=0, it is advantageous to:

a-5) react a 2-heteroaryl-2-hydroxyimino acetic acid alkylester of formula (LV): 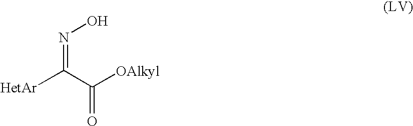
wherein HetAr and alkyl group are as defined above with a 4-aminoquinoline of formula (LVI): 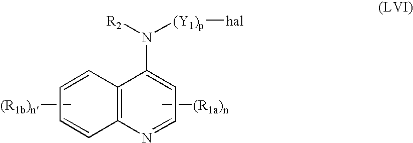
wherein R₁ₐ, R_{1b}, R₂, Y₁, n, n′, and p are as defined above and “hal” represents a halogen atom to afford compound of formula (LVII): 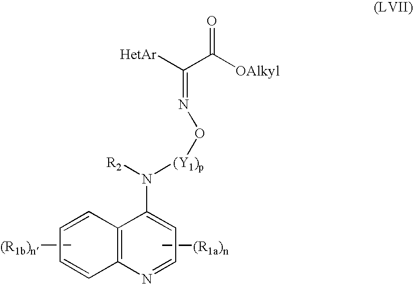
wherein R₁ₐ, R_{1b}, R₂, Y₁, n, n′, p, HetAr and alkyl group are as defined above,

b-5) saponify compound of formula (LVII) to obtain the carboxylic acid derivative of formula (LVIII): 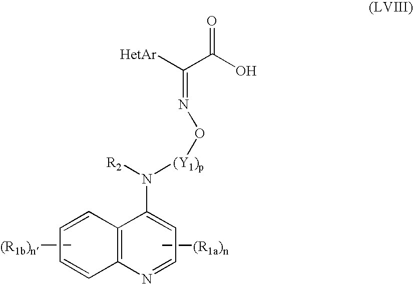
wherein R₁ₐ, R_{1b}, R₂, Y₁, n, n′, p and HetAr are as defined above,

c-5) activate the carboxylic acid function of compound of formula (LVIII) prior to the coupling with a cephalosrin of formula (XLVIII) to obtain hybrid molecules of formula (XXXVd) wherein R₁ₐ, R_{1b}, R₂, R₃, R₄, Y₁, Y₂, U, m, n, and n′ are as defined above, p=1 and p′=p″=0. If needed, deprotection reaction may be carried out by acidic hydrolysis. Step a-5) is advantageously carried out in an organic solvent (for example dimethylformamide) at room temperature, in the presence of a base (such as potassium carbonate) and an iodide salt (e.g. tetrabutylammonium iodide).

Step b-5) is advantageously carried out in an organic solvent miscible with water (such as 1,4-dioxane) in the presence of a strong base (preferably sodium hydroxide).

Step c-5) is advantageously carried out according to the conditions described for step b′ 3).

When a deprotection step is necessary to obtain hybrid molecules of formula (XXXVd) in which R₄═H, it is advantageously carried out in an halogenated solvent, under an inert atmosphere, in the presence of a compound used for trapping the carbocation released (anisole for example). The hydrolysis can be carried out by adding an acid (such as trifluoroacetic acid) at 0° C. followed by stirring at room temperature.

Hybrid molecules of formula (XXXVd) can be converted to pharmacologically acceptable salts such as acid salts by addition of an organic or inorganic acid (e.g. hydrochloric acid) or base salts such as alkali metal salt by addition of a base and an alkali metal salt.

In another method, to prepare aminoquinoline-cephalosporin hybrid molecules having the formula (XXXVf) in which U represents a thioether function, it is advantageous to proceed in the following manner:

a-6) reacting a 4-aminoquinoline having the formula (XLV) in which R₁ₐ, R_{1b}, R₂, Y₁, n, n′, p and p′ are as defined above and U represents a thiol function with a cephalosporin residue having the formula (LIX) in which R₄, Y₂, m and p″ are as defined above, “hal” represents a halogen atom and the amine's protecting group is, for example, a tert-butyloxycarbonyl group: 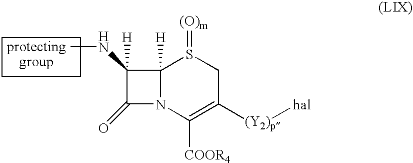
which produces cephalosporins having the formula (LX)

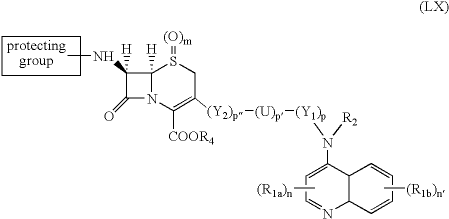

wherein R₁ₐ, R_{1b}, R₂, R₃, R₄, Y₁, Y₂, m, n, n′, p, p′ and p″ are as defined above and U represents a thioether function,

b-6) deprotection of the amine by an acidic treatment which produces cephalosporins having the formula (LXI) 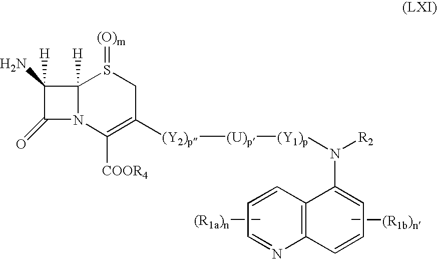
in which R₁ₐ, R_{1b}, R₂, R₃, R₄, Y₁, Y₂, U, m, n, n′, p, p′ and p″ are as defined above,

c-6) the cephalosporin having the formula (LXI) is coupled with an activated 2-heteroaryl-2-alkoxymino acetic acid having the formula (LXII): 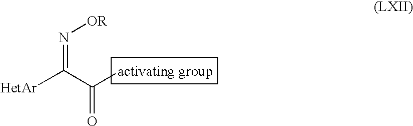
in which R and the heteroaryl HetAr are as defined above and the acid's activating group is for example a sulfanylbenzothiazole moiety.

Step a-6) is advantageously carried out in an amide solvent (for example dimethylformamide) at room temperature, in the presence of a base (such as N,N-diisopropylethylamine) and sodium iodide.

Step b-6) is advantageously carried out in an acidic medium (a mixture of formic/hydrochloric acid for example) at room temperature.

Step c-6) is advantageously carried out in a halogenated solvent (for example dichloromethane) between −10° C. and 25° C., in the presence of a base (such as triethylamine).

When a deprotection step is necessary it is advantageously carried out in a halogenated solvent, in an inert atmosphere, in the presence of a compound used to trap the carbocation released (for example anisole). The hydrolysis can be carried out by the addition of an acid (such as trifluoracetic acid) at 0° C. followed by stirring at room temperature.

Aminoquinoline-Quinolone Hybrid Molecules

It is advantageous to prepare aminoquinoline-quinolone hybrid molecules of formula (XXXVIa) wherein R₁ₐ, R_{1b}, R₂, R₃, R₄, R₆, R₇, Y₁, U, Z, n, n′, p, and p′ are as defined above, and p″=0, by reacting quinolines of formula (XLIII) wherein R₁ₐ, R_{1b}, n, and n′, are as defined above and “hal” represents a halogen atom with an antibiotic A of the family of quinolones having the formula (LXIII): 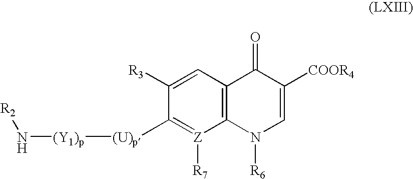
wherein R₂, R₃, R₄, R₆, R₇, Y₁, U, Z, p, and p′ are as defined above. The reaction is advantageously carried out in an organic solvent (such as 2-ethoxyethanol) at hot temperature (preferably above 120° C.).

In another preferred method for the preparation of aminoquinoline-quinolone hybrid molecules having the formula (XXXVIa) in which p″=0, U representing an amine function and Y₁ being a linear, branched, or cyclic C1, C2, C3, C4, C5 or C6 alkyl chain containing an amine function, the coupling of an aminoquinoline having the formula (LXIV): 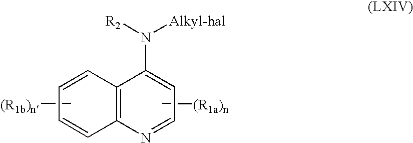
wherein R₁ₐ, R_{1b}, R₂, n, and n′ are as defined above and “hal” represents a halogen atom, is carried out with a quinolone having the formula (LXV): 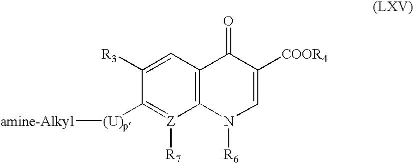
in which R₃, R₄, R₆, R₇ and Z are as defined above. In formulae (LXIV) and (LXV), alkyl groups represent linear, branched, or cyclic C1, C2, C3, C4, C5 or C6 alkyl chains.

The coupling reaction is advantageously carried out in an amide solvent (for example dimethylformamide) in the presence of a base (potassium carbonate for example) and at a temperature of 140° C.

Aminoquinoline-Nitroimidazole Hybrid Molecules

In another method for the preparation of aminoquinoline-nitroimidazole hybrid molecules having the formula (XXXVII) in which p′=p″=0, it is advantageous to react an aminoquinoline having the formula (LVI) wherein R₁ₐ, R_{1b}, R₂, Y₁, n, n′, and p are as defined above and “hal” represents a halogen atom, with 2-methyl-5-nitro-imidazole.

The coupling reaction is advantageously carried out in an amide solvent (for example dimethylformamide) in the presence of a base (potassium carbonate or triethylamine for example) and at a temperature between 70 and 140° C.

In the same way, to prepare aminoquinoline-nitroimidazole hybrid molecules having the formula (XXXVII) in which p=p′=p″=1, Y₂ being a C1, C2, C3, C4, C5 or C6 alkyl chain bearing a hydroxy substituent and U representing an amine function, it is advantageous to carry out a coupling reaction between an aminoquinoline having the formula (XLV) in which R₁ₐ, R_{1b}, R₂, Y₁, n, n′, p and p′ are as defined above and U represents an amine function, and a nitroimidazole residue having the formula (LXVI): 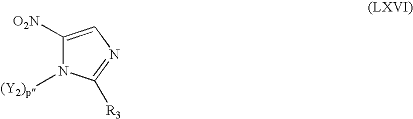
wherein R₃ and p″ are as defined above and Y₂ contains a cyclic ether function.

This coupling reaction is advantageously carried out in an alcoholic solvent (such as ethanol) in the presence of a base (triethylamine for example) and at the reflux temperature of the alcoholic solvent.

Aminoquinoline-Streptogramin Hybrid Molecules

In another method, to prepare aminoquinoline-streptogramin hybrid molecules having the formula (XXXVIII) in which p=p′=p″=1, U representing a thioether function and Y₂ being methylene, it is advantageous to react an aminoquinoline having the formula (XLV) in which R₁ₐ, R_{1b}, R₂, Y₁, n, n′, p and p′ are as defined above and U represents a thiol function, with a streptogramin residue having the formula (LXVII): 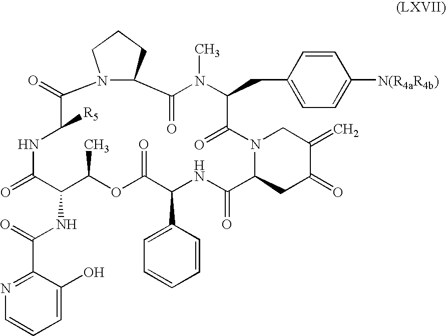
in which R₄ₐ, R_{4b} and R₅ are as defined above.

This coupling reaction is advantageously carried out in an organic solvent (such as acetone) and at low temperature (−20° C. for example).

Aminoquinoline-Diaminopyrimidine Hybrid Molecules

It is advantageous to prepare hybrid molecules containing a 4-aminoquinoline having the formula (XXXIIIa) as Q derivative and a diaminopyrimidine having the formula (XXV) as residue A, in which R₅ is as defined herein before, in the following manner: a-7) coupling of an aminoquinoline having the formula (LVI) in which R₁ₐ, R_{1b}, R₂, Y₁, n, n′ and p are as defined above and “hal” represents a halogen atom, with a derivative having the formula (LXVIII) wherein Y₂, p′ and p″ are as defined above, Y₂ containing an oxy function on a terminal carbon thus forming an aldehyde function and U′ representing a reactive form of the U function defined above (such as an hydroxy function):
(U′)_{p′}—(Y₂)_{p″} (LXVIII)
which produces a 4-aminoquinoline having the formula (LXIX) 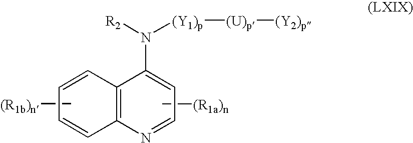
in which R₁ₐ, R_{1b}, R₂, Y₁, Y₂, n, n′, p, p′ and p″ are as defined above and U represents an ether function,

b-7) the aminoquinoline having the formula (LXIX) containing an aldehyde function can then be condensed on a nitrile derivative having the formula (LXX) in which R₅ is as defined previously: 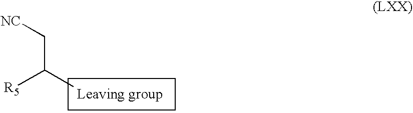
which produces an acrylonitrile intermediate having the formula (LXXI) in which R₁ₐ, R_{1b}, R₂, R₅, Y₁, Y₂, n, n′, p, p′ and p″ are as defined above and U represents an ether function,

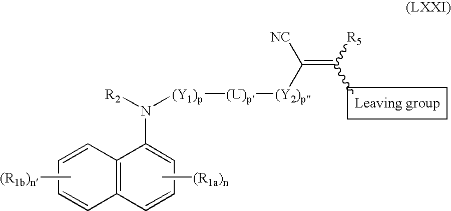

The acrylonitrile intermediate (LXXI) is obtained as a mixture of Z and E isomers,

c-7) the cyclization of the mixture of Z and E isomers of the acrylonitrile intermediate (LXXI) with guanidine leads to aminoquinoline-diaminopyrimidine hybrid molecules having the formula (XXXIX) in which p=p′=p″=1 and U represents an ether function. Step a-7) is advantageously carried out in an amide solvent (such as dimethylformamide) in the presence of a base (e.g. potassium carbonate) and at a moderate temperature (e.g. 60° C.)

Step b-7) is advantageously carried out in an organic solvent (for example dimethylsulfoxide) in the presence of a base (such as potassium tert-butoxide) added in small portions at low temperature (10° C. for example) followed by an stirring at room temperature.

Step c-7) is advantageously carried out in two stages:
the guanidine is put in the presence of a base (such as potassium tert-butoxide) in an alcoholic solvent (e.g. ethanol) at room temperature. The suspension obtained is advantageously filtered on an inert support (celite for example),
the suspension obtained is then filtred ant the filtrate is put in the presence of the mixture of Z and E isomers of the acrylonitrile intermediate (LXXI) in an alcoholic solvent (for example ethanol) at room temperature followed by a reflux during 7 hr.
   Aminoquinoline-Macrolide Hybrid Molecules

In another method, to prepare aminoquinoline-macrolide hybrid molecules having the formula (XLa) in which p″=0, U representing an oxyimine function, it is advantageous to react an aminoquinoline having the formula (LVI) in which R₁ₐ, R_{1b}, R₂, Y₁, n, n′ and p are as defined above and “hal” represents a halogen atom, with a macrolide residue having the formula (LXXII) in which R₃, R₆, R₇, R₁₀ and p′ are as defined above and U is an oxime function:

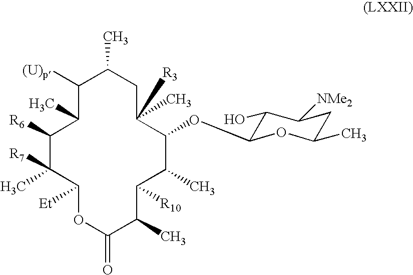

This coupling reaction is advantageously carried out in an amide solvent (such as dimethylformamide) in the presence of a base (ground sodium hydroxide for example) at room temperature.

Aminoquinoline-Glycopeptide Hybrid Molecules

In another method, to prepare aminoquinoline-glycopeptide hybrid molecules having the formula (XLIa) in which p=p′=p″=1, U representing an ether function, it is advantageous to:
a-8) react an aminoquinoline having the formula (LXIX) in which R₁ₐ, R_{1b}, R₂, Y₁, Y₂, U, n, n′, p, p′ and p″ are as defined above, Y₂ containing an oxy function on a terminal carbon thus forming an aldehyde function, with a glycopeptide residue having the formula (LXXIII) in which R₃ and R₄ are as defined above:

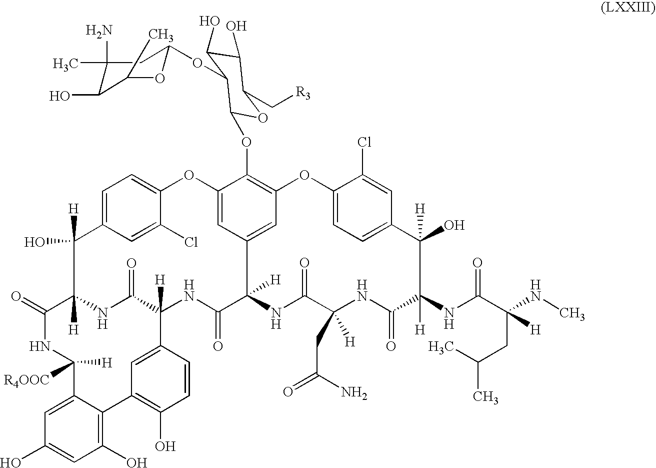

The coupling reaction a-8) is advantageously carried out by firstly putting the glycopeptide in the presence of a base (diisopropylethylamine for example) in an amide solvent (such as dimethylformamide or dimethylacetamide), at room temperature followed by stirring at 70° C. for 2 hr. To this mixture, a solution of a reducing agent (such as sodium cyanoborohydride) in an alcoholic solvent (methanol for example) is then added at 70° C. The mixture is advantageously left under stirring for 2 hr 30 at 70° C. then 20 hr at room temperature.

In the same way, for the preparation of aminoquinoline-glycopeptide hybrid molecules having the formula (XLIA) in which p′=p″=0, it is advantageous to proceed in the following manner:

a-9) react a compound having the formula (XLIII) in which R₁ₐ, R_{1b}, n and n′ are as defined above and “hal” represents a halogen atom, with an amine having the formula (XLIV) wherein R₂, Y₁ and p are as defined above, p′=0 and Y₁ containing on one of its carbon atoms two ether functions (thus forming an acetal function) to afford a 4-aminoquinoline of formula (XLV) in which R₁ₐ, R_{1b}, R₂, Y₁, n, n′ and p are as defined above, p′=0 and Y₁ containing on one of its carbon atoms two ether functions (thus forming an acetal function).

b-9) the latter 4-aminoquinoline of formula (XLV) containing an acetal function is hydrolyzed in acidic medium which produces 4-aminoquinolines having the formula (XLV) in which R₁ₐ, R_{1b}, R₂, Y₁, n, n′ and p are as defined above, p′=0 and Y₁ containing an oxy function on its terminal carbon thus forming an aldehyde function.

c-9) the latter 4-aminoquinoline of formula (XLV) containing an aldehyde function is reacted with a glycopeptide residue having the formula (LXXIII) in which R₃ and R₄ are as defined above.

Step a-9) is advantageously carried out without solvent at hot temperature (such as 110° C.).

The acid hydrolysis b-9) is advantageously carried out in an aqueous solution of acetic acid in the presence of trifluoroacetic acid at moderate temperature (e.g. 70° C.).

The coupling reaction c-9) is advantageously carried out according to the conditions described for the coupling reaction a-8) between the 4-aminoquinoline (LXIX) and the glycopeptide (LXXIII).

Aminoquinoline Oxazolidinone Hybrid Molecules

In another method, to prepare aminoquinoline-oxazolidinone hybrid molecules having the formula (XLIIa) in which p=p′=p″=1, U representing a carbamate function, it is advantageous to react an aminoquinoline having the formula (XLV) in which R₁ₐ, R_{1b}, R₂, Y₁, n, n′, p and p′ are as defined above and U represents an amine function, with a oxazolidinone residue having the formula (LXXIV): 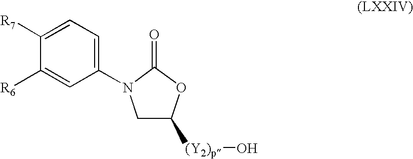
in which R₆, R₇, Y₂ and p″ are as defined above. This coupling reaction is advantageously carried out in a chlorinated solvent (such as dichloromethane) in the presence of triphosgene and a base (triethylamine for example) at room temperature.

In another method to prepare aminoquinoline-oxazolidinone hybrid molecules having the formula (XLIIa) in which p=p′=p″=1, U representing a carbamate function, it is advantageous to react an aminoquinoline having the formula (XLV) in which R₁ₐ, R_{1b}, R₂, Y₁, n, n′, p and p′ are as defined above and U represents a carboxy function, with an oxazolidinone residue having the formula (LXXV): 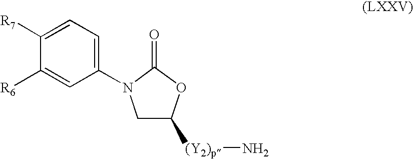
in which R₆, R₇, Y₂ and p″ are as defined above. This coupling reaction is advantageously carried out in an amide solvent (such as dimethylformamide) in the presence of an activator for the U function (for example PyBOP) and of a base (such as N-methylmorpholine) at room temperature.

In the same way, to prepare aminoquinoline-oxazolidinone hybrid molecules having the formula (XLIIb) in which p=p′=p″=1, U representing a carbamate function, it is advantageous to react an oxazolidinone residue having the formula (LXXIV) in which R₆, R₇, Y₂ and p″ are as defined above, with a 2-aminoquinoline having the formula (LXXVI): 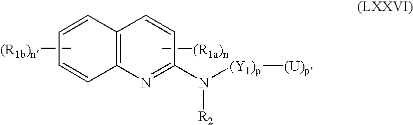
in which R₁ₐ, R_{1b}, R₂, Y₁, n, n′, p, p′ and p″ are as defined above and U represents an amine function, This coupling reaction is advantageously carried out in a chlorinated solvent (such as dichloromethane) in the presence of triphosgene and a base (triethylamine for example) at room temperature.

In another method, to prepare hybrid molecules of formula (XLIIc) wherein R₁ₐ, R_{1b}, R₂, R₃, R₆, Y₁, U, n, n′, p and p′ are as defined above, p″=0 and Y₁ being a linear, branched or cyclic C1, C2, C3, C4, C5 or C6 alkyl chain containing an amide function, it is advantageous to react an aminoquinoline of formula (LXXVII): 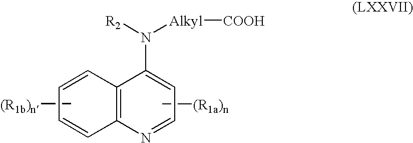
wherein R₁ₐ, R_{1b}, R₂, n and n′ are as defined above with an oxazolidinone of formula (LXXVIII): 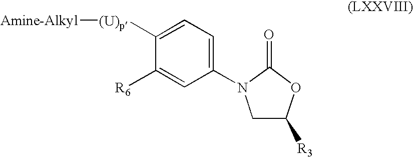
wherein R₃, R₆, U and p′ are as defined above. In the formulae (LXXVII) and (LXXVIII), the alkyl groups are linear, branched or cyclic C1, C2, C3, C4, C5 or C6 alkyl chains.

The reaction is advantageously carried out in a dried organic solvent (e.g. anhydrous dimethylformamide) with an activator of the carboxylic function (such as PyBOP) and with a base (such as N-Methylmorpholine).

In the same way to prepare hybrid molecules of formula (XLIIc) wherein R₁ₐ, R_{1b}, R₂, R₃, R₆, Y₁, U, n, n′, p and p′ are as defined above, p″=0 and Y₁ being a linear, branched or cyclic C1, C2, C3, C4, C5 or C6 alkyl chain containing an amine function, it is advantageous to react an aminoquinoline of formula (LXIV) wherein R₁ₐ, R_{1b}, R₂, n, n′ and Alkyl group are as defined above and hal representing an halogen atom with an oxazolidinone of formula (LXXVIII) wherein R₃, R₆, U, p′ and Alkyl group are as defined above.

The reaction is advantageously carried out in a dried organic solvent (e.g. anhydrous dimethylformamide) with a base (such as triethylamine) and at room temperature.

In another method, hybrid molecules of formula (XLIIc) wherein R₁ₐ, R_{1b}, R₂, R₃, R₆, Y₁, U, n, n′, p and p′ are as defined above and p″=0, may be advantageously prepare by reacting a quinoline of formula (XLIII) in which R₁ₐ and R_{1b}, n and n′ are as defined above and “hal” represents an halogen atom, with an oxazolidinone derivative having the formula (LXXIX): 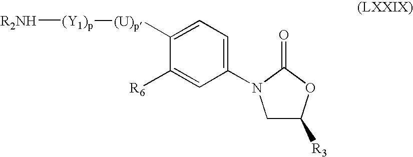
wherein R₂, R₃, R₆, Y₁, U, p and p′ are as defined above.

The coupling reaction is advantageously carried out in an organic solvent (such as 2-ethoxyethanol) and at hot temperature (e.g. at the temperature of reflux of 2-ethoxyethanol).

In order to obtain the hybrid molecules as an acid addition salt, the basic nitrogens are protonated by adding a pharmacologically acceptable acid. Salts formed with inorganic acids (hydrochlorides, hydrobromides, sulfates, nitrates, phosphates) or with organic acids (citrates, tartrates, fumarates, lactates) can be cited as examples of addition salts with pharmacologically acceptable acids. The reaction can be carried out with 2 equivalents of acid added at 0° C.

The compounds having the formula (I) can also be converted into metal salts or addition salts with nitrogen-containing bases according to methods known per se. Salts formed with alkali metals (sodium, potassium, lithium), or with alkaline-earth metals (magnesium, calcium), the ammonium salt or salts of nitrogen-containing bases (triethylamine, diisopropylamine, ethanolamine, procaine, N-benzyl-2-phenylethylamine, tris(hydroxymethyl)-amino-methane, N,N′-dibenzylethylnediamine), can be cited as examples of pharmacologically acceptable salts.

The invention also covers the prodrugs of the hybrid molecules having the formula (I) which are hydrolyzed in vivo to release the active molecule. These prodrugs were prepared by the conventional techniques known to the person skilled in the art.

Advantageously, the invention covers the use of a compound Q as defined previously to covalently bind, for example via a —(Y₁)ₚ—(U)_{p′}—(Y₂)_{p″}— bond as previously defined, a previously defined antibiotic residue A.

Pharmaceutical Uses

In this part: the invention covers the pharmaceutical use of a compound according to the present invention as defined by the formula I. The invention also covers the pharmaceutical use of the excluded compounds 1) except for the disinfection or the treatment of infections due to Mycoplasma sp.

The invention covers the use of a compound as defined above for the manufacture of a pharmaceutical composition, which is intended notably for treating a bacterial infection of an animal, or of a human being or of a treatment of medical material which is contaminated by bacteria, notably of an infection or a bacterial contamination due to Staphylococcus aureus, for example Staphylococcus aureus MSSA (methicillin-sensitive), Staphylococcus aureus MSRA (methicillin-resistant), Staphylococcus aureus NorA (quinolone resistant by efflux), Staphylococcus aureus MsrA (macrolide-resistant by efflux) or Staphylococcus aureus VISA (or GISA) (vancomycin-resistant), Staphylococcus epidermidis for example Staphylococcus epidermidis MSCNS (methicillin-sensitive coagulase negative) or Staphylococcus epidermidis MRCNS (methicillin-resistant coagulase negative), Streptococcus pneumoniae, for example Streptococcus pneumoniae PSSP (penicillin-sensitive) or Streptococcus pneumoniae PRSP (penicillin resistant), Streptococcus pneumoniae mefE (macrolide-resistant by efflux), Streptococcus pyogenes, Enterococcus faecalis, for example Enterococcus faecalis VSE (vancomycin-sensible) or Enterococcus faecalis VRE (vancomycin-resistant), Enterococcus faecium, for example Enterococcus faecium VSE (vancomycin-sensible) or Enterococcus faecium VRE (vancomycin-resistant), Haemophilus influenzae, Moraxella catarrhalis, Escherichia coli; Pseudomonas aeruginosa, Bacillus subtils, Bacillus thuringiensis, Clostridium difficile or Bacteroides fragilis.

The hybrid molecules of the invention as defined in this part can be very advantageously used for the treatment of bacterial infections due to the germs on which they are active.

Thus, hybrid molecules of the invention which are active on Streptococcus pneumoniae can be very advantageously used for the treatment of infections such as acute pneumonia, meningitis, otitis, or sinusitis.

In the same way, the hybrid molecules of the invention which are active on Staphylococcus aureus can be used for the treatment of infections such as skin and/or mucosal infections, nosocomial infections, or osteomylitis.

In the same way, the hybrid molecules of the invention which are active on Staphylococcus epidermidis can be used for the treatment of infections such as nosocomial and iatrogenic infections due to this bacterium.

Nosocomial, urinary, cutaneous, genital, biliary, dental, and otitis-sinusitis or endocarditis infections due to Enterococcus faecalis can be advantageously treated by the hybrid molecules which are active on this bacteria.

In the same way, the hybrid molecules of the invention which are active on Streptococcus pyogenes can be used for the treatment of infections such as bacterial throat infections, other ORL infections, cutaneous infections, scarlet fever, erysipela, impetigo or subcutaneous gangrene.

In the same way, the hybrid molecules of the invention which are active on Haemophilus influenzae can be used for the treatment of ORL infections, and complications of influenza or meningitis.

In the same way, the hybrid molecules of the invention which are active on Moraxella catarrhalis can be used for the treatment of ORL infections due to this bacteria.

Infections due to Escherichia coli such as urinary and abdominal infections or infantile diarrhea can be advantageously treated by the hybrid molecules that are active on this bacterium.

In the same way, the hybrid molecules of the invention which are active on Bacillus sp. can be used for the treatment of alimentary intoxications due to this bacterium.

Infections due to Bacteroides fragilis such as bacteraemia, abscesses and lesions, peritonitis, endocarditis or wound infections can be advantageously treated by the hybrid molecules that are active on this bacterium.

The invention also therefore covers the application of these hybrid molecules of great interest defined above, to develop drugs destined for the agrifood industry and in human and veterinary medicine for the treatment of a bacterial infection or even as bactericide for industrial applications.

Notably, it is advantageous to deliver an efficient quantity of compound according to the present invention for the previously cited treatments and those cited herein after.

The invention yet covers a method of therapeutic treatment of an animal or of a human being having a need for it, wherein it comprises the administration to this subject of a therapeutically efficient quantity of a hybrid compound according to the invention having the previously cited formula (I).

Specific embodiments of this treatment clearly result for the person skilled in the art of the activity of the antibiotics concerned and of the description of the invention taken as a whole including the examples that form an integral part of it. The study of the pharmacological properties of the hybrid molecules having the formula (XXXIVa), (XXXIVc), (XXXVa), (XXXVb), (XXXVd), (XXXVf), (XXXVIa), (XXXVII), (XXXVIII), (XXXIX), (XLa), (XLIa), (XLIIa), (XLIIb) and (XLIIc) given as examples has shown that these hybrid molecules are particularly interesting antimicrobial agents, their antibacterial activity is very high and perfectly unexpected to the person skilled in the art.

Aminoquinoline-β-Lactam Hybrid Molecules

The aminoquinoline-β-lactam hybrid molecules having the formula (XXXIVa), (XXXIVc), (XXXVa) and (XXXVb) have very high antibacterial activity, in particular on Gram+ germs.

For example, the aminoquinoline-penicillin hybrid molecule PA 1007 (example 1), called “peniciquine”, presents antibacterial activity at the same level as that of penicillin G. Given that PA 1007 is a prodrug, this result leads us to predict an excellent activity in vivo after hydrolysis of the ester function by the host enzymes.

The aminoquinoline-cephalosporin hybrid molecules, called “cephaloquines”, are very active in vitro on Staphylococcus aureus, penicillin-sensitive Streptococcus pneumoniae and Streptococcus pyogenes at minimal inhibitory concentrations (MIC) comprised between 0.0015 and 0.78 μg/mL. Even more interesting is the activity of certain of them on two strains of penicillin-resistant Streptococcus pneumoniae PRSP (CIP 104471 and a clinical isolate) at concentrations comprised between 0.006 and 6.25 μg/mL for the MIC and between 0.025 and 12.5 μg/mL for MBC (minimum bactericidal concentration). The most active molecule (MIC: 0.006 μg/mL) proved to be 8 times more effective than ceftriaxone (MIC: 0.05 μg/mL), tested on the same stains. Ceftriaxone is one of the antibiotics which are currently used for treating cases of pneumonia which are due to penicillin-resistant S. pneumoniae germs. The hybrid molecules that have interesting activity on S. pneumoniae PRSP (MIC from 0.006 to 0.39 μg/mL) were also shown to be active on Haemophilus influenzae, another germ responsible for pneumonia, with MIC from 0.78 to 3.12 μg/mL (see example 50, tables III and IV).

The amplification effect of the antibiotic activity of the hybrid molecules is clearly shown by a study of activity of the constituent Q and A structures from an example of aminoquinoline-β-lactam hybrid molecule compared to a 1/1 combination of its sub-structures.

The results are remarkable and perfectly exemplify this amplification: only the hybrid molecule has interesting antibacterial activity. The covalent bond between the two parts therefore brings an important and perfectly unexpected effect for the person skilled in the art (example 50, table V).

Moreover, it has been shown that in the presence of human serum, the aminoquinoline-cephalosporin hybrid molecules such as those tested as examples remained active in vitro not only on S. aureus but also on S. pneumoniae PRSP. In the same conditions ceftriaxone totally loses its antibacterial activity because of its strong binding to the proteins of the serum that is well known to the person skilled in the art (example 50, table VI).

Additionally, a stability study of the hybrid molecules in solution has shown that they were stable not only at physiological pH, pH 7 in solution at 37° C. but also in acidic medium pH 1 (equivalent to the pH of the stomach). To give an example, the half life of the molecule that is the most active on penicillin-resistant S. pneumoniae is 15 hr at pH 1 in solution at 37° C. where ceftriaxone is practically totally degraded in the same conditions in less than 6 hr with a half life less than 2 hr (example 49, tables I and II).

An additional feature of the hybrid molecules of the family of aminoquinoline-β-lactam is their low oral toxicity (100% of survival after two administrations of 100, 200 or 400 mg/kg, table XV example 52) and their non-cytotoxicity determined in a MTT cytotoxicity assay on human lung fibroblast cells at concentrations as high as 500 μg/mL (example 52, table XVI).

Finally, three hybrid molecules of the family aminoquinoline-cephalosporin have been tested in vivo in a murine model of septicaemia due to Staphylococcus aureus MSSA and compared to ceftriaxone. The drugs have been injected subcutaneously at +1 hr and +6 hr post-infection. The excellent antibacterial properties of aminoquinoline-cephalosporin hybrid molecules have been confirmed by this in vivo experiment since calculated ED₅₀ gave for the cephaloquines similar values to two-fold lower values than that of ceftriaxone (example 51, table XIII).

Aminoquinoline-Quinolone Hybrid Molecules

The superiority of the hybrid molecules QA is not limited to the β-lactam family. In fact, examples of aminoquinoline-quinolone hybrid molecules having the formula (XXXVIa) have shown remarkable results in terms of antibacterial activity and this is the case whether on sensitive strains or on resistant strains. Thus the “quinoloquines” PA 1285 (example 27) and PA 1126 (example 24) are very active on sensitive strains such as S. aureus MSSA (methicillin-sensitive) or B. subtilis but also on resistant strains such as S. pneumoniae PRSP, E. faecalis VRE or S. aureus NorA. The activity of PA 1126 on this latter strain is particularly interesting since it is a quinolone resistant strain (MIC of ciprofloxacin>50 μg/mL). With a MIC of 0.18 μg/mL on this same strain, PA 1126 is 280 fold more active than the sub-structure from which it comes (example 50, table VII).

The activity spectrum of the fluoroquinolones is broad. These antibiotics, in spite of their tendency to favor resistance phenomena are essential in the case of emergency or pre and post operative treatments. The quinoloquine PA 1127 (example 25) remains an interesting molecule because it presents a narrow activity spectrum centered on Gram− bacteria.

Aminoquinoline-Nitroimidazole Hybrid Molecules

The activity of aminoquinoline-nitroimidazole hybrid molecules having the formula (XXXVII), such as “nitroimidaquine” PA 1129 (example 28) is of the same level as that of the reference molecule in the nitroimidazole family: metronidazole (example 50, table VIII).

Aminoquinoline-Streptogramin Hybrid Molecules

The aminoquinoline-streptogramin family of hybrid molecules having the formula (XXXVIII) is interesting in the light of its narrow activity spectrum centered on sensitive or resistant Gram+ bacteria. Thus the activity of the aminoquinoline-streptogramin hybrid molecule PA 1182 (example 31), generally called “streptogramiquine” or more specifically called “pristinaquine”, is from 4 to 8 times greater on Gram+ bacteria than that of the antibiotic A of which it is composed (example 50, table IX).

Aminoquinoline-Macrolide Hybrid Molecules

In this family of hybrid molecules having the formula (XLa) called “macroliquines”, exemplified by “erythromyquine” PA 1169 (example 35), the addition of an aminoquinoline to an antibiotic residue from the macrolide family leads to a gain in activity of a factor of 8 on Streptococcus pneumoniae PSSP. Moreover, erythromyquine PA 1169 is active on a strain of Streptococcus pneumoniae that is resistant to macrolides by efflux (MIC of erythromycin: 5 μg/mL, MIC of PA 1169: 1.25 μg/mL) (example 50, table X).

Aminoquinoline-Glycopeptide Hybrid Molecules

The addition of a covalent bond between an aminoquinoline and an antibiotic residue is most remarkable and unexpected on aminoquinoline-glycopeptide hybrid molecules having the formula (XLIa). In fact, on all the tested strains (sensitive or resistant), the antibacterial activity of “vancomyquines” is much superior to that of their constituent sub-structure A: vancomycin. For these hybrid molecules the gain in activity brought by the covalent bond with an aminoquinoline ranges from 4 to 260 (example 50, table XI).

In vivo, hybrid molecules of the family aminoquinoline-glycopeptide have also proved to keep their excellent antibacterial properties. In a murine septicaemia model infection due to Staphylococcus aureus MSSA, PA 1157 necessitates a lower dose (0.09 mg/kg injected twice by subcutaneous route at +1 hr and +6 hr post-infection) compared to vancomycin (0.16 mg/kg injected in the conditions as for PA 1157).

Moreover, examples of this hybrid molecule's family proved to be non-cytotoxic in a MTT cytotoxicity assay on human lung fibroblast cells (example 52, table XVII).

Aminoquinoline-Oxazolidinone Hybrid Molecules

The examples of aminoquinoline-oxazolidinone hybrid molecules having the formula (XLIIa) demonstrate an antibacterial activity equivalent to that of linezolid (the only molecule of this class on the market). It is known to the person skilled in the art that the in vivo activity will be greatly influenced by the pharmacokinetic properties that could be in the case of the aminoquinoline-oxazolidinone hybrid molecules having the formula (XLIIa) better than the reference product (example 50, table XII).

All these properties render the said products of the invention, as well as their salts, hydrates, prodrugs and prodrug salts, able to be used as drugs.

The invention covers compositions, notably by taking advantage of the properties of these hybrid molecules, for the preparation of pharmaceutical compositions.

Notably, the pharmaceutical composition comprises, notably as active principle, at least one compound QA defined above, in a pharmaceutically acceptable excipient.

The pharmaceutical compositions of the invention contain an effective amount of at least one hybrid molecule having the formula (I) as defined above, as well as a pharmaceutically acceptable vehicle. As is known to the person skilled in the art, various forms of excipients can be used adapted to the mode of administration and some of them can promote the effectiveness of the active molecule, e.g. by promoting a release profile rendering this active molecule overall more effective for the treatment desired.

The pharmaceutical compositions of the invention are thus able to be administered in various forms, more specially for example in an injectable, pulverizable or ingestible form, for example via the intramuscular, intravenous, subcutaneous, intradermal, oral, topical, rectal, vaginal, ophthalmic, nasal, transdermal or parenteral route. The present invention notably covers the use of a compound according to the present invention for the manufacture of a composition, particularly a pharmaceutical composition.

Advantageously, the compounds according to the invention can be used in efficient quantities. These quantities are generally comprised between 10 mg and 2 g of active ingredient per day.

The pharmaceutical compositions of the invention contain an effective amount of at least one hybrid molecule having the formula (I) as defined above, and may also contain other pharmacologically active substances. Notably, in the pharmaceutical compositions of the invention, one hybrid molecule QA having the formula (I) can be combined with an resistance enzyme inhibitor such as β-lactamase inhibitors. To give examples of β-lactamase inhibitors that can be cited: clavulanic acid (3-2-hydroxyethylidene)-7-oxo-4-oxa-1-azabicyclo[3.2.0]heptane-2-carboxylic acid), sulbactam sodium (sodium 4,4 dioxide [2S-(2 alpha,5 alpha)]3,3-dimethyl-4,4,7-trioxo-4λ⁶-thia-1-azabicyclo[3,2,0]heptane-2-carboxylate) and tazobactam sodium (sodium [2S-(2 alpha,3,bêta,5 alpha)]-3-methyl-4,4,7-trioxo-3-(1H-[1,2,3]1triazol-1-ylmethyl)-4λ⁶-thia-1-azabicyclo[3,2,0]heptane-2-carboxylate).

The compositions of the invention are particularly appropriate for treating a bacterial infection in man or in an animal or for disinfecting materials, notably medical materials.

The invention is now illustrated by examples which represent currently preferred embodiments which make up a part of the invention but which in no way are to be used to limit the scope of it, the invention being a pioneer within the context of the creation of a novel family of active compounds covalently combining at least one antibiotic and at least one aminoquinoline.

In the examples, all the percentages are given by weight (unless indicated otherwise), the temperature is in degrees Celsius and the pressure is atmospheric pressure, unless indicated otherwise. The chemical products used are commercially available, notably from the Aldrich or Acros companies, unless otherwise indicated.

### EXAMPLES

Examples 1 to 4 Below Exemplify Preparations of Hybrid Molecules of the Family of Aminoquinoline-Penicillins.

### Example 1

### Preparation of an Aminoquinoline-Penicillin, Ref PA 1007

### (2S,5R,6R)-6-{[1-(7-Chloroquinolin-4-yl)-piperidine-4-carbonyl]-amino}-3,3-dimethyl-7-oxo-4-thia-1-aza-bicyclo[3.2.0]heptane-2-carboxylic acid 2,2-dimethyl-propionyloxymethyl ester

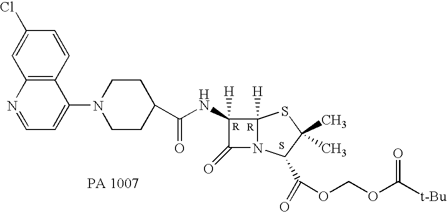

### 1.1. 1-(7-Chloroquinolin-4-yl)-piperidin carboxylic acid

A mixture of 4,7-dichloroquinoline (17.4 g, 0.09 mol), of isonipecotic acid (23.8 g, 0.18 mol) and phenol (46.3 g, 0.49 mol) is heated to 120° C. with stirring over 24 hours. After cooling to room temperature, the reaction medium is diluted with 400 ml of ethyl acetate, filtered over sintered glass and the resulting precipitate is washed with water. This precipitate is then recrystallized by hot dissolution (100° C.) in 300 ml of 10% (w/v) carbonate-containing water and precipitation at 0° C. by addition of a 2M aqueous solution of HCl until pH 5. The precipitate formed is filtered off and then washed successively with water, acetone and diethyl ether before being dried under vacuum. The product is obtained as a white powder (18.4 g, 72%).

¹H NMR (300 MHz, CD₃COOD) δ ppm: 2.11 (2H, dd, J=10.6 Hz, J=13.9 Hz), 2.27 (2H, d, J=13.9 Hz), 2.92 (1H, m), 3.60 (2H, dd, J=10.6 Hz, J=13.4 Hz), 4.20 (2H, d, J=13.4 Hz), 7.19 (1H, d, J=7.0 Hz), 7.65 (1H, dd, J=2.0 Hz, J=9.2 Hz), 8.10 (1H, d, J=9.2 Hz), 8.18 (1H, d, J=2.0 Hz), 8.72 (1H, d, J=7.0 Hz). MS (IS>0) m/z: 291.0 (M+H⁺).

### 1.2. (2S,5R,6R)-6-{[1-(7-Chloroquinolin-4-yl)-piperidin-4-carbonyl]-amino}3,3-dimethyl-7-oxo-4-thia-1-aza-bicyclo[3.2.0]heptane-2-carboxylic acid 2,2-dimethyl-propionyl-oxymethyl ester

3.6 mL of N-methylmorpholine (32.7 mmol) are added to a mixture of 1-(7-chloro-quinolin-4-yl)-piperidine-carboxylic acid (Example 1.1) (3.0 g, 10.3 mmol) and 6-aminopenicillanic acid pivaloyloxymethyl ester tosylate salt POM-APA-Ts (prepared according to the method described by R.-de-Vains et al., Tetrahedron Lett. 2001, 42, 7033-7036) (5.2 g, 10.3 mmol) in 75 mL of DMF. The suspension is left under stirring for 15 minutes before adding the activator PyBOP® (5.4 g, 10.3 mmol). The stirring is continued for 24 hours at room temperature. The reaction medium is then diluted with 100 mL of dichloromethane and washed successively with 100 ml of 10% (w/v) carbonate-containing water, twice 100 ml of water and 100 ml of water saturated with NaCl. The organic phase is dried over magnesium sulfate, filtered and then evaporated. The oil obtained is purified by liquid chromatography on silica gel (SiO₂ 60A C.C 70-200 μm, eluent: ethyl acetate). The cleanest fractions according to TLC revealed under UV are evaporated. PA 1007 is obtained after recrystallization from chloroform/n-hexane as a white powder (1.4 g, 23%).

IR (KBr) cm⁻¹: (C═O) 1786, 1757, 1681, ¹H NMR (300 MHz, CDCl₃) δ ppm: 1.22 (9H, s), 1.53 (3H, s), 1.65 (3H, s), 2.13 (4H, m), 2.43 (1H, m), 2.84 (2H, dd, J=11.4 Hz, J=12.3 Hz), 3.60 (2H, d, J=12.3 Hz), 4.44 (1H, s), 5.58 (1H, d, J=4.0 Hz), 5.75 (1H, dd, J=4.0 Hz, J=8.7 Hz), 5.77 (1H, d, J=5.7 Hz), 5.88 (1H, d, J=5.7 Hz), 6.57 (1H, d, J=8.7 Hz), 6.80 (1H, d, J=5.1 Hz), 7.41 (1H, dd, J=2.0 Hz, J=9.0 Hz), 7.89 (1H, d, J=9.0 Hz), 8.02 (1H, d, J=2.0 Hz), 8.69 (1H, d, J=5.1 Hz). MS (IS>0) m/z: 603.2 (M+H⁺). Elementary analysis: for C₂₉H₃₅ClN₄O₆S 0.5H₂O: % theor. C 56.90, N 9.15; % exper. C 56.80, N 8.83.

### Example 2

### Preparation of an Aminoquinoline-Penicillin, Ref PA 1008

### (2S,5R,6R)-3,3-Dimethyl-7-oxo-6-[3-(quinolin-8-ylamino)-propionyl-amino] thia-1-aza-bicyclo[3.2.0]heptane-2-carboxylic acid 2,2-dimethyl-propionyloxymethyl ester

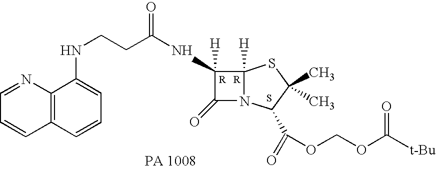

PA 1008 is prepared according to the procedure described in Example 1.2 from 4.3 g of 3-(quinolin-8-ylamino)propionic acid (19.9 mmol) (prepared according to the method described by Z. J. Beresnevicius et al., Chem. Heterocycl. Comp. 2000, 36, 432-438), 10.0 g of POM-APA-Ts (19.9 mmol), 6.5 mL of N-methylmorpholine (59.1 mmol) and 10.3 g of PyBOP® (19.9 mmol). After purification by liquid chromatography on silica gel (SiO₂ 60A C.C 6-35 μm, eluent: n-hexane/ethyl acetate 55/45 v/v) and recrystallization from diethyl ether/n-hexane PA 1008 is obtained as a yellow powder (2.3 g, 22%).

IR (KBr) cm⁻¹: (C═O) 1784, 1755, 1667. ¹H NMR (300 MHz, 298K, CDCl₃) 6, ppm: 1.16 (9H, s), 1.37 (6H, s), 2.64 (2H, t, J=6.6 Hz), 3.61 (2H, m), 4.34 (1H, s), 5.45 (1H, d, J=4.2 Hz), 5.67 (1H, dd, J=4.2 Hz, J=8.7 Hz), 5.70 (1H, d, J=5.4 Hz), 5.80 (1H, d, J=5.4 Hz), 6.34 (1H, broad s), 6.67 (1H, d, J=7.5 Hz), 7.03 (1H, d, J=8.4 Hz), 7.09 (1H, d, J=8.7 Hz), 7.30 (1H, dd, J=4.2 Hz, J=8.1 Hz), 7.32 (1H, dd, =7.5 Hz, J=8.4 Hz), 7.99 (1H, dd, J=1.5 Hz, J=8.1 Hz) 8.66 (1H, dd, J=1.5 Hz, =4.2 Hz). MS (IS>0) m/z: 529.2 (M+H⁺). Elementary analysis: for C₂₆H₃₂N₄O₆S: % theor. C 59.07, N 10.60; % exper. C 59.19, N 10.50.

### Example 3

### Preparation of an Aminoquinoline-Penicillin, Ref PA 1012

### (2S,5R,6R)-6-[2-(7-Chloroquinolin-4-ylamino)-acetylamino]-3,3-dimethyl-7-oxo thia-1-aza-bicyclo[3.2.0]heptane-2-carboxylic acid 2,2-dimethyl-propionyloxymethyl ester

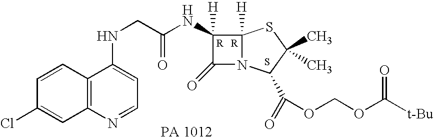

### 3.1. (7-Chloroquinolin-4-ylamino)acetic acid, Ref. PA 1117.

This compound is prepared by modification of the method described by E. O. Titus et al. (J. Org. Chem. 1948, 13, 61). A mixture of 4,7-dichloroquinoline (30.0 g, 0.15 mol), glycine (25.0 g, 0.33 mol) and phenol (80.0 g, 0.85 mol) is heated to 120° C. under stirring over 24 hours. After cooling to room temperature, the reaction medium is diluted with 1 L of diethyl ether and extracted with 1 L of 10% (w/v) carbonate-containing water. The hot aqueous phase (100° C.) is passed over Norit A charcoal, filtered and then brought to pH 5 at 0° C. with a 2 M aqueous solution of HCl. The precipitate formed is filtered off and washed successively with water, acetone and diethyl ether before being dried under vacuum. PA 1117 is obtained as a white powder (27.0 g, 75%).

¹H NMR (300 MHz, CF₃COOD) δ ppm: 4.51 (2H, s), 6.72 (1H, d, J=6.9 Hz), 7.68 (1H, d, J=9.0 Hz), 7.87 (1H, s), 8.10 (1H, d, J=9.0 Hz), 8.30 (1H, d, J=6.9 Hz).

### 3.2. (2S,5R,6R)-6-[2-(7-Chloroquinolin-4-ylamino)acetylamino]-3,3-dimethyl-7-oxo-4-thia-1-aza-bicyclo[3.2.0]heptane-2-carboxylic acid 2,2-dimethyl-propionyloxymethyl ester

PA 1012 is prepared according to the procedure described in Example 1.2 from 1.3 g of (7-chloroquinolin-4-ylamino)-acetic acid (Example 3.1) (5.6 mmol), 2.8 g of POM-APA-Ts (5.6 mmol), 1.8 mL of N-methylmorpholine (16.4 mmol) and 2.9 g of PyBOP® (5.6 mmol). PA 1012 is obtained after purification by liquid chromatography on silica gel (SiO₂ 60A C.C 70-200 μm, eluent: ethyl acetate/chloroform 8/2 v/v) and recrystallization from chloroform/n-hexane as a white powder (0.3 g, 11%).

IR (KBr) cm⁻¹: (C═O) 1784, 1759, 1669. ¹H NMR (300 MHz, CDCl₃) δ ppm: 1.20 (9H, s), 1.39 (3H, s), 1.44 (3H, s), 4.04 (2H, broad s), 4.39 (1H, s), 5.57 (1H, d, J=4.2 Hz), 5.74 (1H, dd, J=4.2 Hz, J=9.0 Hz), 5.75 (1H, d, J=5.4 Hz), 5.85 (1H, d, J=5.4 Hz), 6.21 (1H, broad s), 6.29 (1H, d, J=6.0 Hz), 7.36 (1H, dd, J=1.8 Hz, J=9.0 Hz), 7.53 (1H, d, J=9.0 Hz), 7.77 (1H, d, J=9.0 Hz), 7.95 (1H, d, J=1.8 Hz), 8.51 (1H, d, J=6.0 Hz). MS (IS>0) m/z: 549.2 (M+H⁺). Elementary analysis: for C₂₅H₂₉ClN₄O₆S.1.5H₂O: % theor. C 52.12, N 9.72; % exper. C 52.41, N 9.39.

### Example 4

### Preparation of an Aminoquinoline-Penicillin, Ref PA 1013

### (2S,5R,6R)-6-[3-(7-Chloroquinolin-4-ylamino)-propionylamino]-3,3-dimethyl-7-oxo-4-thia-1-aza-bicyclo[3.2.0]heptane-2-carboxylic acid 2,2-dimethyl-propionyloxymethyl ester

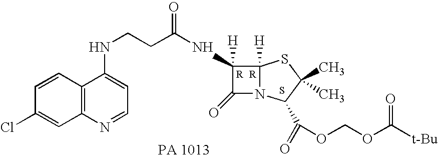

### 4.1. 3-(7-Chloroquinolin-4-ylamino)propionic acid

This compound is prepared by modification of the method described by W. J. Humphlett et al. (J. Am. Chem. Soc. 1951, 73, 61), according to the procedure described in Example 3.2 and from 25.1 g of 4,7-dichloroquinoline (0.13 mol), 23.6 g of β-alanine (0.26 mol) and 66.5 g of phenol (0.71 mol). The product is obtained as a white powder (19.5 g, 62%).

¹H NMR (300 MHz, CF₃COOD) δ ppm: 2.90 (2H, t, J=6.0 Hz), 3.86 (2H, t, J=6.0 Hz), 6.73 (1H, d, J=7.2 Hz), 7.53 (1H, dd, J=1.5 Hz, J=9.0 Hz), 7.72 (1H, d, J=1.5 Hz), 7.96 (1H, d, J=9.0 Hz), 8.14 (1H, d, J=7.2 Hz).

### 4.2. (2S,5R,6R)-6-[3-(7-Chloroquinolin-4-ylamino)-propionylamino]-3,3-dimethyl-7-oxo-4-thia-1-aza-bicyclo[3.2.0]heptane-2-carboxylic acid 2,2-dimethyl-propionyloxymethyl ester

PA 1013 is prepared according to the procedure described in Example 1.2 from 2.2 g of 3-(7-chloroquinolin-4-ylamino)-propionic acid (Example 4.1) (8.0 mmol), 4.1 g of POM-APA-Ts (8.0 mmol), 2.6 mL of N-methylmorpholine (23.6 mmol) and 4.1 g of PyBOP® (8.0 mmol). After several recrystallizations from chloroform/n-hexane, PA 1013 is obtained as a white powder (1.2 g, 27%).

IR (KBr) cm⁻¹: (C═O) 1787, 1760, 1662. ¹H NMR (300 MHz, CDCl₃) δ ppm: 1.23 (9H, s), 1.48 (3H, s), 1.53 (3H, s), 2.73 (2H, m), 3.69 (2H, m), 4.42 (1H, s), 5.55 (1H, d, J=4.2 Hz), 5.71 (1H, dd, J=4.2 Hz, J=8.7 Hz), 5.77 (1H, d, J=5.7 Hz), 5.87 (1H, d, J=5.7 Hz), 6.37 (1H, d, J=5.4 Hz), 6.75 (1H, broad s), 7.37 (1H, dd, =1.8 Hz, J=9.0 Hz), 7.76 (1H, d, J=9.0 Hz), 7.93 (1H, d, J=1.8 Hz), 8.46 (1H, d, =5.4 Hz). MS (IS>0) m/z: 563.3 (M+H⁺). Elementary analysis: for C₂₆H₃₁ClN₄O₆S.0.5H₂O: % theor. C 54.58, N 9.79; % exper. C 54.41, N 9.84.

Examples 5 to 22 exemplify preparations of hybrid molecules of the family of aminoquinoline-cephalosporins.

### Example 5

### Preparation of an Aminoquinoline-Cephalosporin, Ref PA 1046 (6R,7R)-3-Acetoxymethyl-7-[2-(7-chloroquinolin-4-ylamino)-acetylamino]-8-oxo-5-thia-1-aza-bicyclo[4.2.0]oct-2-ene-2-carboxylic acid

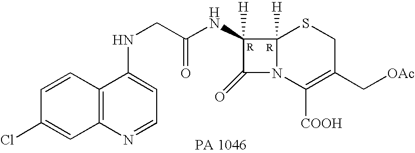

### 5.1. Mixture of (6R,7R-3-acetoxymethyl-7-[2-(7-chloroquinolin-4-ylamino)-acetylamino]-8-oxo-5-thia-1-aza-bicyclo[4.2.0]oct-2-ene-2-carboxylic acid benzhydryl ester and (6R,7R)-3-acetoxymethyl-7-[2-(7-chloroquinolin-4-ylamino)-acetylamino]-8-oxo-5-thia-1-aza-bicyclo-[4.2.0]oct-3-ene-2-carboxylic acid benzhydryl ester: Δ²/Δ³

1-hydroxybenzotriazole HOBT (1.4 g, 10.4 mmol) and N,N′-dicyclohexylcarbodiimide DCC (2.1 g, 10.4 mmol) are added successively to a suspension of (7-chloro-quinolin-4-ylamino)-acetic acid (Example 3.1, PA 1117) (2.9 g, 10.0 mmol) in 80 mL of DMF. The mixture is left under stirring for 30 minutes before adding (6R,7R)-3-acetoxymethyl-7-amino-8-oxo-5-thia-1-aza-bicyclo[4.2.0]oct-2-ene-2-carboxylic acid benzhydryl ester p-toluenesulfonic acid (prepared according to the procedure described by R. G. Micetich et al. Synthesis 1985, 6-7, 693-695) (6.1 g, 10.0 mmol) followed by triethylamine (2.7 mL, 20.0 mmol). The stirring is continued for 24 hours at room temperature. The reaction medium is then diluted with 400 ml of ethyl acetate and then filtered. The filtrate is washed successively with 400 ml of 10% (w/v) carbonate-containing water, twice 400 ml of water and 400 ml of water saturated with NaCl. The organic phase is dried over magnesium sulfate, filtered and then evaporated. The oil obtained is purified by liquid chromatography on silica gel (SiO₂ 60A C.C 6-35 μm, eluent: dichloromethane/ethanol 90/10 v/v). The cleanest fractions according to TLC revealed under UV are evaporated. The product of coupling is obtained as an orangey powder (3.2 g, 48%) as a Δ²/Δ³ 37/63 mixture, used as such in the following step.

### 5.2. (6R,7R-3-Acetoxymethyl-7-[2-(7-chloroquinolin-4-ylamino)-acetylamino]-5,8-dioxo-5λ⁴-thia-1-aza-bicyclo[4.2.0]oct-2-ene-2-carboxylic acid benzhydryl ester

A solution of 3-chloroperoxybenzoic acid (2.6 g, 15.1 mmol) in 250 mL of dichloromethane is added dropwise, over a period of 3 hours, to a solution of the Δ²/Δ³ mixture of Example 5.1 (5.1 g, 7.8 mmol) in 200 mL of dichloromethane, at 0° C. The reaction medium is then washed with a mixture of 400 ml of 5% (w/v) carbonate-containing water and 250 ml of a 6% (w/v) aqueous solution of sodium sulfite. The organic phase is dried over magnesium sulfate, filtered and then evaporated. The powder obtained is washed with ethyl acetate under stirring for 30 minutes, filtered, washed with diethyl ether and dried under vacuum. The oxidation product is obtained as a yellow powder (4.0 g, 76%).

IR (KBr) cm⁻¹: (C═O) 1788, 1738, 1663. ¹H NMR (300 MHz, DMSO) δ ppm: 1.95 (3H, s), 3.60 (1H, d, J=18.9 Hz), 3.93 (1H, d, J=18.9 Hz), 4.11 (2H, m), 4.58 (1H, d, J=13.2 Hz), 4.95 (1H, d, J=4.5 Hz), 5.02 (1H, d, J=13.2 Hz), 6.03 (1H, dd, J=4.5 Hz, J=8.1 Hz), 6.39 (1H, d, J=5.4 Hz), 6.94 (1H, s), 7.28-7.52 (1H, m), 7.83 (2H, broad s), 8.23 (1H, d, J=9.0 Hz), 8.34 (1H, d, J=8.1 Hz), 8.44 (1H, d, J=5.4 Hz). MS (IS>0) m/z: 673.1 (M+H⁺).

### 5.3. (6R,7R)-3-Acetoxymethyl-7-[2-(7-chloroquinolin-4-ylamino)-acetylamino]-8-oxo-5-thia-1-aza-bicyclo[4.2.0]oct-2-ene-2-carboxylic acid benzhydryl ester

1.1 mL of trichlorophosphine (12.6 mmol) is added dropwise to a solution, at −20° C. under argon, of (6R,7R)-3-acetoxymethyl-7-[2-(7-chloroquinolin-4-ylamino)-acetylamino]-5,8-dioxo-5λ⁴-thia-1-aza-bicyclo[4.2.0]-oct-2-ene-2-carboxylic acid benzhydryl ester (Example 5.2) (3.8 g, 5.6 mmol) in 40 mL of dry DMF. The reaction is left under stirring for 1 hour at −20° C. The reaction medium is then diluted with 150 mL of dichloromethane and washed successively with twice 150 ml of water and 150 ml of water saturated with NaCl. The organic phase is dried over magnesium sulfate, filtered and then evaporated. After recrystallization from dichloromethane/diethyl ether, the product is obtained as a beige powder (1.7 g, 46%).

IR (KBr) cm⁻¹: (C═O) 1785, 1735, 1689. ¹H NMR (300 MHz, DMSO) δ ppm: 1.96 (3H, s), 3.56 (1H, d, J=18.3 Hz), 3.69 (1H, d, J=18.3 Hz), 4.37 (2H, m), 4.64 (1H, d, J=13.2 Hz), 4.86 (1H, d, J=13.2 Hz), 5.16 (1H, d, J=5.1 Hz), 5.83 (1H, dd, J=5.1 Hz, J=8.4 Hz), 6.71 (1H, d, J=7.2 Hz), 6.93 (1H, s), 7.27-7.49 (10H, m), 7.82 (1H, dd, J=1.8 Hz, J=9.0 Hz), 8.08 (1H, d, J=1.8 Hz), 8.57 (1H, d, J=9.0 Hz), 8.61 (1H, d, J=7.2 Hz), 9.38 (1H, d, J=8.4 Hz), 9.74 (1H, broad s). MS (IS>0) m/z: 657.2 (M+H⁺).

### 5.4. (6R,7R-3-Acetoxymethyl-7-[2-(7-chloroquinolin-4-ylamino)acetylamino]-8-oxo-5-thia-1-aza-bicyclo[4.2.0]oct-2-ene-2-carboxylic acid

0.8 ml of anisole (7.5 mmol), followed by 1.4 ml of trifluoroacetic acid injected dropwise (19.0 mmol), is added to a solution of (6R,7R-3-acetoxymethyl-7-[2-7-chloro-quinolin-4-ylamino)acetylamino]-8-oxo-5-thia-1-aza-bicyclo[4.2.0] oct-2-ene-2-carboxylic acid benzhydryl ester (1.3 g, 1.9 mmol) (Example 5.3) in 15 mL of dry dichloromethane at 0° C. under argon. The reaction is left under stirring for 1 hour 30 minutes at room temperature. The product, as a triflate salt, is precipitated by adding diethyl ether and filtered off. The powder obtained is washed with water, ethanol and diethyl ether before being dried under vacuum. PA 1046 is obtained as a light beige powder (0.5 g, 54%).

IR (KBr) cm⁻¹: (C═O) 1760, 1664. ¹H NMR (400 MHz, DMSO) δ ppm: 2.01 (3H, s), 3.22 (1H, d, J=17.6 Hz), 3.47 (1H, d, J=17.6 Hz), 4.05 (2H, d, J=6.0 Hz), 4.76 (1H, d, J=12.0 Hz), 4.97 (1H, d, J=4.8 Hz), 4.99 (1H, d, J=12.0 Hz), 5.51 (1H, dd, J=4.8 Hz, J=8.4 Hz), 6.33 (1H, d, =5.6 Hz), 7.49 (1H, dd, J=2.4 Hz, J=9.2 Hz), 7.80 (1H, d, J=6.0 Hz), 7.82 (1H, d, =2.4 Hz), 8.25 (1H, d, J=9.2 Hz), 8.40 (1H, d, J=5.6 Hz), 9.11 (1H, d, J=8.4 Hz). MS (IS>0) m/z: 491.2 (M+H⁺). Elementary analysis: for C₂₁H₁₉ClN₄O₆S.2H₂O: % theor. C 47.86, N 10.63; % exper. C 47.96, N 10.36.

### Example 6

### Preparation of an Aminoquinoline-Cephalosporin, Ref PA 1089

### (6R,7R)-3-Acetoxymethyl-7-[2-(7-chloroquinolin-4-ylamino)-acetylamino]-8-oxo-5-thia-1-aza-bicyclo[4.2.0]oct-2-ene-2-carboxylic acid hydrochloride

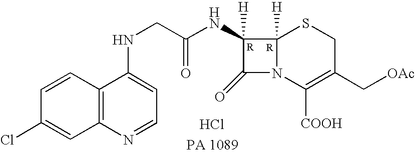

0.8 ml of a solution of 5M HCl in 2-propanol (4.0 mmol) is added dropwise to a solution, at 0° C., of PA 1046 (Example 5.4) (0.5 g, 1.0 mmol) in 40 ml of a 1/1 v/v chloroform/ethanol mixture. After 30 minutes of stirring at 0° C., the product is precipitated using diethyl ether. The precipitate is filtered off, washed with cold ethanol then with diethyl ether and dried under vacuum. PA 1089 is obtained as a light beige powder (0.4 g, 76%).

¹H NMR (300 MHz, DMSO) δ ppm: 2.03 (3H, s), 3.50 (1H, d, J=18.3 Hz), 3.65 (1H, d, J=18.3 Hz), 4.36 (2H, m), 4.70 (1H, d, J=12.9 Hz), 5.00 (1H, d, J=12.9 Hz), 5.12 (1H, d, J=4.8 Hz), 5.74 (1H, dd, J=4.8 Hz, J=7.8 Hz), 6.71 (1H, d, J=6.6 Hz), 7.81 (1H, d, J=9.0 Hz), 8.02 (1H, s), 8.52 (1H, d, J=9.0 Hz), 8.60 (1H, d, J=6.6 Hz), 9.33 (1H, d, J=7.8 Hz), 9.58 (1H, broad s) 13.80 (1H, broad s). Elementary analysis: for C₂₁H₁₉ClN₄O₆S.HCl.1.5H₂O: % theor. C 45.49, N 10.11; % exper. C 45.43, N 10.05.

### Example 7

### Preparation of an Aminoquinoline-Cephalosporin, Ref PA 1088

### (6R,7R-3-Acetoxymethyl-7-[2-7-chloroquinolin-4-ylamino)acetylamino]-5,8-dioxo-5λ⁴-thia-1-aza-bicyclo[4.2.0]oct-2-ene-2-carboxylic acid hydrochloride

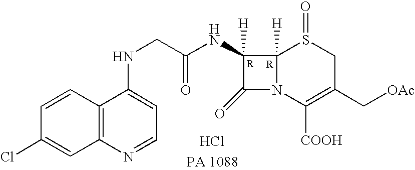

1.2 ml of anisole (10.7 mmol), followed by 2.0 ml of trifluoroacetic acid, injected dropwise (27.0 mmol), is added to a solution of (6R,7R)-3-acetoxymethyl-7-[2-(7-chloro-quinolin-4-ylamino)-acetylamino]-5,8-dioxo-5λ⁴-thia-1-aza-bicyclo[4.2.0]oct-2-ene-2-carboxylic acid benzhydryl ester (Example 5.3) (1.8 g, 2.7 mmol) in 20 mL of dry dichloromethane at 0° C. under argon. The reaction is left under stirring for 1 hour 30 minutes at room temperature. The product, as its triflate salt, is precipitated by adding diethyl ether, then filtered and washed with dichloromethane. The powder obtained is suspended in 20 ml of a 1/1 v/v chloroform/ethanol mixture and cooled to 0° C. Successively, 1.4 ml of a 2M solution of NH₃ in 2-propanol (2.7 mmol) are added to this suspension which is left under stirring for 15 minutes then 1.1 ml of a 5N solution of HCl in 2-propanol (4.0 mmol) are added and the mixture left under stirring for 30 minutes. The product is then precipitated using diethyl ether. The precipitate is filtered off, washed with chloroform, with ethanol and then with diethyl ether and dried under vacuum. PA 1088 is obtained as a slightly yellow powder (0.5 g, 24%).

¹H NMR (400 MHz, DMSO) δ ppm: 2.03 (3H, s), 3.62 (1H, d, J=18.4 Hz), 3.89 (1H, d, J=18.4 Hz), 4.41 (2H, m), 4.61 (1H, d, J=12.9 Hz), 4.92 (1H, d, =4.0 Hz), 5.20 (1H, d, J=12.9 Hz), 5.89 (1H, dd, J=4.0 Hz, J=8.2 Hz), 6.73 (1H, d, =6.5 Hz), 7.79 (1H, d, J=9.0 Hz), 8.08 (1H, s), 8.55 (1H, d, J=9.0 Hz), 8.60 (1H, d, J=6.5 Hz), 8.65 (1H, d, J=8.2 Hz), 9.56 (1H, broad s), 13.76 (broad s). MS (IS>0) m/z: 507.2 (M−Cl)⁺. Elementary analysis: for C₂₁H₁₉ClN₄O₇S.HCl.2H₂O: % theor. C 43.53, N 9.67; % exper. C 43.51, N 9.59.

### Example 8

### Preparation of an Aminoquinoline-Cephalosporin, Ref PA 1092

### (6R,7R-3-Acetoxymethyl-7-[2-7-chloroquinolin-4-ylamino)acetylamino]-5,8-dioxo-5λ¹-thia-1-aza-bicyclo[4.2.0]oct-2-ene-2-carboxylic acid

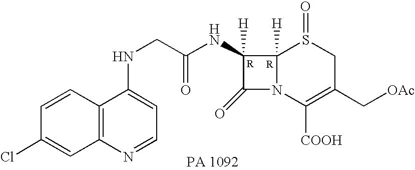

A suspension of PA 1088 (Example 7) (0.5 g, 0.8 mmol) is deprotonated under stirring for 30 minutes in 40 ml of water at room temperature. The product is filtered, washed with ethanol and then with diethyl ether and dried under vacuum. PA 1092 is obtained as a slightly yellow powder (0.2 g, 31%).

¹H NMR (250 MHz, DMSO) δ ppm: 2.00 (3H, s), 3.55 (1H, d, J=18.2 Hz), 3.85 (1H, d, J=18.2 Hz), 4.20 (2H, m), 4.57 (1H, d, J=12.5 Hz), 4.88 (1H, broad s), 5.18 (1H, d, J=12.5 Hz), 5.89 (1H, broad s), 6.51 (1H, broad s), 7.60 (1H, d, J=9.0 Hz), 7.88 (1H, s), 8.29-8.50 (4H, m). Elementary analysis: for C₂₁H₁₉ClN₄O₇S.3.5H₂O: % theor. C 44.25, N 9.83; % exper. C 44.21, N 9.57.

### Example 9

### Preparation of an Aminoquinoline-Cephalosporin, Ref PA 1037

### (6R,7R-3-Acetoxymethyl-7-[3-(7-chloroquinolin-4-ylamino)-propionylamino]-8-oxo-5-thia-1-aza-bicyclo[4.2.0]oct-2-ene-2-carboxylic acid

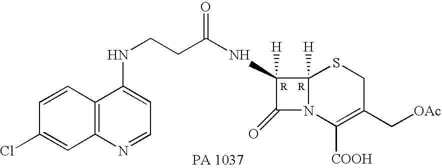

### 9.1. Mixture of (6R,7R-3-acetoxymethyl-7-[3-(7-chloro-quinolin-4-ylamino) propionyl-amino]-8-oxo-5-thia-1-aza-bicyclo[4.2.0]oct-2-ene-2-carboxylic acid benzhydryl ester and (6R,7R-3-acetoxymethyl-7-[3-7-chloroquinolin-4-ylamino)-propionyl-amino]-8-oxo-5-thia-1-aza-bicyclo[4.2.0]oct-3-ene-2-carboxylic acid benzhydryl ester: Δ²/Δ³.

The coupling product is prepared according to the procedure described in Example 5.1 from 5.7 g of 3-7-chloroquinolin-4-ylamino)propionic acid (Example 4.1) (19.8 mmol), 2.8 g of HOBT (20.7 mmol), 4.3 g of DCC (20.7 mmol), 8.7 g of (6R,7R)-3-acetoxymethyl-7-amino-8-oxo-5-thia-1-aza-bicyclo[4.2.0]oct-2-ene-2-carboxylic acid benzhydryl ester p-toluene sulfonic acid (19.8 mmol) and 2.8 mL of triethylamine (19.8 mmol). The coupling product is obtained after purification by liquid chromatography on silica gel (SiO₂ 60A C.C 70-200 μm, eluent: ethyl acetate/ethanol 90/10 v/v in order to remove impurities and then ethyl acetate/ethanol/triethylamine 90/5/5 v/v/v), as an orangey powder (6.1 g, 46%) as a Δ²/Δ³ 20/80 mixture. Used as such in the following step.

### 9.2. (6R,7R)-3-Acetoxymethyl-7-[3-(7-chloroquinolin-4-ylamino)-propionylamino]-5,8-dioxo-5λ⁴-thia-1-aza-bicyclo[4.2.0]oct-2-ene-2-carboxylic acid benzhydryl ester

The oxidation reaction is carried out according to the procedure described in Example 5.2 from 6.4 g of the Δ²/Δ³ mixture of Example 9.1 (9.5 mmol) and 3.3 g of 3-chloroperoxybenzoic acid (19.0 mmol). The product is obtained as a yellow powder (4.9 g, 75%).

IR (KBr) cm⁻¹: (C═O) 1788, 1733, 1647. ¹H NMR (400 MHz, DMSO) δ ppm: 1.98 (3H, s), 2.71 (2H, t, J=6.9 Hz), 3.53 (2H, q, J=6.9 Hz), 3.65 (1H, d, J=18.7 Hz), 3.96 (1H, d, J=18.7 Hz), 4.62 (1H, d, J=13.4 Hz), 4.97 (1H, d, J=4.8 Hz), 5.08 (1H, d, J=13.4 Hz), 5.98 (1H, dd, J=4.8 Hz, J=8.2 Hz), 6.55 (1H, d, J=5.5 Hz), 6.96 (1H, s), 7.26-7.46 (9H, m), 7.47 (1H, dd, J=2.2 Hz, J=9.0 Hz), 7.53 (2H, d, J=7.3 Hz), 7.80 (1H, d, J=2.2 Hz), 8.25 (1H, d, J=9.0 Hz), 8.43 (1H, d, J=5.5 Hz), 8.50 (1H, d, J=8.2 Hz). MS (IS>0) m/z: 687.3 (M+H⁺).

### 9.3. (6R,7R)-3-Acetoxymethyl-7-[3-(7-chloroquinolin-4-ylamino)-propionylamino]-8-oxo-5-thia-1-aza-bicyclo[4.2.0]oct-2-ene-2-carboxylic acid benzhydryl ester

The reduction reaction is carried out according to the procedure described in Example 5.3 from 5.6 g of (6R,7R)-3-acetoxymethyl-7-[3-(7-chloro-quinolin-4-ylamino)propionyl-amino]-5,8-dioxo-5λ⁴-thia-1-aza-bicyclo[4.2.0]oct-2-ene-2-carboxylic acid benzhydryl ester (Example 9.2) (8.2 mmol) and 1.6 mL of trichlorophosphine (18.3 mmol). After recrystallization from dichloromethane/diethyl ether, the product is obtained as a beige powder (5.0 g, 91%).

IR (KBr) cm⁻¹: (C═O) 1783, 1738, 1679. ¹H NMR (400 MHz, DMSO) δ ppm: 1.96 (3H, s), 2.72 (2H, t, J=6.8 Hz), 3.48 (1H, d, J=18.3 Hz), 3.64 (1H, d, J=18.3 Hz), 3.78 (2H, q, J=6.8 Hz), 4.62 (1H, d, J=13.0 Hz), 4.85 (1H, d, J=13.0 Hz), 5.15 (1H, d, J=4.9 Hz), 5.81 (1H, dd, J=4.9 Hz, J=8.3 Hz), 6.92 (1H, d, J=7.2 Hz), 6.92 (1H, s), 7.29-7.49 (10H, m), 7.79 (1H, dd, J=2.1 Hz, J=9.1 Hz), 8.07 (1H, d, J=2.1 Hz), 8.58 (1H, d, J=7.2 Hz), 8.62 (1H, d, J=9.1 Hz), 9.10 (1H, d, J=8.3 Hz), 9.54 (1H, t, J=6.8 Hz). MS (IS>0) m/z: 671.2 (M+H⁺).

### 9.4. (6R,7R)-3-Acetoxymethyl-7-[3-(7-chloroquinolin-4-ylamino)-propionylamino]-8-oxo-5-thia-1-aza-bicyclo[4.2.0]oct-2-ene-2-carboxylic acid

The deprotection reaction is carried out according to the procedure described in Example 5.4 from 3.0 g of (6R,7R)-3-acetoxymethyl-7-[3-(7-chloroquinolin-4-ylamino) -propionyl-amino]-8-oxo-5-thia-1-aza-bicyclo [4.2.0]oct-2-ene-2-carboxylic acid benzhydryl ester (example 9.3) (4.5 mmol), 2.0 ml of anisole (18.4 mmol) and 3.3 ml of trifluoroacetic acid (45.0 mmol). After recrystallization by dissolution in 5% (w/v) bicarbonate-containing water and precipitation at 0° C. with a 2M aqueous solution of HCl until pH 6 PA 1037 is obtained as an ecru powder (0.6 g, 27%).

IR (KBr) cm⁻¹: (C═O) 1773, 1753, 1653. ¹H NMR (400 MHz, DMSO) δ ppm: 2.02 (3H, s), 2.68 (2H, t, J=6.7 Hz), 3.39 (1H, d, J=18.0 Hz), 3.58 (1H, d, J=18.0 Hz), 3.71 (2H, m), 4.68 (1H, d, J=12.7 Hz), 5.00 (1H, d, J=12.7 Hz), 5.07 (1H, d, J=4.9 Hz), 5.70 (1H, dd, J=4.9 Hz, J=8.2 Hz), 6.83 (1H, d, J=6.8 Hz), 7.71 (1H, dd, J=2.1 Hz, J=9.1 Hz), 7.96 (1H, d, J=2.1 Hz), 8.46 (1H, d, J=9.1 Hz), 8.54 (1H, d, J=6.8 Hz), 8.94 (1H, broad s), 9.06 (1H, d, J=8.2 Hz). MS (IS>0) m/z: 505.0 (M+H⁺). Elementary analysis: for C₂₂H₂₁ClN₄O₆S.3H₂O: % theor. C 47.27, N 10.02; % exper. C 47.34, N 9.93.

### Example 10

### Preparation of an Aminoquinoline-Cephalosporin, Ref PA 1063

### (6R,7R)-3-Acetoxymethyl-7-[3-(7-chloroquinolin-4-ylamino)propionyl-amino]-5,8-dioxo-5λ⁴-thia-1-aza-bicyclo[4.2.0]oct-2-ene-2-carboxylic acid

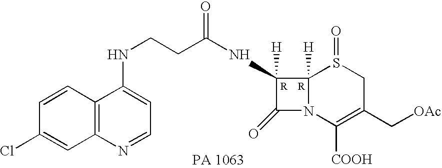

PA 1063 is obtained by the deprotection of (6R,7R-3-acetoxymethyl-7-[3-7-chloro-quinolin-4-ylamino)-propionylamino]-5,8-dioxo-5λ⁴-thia-1-aza-bicyclo[4.2.0]oct-2-ene-2-carboxylic acid benzhydryl ester (Example 9.2) (1.1 g, 1.6 mmol), is carried out according to the procedure described in Example 5.4 with 0.7 ml of anisole (6.2 mmol) and 1.2 ml of trifluoroacetic acid (15.5 mmol). PA 1063 is obtained as an ecru powder (0.6 g, 27%).

IR (KBr) cm⁻¹: (C═O) 1774, 1732, 1647. ¹H NMR (250 MHz, DMSO) δ ppm: 2.01 (3H, s), 2.71 (2H, broad s), 3.55 (1H, d, J=18.6 Hz), 3.59 (2H, broad s), 3.78 (1H, d, J=18.6 Hz), 4.59 (1H, d, J=12.9 Hz), 4.85 (1H, broad s), 5.21 (1H, d, J=12.9 Hz), 5.79 (1H, broad s), 6.67 (1H, broad s), 7.58 (1H, d, J=9.0 Hz), 7.87 (1H, s), 8.11 (1H, broad s), 8.33-8.46 (3H, m). MS (IS>0) m/z: 521.1 (M+H⁺).

### Example 11

### Preparation of an Aminoquinoline-Cephalosporin, Ref PA 1082

### (6R,7R)-3-Acetoxymethyl-7-[3-(7-chloroquinolin-4-ylamino)-propionylamino]-5,8-dioxo-5×4-thia-1-aza-bicyclo[4.2.0]oct-2-ene-2-carboxylic acid hydrochloride.

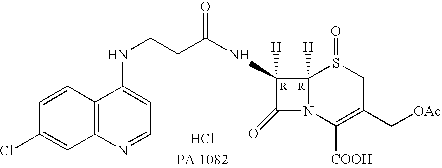

PA 1082 is prepared according to the procedure described in Example 6 from 0.7 g of PA 1063 (Example 10) (1.4 mmol) and 0.5 ml of a 5M solution of HCl in 2-propanol (4.0 mmol). PA 1082 is obtained as an ecru powder (0.4 g, 55%).

¹H NMR (250 MHz, DMSO) δ ppm: 2.02 (3H, s), 2.76 (2H, m) 3.60 (1H, d, J=18.6 Hz), 3.76 (2H, m), 3.85 (1H, d, J=18.6 Hz), 4.58 (1H, d, J=13.1 Hz), 4.89 (1H, d, J=4.2 Hz), 5.20 (1H, d, J=13.1 Hz), 5.83 (1H, dd, J=4.2 Hz, J=7.7 Hz), 6.91 (1H, d, J=7.1 Hz), 7.80 (1H, d, J=8.8 Hz), 8.04 (1H, s), 8.55 (3H, m), 9.43 (1H, broad s), 14.04 (broad s). MS (IS>0) m/z: 521.1 (M−Cl)⁺. Elementary analysis: for C₂₂H₂₁ClN₄O₇S.HCl.1.5H₂O.0.1Et₂O: % theor. C 44.77, N 9.32; % exper. C 44.83, N 9.25.

### Example 12

### Preparation of Aminoquinoline-Cephalosporin, Ref PA 1053

### (6R,7R-3-Acetoxymethyl-7-[4-7-chloroquinolin-4-ylamino)-butyrylamino]-8-oxo-5-thia-1-aza-bicyclo[4.2.0]oct-2-ene-2-carboxylic acid

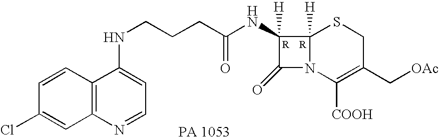

### 12.1. 4-(7-Chloroquinolin-4-yl)-butyric acid

This compound is prepared according to the procedure described in example 1.1 from 30.0 g of 4,7-dichloroquinoline (0.15 mol), 32.8 g of 4-aminobutyric acid (0.32 mol) and 77.0 g of phenol (0.82 mol). The product is obtained as a white powder (32.7 g, 82%).

¹H NMR (300 MHz, CF₃COOD) δ ppm: 2.23 (2H, quint, J=6.9 Hz), 2.71 (2H, t, J=6.9 Hz), 3.71 (2H, t, J=6.9 Hz), 6.81 (1H, d, J=7.5 Hz), 7.64 (1H, dd, J=1.8 Hz, J=9.0 Hz), 7.82 (1H, d, J=1.8 Hz), 8.08 (1H, d, J=9.0 Hz), 8.22 (1H, d, J=7.5 Hz).

### 12.2. Mixture of (6R,7R)-3-acetoxymethyl-7-[4-(7-chloroquinolin-4-ylamino)-butyrylamino]-8-oxo-5-thia-1-aza-bicyclo[4.2.0]oct-2-ene-2-carboxylic acid benzhydryl ester and (6R,7R)-3-acetoxymethyl-7-[4-(7-chloroquinolin-4-ylamino)-butyrylamino]-8-oxo-5-thia-1-aza-bicyclo [4.2.0]oct-3-ene-2-carboxylic acid benzhydryl ester: Δ²/Δ³

The coupling product is prepared according to the procedure described in Example 5.1 from 7.8 g of 4-(7-chloro-quinolinyl)-butyric acid (Example 12.1) (24.5 mmol), 3.5 g of HOBT (25.7 mmol), 5.3 g of DCC (25.7 mmol), 15.1 g of (6R,7R)-3-acetoxymethyl-7-amino-8-oxo-5-thia-1-aza-bicyclo[4.2.0]oct-2-ene-2-carboxylic acid benzhydryl ester p-toluenesulfonic acid (24.5 mmol) and 6.9 mL of triethylamine (49.5 mmol). The coupling product is obtained after purification by liquid chromatography on silica gel (SiO₂ 60A C.C 70-200 μm, eluent: ethyl acetate/ethanol/triethylamine 90/9/1 v/v/v) as an orangey powder (3.3 g, 20%) as a Δ²/Δ³ 22/78 mixture. Used as such in the following step.

### 12.3. (6R,7R-3-Acetoxymethyl-7-[4-(7-chloroquinolin-4-ylamino)-butyrylamino]-5,8-dioxo-5λ⁴-thia-1-aza-bicyclo[4.2.0]oct-2-ene-2-carboxylic acid benzhydryl ester

The oxidation reaction is carried out according to the procedure described in example 5.2 from 3.3 g of the Δ²/Δ³ mixture of example 12.2 (4.8 mmol) and 1.7 g of 3-chloroperoxybenzoic acid (9.6 mmol). The product is obtained as an orange-yellow powder (2.5 g, 74%).

IR (KBr) cm⁻¹: (C═O) 1791, 1735, 1655. ¹H NMR (300 MHz, DMSO) δ ppm: 1.92 (2H, quint, J=7.2 Hz), 2.01 (3H, s), 2.43 (2H, t, J=7.2 Hz), 3.33 (2H, m), 3.65 (1H, d, J=18.9 Hz), 3.96 (1H, d, J=18.9 Hz), 4.61 (1H, d, J=13.2 Hz), 4.96 (1H, d, J=4.2 Hz), 5.07 (1H, d, J=13.2 Hz), 5.95 (1H, dd, J=4.2 Hz, J=7.8 Hz), 6.57 (1H, d, J=5.7 Hz), 6.95 (1H, s), 7.27-7.54 (11H, m), 7.66 (1H, broad s), 7.80 (1H, d, J=2.1 Hz), 8.30 (1H, d, J=9.0 Hz), 8.32 (1H, d, J=7.8 Hz), 8.43 (1H, d, J=5.7 Hz). MS (IS>0) m/z: 701.3 (M+H⁺).

### 12.4. (6R,7R)-3-Acetoxymethyl-7-[4-(7-chloroquinolin-4-ylamino)-butyrylamino]-8-oxo-5-thia-1-aza-bicyclo[4.2.0]oct-2-ene-2-carboxylic acid benzhydryl ester

The reduction reaction is carried out according to the procedure described in example 5.3 from 2.9 g of (6R,7R)-3-acetoxymethyl-7-[4-(7-chloroquinolin-4-ylamino)-butyrylamino]-5,8-dioxo-5λ⁴-thia-1-aza-bicyclo [4.2.0]oct-2-ene-2-carboxylic acid benzhydryl ester (example 12.3) (4.1 mmol) and 0.8 mL of trichlorophosphine (9.1 mmol). After recrystallization from dichloromethane/diethyl ether, the product is obtained as a beige powder (2.5 g, 89%).

IR (KBr) cm⁻¹: (C═O) 1784, 1730, 1661. ¹H NMR (300 MHz, DMSO) δ ppm: 1.92 (2H, quint, J=7.2 Hz), 1.96 (3H, s), 2.38 (2H, t, J=7.2 Hz), 3.53 (2H, m), 3.53 (1H, d, J=18.6 Hz), 3.67 (1H, d, J=18.6 Hz), 4.62 (1H, d, J=12.9 Hz), 4.86 (1H, d, J=12.9 Hz), 5.16 (1H, d, J=4.8 Hz), 5.79 (1H, dd, J=4.8 Hz, J=8.1 Hz), 6.91 (1H, d, J=6.9 Hz), 6.92 (1H, s), 7.28-7.49 (10H, m), 7.78 (1H, dd, J=1.8 Hz, J=9.0 Hz), 8.04 (1H, d, J=1.8 Hz), 8.56 (1H, d, J=6.9 Hz), 8.63 (1H, d, J=9.0 Hz), 8.97 (1H, d, J=8.1 Hz), 9.56 (1H, broad s). MS (IS>0) m/z: 685.2 (M+H⁺).

### 12.5. (6R,7R-3-Acetoxymethyl-7-[4-(7-chloroquinolin-4-ylamino)-butyrylamino]-8-oxo-5-thia-1-aza-bicyclo[4.2.0]α-2-ene-2-carboxylic acid

The deprotection reaction is carried out according to the procedure described in example 5.4 from 0.8 g of (6R,7R-3-acetoxymethyl-7-[4-(7-chloroquinolin-4-ylamino) -butyrylamino]-8-oxo-5-thia-1-aza-bicyclo[4.2.0] oct-2-ene-2-carboxylic acid benzhydryl ester (example 12.4) (1.2 mmol), 0.5 ml of anisole (4.8 mmol) and 0.9 ml of trifluoroacetic acid (12.1 mmol). After recrystallization by dissolution in 5% (w/v) bicarbonate-containing water and precipitation at 0° C. with a 2M aqueous solution of HCl until pH 6 PA 1053 is obtained as a white powder (0.3 g, 35%).

IR (KBr) cm⁻¹: (C═O) 1769, 1737, 1653. ¹H NMR (300 MHz, DMSO) δ ppm: 1.91 (2H, m), 2.02 (3H, s), 2.37 (2H, t, J=7.2 Hz), 3.41 (2H, m), 3.44 (1H, d, J=18.3 Hz), 3.61 (1H, d, J=18.3 Hz), 4.69 (1H, d, J=12.9 Hz), 5.00 (1H, d, J=12.9 Hz), 5.09 (1H, d, J=4.8 Hz), 5.68 (1H, dd, J=4.8 Hz, J=8.1 Hz), 6.73 (1H, d, J=6.0 Hz), 7.64 (1H, d, J=9.0 Hz), 7.89 (1H, s), 8.41 (1H, d, J=9.0 Hz), 8.52 (2H, broad m), 8.90 (1H, d, J=6.0 Hz). MS (IS>0) m/z: 519.2 (M+H⁺). Elementary analysis: for C₂₃H₂₃ClN₄O₆S.2H₂O: % theor. C 49.77, N 10.10; % exper. C 49.79, N 9.74.

### Example 13

### Preparation of an Aminoquinoline-Cephalosporin, Ref PA 1054

### (6R,7R)-3-Acetoxymethyl-7-{[1-(7-chloroquinolin-4-yl)-piperidine-4-carbonyl]-amino}-8-oxo-5-thia-1-aza-bicyclo[4.2.0]oct-2-ene-2-carboxylic acid

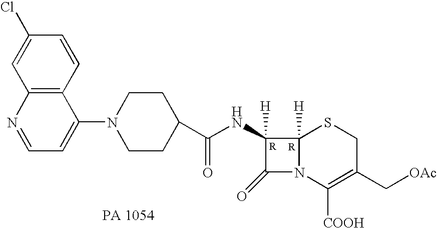

### 13.1. Mixture of (6R,7R)-3-acetoxymethyl-7-{[1-(7-chloroquinolin-4-yl)-piperidine-4-carbonyl]-amino}-8-oxo-5-thia-1-aza-bicyclo[4.2.0]oct-2-ene-2-carboxylic acid benzhydryl ester and (6R,7R)-3-acetoxymethyl-7-{[1-(7-chloroquinolin-4-yl)-piperidine carbonyl]-amino}-8-oxo-5-thia-1-aza-bicyclo[4.2.0]oct-3-ene-2-carboxylic acid benzhydryl ester: Δ²/Δ³

2.1 mL of N-methylmorpholine (19.4 mmol) are added to a mixture of 1-(7-chloro-quinolin-4-yl)-piperidine-carboxylic acid (example 1.1) (1.2 g, 3.9 mmol) and (6R,7R-3-acetoxymethyl-7-amino-8-oxo-5-thia-1-aza-bicyclo[4.2.0]oct-2-ene-2-carboxylic acid benzhydryl ester p-toluene sulfonic acid (2.4 g, 3.9 mmol) in 40 mL of DMF. The suspension is left under stirring for 15 minutes before adding the PyBOP® activator (2.0 g, 3.9 mmol). The stirring is continued for 24 hours at room temperature. The reaction medium is then diluted with 50 mL of dichloromethane and then washed successively with 50 ml of 5% (w/v) carbonate-containing water, twice 50 ml of water and 50 ml of water saturated with NaCl. The organic phase is dried over magnesium sulfate, filtered and then evaporated. The coupling product is obtained as an orangey powder (2.5 g, 90%) as a Δ²/Δ³ 32/68 mixture. Used as such in the following step.

### 13.2. (6R,7R)-3-Acetoxymethyl-7-{[1-(7-chloroquinolin-4-yl)-piperidine-4-carbonyl]-amino}-5,8-dioxo-5λ⁴-thia-1-aza-bicyclo[4.2.0]oct-2-ene-2-carboxylic acid benzhydryl ester

A solution of 3-chloroperoxybenzoic acid (4.9 g, 28.4 mmol) in 100 mL of dichloromethane is added dropwise, over a period of 3 hours, to a solution of the Δ²/Δ³ mixture from example 13.1 (10.1 g, 14.2 mmol) in 100 mL of dichloromethane at 0° C. The reaction medium is then washed with a mixture of 100 ml of 5% (w/v) bicarbonate-containing water and 100 ml of a 5% (w/v) aqueous solution of sodium sulfite. The organic phase is dried over magnesium sulfate, filtered and then evaporated. The product is then purified by liquid chromatography on silica gel (SiO₂ 60A C.C 70-200 μm, eluent: dichloromethane/ethanol 90/10 v/v). The cleanest fractions, according to TLC revealed under UV, are evaporated. The product is obtained as an ecru powder (4.0 g, 38%).

IR (KBr) cm⁻¹: (C═O) 1787, 1733, 1653. ¹H NMR (400 MHz, CDCl₃) δ ppm: 2.05 (3H, s), 2.10 (4H, m), 2.50 (1H, m), 2.87 (2H, m), 3.28 (1H, d, J=19.2 Hz), 3.88 (1H, d, J=19.2 Hz), 3.63 (2H, d, J=12.0 Hz), 4.56 (1H, d, J=4.8 Hz), 4.78 (1H, d, J=14.4 Hz), 5.32 (1H, d, J=14.4 Hz), 6.18 (1H, dd, J=4.8 Hz, J=9.6 Hz), 6.83 (1H, d, J=5.2 Hz), 6.97 (1H, s), 6.97 (1H, d, J=9.6 Hz), 7.27-7.49 (11H, m), 7.92 (1H, d, J=9.2 Hz), 8.05 (1H, d, J=2.0 Hz), 8.71 (1H, d, J=5.2 Hz). MS (IS>0) m/z: 727.3 (M+H⁺).

### 13.3. (6R,7R-3-Acetoxymethyl-7-[1-(7-chloroquinolin-4-yl)-piperidine 1 carbonyl)-amino)-8-oxo-5-thia-1-aza-bicyclo[4.2.0]oct-2-ene-2-carboxylic acid benzhydryl ester

0.2 mL of trichlorophosphine (1.9 mmol) is added dropwise to a solution of (6R,7R)-3-acetoxymethyl-7-{[1-(7-chloroquinolin-4-yl)-piperidine-4-carbonyl]-amino}-5,8-dioxo-5λ⁴-thia-1-aza-bicyclo[4.2.0]oct-2-ene-2-carboxylic acid benzhydryl ester (example 13.2) (0.6 g, 0.9 mmol) in 6 mL of dry DMF at −20° C. under argon. The reaction is left under stirring for 1 hour at −20° C. The reaction medium is then diluted with 20 mL of dichloromethane and then washed successively with twice 20 ml of water and 20 ml of water saturated with NaCl. The organic phase is dried over magnesium sulfate, filtered and then evaporated. After recrystallization from dichloromethane/diethyl ether, the product is obtained as an ecru powder (0.5 g, 83%).

IR (KBr) cm⁻¹: (C═O) 1783, 1732, 1672. ¹H NMR (400 MHz, CDCl₃) δ ppm: 2.02 (3H, s), 2.11-2.23 (4H, m), 3.07 (1H, m), 3.20 (1H, d, J=18.8 Hz), 3.45 (2H, m), 3.50 (1H, d, J=18.8 Hz), 4.02 (2H, m), 4.48 (1H, d, J=14.1 Hz), 4.95 (1H, d, J=14.1 Hz), 4.99 (1H, d, J=4.9 Hz), 5.94 (1H, dd, J=4.9 Hz, J=8.5 Hz), 6.48 (1H, d, J=6.7 Hz), 6.82 (1H, s), 7.30-7.55 (12H, m), 7.91 (1H, d, J=9.2 Hz), 8.29 (1H, d, J=8.5 Hz), 8.33 (1H, d, J=6.7 Hz), 8.48 (1H, d, J=1.8 Hz). SM (IS>0) m/z: 711.2 (M+H⁺).

### 13.4. (6R,7R)-3-Acetoxymethyl-7-{[1-(7-chloroquinolin-4-yl)-piperidine-4-carbonyl]-amino}-8-oxo-5-thia-1-aza-bicyclo[4.2.0]oct-2-ene-2-carboxylic acid

0.3 ml of anisole (2.5 mmol) is added, followed by 0.5 ml of trifluoroacetic acid injected dropwise (6.3 mmol), to a solution of (6R,7R)-3-acetoxymethyl-7-{[1-(7-chloro-quinolin 4-yl)-piperidin-4-carbonyl]-amino)-8-oxo-5-thia-1-aza-bicyclo[4.2.0]oct-2-ene-2-carboxylic acid benzhydryl ester (0.5 g, 0.6 mmol) (example 13.3) in 10 mL of dry dichloromethane at 0° C. under argon. The reaction is left under stirring for 1 hour 30 minutes at room temperature. The product, as a triflate salt, is precipitated by adding diethyl ether and filtered. The powder obtained is washed with water, acetone and diethyl ether before being dried under vacuum. PA 1054 is obtained as an ecru powder (0.2 g, 44%).

IR (KBr) cm⁻¹: (C═O) 1763, 1737, 1648. ¹H NMR (400 MHz, DMSO) δ ppm: 1.86-1.98 (4H, m), 2.02 (3H, s), 2.58 (1H, m), 2.85 (2H, m), 3.30 (1H, d, J=17.2 Hz), 3.53 (1H, d, J=17.2 Hz), 3.56 (2H, m), 4.75 (1H, d, J=12.4 Hz), 5.01 (1H, d, J=12.4 Hz), 5.03 (1H, d, J=4.4 Hz), 6.03 (1H, dd, J=4.4 Hz, J=8.0 Hz), 7.02 (1H, d, J=5.0 Hz), 7.56 (1H, dd, J=2.0 Hz, J=9.2 Hz), 7.97 (1H, d, J=2.0 Hz), 8.01 (1H, d, J=9.2 Hz), 8.69 (1H, d, J=5.0 Hz), 8.89 (1H, d, J=8.0 Hz). MS (IS>0) m/z: 545.2 (M+H⁺).

### Example 14

### Preparation of an Aminoquinoline-Cephalosporin, Ref PA 1074

### (6R,7R-3-Acetoxymethyl-7-{[1-(7-chloroquinolin-4-yl)-piperidine-4-carbonyl]-amino}-8-oxo-5-thia-1-aza-bicyclo[4.2.0]oct-2-ene-2-carboxylic acid hydrochloride

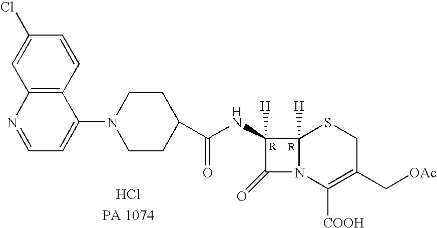

0.2 ml of a solution of 5M HCl in 2-propanol (1.0 mmol) is added dropwise to a solution of PA 1054 (example 13.4) (0.5 g, 0.8 mmol) in 40 ml of a mixture of chloroform/ethanol 1/1 v/v at 0° C. After 20 minutes of stirring at 0° C., the product is precipitated using diethyl ether. The precipitate is filtered off, washed with cold acetone and then with diethyl ether and dried under vacuum. PA 1074 is obtained as an ecru powder (0.3 g, 54%).

IR (KBr) cm⁻¹: (C═O) 1779, 1736, 1668. ¹H NMR (300 MHz, DMSO) δ ppm: 1.79-1.99 (4H, m), 2.03 (3H, s), 2.74 (1H, m), 3.43 (2H, m), 3.55 (1H, d, J=18.3 Hz), 3.64 (1H, d, J=18.3 Hz), 4.12 (2H, d, J=12.6 Hz), 4.68 (1H, d, J=12.9 Hz), 5.00 (1H, d, J=12.9 Hz), 5.12 (1H, d, J=4.5 Hz), 5.69 (1H, dd, J=4.5 Hz, J=8.1 Hz), 7.20 (1H, d, J=7.2 Hz), 7.68 (1H, dd, J=1.5 Hz, J=9.0 Hz), 8.11 (1H, d, J=1.5 Hz), 8.15 (1H, d, J=9.0 Hz), 8.65 (1H, d, J=7.2 Hz), 8.98 (1H, d, J=8.1 Hz). MS (IS>0) m/z: 545.2 (M+H⁺). Elementary analysis: for C₂₅H₂₅ClN₄O₆S.HCl.2.5H₂O: % theor. C 47.92, N 8.94; % exper. C 47.89, N 8.92.

### Example 15

### Preparation of an Aminoquinoline-Cephalosporin, Ref PA 1100

### (6R,7R)-3-Acetoxymethyl-7-[2-(7-trifluoromethylquinolin-4-ylamino)-acetylamino]-8-oxo-5-thia-1-aza-bicyclo[4.2.0]oct-2-ene-2-carboxylic acid

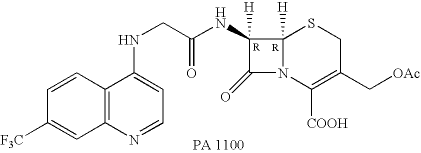

### 15.1. (7-Trifluoromethylquinolin-4-ylamino)acetic acid

This compound is prepared according to the procedure described in example 1.1 from a mixture of 2.5 g of 4-chloro-7-trifluoromethyl)quinoline (10.8 mmol), 1.8 g of glycine (23.7 mmol) and 5.7 g of phenol (60.4 mmol) heated for 24 hours at 150° C. The product is obtained as a white powder (1.8 g, 62%).

¹H NMR (300 MHz, DMSO) δ ppm: 4.10 (2H, d, J=6.0 Hz), 6.48 (1H, d, J=5.4 Hz), 7.72 (1H, dd, J=1.8 Hz, J=9.0 Hz), 7.83 (1H, t, J=6.0 Hz), 8.11 (1H, d, J=1.8 Hz), 8.43 (1H, d, J=9.0 Hz), 8.52 (1H, d, J=5.4 Hz).

### 15.2. Mixture of (6R,7R)-3-acetoxymethyl-7-[2-(7-trifluoromethylquinolin-4-ylamino)-acetylamino]-8-oxo-5-thia-1-aza-bicyclo[4.2.0]oct-2-ene-2-carboxylic acid benzhydryl ester and (6R,7R)-3-acetoxymethyl-7-[2-(7-trifluoromethylquinolin-4-ylamino)acetylamino]-8-oxo-5-thia-1-aza-bicyclo[4.2.0]oct-3-ene-2-carboxylic acid benzhydryl ester: Δ²/Δ³

The coupling product is prepared according to the procedure described in example 13.1 from 0.7 g of (7-trifluoromethylquinolin-4-ylamino)-acetic acid (example 15.1) (2.6 mmol), 1.6 g of (6R,7R)-3-acetoxymethyl-7-amino-8-oxo-5-thia-1-aza-bicyclo[4.2.0]oct-2-ene-2-carboxylic acid benz-hydryl ester p-toluene sulfonic acid (2.6 mmol), 1.4 mL of N-methylmorpholine (13.0 mmol) and 1.3 g of PyBOP® (2.6 mmol). The coupling product is obtained after purification by liquid chromatography on silica gel (SiO₂ 60A C.C 6-35 μm, eluent: ethyl acetate/triethylamine/ethanol 96/3/1 v/v/v) as a light beige powder (0.6 g, 32%) as a Δ²/Δ³ 31/69 mixture. Used as such in the following step.

### 15.3. (6R,7R)-3-Acetoxymethyl-7-[2-(7-trifluoromethylquinolin-4-ylamino)-acetylamino]-5,8-dioxo-5×4-thia-1-aza-bicyclo[4.2.0]oct-2-ene-2-carboxylic acid benzhydryl ester

The oxidation reaction is carried out according to the procedure described in example 5.2 from 0.6 g of the Δ²/Δ³ mixture from example 15.2 (0.8 mmol) and 0.3 g of 3-chloroperoxybenzoic acid (1.7 mmol). The product is obtained as a yellow powder (0.5 g, 91%).

IR (KBr) cm⁻¹: (C═O) 1786, 1734, 1668. ¹H NMR (300 MHz, DMSO) δ ppm: 1.95 (3H, s), 3.60 (1H, d, J=18.6 Hz), 3.93 (1H, d, J=18.6 Hz), 4.15 (2H, m), 4.57 (1H, d, J=13.5 Hz), 4.95 (1H, d, J=4.8 Hz), 5.02 (1H, d, J=13.5 Hz), 6.04 (1H, dd, J=4.8 Hz, J=9.0 Hz), 6.51 (1H, d, J=5.4 Hz), 6.94 (1H, s), 7.26-7.52 (10H, m), 7.75 (1H, dd, J=1.5 Hz, J=8.7 Hz), 8.01 (1H, broad s), 8.13 (1H, d, J=1.5 Hz), 8.38 (1H, d, J=9.0 Hz), 8.40 (1H, d, J=8.7 Hz), 8.56 (1H, d, J=5.4 Hz). MS (IS>0) m/z: 707.2 (M+H⁺).

### 15.4. (6R,7R)-3-Acetoxymethyl-7-[2-(7-trifluoromethylquinolin-4-ylamino)-acetylamino]-8-oxo-5-thia-1-aza-bicyclo[4.2.0]oct-2-ene-2-carboxylic acid benzhydryl ester

The reduction reaction is carried out according to the procedure described in example 5.3 from 0.4 g of (6R,7R)-3-acetoxymethyl-7-[2-(7-trifluoromethylquinolin 4-ylamino)-acetylamino]-5,8-dioxo-5×4-thia-1-aza-bicyclo[4.2.0]oct-2-ene-2-carboxylic acid benzhydryl ester (example 15.3) (0.6 mmol) and 0.1 mL of trichlorophosphine (1.3 mmol). After recrystallization from dichloromethane/diethyl ether, the product is obtained as a beige powder (0.2 g, 54%).

¹H NMR (300 MHz, DMSO) δ ppm: 1.96 (3H, s), 3.35 (2H, m), 4.37 (2H, m), 4.64 (1H, d, J=12.9 Hz), 4.93 (1H, broad s), 4.96 (1H d, J=12.9 Hz), 5.78 (1H, broad s), 6.55 (1H, broad s), 6.87 (1H, s), 7.25-7.43 (10H, m), 7.62 (1H, broad s), 8.29 (1H, broad s), 8.39 (1H, broad s), 8.56 (1H, broad s), 8.93 (1H, broad s), 9.32 (1H, broad s).

### 15.5. (6R,7R)-3-Acetoxymethyl-7-[2-(7-trifluoromethylquinolin-4-ylamino)-acetylamino]-8-oxo-5-thia-1-aza-bicyclo[4.2.0]oct-2-ene-2-carboxylic acid

The deprotection reaction is carried out according to the procedure described in example 5.4 from 0.2 g of (6R,7R)-3-acetoxymethyl-7-[2-(7-trifluoromethylquinolin-4-ylamino)-acetylamino]-8-oxo-5-thia-1-aza-bicyclo[4.2.0]oct-2-ene-2-carboxylic acid benzhydryl ester (example 15.4) (0.3 mmol), 0.1 ml of anisole (1.3 mmol) and 0.2 ml of trifluoroacetic acid (3.2 mmol). After successive washings with water, acetonitrile and diethyl ether PA 1100 is obtained as a yellow powder (0.1 g, 54%).

IR (KBr) cm⁻¹: (C═O) 1772, 1734, 1674. ¹H NMR (300 MHz, DMSO) δ ppm: 2.03 (3H, s), 3.49 (1H, d, J=18.0 Hz), 3.63 (1H, d, J=18.0 Hz), 4.17 (2H, d, J=5.7 Hz), 4.69 (1H, d, J=12.8 Hz), 5.00 (1H, d, J=12.8 Hz), 5.11 (1H, d, J=4.8 Hz), 5.73 (1H, dd, J=4.8 Hz, J=8.4 Hz), 6.54 (1H, d, J=5.4 Hz), 7.83 (1H, d, J=9.0 Hz), 8.16 (1H, s), 8.43 (1H, broad s), 8.51 (1H, d, J=9.0 Hz), 8.58 (1H, broad s), 9.25 (1H, d, J=8.4 Hz). MS (IS>0) m/z: 525.3 (M+H⁺). Elementary analysis: for C₂₂H₁₉F₃N₄O₆S.3.5H₂O: % theor. C 44.97, N 9.54; % exper. C 44.94, N 9.15.

### Example 16

### Preparation of an Aminoquinoline-Cephalosporin, Ref PA 1101

### (6R,7R-3-Acetoxymethyl-7-[2-2-methylquinolin-4-ylamino)acetylamino]-8-oxo-5-thia-1-aza-bicyclo[4.2.0]oct-2-ene-2-carboxylic acid

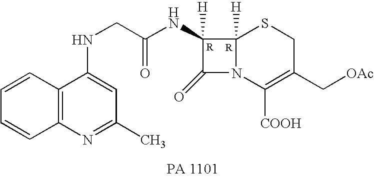

### 16.1. (2-Methylquinolin-4-ylamino)-acetic acid

This compound is prepared according to the procedure described in example 1.1 from a mixture of 4.8 g of 4-chloro-quinaldine (27.3 mmol), 4.5 g of glycine (60.0 mmol) and 14.6 g of phenol (155.0 mmol) heated for 24 hours at 150° C. The product is obtained as a white powder (3.8 g, 64%).

¹H NMR (300 MHz, CF₃COOD) δ ppm: 2.60 (3H, s), 4.37 (2H, s), 6.42 (1H, s), 7.59 (1H, t, J=7.2 Hz), 7.66 (1H, d, J=8.4 Hz), 7.80 (1H, t, J=7.5 Hz), 7.95 (1H, d, J=8.7 Hz).

### 16.2. Mixture of (6R,7R)-3-acetoxymethyl-7-[2-(2-methylquinolin-4-ylamino]-acetylamino]-8-oxo-5-thia-1-aza-bicyclo[4.2.0]oct-2-ene-2-carboxylic acid benzhydryl ester and (6R,7R)-3-acetoxymethyl-7-[2-(2-methylquinolin-4-ylamino)-acetylamino]-8-oxo-5-thia-1-aza-bicyclo[4.2.0]oct-3-ene-2-carboxylic acid benzhydryl ester: Δ²/Δ³

The coupling product is prepared according to the procedure described in example 13.1 from 0.7 g of (2-methyl-quinolin-4-ylamino)-acetic acid (example 16.1) (3.5 mmol), 2.2 g of (6R,7R)-3-acetoxymethyl-7-amino-8-oxo-5-thia-1-aza-bicyclo[4.2.0]oct-2-ene-2-carboxylic acid benzhydryl ester p-toluene sulfonic acid (3.5 mmol), 1.9 mL of N-methylmorpholine (17.5 mmol) and 1.8 g of PyBOP® (3.5 mmol). The coupling product is obtained after purification by liquid chromatography on silica gel (SiO₂ 60A C.C 6-35 μm, eluent: ethyl acetate/triethylamine/ethanol 95/3/2 v/v/v) as an orangey powder (1.3 g, 58%) as a Δ²/Δ³ 21/79 mixture. Used as such in the following step.

### 16.3. (6R,7R-3-Acetoxymethyl-7-[2-(2-methylquinolin-4-ylamino)acetylamino]-5,8-dioxo-5λ¹-thia-1-aza-bicyclo[4.2.0]oct-2-ene-2-carboxylic acid benzhydryl ester

The oxidation reaction is carried out according to the procedure described in example 5.2 from 1.3 g of the Δ²/Δ³ mixture of example 16.2 (2.0 mmol) and 0.7 g 3-chloroperoxybenzoic acid (4.0 mmol). The product is obtained as an orange powder (1.1 g, 83%).

IR (KBr) cm⁻¹: (C═O) 1792, 1734, 1652. ¹H NMR (300 MHz, DMSO) δ ppm: 1.95 (3H, s), 2.52 (3H, s), 3.62 (1H, d, J=18.9 Hz), 3.95 (1H, d, J=18.9 Hz), 4.13 (2H, m), 4.58 (1H, d, J=13.5 Hz), 4.96 (1H, d, J=4.2 Hz), 5.03 (1H, d, J=13.5 Hz), 6.04 (1H, dd, J=4.2 Hz, J=8.7 Hz), 6.34 (1H, s), 6.94 (1H, s), 7.26-7.54 (11H, m), 7.64 (1H, t, J=7.5 Hz), 7.75 (1H, d, J=7.8 Hz), 7.89 (1H, m), 8.17 (1H, d, J=8.7 Hz), 8.42 (1H, d, J=8.7 Hz). MS (IS>0) m/z: 653.2 (M+H⁺).

### 16.4. (6R,7R)-3-Acetoxymethyl-7-[2-(2-methylquinolin-4-ylamino)-acetylamino]-8-oxo-5-thia-1-aza-bicyclo[4.2.0]oct-2-ene-2-carboxylic acid benzhydryl ester

The reduction reaction is carried out according to the procedure described in example 5.3 from 2.3 g of (6R,7R)-3-acetoxymethyl-7-[2-(2-methyl-quinolin-4ylamino)-acetylamino]-5,8-dioxo-5λ⁴-thia-1-aza-bicyclo [4.2.0]oct-2-ene-2-carboxylic acid benzhydryl ester (example 16.3) (3.5 mmol) and 0.7 mL of trichlorophosphine (7.8 mmol). After recrystallization from dichloromethane/diethyl ether, the product is obtained as a beige powder (1.8 g, 82%).

IR (KBr) cm⁻¹: (C═O) 1784, 1733, 1639. ¹H NMR (300 MHz, DMSO) δ ppm: 1.96 (3H, s), 2.73 (3H, s), 3.58 (1H, d, J=18.3 Hz), 3.70 (1H, d, J=18.3 Hz), 4.33 (2H, m), 4.63 (1H, d, J=12.9 Hz), 4.87 (1H, d, J=12.9 Hz), 5.19 (1H, d, J=5.1 Hz), 5.85 (1H, dd, J=5.1 Hz, J=8.1 Hz), 6.61 (1H, s), 6.93 (1H, s), 7.28-7.50 (10H, m), 7.70 (1H, m), 7.94 (2H, m), 8.46 (1H, d, J=8.7 Hz), 9.37 (1H, d, J=8.1 Hz), 9.42 (1H, t, d, J=6.0 Hz). MS (IS>0) m/z: 637.2 (M+H⁺).

### 16.5. (6R,7R)-3-Acetoxymethyl-7-[2-(2-methylquinolin-4-ylamino)acetylamino]-8-oxo-5-thia-1-aza-bicyclo[4.2.0]oct-2-ene-2-carboxylic acid

The deprotection reaction is carried out according to the procedure described in example 5.4 from 0.8 g of (6R,7R-3-acetoxymethyl-7-[2-(2-methyl-quinolin-4-ylamino) -acetylamino]-8-oxo-5-thia-1-aza-bicyclo[4.2.0]oct-2-ene-2-carboxylic acid benzhydryl ester (example 16.4) (1.2 mmol), 0.5 ml of anisole (4.9 mmol) and 0.9 ml of trifluoroacetic acid (12.4 mmol). After successive washings with water, acetone and diethyl ether PA 1101 is obtained as a yellow powder (0.2 g, 27%).

IR (KBr) cm⁻¹: (C═O) 1772, 1736, 1652. ¹H NMR (400 MHz, DMSO) δ ppm: 2.01 (3H, s), 2.59 (3H, s), 3.38 (1H, d, J=17.6 Hz), 3.58 (1H, d, J=17.6 Hz), 4.21 (2H, m), 4.74 (1H, d, J=12.4 Hz), 5.01 (1H, d, J=12.4 Hz), 5.06 (1H, d, J=4.8 Hz), 5.64 (1H, dd, J=4.8 Hz, J=8.0 Hz), 6.46 (1H, s), 7.58 (1H, t, J=7.4 Hz), 7.80 (1H, t, J=7.5 Hz), 7.89 (1H, d large, J=7.8 Hz), 8.33 (1H, d, J=8.6 Hz), 8.77 (1H, broad s), 9.26 (1H, d, J=8.0 Hz). MS (IS>0) m/z: 471.2 (M+H⁺). Elementary analysis: for C₂₂H₂₂N₄O₆S.2.5H₂O: % theor. C 51.25, N 10.87; % exper. C 51.00, N 10.79.

### Example 17

### Preparation of an Aminoquinoline-Cephalosporin, Ref P4 1191

### (6R,7R)-3-Acetoxymethyl-7-[4-morpholin-4-yl-quinoline-2-carbonyl)-amino]-8-oxo-5-thia-1-aza-bicyclo[4.2.0]oct-2-ene-2-carboxylic acid

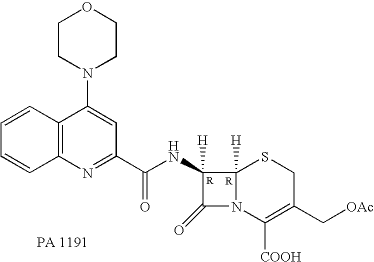

### 17.1 4-Chloroquinoline-2-carboxylic acid ethyl ester

A mixture of 4-hydroxyquinoline-2-carboxylic acid ethyl ester (10.0 g, 46.0 mmol) and phosphorus oxychloride (43 mL, 460.0 mmol) is heated to reflux for 2.5 hr. After cooling to room temperature, the mixture is concentrated to dryness by a tube to tube before the slow addition of 26 mL of water then 44 mL of 28% ammonia. The product is then extracted with 500 mL of boiling ethyl acetate. The organic phase is evaporated to dryness. After one recrystallization from methanol/water, the product is obtained as a white powder (9.3 g, 86%).

¹H NMR (250 MHz, DMSO) δ ppm: 1.48 (3H, t, J=7.0 Hz), 4.55 (2H, q, J=7.0 Hz), 7.74 (1H, dt, J=1.1 Hz, J=6.3 Hz), 7.84 (1H, dt, J=1.4 Hz, J=7.0 Hz), 8,25 (1H, s), 8.30 (1H, m), 8.34 (1H, m). MS (IS>0) m/z: 235.9 (M+H⁺).

### 17.2. Morpholin-4-yl-(4-morpholin-4-yl-quinolin-2-yl)-methanone

A mixture of 4-chloro-quinoline-2-carboxylic acid ethyl ester (example 17.1) (4.5 g, 19 mmol) and morpholine (16 mL, 190.0 mmol) is heated at reflux under argon for 16 hr. The reaction medium is then diluted with 200 mL of dichloromethane and washed successively 3 times with 200 mL of water then 200 mL of a saturated aqeous NaCl solution. The organic phase is dried over magnesium sulfate, filtered and dried under vacuum. The product is obtained as a white powder (5.5 g, 88%).

¹H NMR (250 MHz, CDCL₃) δ ppm: 3.30 (4H, s), 3.74 (4H, d, J=2.9 Hz), 3.86 (4H, s), 3.99 (4H, t, J=4.3 Hz), 7.20 (1H, s), 7.57 (1H, t, J=7.5 Hz), 7.71 (1H, t, J=7.1), 8.00 (1H, m), 8.04 (1H, m). MS (IS>0) m/z: 328.0 (M+H⁺).

### 17.3. 4-Morpholin-4-yl-quinoline-2-carboxylic acid

A 2.7M of aqueous sodium hydroxide solution (160.0 mmol) is added to a solution of morpholin-4-yl-(4-morpholin-4-yl-quinolin-2-yl)methanone (example 17.2) (5.2 g, 16.0 mmol) in 60 mL of ethanol. The medium is left for 15 hr with stirring. The resulting white precipitate is filtered and dried under vacuum (3.1 g, 75%).

¹H NMR (250 MHz, DMSO) δ ppm: 3,15 (4H, t, J=4.1 Hz), 3.88 (4H, t, J=4.5 Hz), 7.51 (1H, t, J=7.1 Hz), 7.65 (1H, t, J=7.16 Hz), 7.66 (1H, s), 8.00 (1H, d, J=8.0 Hz), 8.24 (1H, d, J=8.4 Hz).

### 17.4. Mixture of (6R,7R)-3-acetoxymethyl-7-[(4-morpholin-4-yl-quinoline-2-carbonyl)-amino-8-oxo-5-thia-1-aza-bicylo[4.2.0]oct-2-ene-2-carboxylic acid benzhydryl ester and (6R,7R)-3-acetoxymethyl-7-[(4-morpholin4ylquinoline-2-carbonyl)-amino]-8-oxo-5-thia-1-aza-bicylo[4.2.0]oct-3-ene-2-carboxylic acid benzhydryl ester: Δ²/Δ³

The coupling product is prepared according to the procedure described in example 13.1 from 3.2 g of 4-morpholin ylquinoline-2-carboxylic acid (example 17.3) (12.4 mmol), 7.5 g of (6R,7R-3-acetoxymethyl-7-amino-8-oxo-5-thia-1-aza-bicyclo[4.2.0]oct-2-ene-2-carboxylic acid benzhydryl ester p-toluene sulfonic acid (12.4 mmol), 4.0 mL de N-methylmorpholine (37.2 mmol) and 6.4 g of PyBOP (12.4 mmol). The coupling product is obtained after purification by liquid chromatography on silica gel (SiO₂ 60A C.C 70-200 μm, eluent: dichloromethane/ethyl acetate 80/20 v/v) as an orangey powder (3.7 g, 45%) as a Δ²/Δ³ 23/77 mixture, used as such in the following step.

### 17.5. (6R,7R-3-Acetoxymethyl-7-[(4-morpholin-4-yl-quinoline-2-carbonyl)-amino]-5,8-dioxo-5λ⁴-thia-1-aza-bicyclo[4.2.0]oct-2-ene-2-carboxylic acid benzhydryl ester

The oxidation reaction is carried out according to the procedure described in example 5.2 from 3.2 g of the Δ²/Δ³ mixture of example 17.4 (4.8 mmol) and 2.2 g of 3-chloroperoxybenzoic acid (13.0 mmol). The product is purified by liquid chromatography on silica gel (SiO₂ 60A C.C 70-200 μm, eluent: dichloromethane/ethyl acetate 80/20 (v/v) as a yellow powder (1.1 g, 33%).

¹H NMR (300 MHz, CDCl₃) δ ppm: 2.00 (3H, s), 3.25 (1H, d, J=18.9 Hz), 3.33 (4H, t, J=4.8 Hz), 3.90 (1H, d, J=18.9 Hz), 4.00 (4H, t, J=4.5 Hz), 4.64 (1H, d, J=4.8 Hz), 4.82 (1H, d, J=14.1 Hz), 5.35 (1H, d, J=14.1 Hz), 6.35 (1H, dd, J=10.2 Hz, J=4.8 Hz), 7.00 (1H, s), 7.30-7.37 (10H, m), 7.51 (1H, s), 7.52 (1H, m), 7.75 (1H,m), 8.03 (1H, d, J=8.4 Hz), 8.12 (1H, d, J=8.4 Hz), 9.37 (1H, d, J=10.5 Hz).

### 17.6. (6R,7R)-3-Acetoxymethyl-7-[(4-morpholin-4-yl-quinoline-2-carbonyl)-amino]-8-oxo-5-thia-1-aza-bicylo[4.2.0]oct-2-ene-2-carboxylic acid benzhydryl ester

The reduction reaction is carried out according to the procedure described in example 5.3 from 0.8 g of (6R,7R)-3-acetoxymethyl-7-[(4-morpholin-4-yl-quinoline-2-carbonyl)-amino]-5,8-dioxo-5λ⁴-thia-1-aza-bicyclo[4.2.0]oct-2-ene-2-carboxylic acid benzhydryl ester (example 17.5) (1.1 mmol) and 0.2 mL of trichlorophosphine (2.4 mmol). The product is obtained after solubilization in dichloromethane and addition of diethyl ether until the product comes out of solution as a brown oil. The supernatant is eliminated and the oil is dried under vacuum (0.7 g, 91%). ¹H NMR (300 MHz, CDCl₃) δ ppm: 2.04 (3H, s), 3.32 (4H, t, J=4.1 Hz), 3.42 (1H, d, J=15.9 Hz), 3.65 (1H, d, J=15.9 Hz), 3.98 (4H, t, J=4.7 Hz), 4.82 (1H, d, J=13.5 Hz), 5.05 (1H, d, J=13.5 Hz), 5.12 (1H, d, J=4.9 Hz), 6.07 (1H, m), 6.98 (1H, s), 7.27-7.33 (10H, m), 7.59 (1H, t, J=8.1 Hz), 7.71 (1H, m), 7.73 (1H, s), 8.02 (1H, d, J=8.4 Hz), 8.20 (1H, m).

### 17.7. (6R,7R)-3-Acetoxymethyl-7-[(4-morpholin-4-yl-quinoline-2-carbonyl)-amino]-8-oxo-5-thia-1-aza-bicylo[4.2.0]oct-2-ene-2-carboxylic acid

The deprotection reaction is carried out according to the procedure described in example 5.4 from 0.7 g of (6R-7R-3-acetoxymethyl-7-[(4-morpholin-4-yl-quinoline-2-carbonyl)-amino]-8-oxo-5-thia-1-aza-bicylo[4.2.0]oct-2-ene-2-carboxylic acid benzhydryl ester (example 17.6) (1.0 mmol), 0.4 mL of anisole (4.0 mmol) and 0.7 mL of trifluoroacetic acid (10.0 mmol). The addition of hexane to the reaction medium leads to the formation of an oil. The supernatant is eliminated and the oil is triturated with cold water until a yellow precipitate is obtained which is filtered, washed with hexane then with ether, and dried under vacuum. PA 1191 is obtained as a yellow powder (10 mg, 2%).

¹H NMR (300 MHz, DMSO) δ ppm: 2.04 (3H, s), 3.30 (4H, m), 3.55 (1H, d, 7=18.2 Hz), 3.69 (1H, d, J=18.1 Hz), 3.90 (4H, s), 4.70 (1H, d, J=12.8 Hz), 5.03 (1H, d, J=120. Hz), 5.27 (1H, d, J=4.9 Hz), 5.95 (1H, m), 7.59 (1H, s), 7.67 (1H, t, J=7.4 Hz), 7.82 (1H, t, J=8.0 Hz), 8.11 (2H, d, J=8.2 Hz), 9.23 (1H, d, J=9.2 Hz). MS (IS>0) m/z: 513.4 (M+H⁺). Elementary analysis: for C₂₄H₂₄N₄O₇S.0.25 AcOEt 0.8H₂O: % theor. C 54.69, N 10.21; % exper. C 54.64, N 10.17.

### Example 18

### Preparation of an Aminoquinoline-Cephalosporin, Ref PA 1192

### (6R,7R)-3-Acetoxymethyl-7d[(4-(2-diethylamino-ethylamino)-quinoline-2-carbonyl]-amino}-8-oxo-5-thia-1-aza-bicyclo[4.2.0]oct-2-ene-2-carboxylic acid

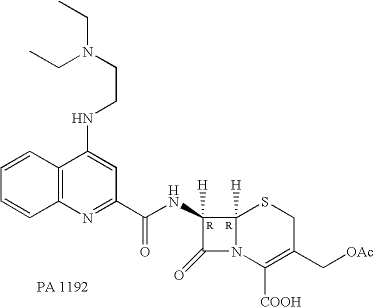

### 18.1. 4-(2-Diethylamino-ethylamino)-quinoline-2-carboxylic acid (2-diethylamino-ethyl)amide

This compound is prepared according to the procedure described in example 17.2 from 1.5 g of 4-chloro-quinoline-2-carboxylic acid ethyl ester (example 17.1) (6.4 mmol) and 9 mL of N,N-diethylethylenediamine (64.0 mmol). The product is obtained as brown oil (2.6 g, 100%).

¹H NMR (250 MHz, CDCl₃) δ ppm: 1.06 (12H, m), 2.61 (8H, m), 2.70 (2H, t, J=6.6 Hz), 2.80 (2H, t, J=6.0 Hz), 3.34 (2H, dd, J=10.3 Hz, J=4.7 Hz), 3.52 (2H, dd, J=13.0 Hz, J=6.5 Hz), 6.21 (1H, s), 7.31 (1H, s), 7.44 (1H, t, J=6.9 Hz), 7.62 (1H, t, J=7.0 Hz), 7.73 (1H, d, 7=8.3 Hz), 7.93 (1H, d, J=8.4 Hz), 8.60 (1H, s).

### 18.2. 4-2-Diethylamino-ethylamino)-quinoline-2-carboxylic acid

This product is prepared according to the procedure described in example 17.3 from 3.3 g of 4-2-diethylamino-ethylamino)quinoline-2-carboxylic acid (2-diethylamino-ethyl) amide (example 18.1) (8.6 mmol) and 34 mL of an aqueous solution of 2.5 M sodium hydroxide (86 mmol). Heating to reflux is maintained for 10 days. After cooling to room temperature, the reaction medium is diluted with 100 mL of ethanol and 100 mL of water then it is washed with 200 mL of dichloromethane. The pH of the aqueous phase is then brought to 7, at 0° C., with an aqueous solution of 1N HCl. The aqueous phase is evaporated to dryness and the product is extracted with 40 mL of DMF with stirring. The suspension is filtered and the filtrate is evaporated to dryness under vacuum. The product is obtained as orange oil (2.5 g, 100%).

¹H NMR (250 MHz, DMSO) δ ppm: 1.24 (6H, t, J=7.0 Hz), 3.22 (4H, q, J=6.6 Hz), 3.49 (2H, m), 4.02 (2H, m), 7.29 (1H, s), 7.65 (1H, m), 7.92 (1H, t, J=10.6 Hz), 8.31 (1H, d, J=8.5 Hz), 8.82 (1H, d, J=8.5 Hz), 9.63 (1H, s).

### 18.3. Mixture of (6R,7R)-3-acetoxymethyl-7-{4-(2-diethylamino-ethylamino)-quinoline-2-carbonyl)-amino}-8-oxo-5-thia-1-aza-bicyclo[4.2.0]oct-2-ene-2-carboxylic acid benzhydryl ester and (6R,7R)-3-acetoxymethyl-7-{[4-(2-diethylamino-ethylamino)quinoline-2-carbonyl)-amino}-8-oxo-5-thia-1-aza-bicyclo[4.2.0]oct-2-ene-2-carboxylic acid benzhydryl ester: Δ²/Δ³

The coupling product is prepared according to the procedure described in example 13.1 from 1.6 g of 4-(2-diethylamino-ethyl(amino)-quinoline-2-carboxylic acid (example 18.2) (3.2 mmol), 2.0 g of (6R,7R-3-acetoxymethyl-7-amino-8-oxo-5-thia-1-aza-bicyclo[4.2.0]oct-2-ene-2-carboxylic acid benzhydryl ester p-toluene sulfonic acid (3.2 mmol), 1 mL de N-methylmorpholine (10.0 mmol) and 1.7 g of PyBOP (3.2 mmol). The coupling product is obtained after purification by liquid chromatography on silica gel (Si0₂ 60A C.C 70-200 μm, eluent: dichloromethane/ethanol 90/10 v/v) as an orange oil (1.1 g, 52%) as a Δ²/Δ³ 45/55 mixture, used as such in the following step.

### 18.4. (6R,7R-3-acetoxymethyl-7-4-(2-diethylamino-ethylamino)quinoline-2-carbonyl)-amino-5,8-oxo-5λ⁴-thia-1-aza-bicyclo[4.2.0]oct-2-ene-2-carboxylic acid benzhydryl ester

The oxidation reaction is carried out according to the procedure described in example 5.2 from 1.1 g of the Δ²/Δ³ mixture of example 18.3 (1.6 mmol) and 0.7 g of 3-chloroperoxybenzoic acid (4.1 mmol). The oxidation product is obtained after recrystallization from dichloromethane/ether as an orange powder (0.5 g, 39%).

¹H NMR (300 MHz, CDCl₃,) δ ppm: 1.37 (6H, q, J=3.3 Hz), 2.07 (3H, s), 3.26 (1H, d, J=18.3 Hz), 3.45 (4H, t, J=6.3 Hz), 3.57(1H, d, J=16.8 Hz), 3.70 (2H, m), 3.88 (2H, m), 4.62 (1H, d, J=3.6 HZ), 4.78 (1H, d, J=13.8 Hz), 5.35 (1H, d, J=14.1 Hz), 6.34 (1H, dd, J=10.2 Hz, J=5.1 Hz), 7.01 (1H, s), 7.15-7.43 (12H, m), 7.61 (1H, t, J=7.8 Hz), 7.96 (2H, m), 9.47 (2H, d, J=10.8 Hz).

### 18.5. (6R,7R)-3-Acetoxymethyl-7-{[4-(2-diethylamino-ethylamino)quinoline-2-carbonyl]-amino}-8-oxo-5-thia-1-aza-bicyclo[4.2.0]oct-2-ene-2-carboxylic acid benzhydryl ester

The reduction reaction is carried out according to the procedure described in example 5.3 from 0.5 g of (6R,7R-3-acetoxymethyl-7-[(4-diethylamino-quinoline-2-carbonyl)-amino]-5,8-dioxo-5λ⁴-thia-1-aza-bicyclo[4.2.0]oct-2-ene-2-carboxylic acid benzhydryl ester (example 18.4) (0.6 mmol) and 0.1 mL of trichlorophosphine (1.4 mmol). After recrystallization from dichloromethane/ether, the reduction product is obtained as an orange oil (0.2 g, 51%).

¹H NMR (300 MHz, DMSO) δ ppm: 1.24 (6H, s), 1.98 (3H, s), 3.24 (4H,s), 3.42 (2H, d, J=1.2 Hz), 3.63 (1H, d, J=18.3 Hz), 3.73 (1H, d, J=16.5 Hz), 3.84 (1H, s), 4.68 (1H, d, J=13.5 Hz), 4.90 (1H, d, J=12.9 Hz), 5.33 (1H, d, J=4.2 Hz), 6.04 (1H, m), 6.92 (1H, s), 7.19-7.50 (11H, m), 7.60 (1H, m), 7.78 (1H, m), 7.98 (1H, m), 8.46 (1H, m).

### 18.6. (6R,7R)-3-Acetoxymethyl-7-{[(4-(2-diethylamino-ethylamino)quinoline-2-carbonyl]-amino-8-oxo-5-thia-1-aza-bicyclo[4.2.0]oct-2-ene-2-carboxylic acid

The deprotection reaction is carried out according to the procedure described in example 5.4 from 0.2 g of (6R,7R-3-acetoxymethyl-7-[4-(2-diethylamino-ethylamino) -quinoline-2-carbonyl]-amino}-8-oxo-5-thia-1-aza-bicylo[4.2.0]oct-2-ene-2-carboxylic acid benzhydryl ester (example 18.5) (0.3 mmol), 0.1 mL of anisole (1.3 mmol) and 0.2 mL of trifluoroacetic acid (3.2 mmol). After cooling to room temperature, the reaction mixture is filtered. The filtrate is precipitated using ether and the new filtered precipitate is washed with dichloromethane. The latter is solubilized in water and brought to pH 5 with an aqueous solution of 5% NaHCO₃ (w/v). The aqueous phase is evaporated to dryness and the product is extracted with 40 mL of DMF with stirring. The suspension is filtered and the filtrate is evaporated to dryness under vacuum. The product is obtained as an orange powder (50 mg, 29%).

¹H NMR (300 MHz, DMSO) δ ppm: 1.16 (6H, s), 2.03 (3H, s), 3.03 (4H,m), 3.33-3.49 (3H, m), 3.65 (2H, d, J=19.2 Hz), 4.75 (1H, d, J=11.7 Hz), 5.03 (1H, d, J=13.2 Hz), 5.24 (1H, m), 5.96 (1H, m), 7.20 (1H, s), 7.56 (1H, m), 7.81 (2H, m), 7.93 (1H, d, J=7.2 Hz), 8.30 (1H, d, J=7.8 Hz), 9.03 (1H, d, J=9.3 Hz). Elementary analysis: for C₂₆H₃₁N₅O₆S.8.5H₂O: % theor. C 44.95, N 10.08; % exper. C 44.97, N 9.68.

### Example 19

### Preparation of an Aminoquinoline-Cephalosporin, Ref PA 1199

### (6R,7R)-7-[2-(2-Amino-thiazol-4-yl)-2-methoxyimino-acetylamino]-3-[2-(7-chloro-quinolin 4-ylamino)-ethylsulfanylmethyl]-8-oxo-5-thia-1-aza-bicyclo[4.2.0]oct-2-ene-2-carboxylic acid

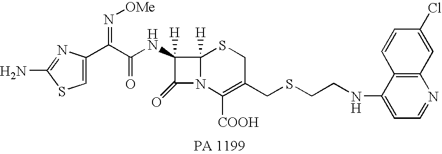

### 19.1. (6R,7R)-7-tert Butoxycarbonylamino-3-[2-(7-chloro-quinolin-4-ylamino) -ethylsulfanylmethyl]-8-oxo-5-thia-1-aza-bicyclo[4.2.0]oct-2-ene-2-carboxylic acid benzhydrylester

Sodium iodide (0.2 g, 1.5 mmol) is added to a solution of 7-tert-butoxycarbonylamino-3-chloromethyl-8-oxo-5-thia-1-aza-bicyclo[4.2.0]oct-2-ene-2-carboxylic acid benzhydrylester under argon (prepared according to the method described by H. A. Albrecht et al., J. Med. Chem. 1994, 37, 400-407) (0.8 g, 1.5 mmol) in 10 mL of dimethylformamide.

After 30 min of stirring, 0.4 g of 2-(7-chloro-quinolin-4-ylamino)ethanethiol (prepared according to the method described by J. Lhomme et al., Tetrahedron 1989, 45, 6455-6466) (1.5 mmol) are added to the mixture followed by 0.2 mL of N,N-diisopropylethylamine (1.5 mmol). The stirring is continued for 17 hr at room temperature. The reaction medium is then diluted with 50 mL of chloroform then it is washed successively with twice 50 mL of water and 50 mL of saturated aqueous NaCl solution. The organic phase is dried over magnesium sulfate, filtered then evaporated. The product is obtained after purification by liquid chromatography on silica gel (SiO₂ 60 Å C.C 6-35 μm, eluent: ethyl acetate/dichloromethane 90/10 (v/v) as a white powder (0.1 g, 12%). ¹H NMR (250 MHz, CDCl₃) δ ppm: 1.48 (9H, s), 2.84 (2H, m), 3.08 (1H, d, J=13.7 Hz), 3.37-3.71 (4H, m), 4.06 (1H, d, J=13.7 Hz), 5.67 (2H, m), 6.32 (1H, d, J=5.9 Hz), 6.90 (1H, s), 7.28-7.41 (12H, m), 7.78 (1H, d, J=8.9 Hz), 8.07 (1H, d, J 2.0 Hz), 8.36 (1H, d, A=5.7 Hz). MS (DCI/NH₃>0) m/z: 717 (M+H⁺).

### 19.2. (6R,7R)-7-Amino-3-chloromethyl-8-oxo-5-thia-1-aza-bicyclo[4.2.0]oct-2-ene-2-carboxylic acid

0.05 mL of 12M hydrochloric acid is injected at room temperature to a solution of (6R,7R)-7-tert-butoxycarbonylamino-3-[2-(7-chloroquinolin-4-ylamino)-ethylsulfanylmethyl]-8-oxo-5-thia-1-aza-bicyclo[4.2.0]oct-2-ene-2-carboxylic acid benzhydrylester (0.1 g, 0.2 mmol) (example 19.1) in 0.5 mL of formic acid. After 1 hr of stirring, the medium is precipitated by addition of 10 mL of a 2/1 v/v ethyl acetate/acetone mixture. The precipitate formed is filtered, washed with dichloromethane then using diethyl ether before being dried under vacuum. The product is obtained as a white powder (0.1 g, 82%).

¹H NMR (250 MHz, DMSO) δ ppm: 2.89 (2H, m), 3,5 (6H, m), 5.11 (1H, d, J=4.9 Hz), 5.23 (1H, m), 6.89 (1H, d, J=7.0 Hz), 7.79 (1H, d, J=9.0 Hz), 8.12 (1H, d, J=1.7 Hz), 8.55 (1H, d, J=7.0 Hz) 8.74 (1H, d, J=9.0 Hz), 9.77 (1H, broad s). MS (IS>0) m/z: 451.15 (M+H⁺).

### 19.3. (6R,7R)-7-[2-(2-Amino-thiazol-4-yl)-2-methoxyimino-acetylamino]-3-[2-(7-chloro-quinolin-4-ylamino)-ethylsulfanylmethyl]-8-oxo-5-thia-1-aza-bicyclo[4.2.0]oct-2-ene-2-carboxylic acid

40 μL of triethylamine (0.3 mmol) then 50 mg of (2-amino-thiazol-4-yl)methoxyimino-thioacetic acid S-benzothiazol-2-yl ester (0.1 mmol) are successively added to a suspension of (6R,7R-7-amino-3-chloromethyl-8-oxo-5-thia-1-aza-bicyclo[4.2.0]oct-2-ene-2-carboxylic acid (62 mg, 0.1 mmol) (example 19.2) at −5° C./−10° C. in 5 mL of dichloromethane. After 1 hr of stirring at room temperature, the medium is diluted with 10 mL of distilled water. The emulsion is filtered and the precipitate is washed successively with cold water (6° C.), cold ethanol (6° C.), dichloromethane then diethyl ether before being dried under vacuum. PA 1199 is obtained as a white powder (38 mg, 47%).

¹H NMR (250 MHz, DMSO) δ ppm: 2.80 (2H, m), 3.08 (2H, m), 3.50 (4H, m), 3.83 (3H, s), 5.12 (1H, d, J=4.5 Hz), 5.70 (1H, m), 6.61 (1H, d, J=4.5 Hz), 6.75 (1H, s), 7.22 (2H, s), 7.50 (1H, d, J=9.2 Hz), 7.81 (1H, s), 8.36 (2H, m) 9.59 (1H, d, J=7.6 Hz). MS (IS>0) m/z: 634.05 (M+H⁺).

### Example 20

### Preparation of an Aminoquinoline-Cephalosporin, Ref PA 1273

### Sodium (6R,7R)-7-[2-(7-chloroquinolin-4-ylamino)-acetylamino]-3-methyl-8-oxo-5-thia-1-aza-bicyclo[4.2.0]oct-2-ene-2-carboxylate.

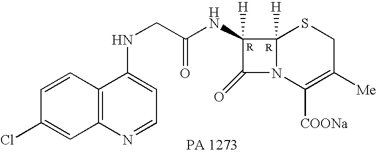

### 20.1. (6R,7R)-7-[2-(7-chloroquinolin-4-ylamino)-acetylamino]-3-methyl-8-oxo-5-thia-1-aza-bicyclo[4.2.0]oct-2-ene-2-carboxylic acid benzhydryl ester hydrochloride salt.

To a mixture under argon of (7-chloro-quinolin-4-ylamino)-acetic acid (example 3.1) (1.7 g, 7.2 mmol) and (6R,7R)-7-amino-3-methyl-8-oxo-5-thia-1-aza-bicyclo[4.2.0]oct-2-ene-2-carboxylic acid benzhydryl ester p-toluene sulfonic acid (prepared according to the method described by R. G. Micetich et al., Syhtesis 1985, 6-7, 693-695) (4.0 g, 7.2 mmol) in 25 mL of tetrahydrofuran and cooled at −30° C. is added phosphorus oxychloride (1.0 mL, 10.5 mmol) followed by 2,4,6-collidine (3.5 mL, 26.1 mmol). This mixture is stirred at −20° C. for 3 hr and then quenched with wet tetrahydrofuran and dilute with 100 mL of dichloromethane. This mixture is washed successively with 100 mL of water and 100 mL of an 1N aqueous hydrochloric acid solution. The organic layer is concentrated under vacuum until the product begins to precipitate. The mixture is then put at −30° C. overnight. The precipitate thus obtained is filtered off, washed with dichloromethane and diethyl ether and then dried under vacuum. The title compound is obtained as a white powder (2.5 g, 53%).

¹H NMR (250 MHz, DMSO) δ ppm: 2.02 (3H, s), 3.44 (1H, d, J=17.9 Hz), 3.62 (1H, d, J=17.9 Hz), 4.39 (2H, m), 5.14 (1H, d, J=4.6 Hz), 5.73 (1H, dd, J=4.6 Hz, J=8.0 Hz), 6.74 (1H, broad s), 6.90 (1H, s), 7.27-7.52 (10H, m), 7.83 (1H, d, J=8.9 Hz), 8.07 (1H, s), 8.58 (1H, d, J=8.9 Hz), 8.60 (1H, broad s), 9.37 (1H, d, J=8.0 Hz), 9.78 (1H, broad s). MS (IS>0) m/z: 599.3 (M−Cl⁻)⁺.

### 20.2. Sodium (6R,7R)-7-[2-(7-chloroquinolin-4-ylamino)-acetylamino]-3-methyl-8-oxo-5-thia-1-aza-bicyclo[4.2.0]oct-2-ene-2-carboxylate

The deprotection reaction is carried out according to the procedure described in example 5.4 from 2.5 g of (6R,7R)-7-[2-(7-chloroquinolin-4-ylamino)-acetylamino]-3-methyl-8-oxo-5-thia-1-aza-bicyclo[4.2.0]oct-2-ene-2-carboxylic acid benzhydryl ester hydrochloride salt (example 20.1) (3.9 mmol), 1.7 mL of anisole (15.4 mmol) and 1.7 mL of trifluoroacetic acid (23.2 mmol). The sodium salt is prepared as follows: the product is suspended in 15 mL of methanol at 0° C. and triethylamine (1.0 mL, 7.0 mmol) in solution in 1 mL of methanol is added to it, followed by a solution of sodium 2-ethylhexanoate (0.4 g, 2.3 mmol) in a mixture of methanol (1 mL) and ethyl acetate (1 mL). The mixture is stirred 20 min at 0° C., and then 10 mL of ethyl acetate is added to get turbidity in the solution. The stirring is continued at the same temperature for 25 min. Ethyl acetate (60 mL) is added in 40 min and the stirring further continued at the same temperature for 1 h. The mixture is then put at −30° C. overnight, after that the precipitate is filtered off and washed with ethylacetate to obtain after drying PA 1273 as a pale yellow powder (0.9 g, 49%).

¹H NMR (250 MHz, DMSO) δ ppm: 1.89 (3H, s), 3.05 (1H, d, J=17.5 Hz), 3.39 (1H, d, J=17.5 Hz), 4.04 (2H, broad s), 4.88 (1H, d, J=4.4 Hz), 5.41 (1H, dd, J=4.4 Hz, J=8.0 Hz), 6.31 (1H, d, J=5.3 Hz), 7.48 (1H, d, J=9.0 Hz), 7.82 (2H, broad s), 8.25 (1H, d, J=9.0 Hz), 8.38 (1H, d, J=5.3 Hz), 9.07 (1H, d, J=8.0 Hz). MS (IS>0) m/z: 433.3 (M−Na⁺+2H⁺). Elementary analysis: for C₁₉H₁₆ClN₄NaO₄S.2.9H₂O.0.15AcOEt: % theor. C 45.24, N 10.76; % exper. C 45.22, N 10.77.

### Example 21

### Preparation of an Aminoquinoline-Cephalosporin, Ref PA 1272

### Racemic mixture of sodium (6R,7R,11R/11S)-3-acetoxymethyl-7-[2-(7-chloroquinolin-4-ylamino)-propionylamino]-8-oxo-5-thia-1-aza-bicyclo[4.2.0]oct-2-ene-2-carboxylate

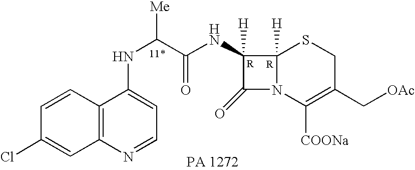

### 21.1. 2-(7-Chloroquinolin-4-ylamino)-propionic acid

This compound is prepared according to the procedure described in example 1.1 starting from 1.0 g of 4,7-dichloroquinoline (5.1 mmol), 1.0 g of alanine (11.2 mmol) and 2.7 g of phenol (28.5 mmol) heated for 24 hours at 120° C. The title compound is obtained as a white powder (0.2 g, 20%).

¹H NMR (250 MHz, TFA) δ ppm: 1.56 (3H, broad d, J=6.0 Hz), 4.50 (1H, m), 6.51 (1H, s), 7.44 (1H, d, J=8.1 Hz), 7.63 (1H, s), 7.90 (1H, d, J=8.7 Hz), 8.07 (1H, s).

### 21.2. Racemic mixture of (6R,7R,11R/11S)-3-acetoxymethyl-7-[2-(7-chloroquinolin-4-ylamino)-propionylamino]-8-oxo-5-thia-1-aza-bicyclo[4.2.0]oct-2-ene-2-carboxylic acid benzhydryl ester hydrochloride salt

This compound is prepared according to the procedure described in example 20.1 starting from 1.6 g of 2-(7-chloro-quinolin-4-ylamino)-propionic acid (example 21.1) (6.5 mmol), 4.0 g of (6R,7R)-3-acetoxymethyl-7-amino-8-oxo-5-thia-1-aza-bicyclo-[4.2.0]oct-2-ene-2-carboxylic acid benzhydryl ester p-toluene sulfonic acid (6.6 mmol), 0.9 mL of phosphorus oxychloride (9.5 mmol) and 3.1 mL of 2,4,6-collidine (23.6 mmol). The title compound is obtained as a white powder (2.4 g, 52%)

¹H NMR (250 MHz, DMSO) δ ppm: 1.74 (6H, m), 2.07 (3H, s), 2.09 (3H, s), 3.68 (4H, m), 4.80 (4H, m), 4.96 (1H, d, J=13.0 Hz), 4.98 (1H, d, J=13.0 Hz), 5.28 (1H, d, J=5.0 Hz), 5.30 (1H, d, J=5.0 Hz), 5.88 (2H, m), 6.73 (1H, d, J=7.0 Hz), 6.80 (1H, d, J=7.0 Hz), 7.04 (2H, s), 7.37-7.62 (20H, m), 7.97 (2H, dd, J=1.3 Hz, d, J=9.1 Hz), 8.25 (2H, m), 8.81 (2H, m), 8.94 (2H, d, J=9.1 Hz), 9.33 (1H, d, J=7.0 Hz), 9.39 (1H, d, J=7.0 Hz), 9.57 (1H, d, J=7.8 Hz), 8.94 (2H, d, J=7.8 Hz). MS (IS>0) m/z: 824.1 (M−Cl⁻)⁺.

### 21.3. Racemic mixture of sodium (6R,7R,11R/11S)-3-acetoxymethyl-7-[2-(7-chloro-quinolin-4-ylamino)-propionylamino]-8-oxo-5-thia-1-aza-bicyclo[4.2.0]oct-2-ene-2-carboxylate

The deprotection reaction is carried out according to the procedure described in example 5.4 starting from 2.4 g of the racemic mixture of (6R,7R,11R/11S)-3-acetoxymethyl-7-[2-(7-chloroquinolin-4-ylamino)-propionylamino]-8-oxo-5-thia-1-aza-bicyclo[4.2.]oct-2-ene-2-carboxylic acid benzhydryl ester hydrochloride salt (example 21.2) (3.4 mmol), 1.5 mL of anisole (13.6 mmol) and 2.5 mL of trifluoroacetic acid (34.0 mmol). The sodium salt is prepared according to the procedure described in example 20.2 with triethylamine (0.6 mL, 4.4 mmol) and sodium 2-ethylhexanoate (0.3 g, 2.2 mmol) to obtain PA 1273 as a pale yellow powder (0.9 g, 51%).

¹H NMR (250 MHz, DMSO) δ ppm: 1.52 (3H, d, J=7.0 Hz), 1.53 (3H, d, J=7.0 Hz), 1.99 (3H, s), 2.01 (3H, s), 3.11-3.51 (4H, m), 4.29 (2H, q, J=7.0 Hz), 4.72 (1H, d, J=11.3 Hz), 4.76 (1H, d, J=11.3 Hz), 4.98 (4H, m), 5.44 (2H, m), 6.29 (1H, d, J=5.5 Hz), 6.40 (1H, d, J=5.5 Hz), 7.33 (2H, m), 7.49 (2H, dd, J=2.0 Hz, J=9.0 Hz), 7.80 (2H, d, J=2.0 Hz), 8.41 (4H, m), 9.07 (1H, d, J=8.0 Hz), 9.27 (1H, d, J=8.0 Hz). MS (IS>0) m/z: 505.4 (M−Na⁺+2H⁺). Elementary analysis: for C₂₂H₂₀ClN₄NaO₆S,2.3H₂O.0.22AcOEt: % theor. C 46.75, N 9.53; % exper. C 46.78, N 9.54.

### Example 22

### Preparation of an Aminoquinoline-Cephalosporin, Ref PA 1283

### Sodium (6R,7R)-3-acetoxymethyl-7-{2-(2-aminothiazol-4-yl)-2-[2(Z)-(7-chloro-quinolin-4-ylamino)-ethoxyimino]-acetylamino}-8-oxo-5-thia-1-aza-bicyclo[4.2.0]oct-2-ene-2-carboxylate

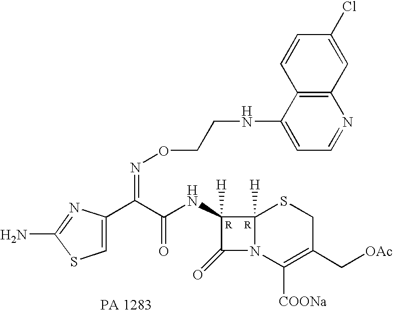

### 22.1. [2(Z)-(7-Chloroquinolin-4-ylamino)-ethoxyimino]-[2-(tritylamino)-thiazol-4-yl]-acetic acid ethyl ester

Hydroxyimino-[2-(tritylamino)-thiazol-4-yl]-acetic acid ethyl ester (16.5 g, 36.1 mmol) and (2-bromoethyl)-(7-chloro-quinolin-4-yl)-amine (9.6 g, 33.6 mmol) are dissolved in 150 mL of anhydrous dimethylformamide. To this stirred mixture is added potassium carbonate (15.0 g, 108.2 mmol) followed by tetrabutylammonium iodide (3.0 g, 8.2 mmol). The mixture is stirred for 24 h and then filtered. The filter cake is washed with dimethylformamide. The filtrate is diluted with 300 mL of ethylacetate and washed successively with 300 mL of water, 300 mL of saturated aqueous sodium bicarbonate and 300 mL of brine. The organic layer is dried over magnesium sulfate, filtered and concentrated under vacuum. The residual oil is purified by flash-chromatography using a dichloromethane/ethanol gradient to afford the title compound as an orange powder (4.9 g, 22%).

¹H NMR (250 MHz, CDCl₃) δ ppm: 1.28 (3H, t, J=7.0 Hz), 3.60 (2H, q, J=5.0 Hz), 4.35 (2H, q, J=7.0 Hz), 4.57 (2H, t, J=5.0 Hz), 5.71 (1H, broad t, J=5.0 Hz), 6.39 (1H, q, J=5.3 Hz), 6.52 (1H, s), 6.97 (1H, s), 7.25 (16H, m), 7.82 (1H, d, J=9.0 Hz), 7.94 (1H, d, J=2.2 Hz), 8.40 (1H, d, J=5.3 Hz).

### 22.2. [2(Z)-(7-Chloroquinolin-4-ylamino)-ethoxyimino]-[2-(tritylamino)-thiazol-4-yl]-acetic acid

[2(Z)-(7-Chloroquinolin-4-ylamino)-ethoxyimino]-[2-(tritylamino)-thiazol-4-yl]-acetic acid ethyl ester (3.88 g, 5.9 mmol) (example 22.1) is dissolved in 40 mL of 1,4-dioxane. To this solution is added a 2M aqueous NaOH solution (14.6 mL, 29.3 mmol). The mixture is heated to reflux for 5 hr and then filtered after cooling to room temperature. The precipitate is washed with water and dichloromethane before drying under vacuum to afford the title compound as a white powder (3.4 g, 92%)

¹H NMR (250 MHz, CDCl₃) δ ppm: 1.28 (3H, t, J=7.0 Hz), 3.60 (2H, q, J=5.0 Hz), 4.35 (2H, q, J=7.0 Hz), 4.57 (2H, t, J=5.0 Hz), 5.71 (1H, broad t, J=5.0 Hz), 6.39 (1H, q, J=5.3 Hz), 6.52 (1H, s), 6.97 (1H, s), 7.25 (16H, m), 7.82 (1H, d, J=9.0 Hz), 7.94 (1H, d, J=2.2 Hz), 8.40 (1H, d, J=5.3 Hz). MS (IS>0) m/z: 634.3 (M+H⁺).

### 22.3. (6R,7R)-3-Acetoxymethyl-7-{2-[2(Z)-(7-chloroquinolin-4-ylamino)-ethoxyimino]-2-[2-(tritylamino)-thiazol-4-yl]-acetylamino}-8-oxo-5-thia-1-aza-bicyclo[4.2.0]oct-2-ene-2-carboxylic acid benzhydryl ester

This compound is prepared according to the procedure described in example 20.1 starting from 1.7 g of [2(Z)-(7-chloroquinolin-4-ylamino)-ethoxyimino]-[2-(tritylamino)-thiazol-4-yl]-acetic acid (example 22.2) (2.8 mmol), 1.7 g of (6R,7R)-3-acetoxymethyl-7-amino-8-oxo-5-thia-1-aza-bicyclo-[4.2.0]oct-2-ene-2-carboxylic acid benzhydryl ester p-toluene sulfonic acid (2.8 mmol), 0.4 mL of phosphorus oxychloride (4.0 mmol) and 1.3 mL of 2,4,6-collidine (9.9 mmol). The title compound is obtained as a white powder after purification by flash-chromatography using a (dichloromethane+0.1% trifluoroacetic acid)/(ethanol+0.1% trifluoroacetic acid) gradient (1.5 g, 51%)

¹H NMR (250 MHz, DMSO) δ ppm: 1.93 (3H, s), 2.79 (1H, d, J=18.3 Hz), 3.46 (1H, d, J=18.3 Hz), 3.82 (2H, broad s), 4.30 (2H, broad s), 4.40 (1H, d, J=13.4 Hz), 4.85 (1H, d, J=13.4 Hz), 5.10 (1H, d, J=4.8 Hz), 5.74 (1H, dd, J=4.8 Hz, J=8.0 Hz), 6.88 (3H, m), 7.22-7.54 (26H, m), 7.88 (1H, s), 8.39 (1H, d, J=8.8 Hz), 8.56 (1H, broad s), 8.88 (1H, s), 9.11 (1H, broad s), 9.42 (1H, d, J=8.3 Hz). MS (IS>0) m/z: 1054.7 (M+H⁺).

### 22.4. Sodium (6R,7R)-3-acetoxymethyl-7-{2-(2-aminothiazol-4-yl)-2-[2(Z)-(7-chloro-quinolin-4-ylamino)-ethoxyimino]-acetylamino}-8-oxo-5-thia-1-aza-bicyclo[4.2.0]oct-2-ene-2-carboxylate

(6R,7R)-3-Acetoxymethyl-7-{2-[2(Z)-(7-chloroquinolin-4-ylamino)-ethoxyimino]-2-[2-(tritylamino)-thiazol-4-yl]-acetylamino}-8-oxo-5-thia-1-aza-bicyclo[4.2.0]oct-2-ene-2-carboxylic acid benzhydryl ester (1.0 g, 0.9 mmol) (example 22.3) is dissolved under argon in 10 mL of anhydrous dichloromethane. This solution is cooled to 0° C. before the addition of trifluoroacetic acid (0.7 mL, 9.2 mmol) followed by anisole (0.4 mL, 3.7 mmol). The resulting mixture is stirred for 1 hr 45 at room temperature. Diethyl ether is then added to the solution and the resulting precipitate is filtered off. This precipitate is then dissolved in 90% aqueous formic acid (2.7 mL, 71.0 mmol) and the solution is stirred for 2 hr at room temperature. The insoluble material is filtered off. The filtrate is diluted with 10 mL of water, cooled to 0° C. and adjusted to pH 4-5 with 28-30% aqueous ammonium hydroxide. The resulting precipitate is filtered and washed successively with water, ethanol, acetone, dichloromethane and diethyl ether before drying under vacuum. The sodium salt is then prepared according to the procedure described in example 20.2 with triethylamine (0.13 mL, 0.9 mmol) and sodium 2-ethylhexanoate (0.07 g, 0.45 mmol) to obtain PA 1283 as a beige powder (0.2 g, 40%).

¹H NMR (250 MHz, DMSO) δ ppm: 1.97 (3H, s), 2.75 (1H, d, J=18.3 Hz), 3.43 (1H, d, J=18.3 Hz), 3.63 (2H, m), 4.30 (2H, broad s), 4.59 (1H, d, J=12.0 Hz), 4.99 (2H, m), 5.62 (1H, dd, J=4.6 Hz, J=8.1 Hz), 6.54 (1H, d, J=5.5 Hz), 6.80 (1H, s), 7.29 (3H, broad s), 7.47 (1H, dd, J=2.0 Hz, J=9.1 Hz), 7.77 (1H, d, J=2.0 Hz), 8.20 (1H, d, J=9.1 Hz), 8.41 (1H, d, J=5.5 Hz), 9.55 (1H, d, J=8.1 Hz). MS (IS>0) m/z: 646.4 (M−Na⁺+2H⁺). Elementary analysis: for C₂₆H₂₃ClN₇NaO₇S₂,4.5H₂O.0.14AcOEt: % theor. C 41.99, N 12.90; % exper. C 42.03, N 12.90.

Examples 23-27 Below Exemplify the Preparation of Hybrid Molecules of the Family of Aminoquinoline-Quinolones

### Example 23

### Aminoquinoline-Quinolone Hybrid Molecule, Ref PA 1123

### 7-[4-(7-Chloroquinolin-4-yl)-piperazin-1-yl]-1-cyclopropyl-6-fluoro-1,4-dihydro-quinoline-3-carboxylic acid

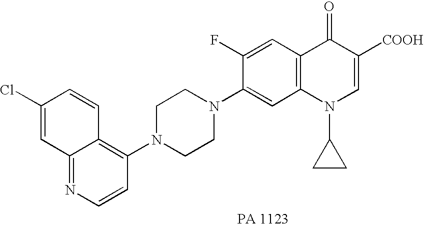

A suspension of 4,7-dichloroquinoline (0.6 g, 2.9 mmol), ciprofloxacin (0.6 g, 2.0 mmol) and potassium carbonate (0.1 g, 9.8 mmol) in 13 mL of dimethylacetamide is heated to 140° C. for 24 hours. After cooling to room temperature, the resulting suspension is filtered. The filtrate is precipitated using diethyl ether and the precipitate formed is filtered then washed with water. It is then stirred once more with 100 mL of chloroform for 1 hr before being filtered once more and dried under vacuum. PA 1123 is obtained as a yellow powder (0.3 g, 35%).

¹H NMR (300 MHz, DMSO) δ ppm: 1.23 (2H, m), 1.33 (2H, m), 3.72 (4H, m), 3.85 (1H, m), 4.04 (4H, m), 7.25 (1H, d, J=6.9 Hz), 7.59 (1H, d, J=7.5 Hz), 7.73 (1H, dd, J=2.1 Hz, J=9.3 Hz), 7.98 (1H, d, J=13.2 Hz), 8.11 (1H, d, J=2.1 Hz), 8.30 (1H, d, J=9.3 Hz), 8.69 (1H, s), 8.76 (1H, d, J=6.9 Hz). MS (IS>0) m/z: 493.2 (M+H⁺). Elementary analysis: for C₂₆H₂₂ClFN₄O₃.0.5H₂O: % theor. C 62.21. N 11.12; % exper. C 62.30. N 11.26.

### Example 24

### Aminoquinoline-Quinolone Hybrid Molecule, Ref PA 1126

### 7-[4-(7-Chloroquinolin-4-yl)-piperazin-1-yl]-1-cyclopropyl-6-fluoro-1,4-dihydroquinoline-3-carboxylic acid hydrochloride

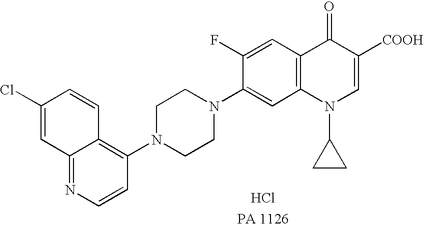

0.4 mL of a solution of 5M HCl in 2-propanol (2.0 mmol) is added dropwise to a solution of PA1123 (example 23) (0.1 g, 0.2 mmol) in 10 mL of chloroform at 0° C. After 1 hr of stirring at 0° C. the product is precipitated using diethyl ether and filtered. The solid is then stirred once more with 100 mL of chloroform for 3 hr then filtered, washed with ethanol and diethyl ether before being dried under vacuum. PA 1126 is obtained as a yellow powder (0.1 g, 77%).

¹H NMR (300 MHz, DMSO) δ ppm: 1.22 (2H, m), 1.32 (2H, m), 3.73 (4H, m), 3.83 (1H, m), 4.08 (4H, m), 7.26 (1H, d, J=6.9 Hz), 7.58 (1H, d, J=7.5 Hz), 7.74 (1H, J=8.7 Hz), 7.93 (1H, d, J=13.5 Hz), 8.15 (1H, s), 8.31 (1H, d, J=8.7 Hz), 8.69 (1H, s), 8.75 (1H, d, J=6.6 Hz). MS (IS>0) m/z: 493.2 (M+H⁺). Elementary analysis: for C₂₆H₂₂ClFN₄O₃—HCl0.2.5H₂O: % theor. C 54.36, N 9.75; % exper. C 54.10, N 9.50.

### Example 25

### Aminoquinoline-Quinolone Hybrid Molecule, Ref PA 1127

### 7-{4-[2-(7-Chloroquinolin-4-ylamino)-ethyl]-piperazin-1-yl}-1-cyclopropyl-6-fluoro-4-oxo-1,4-dihydroquinoline-3-carboxylic acid hydrochloride

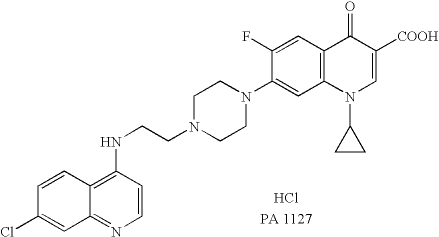

A suspension under argon of (2-bromoethyl)(7-chloroquinolin-4-yl)-amine (0.5 g, 1.8 mmol), ciprofloxacin (0.4 g, 1.2 mmol), and potassium carbonate (0.8 g, 5.9 mmol) in 10 mL of dimethylformamide is heated to 140° C. under stirring for 24 hr. After cooling to room temperature, the suspension is filtered. The solid is solubilized in 20 mL of water and the solution is returned to a neutral pH with an aqueous solution of 1M HCl. The precipitate formed is filtered and then washed with water, with ethanol, and then with diethyl ether. The product obtained is then replaced in suspension in a 1:1 v/v chloroform/ethanol mixture cooled to 0° C. and added dropwise to 1.2 mL of a solution of 5M HCl in 2-propanol (5.9 mmol). After 1 hr of stirring at 0° C. the product is precipitated using diethyl ether and filtered. The solid is then stirred once more with 50 mL of dichloromethane for 17 hr then filtered, washed with dichloromethane and diethyl ether before being dried under vacuum. PA 1127 is obtained as a beige powder (0.1 g, 9%).

¹H NMR (300 MHz, DMSO) δ ppm: 1.21 (2H, m), 1.31 (2H, m), 3.42-4.09 (13H, m), 7.10 (1H, d, J=7.2 Hz), 7.64 (1H, d, J=7.5 Hz), 7.82 (1H, dd, J=1.8 Hz, I=9.0 Hz), 7.98 (1H, d, J=12.9 Hz), 8.10 (1H, d, J=1.8 Hz), 8.69 (1H, s), 8.70 (1H, d, J=7.2 Hz), 8.83 (1H, d, J=9.0 Hz), 9.80 (1H, s), 11.80 (1H, s), 14.50 (1H, s). MS (IS>0) m/z: 536.2 (M−Cl)⁺. Elementary analysis: for C₂₈H₂₇ClFN₅O₃.HCl.6H₂O: % theor. C 46.89, N 9.77; % exper. C 47.22, N 9.63.

### Example 26

### Preparation of an Aminoquinoline-Quinolone, Ref PA 1284

### 1-Cyclopropopyl-6-fluoro-4-oxo-7-[4-(7-trifluoromethylquinolin-4-yl)-piperazin-1-yl]-1,4-dihydroquinoline-3-carboxylic acid hydrochloride salt

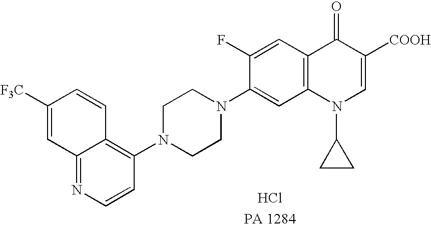

A mixture of 4-chloro-7-trifluoromethylquinoline (0.6 g, 2.4 mmol) and ciprofloxacine (0.7 g, 2.1 mmol) in 20 mL of 2-ethoxyethanol is heated to 135° C. for 17 hours. After cooling to room temperature, the suspension is filtered and the precipitate is washed with 30 mL of 2-ethoxyethanol and then 50 mL of water to obtain after drying under vacuum PA 1284 as an orange powder (0.3 g, 50%).

¹H NMR (250 MHz, DMSO) δ ppm: 1.22-1.33 (4H, m), 3.73 (4H, broad s), 3.84 (1H, broad s), 4.05 (4H, broad s), 7.35 (1H, d, J=6.8 Hz), 7.58 (1H, d, 7=7.4 Hz), 7.97 (2H, m), 8.48 (2H, m), 8.69 (1H, s), 8.86 (1H, d, J=6.8 Hz), 15.20 (1H, broad s). MS (IS>0) m/z: 527.4 (MH⁺). Elementary analysis: for C₂₇H₂₂F₄N₄O₃.HCl.1.5H₂O: % theor. C 54.96, N 9.50; % exper. C 55.03, N 9.34.

### Example 27

### Preparation of an Aminoquinoline-Quinolone, Ref PA 1285

### 7-[4-(8-Chloroquinolin-4-yl)-piperazin-1-yl]-1-cyclopropopyl-6-fluoro-4-oxo-1,4-dihydroquinoline-3-carboxylic acid hydrochloride salt

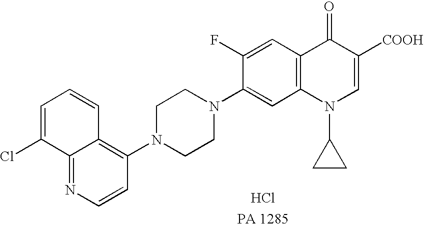

This compound is prepared according to the procedure described in example 26 starting from 4,8-dichloroquinoline (0.8 g, 4.2 mmol) and ciprofloxacine (0.4 g, 1.2 mmol). PA 1285 is obtained as an orange powder (0.7 g, 63%).

¹H NMR (250 MHz, DMSO) δ ppm: 1.18-1.45 (4H, m), 3.85 (4H, broad s), 3.95 (1H, broad s), 4.19 (4H, broad s), 7.43 (1H, d, J=6.7 Hz), 7.69 (1H, d, J=7.4 Hz), 7.81 (1H, t, J=8.0 Hz), 8.07 (1H, d, J=13.5 Hz), 8.30 (1H, d, J=8.0 Hz), 8.38 (1H, d, J=8.0 Hz), 8.79 (2H, m). MS (IS>0) m/z: 493.6 (MH⁺). Elementary analysis: for C₂₆H₂₂ClFN₄O₃—HCl.2.7H₂O: % theor. C 54.02, N 9.69; % exper. C 54.04, N 9.56.

Examples 28 Through 30 Below Exemplify the Creation of Hybrid Molecules in the Aminoquinoline-Nitroimidazole Family.

### Example 28

### Aminoquinoline-Nitroimidazole Hybrid Molecule, Ref PA 1129

### (7-Chloroquinolin-4-yl)-[2-(2-methyl-5-nitroimidazol-1-yl)-ethyl]-amine

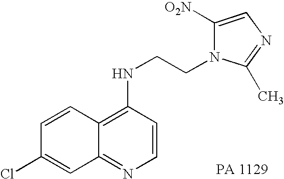

0.8 mL of triethylamine (5.5 mmol) is injected into a solution of (2-bromoethyl)-(7-chloroquinolin-4-yl)-amine (prepared according to the method described by B. Meunier et al. in patent application FR 2862304) (0.7 g, 2.5 mmol) and 2-methyl-5-nitro-imidazole (0.3 g, 2.5 mmol) in 10 mL of dimethylformamide. The mixture is heated to 140° C. for 24 hr. After cooling to room temperature, the reaction medium is diluted with 200 mL of dichloromethane and then washed with twice 200 mL of water followed by 200 mL of NaCl-saturated water. The organic phase is dried over magnesium sulfate, filtered, then concentrated in a rotary evaporator until the product starts to precipitate. Precipitation is continued at 6° C. for 24 hr and the filtered product is washed with cold dichloromethane (6° C.) and then with diethyl ether before being dried under vacuum. PA 1129 is obtained as a white powder (0.1 g, 7%).

¹H NMR (300 MHz, DMSO) δ ppm: 2.21 (3H, s), 3.73 (2H, q, J=5.7 Hz), 4.25 (2H, t, J=5.7 Hz), 6.64 (1H, d, J=5.4 Hz), 7.43 (1H, t, J=5.7 Hz), 7.49 (1H, dd, J=2.1 Hz, J=9.0 Hz), 7.81 (1H, d, J=2.1 Hz), 8.13 (1H, d, J=9.0 Hz), 8.37 (1H, s), 8.43 (1H, d, J=5.4 Hz). MS (DCI/NH₃>0) m/z: 332 (M+H⁺). Elementary analysis: for C₁₅H₁₄ClN₅O₂: % theor. C 54.30, N 21.11; % exper. C 54.07, N 21.41.

### Example 29

### Aminoquinoline-Nitroimidazole Hybrid Molecule, Ref PA 1130

### [2-(2-Methyl-5-nitroimidazol-1-yl)-ethyl]-(7-trifluoromethylquinolinyl-4-yl)-amine

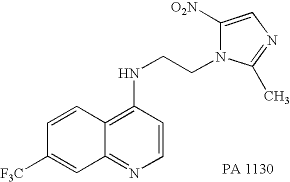

A suspension under argon of (2-bromoethyl)(7-trifluoromethylquinolin-4-yl)-amine (prepared according to the method described by B. Meunier et al. in patent application FR 2862304) (0.5 g, 1.7 mmol), of 2-methyl-5-nitroimidazole (0.2 g, 1.8 mmol) and potassium carbonate (1.2 g, 8.8 mmol) in 20 mL of dimethylformamide is heated to 70° C. for 24 hr. The treatment is then identical to the one described for PA 1129 (example 20). PA 1130 is obtained as a white powder (0.1 g, 24%).

¹H NMR (300 MHz, DMSO) δ ppm: 2.21 (3H, s), 3.76 (2H, q, J=5.4 Hz), 4.27 (2H, t, J=5.4 Hz), 6.75 (1H, d, J=5.4 Hz), 7.58 (1H, t, J=5.4 Hz), 7.73 (1H, d, J=8.7 Hz), 8.10 (1H, s), 8.34 (1H, d, J=8.7 Hz), 8.38 (1H, s), 8.54 (1H, d, J=5.4 Hz), MS (DCI/NH₃>0) m/z: 366 (M+H⁺). Elementary analysis: for C₁₆H₁₄F₃N₅O₂.0.5H₂O: % theor. C 51.34, N 18.71; % exper. C 51.13, N 18.73.

### Example 30

### Aminoquinoline-Nitroimidazole Hybrid Molecule, Ref PA 1173

### 1-[2-(7-Chloroquinolin-4-ylamino)-ethylamino]-3-(2-methyl-5-nitroimidazol-1-yl)-propan-2-ol

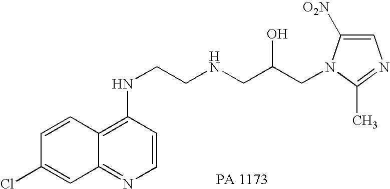

0.2 mL of triethylamine (1.3 mmol) is injected into a suspension under argon of N¹-(7-chloroquinolin-4-yl)-ethane-1,2-diamine (prepared according to the method described by B. Meunier et al., ChemBioChem 2000, 1, 281-283) (0.7 g, 3.4 mmol) and 2-methyl-5-nitro-1-oxiranyl-1H-imidazole (prepared according to the method described by E. Grunberg et al., 3. Med. Chem. 1974, 17, 1019-1020) (0.6 g, 3.2 mmol) in 10 mL of absolute ethanol. The mixture is brought to reflux for 5 hr. After cooling to room temperature, the reaction medium is concentrated to dryness using a rotary evaporator and purified by liquid chromatography on silica gel (SiO₂ 60 Å C.C 6-35 μm, eluent: dichloromethane/methanol/30% ammonia 88:10:2 v/v/v). After recrystallization from ethanol/water at 6° C., PA 1173 is obtained as a white powder (0.2 g, 16%).

¹H NMR (300 MHz, DMSO) δ ppm: 2.02 (1H, broad s), 2.44 (3H, s), 2.66 (1H, dd, J=12.1 Hz, J=6.2 Hz), 2.67 (1H, dd, J=12.1 Hz, J=5.1 Hz), 2.86 (2H, t, J=6.4 Hz), 3.38 (2H, q, J=6.4 Hz), 3.83 (1H, m), 4.15 (1H, dd, J=14.2 Hz, J=9.2 Hz), 4.49 (1H, dd, J=14.2 Hz, J=3.1 Hz), 5.15 (1H, d, J=5.3 Hz), 6.53 (1H, d, J=5.4 Hz), 7.25 (1H, broad t, J=6.4 Hz), 7.45 (1H, dd, J=9.0 Hz, J=2.3 Hz), 7.78 (1H, d, J=2.3 Hz), 8.02 (1H, s), 8.26 (1H, d, J=9.0 Hz), 8.40 (1H, d, J=5.4 Hz). MS (DCI/NH₃>0) m/z: 405 (M+H⁺). Elementary analysis: for C₁₈H₂₁ClN₆O₃.0.1EtOH.0.6H₂O: % theor. C 52.01, N 20.00; % exper. C 51.98, N 19.94.

Example 31 Below Exemplifies the Creation of Hybrid Molecules in the Aminoquinoline-Streptogramin Family.

### Example 31

### Aminoquinolin Streptogramin Hybrid Molecule, Ref PA 1182

### 5δ-{1-[2-(7-Chloroquinolin-4-ylamino)-ethylamino]-methylsulfanyl}pristinamycin I_{A}

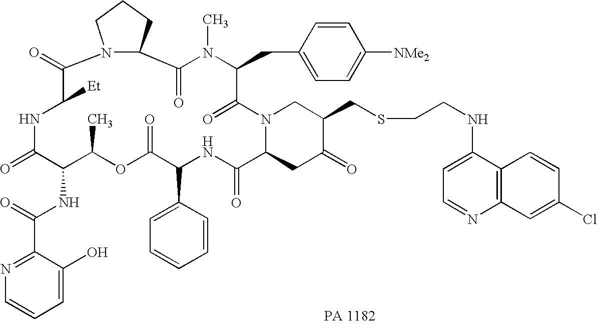

A suspension of 2-(7-chloroquinolin-4-ylamino)-ethanethiol (prepared according to the method described by 1. Lhomme et al., Tetrahedron 1989, 45, 6455-6466) (0.2 g, 0.8 mmol) in 5 mL of acetone is added, in small increments over 1 hr 30, to a solution under argon and at −20° C. of “5δ-methylenepristinamycin I_{A}” (prepared according to the method described by J.-P. Bastart et al. in patent EP 0432029A1) (0.6 g, 0.7 mmol) in 20 mL of acetone. The mixture is kept at −20° C. under stirring for 5 hr 30. The suspension obtained is filtered and the precipitate is washed with acetone. After concentration in a rotary evaporator, the filtrate is purified by liquid chromatography on silica gel (SiO₂ 60 Å C.C 6-35 μm, eluent: dichloromethane/methanol/30% ammonia 92:6:2 v/v/v). PA 1182 is obtained as a pale yellow powder (0.3 g, 44%).

¹H NMR (250 MHz, CDCl₃) δ ppm: 0.58 (1H, dd, J=5.9 Hz, J=14.8 Hz), 0.90 (3H, t, J=7.4 Hz), 1.09-1.36 (5H, m), 1.50-1.72 (3H, m), 2.00-2.43 (5H, m), 2.62-2.73 (2H, m), 2.83-3.03 (9H, m), 3.20-3.28 (5H, m), 3.53-3.61 (2H, m), 4.56 (1H, dd, J=6.4 Hz, J=8.2 Hz), 4.81-4.92 (3H, m), 5.20-5.31 (2H, m), 5.83 (1H, d, J=9.1 Hz), 5.90 (1H, dd, J=1.5 Hz, J=6.4 Hz), 6.20 (1H, broad s), 6.45 (1H, d, J=5.7 Hz), 6.50 (1H, d, J=9.8 Hz), 6.58 (2H, d, J=8.6 Hz), 7.02 (2H, d, J=8.6 Hz), 7.16 (2H, m), 7.28 (3H, m), 7.35 (1H, dd, J=2.1 Hz, J=9.0 Hz), 7.47 (2H, m), 7.86 (1H, dd, J=2.1 Hz, J=3.6 Hz), 7.91 (1H, d, J=9.0 Hz), 8.02 (1H, d, J=2.1 Hz), 8.43 (1H, d, J=9.9 Hz), 8.50 (1H, d, J=5.7 Hz), 8.80 (1H, d, J=9.1 Hz), 11.65 (1H, broad s). MS (IS>0) m/z: 1117.6 (M+H⁺). Elementary analysis: for C₅₇H₆₅ClN₁₀O₁₀S.0.1Et₂O.1.7H₂O: % theor. C 59.65, N 12.12; % exper. C 59.71, N 12.13.

Examples 32 Through 34 Below Exemplify the Creation of Hybrid Molecules in the Aminoquinoline-Diaminopyrimidine Family.

### Example 32

### Aminoquinoline-Diaminopyrimidine Hybrid Molecule, Ref PA 1154

### 5-{4-[2-(7-Chloroquinolin-4-ylamino)-ethoxy]-benzyl}-pyrimidine-2,4-diamine

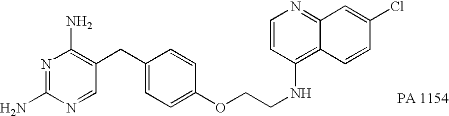

### 32.1. 4-[2-(7-Chloroquinolin-4-ylamino)-ethoxy]-benzaldehyde

A suspension under argon of (2-bromoethyl)-(7-chloroquinolin-4-yl)-amine (5.0 g, 17.5 mmol), 4-hydroxybenzaldehyde (3.0 g, 24.5 mmol), and potassium carbonate (7.3 g, 52.5 mmol) in 60 mL of dimethylformamide is heated to 60° C. for 24 hr. After cooling to room temperature, the reaction medium is diluted with 200 mL of dichloromethane and washed with 3 times 200 mL of water. The organic layer is dried on magnesium sulfate, filtered, and then concentrated in a rotary evaporator. The oil obtained is purified by liquid chromatography on silica gel (SiO₂ 60 Å C.C 6-35 μm, eluent: ethyl acetate/ethanol/triethylamine 90:5:5 v/v/v). The product is obtained as a slightly yellowish powder (2.8 g, 49%).

¹H NMR (300 MHz, CDCl₃) δ ppm: 3.83 (2H, q, J=5.1 Hz), 4.41 (2H, t, J=5.1 Hz), 5.38 (1H, broad t), 6.54 (1H, d, J=5.4 Hz), 7.08 (1H, broad d, J=8.7 Hz), 7.42 (1H, dd, J=2.1 Hz, J=9.0 Hz), 7.72 (1H, d, J=9.0 Hz), 7.89 (1H, broad d, J=8.7 Hz), 8.01 (1H, d, J=2.1 Hz), 8.62 (1H, d, J=5.4 Hz), 9.93 (1H, s). MS (DCI/NH₃>0) m/z: 327 (M+H⁺).

### 32.2. Mixture of Z and E isomers or 2-{4-[2-(7-chloroquinolin-4-ylamino)ethoxy]-benzyl}3-phenylamino-acrylonitrile

0.4 g of potassium tert-butoxide (3.7 mmol) is added in small increments, over 5 min, to a solution under argon and at 10° C. of 4-[2-7-chloroquinolin-4-ylamino)ethoxy]-benzaldehyde (example 32.1) (1.1 g, 3.3 mmol) and anilinopropionitrile (0.5 g, 3.6 mmol) in 10 mL of dry dimethylsulfoxide. Stirring is continued at a temperature of 10° C. for 1 hr. Then the cold bath is removed and stirring is continued at room temperature for 20 hr. 200 mL of water is added to the raw reaction mixture and the product is extracted with 3 times 200 mL of ethyl acetate. The combined organic layers are washed again with 3 times 200 mL of water before being concentrated in a rotary evaporator. After recrystallization from ethanol at −18° C., the product is obtained as a Z and E mixture, in the form of a white powder (0.6 g, 40%).

¹H NMR (300 MHz, CDCl₃) δ ppm: 3.42 and 3.55 (2H, 2s), 3.69 (2H, q, J=5.4 Hz), 4.23 (2H, t, J=5.4 Hz), 6.60 (1H, d, J=5.7 Hz), 6.89-7.07 (3H, m), 7.17-7.30 (6H, m), 7.46 (1H, dd, J=2.4 Hz, 19.0 Hz), 7.50 (1H, broad t, J=5.4 Hz), 7.64 (1H, d, J=12.9 Hz), 7.79 (1H, d, J=2.4 Hz), 8.29 (1H, d, J=9.0 Hz), 8.42 (1H, d, J=5.4 Hz), 9.12 (1H, d, J=12.9 Hz). MS (DCI/NH₃>0) m/z: 455 (M+H⁺).

### 32.3. 5-4-[2-(7-Chloroquinolin-4-ylamino)-ethoxy]-benzyl)pyrimidine-2,4-diamine

0.4 g of potassium tert-butoxide (3.3 mmol) is added to a solution under argon of guanidine hydrochloride (0.3 g, 3.3 mmol) in 5 mL of absolute ethanol. The suspension is stirred for 1 hr before being filtered on celite. The filtrate is injected into a suspension under argon of a mixture of the Z and E isomers of 254-[2-(7-chloroquinolin-4-ylamino)-ethoxy]-benzyl)-3-phenylamino-acrylonitrile (example 32.2) (0.5 g, 1.1 mmol), in 3 mL of absolute ethanol, and the resulting mixture is heated to reflux for 3 hr. After cooling to room temperature, the suspension is filtered and the precipitate is washed successively with water, ethanol, and diethyl ether. PA 1154 is obtained as a white powder (0.1 g, 26%).

¹H NMR (500 MHz, DMSO) δ ppm: 3.52 (2H, s), 3.68 (2H, q, J=5.4 Hz), 4.20 (2H, t, J=5.4 Hz), 5.66 (2H, s), 6.02 (2H, s), 6.59 (1H, d, J=5.4 Hz), 6.87 (2H, d, J=8.4 Hz), 7.12 (2H, d, J=8.4 Hz), 7.47 (3H, m), 7.79 (1H, d, J=2.1 Hz), 8.29 (1H, d, J=9.0 Hz), 8.41 (1H, d, J=5.4 Hz). MS (DCI/NH₃>0) m/z: 421 (M+H⁺). Elementary analysis: for C₂₂H₂₁ClN₆O.0.2H₂O: % theor. C 62.24, N 19.80; % exper. C 62.20, N 19.45.

### Example 33

### Aminoquinoline-Diaminopyrimidine Hybrid Molecule, Ref PA 1161

### 5-{4-[2-(7-Chloroquinolin-4-ylamino)ethoxy]-3-methoxy-benzyl)pyrimidine-2,4-diamine

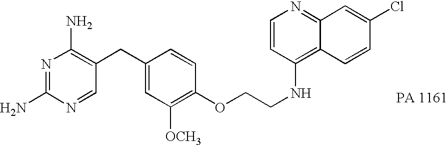

### 33.1. 4-[2-(7-Chloroquinolin-4-ylamino)-ethoxy]-3-methoxy-benzaldehyde

This compound is prepared according to the procedure described in example 32.1, from 1.2 g of (2-bromoethyl)-(7-chloroquinolin-4-yl)-amine (4.3 mmol), 0.9 g of vaniline (6.0 mmol), and 1.8 g of potassium carbonate (12.8 mmol) in 20 mL of dimethylformamide. The product is obtained, without purification by liquid chromatography on silica gel but after the solid is washed with ethanol, as a white powder (1.0 g, 69%).

¹H NMR (300 MHz, DMSO) δ ppm: 3.73 (2H, q, J=5.1 Hz), 3.81 (3H, s), 4.37 (2H, m), 6.54 (1H, d, J=5.4 Hz), 7.23 (1H, d, J=8.1 Hz), 7.40 (1H, d, J=1.8 Hz), 7.47 (1H, dd, J=2.1 Hz, J=9.0 Hz), 7.51-7.56 (2H, m), 7.80 (1H, d, J=2.1 Hz), 8.28 (1H, d, J=9.0 Hz), 8.44 (1H, d, J=5.4 Hz), 9.84 (1H, s). MS (DCI/NH₃>0) m/z: 357 (M+H⁺).

### 33.2. Mixture of the Z and E isomers of 2-{4-[2-(7-chloroquinolin-4-ylamino)-ethoxy]-3-methoxy-benzyl}-3-phenylamino-acrylonitrile

This compound is prepared according to the procedure described in example 32.2, from 0.5 g of 4-[2-(7-chloroquinolin-4-ylamino)-ethoxy]-3-methoxy-benzaldehyde (example 33.1) 0.2 g of anilinopropionitrile (1.5 mmol), and 0.2 g of potassium tert-butoxide (1.5 mmol) in 5 mL of dry dimethylsulfoxide. After recrystallization at 6° C. from ethanol with few drops of water, the product is obtained as a mixture of the Z and E isomers, in the form of a white powder (0.3 g, 46%).

¹H NMR (300 MHz, DMSO) δ ppm: 3.42 and 3.56 (2H, 2s), 3.70 (5H, m), 4.21 and 4.35 (2H, 2t, J=5.4 Hz), 6.61 (1H, d, J=5.4 Hz), 6.78 (1H, d, J=8.1 Hz), 6.89-6.98 (3H, m), 7.17-7.30 (4H, m), 7.45-7.53 (2H, m), 7.64 and 7.62 (1H, 2d, J=12.9 Hz), 7.80 (1H, d, J=2.1 Hz), 8.28 (1H, d, J=9.3 Hz), 8.42 (1H, d, 17=5.4 Hz), 9.08 and 9.10 (1H, 2d, J=12.9 Hz), MS (DCI/NH₃>0) m/z: 485 (M+H⁺).

### 33.3. 5-{4-[2-(7-Chloroquinolin-4-ylamino)ethoxy]-3-methoxy-benzyl}pyrimidine-2,4-diamine

PA 1161 is prepared according to the procedure described in example 32.3, from 0.3 g of guanidine hydrochloride (3.1 mmol), 0.4 g of potassium tert-butoxide (3.1 mmol), and 0.5 g of 2d-4-[2-(7-chloroquinolin-4-yl-amino)-ethoxy]-3-methoxy-benzyl}-3-phenylamino-acrylonitrile (example 33.2) (1.0 mmol) in 3 mL of absolute ethanol. After reflux in ethanol, the product is filtered while hot and washed with methanol. PA 1161 is obtained as a white powder (0.1 g, 21%).

¹H NMR (500 MHz, DMSO) δ ppm: 3.52 (2H, s), 3.66 (2H, q, J=5.5 Hz), 3.70 (3H, s), 4.19 (2H, t, J=5.5 Hz), 5.67 (2H, s), 6.04 (2H, s), 6.60 (1H, d, J=5.4 Hz), 6.70 (1H, dd, J=1.7 Hz, J=8.1 Hz), 6.88 (1H, d, J=1.7 Hz), 6.90 (1H, d, =8.1 Hz), 7.47 (3H, m), 7.80 (1H, d, J=2.2 Hz), 8.28 (1H, d, J=9.1 Hz), 8.42 (1H, d, J=5.4 Hz). MS (DCI/NH₃>0) m/z: 451 (M+H⁺). Elementary analysis: for C₂₃H₂₃CIN₆O₂.1MeOH.1.3H₂O: % theor. C 56.80, N 16.49; % exper. C 56.81, N 16.46.

### Example 34

### Aminoquinoline-Diaminopyrimidine Hybrid Molecule, Ref PA 1187

### 5-{3-[2-(7-Chloroquinolin-4-ylamino)-ethoxy]-4,5-dimethoxy-benzyl}pyrimidine-2,4-diamine

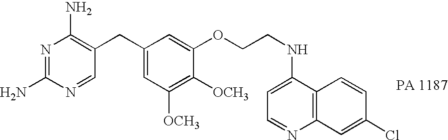

### 34.1. 3-[2-(7-Chloroquinolin-4-ylamino)-ethoxy]-4,5-dimethoxy-benzaldehyde

This compound is prepared according to the procedure described in example 32.1, from 2.6 g of (2-bromoethyl)-(7-chloroquinolin-4-yl)-amine (9.1 mmol), 5-hydroxy-veratraldehyde (11.0 mmol) and 3.8 g of potassium carbonate (27.4 mmol) in 30 mL of dimethylformamide. The product is obtained as a white powder after purification by liquid chromatography on silica gel (SiO₂ 60 Å C.C 6-35 μm, eluent: dichloromethane/methanol/30% ammonia 88:10:2 v/v/v) (1.5 g, 43%).

¹H NMR (250 MHz, DMSO) δ ppm: 3.71 (3H, s), 3.75 (2H, m), 3.84 (3H, s), 4.35 (2H, m), 6.64 (1H, d, J=5.2 Hz), 7.24 (1H, s), 7.30 (1H, s), 7.46 (1H, d, J=8.9 Hz), 7.51 (1H, m), 7.79 (1H, s), 8.27 (1H, d, 7=8.9 Hz), 8.42 (1H, d, J=5.2 Hz), 9.85 (1H, s). MS (FAB>0) m/z: 387 (M+H⁺).

### 34.2. Mixture of the Z and E isomers of 2-{3-[2-(7-chloroquinolin-4-ylamino) ethoxy]-4,5-dimethoxy-benzyl}-3-phenylamino-acrylonitrile

This compound is prepared according to the procedure described in example 32.2, from 1.5 g of 3-[2-7-chloroquinolin-4-ylamino)ethoxy]-4,5-dimethoxy-benzaldehyde (example 34.1) (3.9 mmol), 0.6 g of anilinopropionitrile (4.2 mmol), and 0.5 g of potassium tert-butoxide (4.4 mmol) in 5 mL of dry dimethylsulfoxide. After recrystallization at 6° C. from ethanol with few drops of water, the product is obtained as a mixture of the Z and E isomers, in the form of a white powder (1.1 g, 53%).

¹H NMR (250 MHz, DMSO) δ ppm: 3.41 and 3.55 (2H, 2s), 3.58 (3H, s), 3.72 (5H, m), 4.22 and 4.36 (2H, 2m), 6.60 (3H, m), 6.93 (1H, m), 7.19-7.30 (4H, m), 7.42-7.52 (2H, m), 7.65 and 7.66 (1H, 2d, J=12.9 Hz), 7.79 (1H, d, J=2.1 Hz), 8.27 (1H, d, J=9.1 Hz), 8.40 (1H, d, J=5.3 Hz), 9.10 and 9.12 (1H, 2d, J=12.9 Hz). MS (DCI/NH₃>0) m/z: 515 (M+H⁺).

### 34.3. 5-{3-[2-(7-Chloroquinolin-4-ylamino)-ethoxy]-4,5-dimethoxy-benzyl}-pyrimidine-2,4-diamine

PA 1187 is prepared according to the procedure described in example 32.3, from 0.3 g of guanidine hydrochloride (3.1 mmol), 0.3 g of potassium tert-butoxide (3.1 mmol), and 0.5 g of 2-3-[2-(7-chloroquinolin-4-ylamino)-ethoxy]-4,5-dimethoxy-benzyl)-3-phenylamino-acrylonitrile (example 34.2) (1.0 mmol) in 6 mL of absolute ethanol. Reflux in ethanol is continued for 20 hr. After cooling to room temperature, the product is extracted with chloroform in a biphasic chloroform/water medium. Concentration of the organic layer under vacuum allows PA 1187 to be obtained as a white powder (0.3 g, 65%).

¹H NMR (250 MHz, DMSO) δ ppm: 3.50 (2H, s), 3.55 (3H, s), 3.69 (5H, m), 4.19 (2H, t, J=5.2 Hz), 5.69 (2H, s), 6.08 (2H, s), 6.58 (2H, s), 6.60 (1H, d, J=5.4 Hz), 7.47 (3H, m), 7.79 (1H, d, J=2.1 Hz), 8.26 (1H, d, J=9.1 Hz), 8.41 (1H, d, J=5.4 Hz). MS (DCI/NH₃>0) m/z: 481 (M+H⁺). Elementary analysis: for C₂₄H₂₅ClN₆O₃.1.7H₂O: % theor. C 56.34, N 16.43; % exper. C 56.41, N 16.03.

Example 35 Below Exemplifies the Creation of Hybrid Molecules in the Aminoquinoline-Macrolide Family.

### Example 35

### Aminoquinoline-Macrolide, Hybrid Molecule, Ref PA 1169

### 10{O-[3-(7-Chloroquinolin-4-ylamino)-propyl]-oxime}-erythromycin

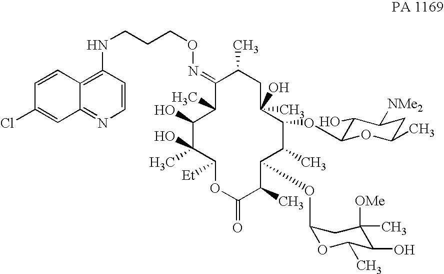

A suspension under argon of 10-oxime erythromycin A (prepared according to the method described by U. Takehiro Amano et al. in U.S. Pat. No. 5,274,085) (1.0 g, 1.3 mmol), (2-bromoethyl)-(7-chloroquinolin-4-yl)-amine (0.4 g, 1.5 mmol), and ground sodium hydroxide (0.1 g, 1.5 mmol) in 10 mL of dry dimethylformamide is stirred at room temperature for 3 hr. The reaction medium is then diluted with 50 mL of chloroform and washed with 3 times 100 mL of water. The organic layer is dried over sodium sulfate, filtered, and then concentrated in a rotary evaporator. The product is then purified by liquid chromatography on silica gel (SiO₂ 60 Å C.C 6-35 μm, eluent: dichloromethane/methanol/30% ammonia 93:5:2 v/v/v). After recrystallization from a 1:1 v/v propan-2-ol/water mixture at 6° C., PA 1169 is obtained as a white powder (0.3 g, 22%).

¹H NMR (250 MHz, CDCl₃) δ ppm: 0.85 (3H, m), 0.90-1.39 (24H, m), 1.40-1.79 (8H, m), 1.80-2.50 (16H, m), 2.71 (1H, q, J=6.9 Hz), 2.80-3.10 (3H, m), 3.22 (1H, m), 3.30 (3H, s), 3.40-3.80 (6H, m), 3.99 (2H, m), 4.20 (2H, m), 4.40 (2H, m), 4.85 (1H, d, J=4.5 Hz), 5.07 (1H, d, J=9.0 Hz), 5.67 (1H, broad s), 6.43 (1H, d, J=5.5 Hz), 7.35 (1H, dd, J=2.1 Hz, J=9.0 Hz), 7.81 (1H, d, J=9.0 Hz), 7.97 (1H, d, J=2.1 Hz), 8.51 (1H, d, J=5.5 Hz). MS (DCI/NH₃>0) m/z: 967 (M+H⁺). Elementary analysis: for C₄₉H₇₉ClN₄O₁₃.H₂O: % theor. C 59.71, N 5.68; % exper. C 59.85, N 5.46.

Examples 36 Through 41 Below Exemplify the Creation of Hybrid Molecules in the Aminoquinoline-Glycopeptide Family

### Example 36

### Aminoquinoline-Glycopeptide Hybrid Molecule, Ref PA 1157

### N-4-{4-[2-(7-Chloroquinolin-4-ylamino)ethoxy]-benzyl}-vancomycin

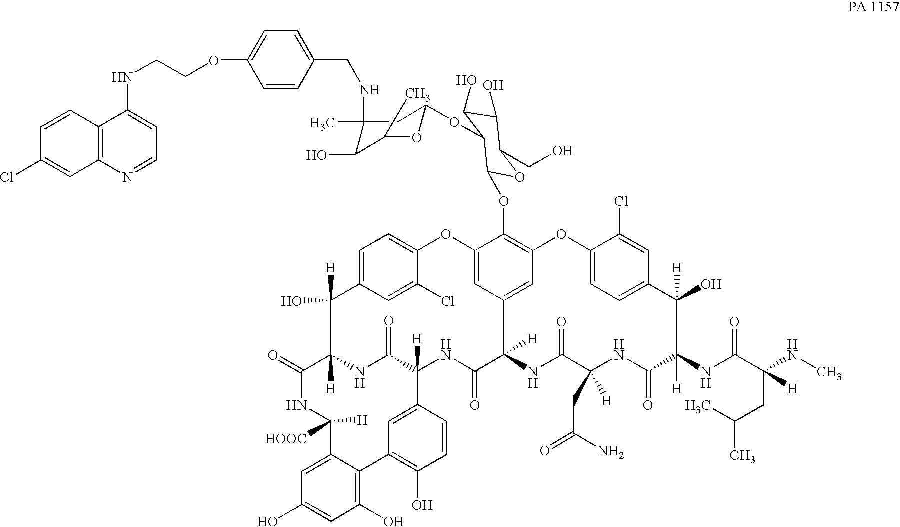

0.2 mL of diisopropylethylamine (1.1 mmol) is added to a solution under argon and at 70° C. of 4-[2-(7-chloroquinolin-4-ylamino)-ethoxy]-benzaldehyde (example 32.1) (0.2 g, 0.7 mmol) in 24 mL of dimethylacetamide. After this mixture is stirred for 2 hr at 70° C., a solution of sodium cyanoborohydride (0.1 g, 2.1 mmol) in 2 mL of methanol is added. The mixture is stirred for 2 hr 30 at 70° C. then for 20 hr at room temperature. The suspension obtained is centrifuged and the supernatant is precipitated with acetonitrile. This new precipitate is centrifuged and washed successively with acetonitrile and then with diethyl ether. It is then purified via semi-preparatory HPLC: 10 micron C18 column (21.2×150 mm), isocratic gradient with 19% eluent B for 45 min (eluent A: 0.1% aqueous trifluoroacetic acid; eluent B: 9:1 v/v acetonitrile/0.1% aqueous trifluoroacetic acid), flow rate 15 mL/min, dual detection at 280 and 330 nm. After lyophilization of the collected fractions, the trifluoroacetic acid salt of PA 1157 is obtained as a white powder (25 mg, 3%).

¹H NMR (500 MHz, DMSO d6) δ ppm: 0.86 (3H, d, J=6.0 Hz), 0.91 (3H, d, J=6.0 Hz), 1.13 (3H, d, J=6.2 Hz), 1.47 (3H, s), 1.56-1.69 (3H, m), 1.81 (1H, broad d, J=12.8 Hz), 2.09-2.18 (2H, m), 2.57 (1H, m), 2.65 (3H, s), 3.30 (2H, m), 3.45-3.60 (4H, m), 3.70 (1H, broad d, J=9.1 Hz), 3.94 (5H, m), 4.12 (1H, broad s), 4.21 (1H, d, J=11.7 Hz), 4.31 (3H, m), 4.43 (1H, d, J=5.6 Hz), 4.46 (1H, m), 4.68 (1H, m), 4.96 (1H, broad s), 5.12 (1H, d, J=6.0 Hz), 5.15 (1H, broad s), 5.18 (1H, s), 5.21 (1H, broad s), 5.28 (1H, broad s), 5.35 (1H, d, J=7.6 Hz), 5.38 (1H, broad d, J=4.2 Hz), 5.61 (1H, s), 5.77 (1H, d, J=7.7 Hz), 5.84 (1H, broad s), 6.00 (1H, d, 7=6.0 Hz), 6.04 (1H, broad s), 6.25 (1H, d, J=1.7 Hz), 6.41 (1H, d, J=1.7 Hz), 6.57 (1H, broad s), 6.72 (2H, m), 6.78 (1H, d, J=8.8 Hz), 6.97-7.25 (8H, m), 7.34 (1H, d, J=8.3 Hz), 7.38 (2H, d, J=8.6 Hz), 7.47 (2H, m), 7.57 (1H, d, J=8.4 Hz), 7.75 (1H, dd, J=9.1 Hz, J=1.8 Hz), 7.86 (1H, s), 7.98 (1H, s), 8.08 (1H, broad s), 8.53-8.67 (6H, m), 9.13 (1H, s), 9.20 (1H, s), 9.25 (1H, broad s), 9.48 (1H, s). MS (IS>0) m/z: 1761.0 (M+H⁺), 881.1 (M+2H⁺).

### Example 37

### Aminoquinoline-Glycopeptide Hybrid Molecule, Ref PA 1158

### N-4-[4(7-Chloroquinolin-4-ylamino)-butyl]-vancomycin

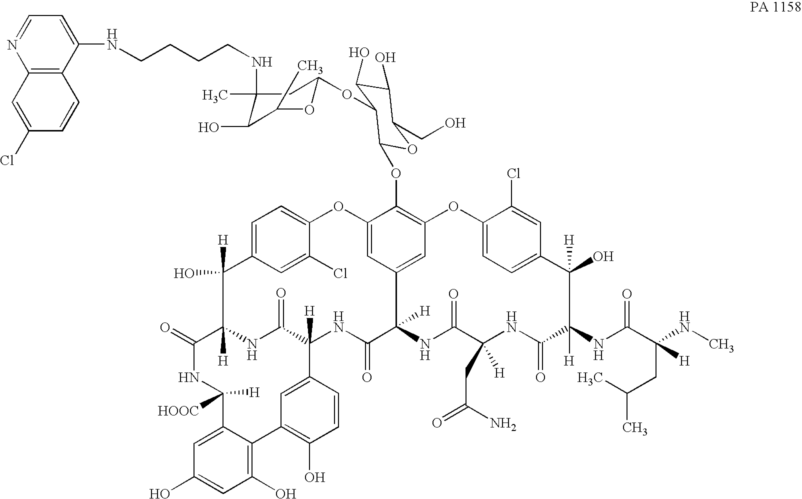

### 37.1. (7-Chloroquinolin-4-yl)-(4,4-diethoxy-butyl)-amine

A suspension under argon of 4.7-dichloroquinoline (2.0 g, 10.0 mmol), in 5.2 mL of 4-aminobutyraldehyde diethylacetal (30.0 mmol) is heated to 110° C. for 29 hr. After cooling to room temperature, the reaction medium is diluted with 50 mL of dichloromethane and 100 mL of a solution of 5% carbonated water. The organic layer is separated and the aqueous phase is re-extracted with 3 times 50 mL of dichloromethane. The combined organic layers are dried over magnesium sulfate, filtered, then concentrated under vacuum. After recrystallization from hexane at −18° C., the product is obtained as a white powder (2.2 g, 69%).

¹H NMR (300 MHz, DMSO) δ ppm: 1.10 (6H, t, J=6.9 Hz), 1.65 (4H, m), 3.27 (2H, m), 3.52 (2H, m), 3.56 (2H, m), 4.15 (1H, t, J=5.1 Hz), 6.47 (1H, d, J=5.4 Hz), 7.32 (1H, t, J=5.1 Hz), 7.44 (1H, dd, J=1.5 Hz, J=9.0 Hz), 7.77 (1H, d, J=1.5 Hz), 8.27 (1H, d, J=9.0 Hz), 8.38 (1H, d, J=5.4 Hz). MS (DCI/NH₃>0) m/z: 323 (M+H⁺).

### 37.2. 4-7-Chloroquinolin-4-ylamino)-butyraldehyde

1 mL of trifluoroacetic acid (13.0 mmol) is added to a solution under argon of (7-chloro-quinolin-4-yl)-(4,4-diethoxy-butyl)-amine (example 37.1) (0.3 g, 0.9 mmol) in 5 mL of 80% aqueous acetic acid. The mixture is heated to 70° C. for 1 hr 30. After cooling to room temperature, the medium is evaporated to dryness. The product is obtained as a yellow powder (0.3 g, 100%).

¹H NMR (300 MHz, DMSO) δ ppm: 1.92 (2H, m), 2.64 (2H, t, J=6.9 Hz), 3.53 (2H, q, J=6.9 Hz), 6.93 (1H, d, J=7.2 Hz), 7.80 (1H, dd, J=1.8 Hz, J=9.3 Hz), 7.96 (1H, d, J=1.8 Hz), 8.53 (1H, d, J=9.3 Hz), 8.58 (1H, d, J=7.2 Hz), 9.40 (1H, broad t), 9.71 (1H, s). MS (IS>0) m/z: 249.1 (M+H⁺).

### 37.3. N-4-[4-(7-Chloro-quinolin-4-ylamino)-butyl]-vancomycin

PA 1158 is prepared according to the procedure described in example 36, from 100 mg of vancomycin hydrochloride (0.1 mmol), 32 mg of 4-(7-chloro-quinolin-4-ylamino) butyraldehyde (example 37.2) (0.1 mmol), 0.04 mL of diisopropylethylamine (0.2 mmol), and 17 mg of sodium cyanoborohydride (0.3 mmol) in 3 mL of dry dimethylformamide. The product is purified by semi-preparatory HPLC with an isocratic gradient with 17% eluent. B for 45 min and a flow rate of 17 mL/min. After lyophilization of the collected fractions, the trifluoroacetic acid salt of PA 1158 is obtained as a white powder (10 mg, 9%).

¹H NMR (500 MHz, DMSO d6) δ ppm: 0.86 (3H, d, J=6.0 Hz), 0.92 (3H, d, J=6.0 Hz), 1.10 (3H, d, J=6.1 Hz), 1.36 (3H, s), 1.58-1.76 (7H, m), 1.82 (1H, broad d, J=12.6 Hz), 1.99 (1H, m), 2.17 (1H, m), 2.56 (1H, m), 2.65 (3H, s), 2.82 (2H, m), 3.28 (2H, m), 3.31 (1H, broad s), 3.47 (1H, m), 3.53-3.59 (4H, m), 3.70 (1H, broad d, J=10.5 Hz), 3.96 (1H, broad s), 4.10 (1H, broad s), 4.20 (1H, broad d, J=10.8 Hz), 4.27 (1H, broad s), 4.44 (2H, m), 4.65 (1H, m), 4.95 (1H, broad s), 5.11-5.20 (4H, m), 5.29-5.32 (2H, m), 5.36 (1H, broad s), 5.61 (1H, s), 5.76 (2H, m), 5.98 (1H, broad s), 6.02 (1H, broad s), 6.25 (1H, d, J=1.6 Hz), 6.41 (1H, d, J=1.6 Hz), 6.57 (1H, broad s), 6.71 (2H, m), 6.78 (1H, d, J=8.7 Hz), 6.92 (1H, d, J=7.2 Hz), 7.04-7.33 (5H, m), 7.46-7.49 (3H, m), 7.57 (1H, d, J=8.2 Hz), 7.80 (1H, dd, J=9.1 Hz, J=1.7 Hz), 7.85 (1H, s), 8.01 (2H, m), 8.32 (1H, broad s), 8.53-8.58 (4H, m), 8.68 (1H, broad s), 9.01 (1H, broad s), 9.11 (1H, s), 9.19 (1H, s), 9.31 (1H, broad s), 9.42 (1H, broad s), 9.48 (1H, broad s). MS (IS>0) m/z: 842.0 (M+2H⁺).

### Example 38

### Aminoquinoline-Glycopeptide Hybrid Molecule, Ref PA 1159 N-4-[4-7-Chloroquinolin-4-ylamino)-ethyl]-vancomycin

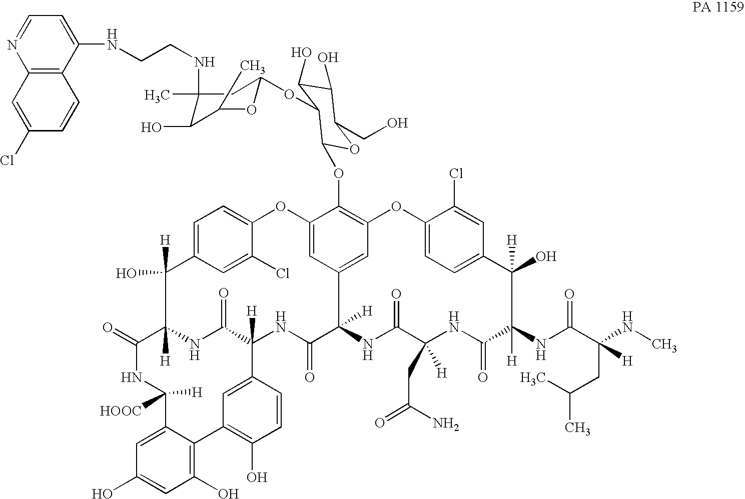

### 38.1. (7-Chloroquinolin-4-yl)-(2,2-dimethoxy-ethyl)-amine

This compound is prepared according to the procedure described in example 37.1, from 2.0 g of 4,7-dichloroquinoline (10.0 mmol) and 3.3 mL of aminoacetaldehyde dimethylacetal (30.0 mmol). After recrystallization from dichloromethane/hexane, the product is obtained as a beige powder (2.3 g, 87%).

¹H NMR (300 MHz, DMSO) δ ppm: 3.33 (6H, s), 3.41 (2H, t, J=5.7 Hz), 4.63 (1H, t, J=5.7 Hz), 6.56 (1H, d, J=5.4 Hz), 7.34 (1H, t, J=5.7 Hz), 7.46 (1H, dd, J=2.1 Hz, J=9.0 Hz), 7.79 (1H, d, J=2.1 Hz), 8.27 (1H, d, J=9.0 Hz), 8.41 (1H, d, J=5.4 Hz). MS (DCI/NH₃>0) m/z: 267 (M+H⁺).

### 38.2. (7-Chloroquinolin-4-ylamino)acetaldehyde

This compound is prepared according to the procedure described in example 37.2, from 0.3 g of (7-chloroquinolin-4-yl)(2,2-dimethoxy-ethyl)-amine (example 38.1) (1.1 mmol), in 5 mL of 80% aqueous acetic acid and 1 mL of trifluoroacetic acid (13.0 mmol). After the reaction mixture is evaporated to dryness, the product is obtained as a red powder (0.4 g, 100%).

¹H NMR (300 MHz, DMSO) δ ppm: 4.67 (2H, d, J=5.7 Hz), 6.81 (1H, d, J=7.2 Hz), 7.83 (1H, dd, J=2.1 Hz, J=9.0 Hz), 8.01 (1H, d, J=2.1 Hz), 8.52 (1H, a, J=9.0 Hz), 8.59 (1H, d, J=7.2 Hz), 9.56 (1H, broad t), 9.65 (1H, s). MS (IS>0) m/z: 221.1 (M+H⁺).

### 38.3. N-4-[4(7-Chloroquinolin-4-ylamino)-ethyl]-vancomycin

PA 1159 is prepared according to the procedure described in example 36, from 100 mg of vancomycin hydrochloride (0.1 mmol), 24 mg of (7-chloro-quinolin-4-ylamino)-acetaldehyde (example 38.2) (0.1 mmol), 0.05 mL of diisopropylethylamine (0.3 mmol), and 13 mg of sodium cyanoborohydride (0.2 mmol) in 3 mL of dry dimethylformamide. The product is purified by semi-preparatory HPLC with an isocratic gradient with 16% eluent B for 45 min and a flow rate of 17 mL/min. After lyophilization of the collected fractions, the trifluoroacetic acid salt of PA 1159 is obtained as a white powder (9 mg, 8%).

¹H NMR (500 MHz, DMSO d6) δ ppm: 0.86 (3H, broad d, J=4.6 Hz), 0.91 (3H, broad d, J=4.6 Hz), 1.11 (3H, d, J=5.0 Hz), 1.38 (3H, s), 1.60-1.76 (3H, m), 1.89 (1H, m), 2.01 (1H, m), 2.17 (1H, m), 2.55 (1H, m), 2.65 (3H, s), 3.13 (2H, m), 3.25-3.50 (4H, m), 3.50-3.62 (2H, m), 3.69 (1H, broad d, J=10.1 Hz), 3.83 (2H, broad s), 3.96 (1H, broad s), 4.08 (1H, broad s), 4.21 (1H, broad d, J=10.8 Hz), 4.27 (1H, broad s), 4.44 (2H, m), 4.69 (1H, d, J=5.6 Hz), 4.96 (1H, broad s), 5.11-5.20 (4H, m), 5.31 (2H, broad s), 5.37 (1H, broad s), 5.60 (1H, s), 5.76 (1H, d, J=6.6 Hz), 5.87 (1H, broad s), 5.99 (1H, broad s), 6.03 (1H, broad s), 6.26 (1H, s), 6.41 (1H, s), 6.57 (1H broad s), 6.71-6.77 (3H, m), 6.90 (1H, d, J=6.1 Hz), 7.03-7.57 (8H, m), 7.79 (1H, d, J=8.6 Hz), 7.84 (1H, s), 8.02 (1H, s), 8.39-8.78 (7H, m), 9.10 (1H, broad s), 9.12 (1H, s), 9.20 (1H, s), 9.49 (1H, s). MS (IS>0) m/z: 827.0 (M+2H⁺).

### Example 39

### Preparation of an Aminoquinoline-Glycopeptide, Ref PA 1274

### N-4-{4-[2-(2-Methylquinolin-4-ylamino)-ethoxy]-benzyl}-vancomycin dihydrochloride salt

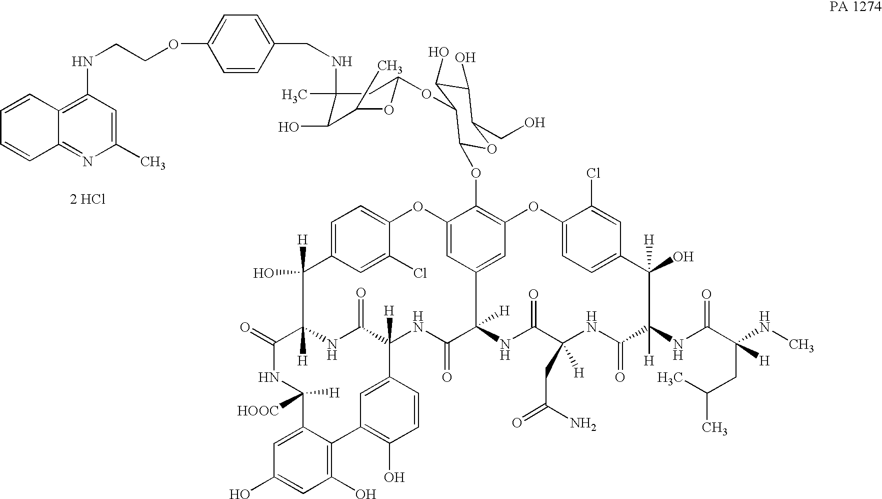

### 39.1. 2-(2-Methylquinolin-4-ylamino)-ethanol

A mixture of 4-chloroquinaldine (10 g, 56.3 mmol) and ethanolamine (13.6 mL, 225.2 mmol) is heated to 150° C. for 15 min and then 190° C. for 30 min. After cooling to room temperature, the cake is suspended in 50 mL of 10% aqueous sodium hydroxide and stirred for 30 min. The resulting precipitate is filtered, washed three times with 100 mL of water and recrystallised in hot methanol to afford the title compound as a white powder (12.85 g, 100%).

¹H NMR (250 MHz, DMSO) δ ppm: 2.45 (3H, s), 3.30 (2H, m), 3.65 (2H, q, J=5.8 Hz), 3.84 (1H, t, J=5.6 Hz), 6.37 (1H, s), 6.94 (1H, d, J=5.0 Hz), 7.30 (1H, t, J=7.5 Hz), 7.54 (1H, t, J=7.5 Hz), 7.67 (1H, d, J=7.5 Hz), 8.11 (1H, d, J=7.5 Hz). MS (IS>0) m/z: 203.1 (M+H⁺).

### 39.2. (2-Bromoethyl)-(2-methylquinolin-4-yl)-amine

Hydrobromic acid (21.4 mL, 395 mmol) is added dropwise to 2-(2-methylquinolin-4-ylamino)-ethanol (12.85 g, 63.5 mmol) (example 39.1) at 0° C. followed by sulfuric acid (7.2 ml, 136 mmol). The mixture is heated to 165° C. for 3 hr and then poured in 300 mL of cold water. The mixture is adjusted to pH 8 with NaHCO₃ and extracted with boiling toluene (50 mL). The toluene layer is cooled to room temperature and then left at −30° C. for 12 hr. The resulting precipitate is filtered, affording after drying under vacuum the title compound as a white powder (11.0 g, 65%).

¹H NMR (250 MHz, DMSO) δ ppm: 2.47 (3H, s), 3.71 (4H, m), 6.45 (1H, s), 7.34 (2H, m), 7.57 (1H, t, J=7.5 Hz), 7.69 (1H, d, J=7.5 Hz), 8.10 (1H, d, J=7.5 Hz). (DCI/NH₃>0) m/z: 267 (M+H⁺).

### 39.3. 4-[2-(2-Methylquinolin-4-ylamino)-ethoxy]-benzaldehyde

This compound is prepared according to the procedure described in example 32.1 starting from 11.0 g of (2-bromoethyl)-(2-methylquinolin-4-yl)-amine (41.4 mmol) (example 39.2), 6.1 g of 4-hydroxybenzaldehyde (49.6 mmol) and 17.2 g of potassium carbonate (124.2 mmol). The title compound is obtained as a beige powder after crystallisation in dichloromethane (6.7 g, 53%)

¹H NMR (250 MHz, DMSO) δ ppm: 2.50 (3H, s), 3.83 (2H, q, J=5.4 Hz), 4.49 (1H, t, J=5.4 Hz), 6.60 (1H, s), 7.28 (2H, d, J=8.5 Hz), 7.35 (1H, t, J=5.4 Hz), 7.46 (1H, t, J=7.5 Hz), 7.67 (1H, t, J=7.5 Hz), 7.80 (1H, d, J=7.5 Hz), 7.98 (2H, d, J=8.5 Hz), 8.27 (1H, d, J=7.5 Hz), 9.99 (1H, s). MS (DCI/NH₃>0) m/z: 307 (M+H⁺).

### 39.4. N-4-{4-[2-(2-Methylquinolin-4-ylamino)-ethoxy]-benzyl}-vancomycin dihydrochloride salt

This compound is prepared according to the procedure described in example 36 starting from 25.1 g of vancomycin hydrochloride (16.9 mmol), 6.7 g of 4-[2-(2-methylquinolin-4-ylamino)-ethoxy]-benzaldehyde (22.0 mmol) (example 39.3), 6.4 mL of N,N-diisopropylethylamine (38.8 mmol) and 4.3 g of sodium cyanoborohydride (67.6 mmol). The dihydrochloride salt is obtained as follows: the trifluoroacetic acid salt obtained after lyophilisation of the collected fractions purified by semi-preparative HPLC (0.7 g) is dissolved in water and the solution is adjusted to pH 9 at 0° C. The resulting precipitate isolated after centrifugation for 10 min at 4500 rpm and several washes with water (0.3 g, 0.2 mmol) is suspended in 50 mL of water and cooled to 0° C. To this suspension is added 3.7 mL of a 0.1 N aqueous hydrochloric acid solution (0.4 mmol) and the resulting solution is lyophilized affording PA 1274 as a white powder (0.35 g, 1%, purity 99%) RMN ¹H (500 MHz, DMSO d6) δ ppm: 0.87 (3H, d, J=6.4 Hz), 0.92 (3H, d, J=6.4 Hz), 1.12 (3H, d, J=6.0 Hz), 1.48-1.61 (5H, m), 1.70 (1H, m), 1.85 (1H, broad d, J=12.4 Hz), 2.06-2.16 (2H, m), 2.44 (3H, m), 2.61 (3H, s), 3.30-3.60 (6H, m), 3.70 (1H, broad d, 19.1 Hz), 3.87-3.95 (4H, m), 4.15 (1H, broad s), 4.21 (1H, broad d, J=11.0 Hz), 4.31 (3H, m), 4.44 (2H, broad d, J=5.5 Hz), 4.67 (1H, m), 4.90 (1H, broad s), 5.11 (1H, d, J=5.3 Hz), 5.16-5.20 (3H, m), 5.29 (1H, s), 5.38 (2H, m), 5.59 (1H, s), 5.76 (1H, d, J=7.6 Hz), 5.88 (1H, broad s), 5.99 (1H, d, J=6.0 Hz), 6.26 (1H, s), 6.43 (1H, s), 6.62-6.69 (2H, m), 6.73 (2H, m), 6.78 (2H, m), 6.97 (3H, m), 7.18 (1H, s), 7.27 (1H, d, J=8.2 Hz), 7.33 (1H, d, J=8.2 Hz), 7.42-7.48 (4H, m), 7.55-7.58 (2H, m), 7.81 (1H, t, J=7.6 Hz), 7.90 (2H, t, J=8.2 Hz), 8.13 (2H, broad s), 8.47 (1H, d, J=8.4 Hz), 8.52 (1H, broad s), 8.65 (1H, broad s), 8.74 (1H, broad s), 9.14-9.18 (2H, m), 9.46 (1H,s). SM (IS>0) m/z: 1741.1 (M+H⁺), 871.0 (M+2H⁺). Elementary analysis: for C₈₅H₉₃Cl₂N₁₁O₂₅.2HCl.11.5H₂O: % theor. C 50.54, N 7.63; % exper. C 50.54, N 7.60.

### Example 40

### Preparation of an Aminoquinoline-Glycopeptide, Ref PA 1275

### N-4-{4-[2-(2-Trifluoromethylquinolin-4-ylamino)-ethoxy]-benzyl}-vancomycin dihydrochloride salt

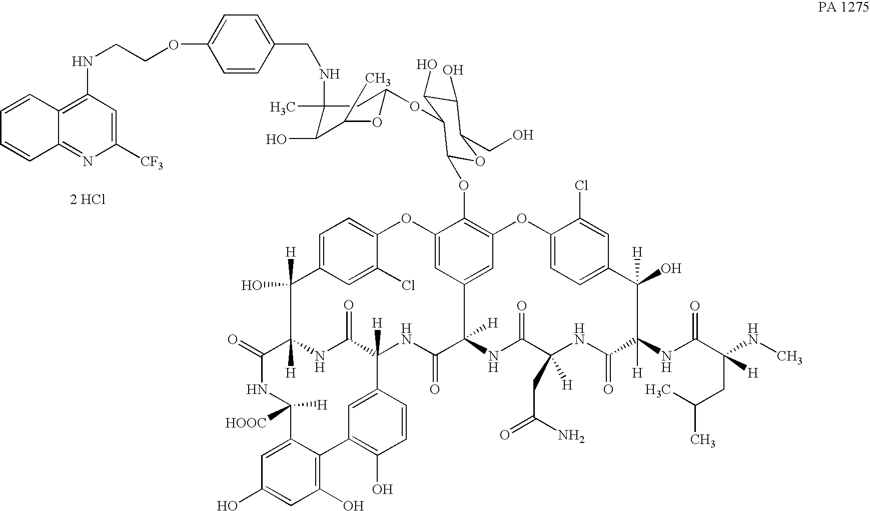

### 40.1. 2-(2-Trifluoromethylquinolin-4-ylamino)-ethanol

This compound is prepared according to the procedure described in example 39.1 starting from 4-chloro-2-(trifluoromethyl)quinoline (10 g, 43.2 mmol) and ethanolamine (7.8 mL, 129.5 mmol). The title compound is obtained as a white powder after recrystallisation in hot ethanol (9.8 g, 88%).

¹H NMR (250 MHz, DMSO) δ ppm: 3.46 (2H, q, J=5.7 Hz), 3.66 (2H, q, J=5.7 Hz), 4.89 (1H, t, J=5.6 Hz), 6.80 (1H, s), 7.56 (1H, t, J=7.1 Hz), 7.73 (2H, m), 7.88 (1H, d, J=7.5 Hz), 8.31 (1H, d, J=7.5 Hz). MS (IS>0) m/z: 257.4 (M+H⁺).

### 40.2. (2-Bromoethyl)-(2-trifluoromethylquinolin-4-yl)-amine

This compound is prepared according to the procedure described in example 39.2 starting from hydrobromic acid (19.2 mL, 354 mmol), 2-(2-trifluoromethylquinolin-4-ylamino)-ethanol (9.8 g, 38.1 mmol) (example 40.1) and sulfuric acid (6.4 ml, 120 mmol). The title compound is obtained as a white powder (9.7 g, 80%).

¹H NMR (250 MHz, DMSO) δ ppm: 3.73 (2H, m), 3.84 (2H, t, J=5.8 Hz), 6.87 (1H, s), 7.60 (1H, t, J=8.2 Hz), 7.76 (1H, t, J=7.5 Hz), 7.90 (2H, m), 8.31 (1H, d, J=7.5 Hz). (DCI/NH₃>0) m/z: 321 (M+H⁺).

### 40.3. 4-[2-(2-Trifluoroethylquinolin-4-ylamino)-ethoxy]-benzaldehyde

This compound is prepared according to the procedure described in example 32.1 starting from (2-bromoethyl)-(2-trifluoromethylquinolin-4-yl)-amine (9.8 g, 30.3 mmol) (example 40.2), 4-hydroxybenzaldehyde (5.2 g, 42.4 mmol) and potassium carbonate (16.7 g, 121.0 mmol). The title compound is obtained as a beige powder after crystallisation in dichlormethane/n-hexane (5.7 g, 52%)

¹H NMR (250 MHz, DMSO) δ ppm: 3.84 (2H, q, J=5.1 Hz), 4.40 (1H, t, J=5.1 Hz), 6.93 (1H, s), 7.14 (2H, d, J=8.7 Hz), 7.58 (1H, t, J=7.1 Hz), 7.74 (1H, t, J=6.9 Hz), 7.90 (4H, m), 8.34 (1H, d, J=8.5 Hz), 9.86 (1H, s). MS (DCI/NH₃>0) m/z: 361 (M+H⁺).

### 40.4. N-4-{4-[2-(2-Trifluoromethylquinolin-4-ylamino)-ethoxy]-benzyl}-vancomycin dihydrochloride salt

This compound is prepared according to the procedure described in example 36 starting from vancomycin hydrochloride (18.1 g, 12.2 mmol), 4-[2-(2-trifluoromethylquinolin-4-ylamino)-ethoxy]-benzaldehyde (5.7 g, 15.9 mmol) (example 40.3), N,N-diisopropylethylamine (4.9 mL, 28.1 mmol) and sodium cyanoborohydride (3.1 g, 48.8 mmol). The dihydrochloride salt is prepared according to the procedure described in example 39.4 with 5.3 mL of a 0.1 N aqueous hydrochloric acid solution (0.5 mmol. PA 1275 is obtained as a white powder (0.5 g, 2%, purity>99%)

RMN ¹H (500 MHz, DMSO d6) δ ppm: 0.87 (3H, d, J=5.7 Hz), 0.93 (3H, d, J=5.6 Hz), 1.13 (3H, d, J=6.1 Hz), 1.51 (3H, s), 1.64-1.71 (3H, m), 1.85 (1H, broad d, J=12.4 Hz), 2.08-2.17 (2H, m), 2.51 (1H, m), 2.61 (2H, s), 3.29 (2H, m), 3.37-3.54 (4H, m), 3.60 (1H, m), 3.70 (1H, m), 3.82-3.96 (5H, m), 4.18 (2H, m), 4.29 (3H,m), 4.44 (2H, d, J=5.6 Hz), 4.67 (1H, m), 4.93 (1H, broad s), 5.11-5.22 (4H, m), 5.30 (1H, broad s), 5.39 (2H, m), 5.62 (1H, s), 5.78 (2H, d, J=7.5 Hz), 6.00 (2H, d, J=5.9 Hz), 6.26 (1H, d, J=1.8 Hz), 6.43 (1H, d, J=1.8 Hz), 6.69-6.79 (3H, m), 6.95-6.99 (4H, m), 7.18 (1H, s), 7.24 (1H, d, J=8.3 Hz), 7.34 (1H, d, J=8.3 Hz), 7.45 (2H, d, J=8.6 Hz), 7.49 (2H, d, J=8.1 Hz), 7.58 (2H, m), 7.75 (1H, t, J=7.6 Hz), 7.90 (2H, m), 8.02 (2H, t, J=5.5 Hz), 8.39 (1H, d, J=8.4 Hz), 8.55 (1H, broad s), 8.68 (1H, broad s), 8.86 (1H, broad s), 9.12 (1H, s), 9.19 (1H, s), 9.46 (1H,s). SM (IS>0) m/z: 1795.3 (M+H⁺), 897.8 (M+2H⁺). Elementary analysis: for C₈H₉₀Cl₂F₃N₁₁O₂₅.2HCl.9.7H₂O: % theor. C 50.00, N 7.55; % exper. C 50.03, N 7.51.

### Example 41

### Preparation of an Aminoquinoline-Glycopeptide, Ref PA 1276

### N-4-{4-[2-(7-Trifluoromethylquinolin-4-ylamino)-ethoxy]-benzyl}-vancomycin dihydrochloride salt

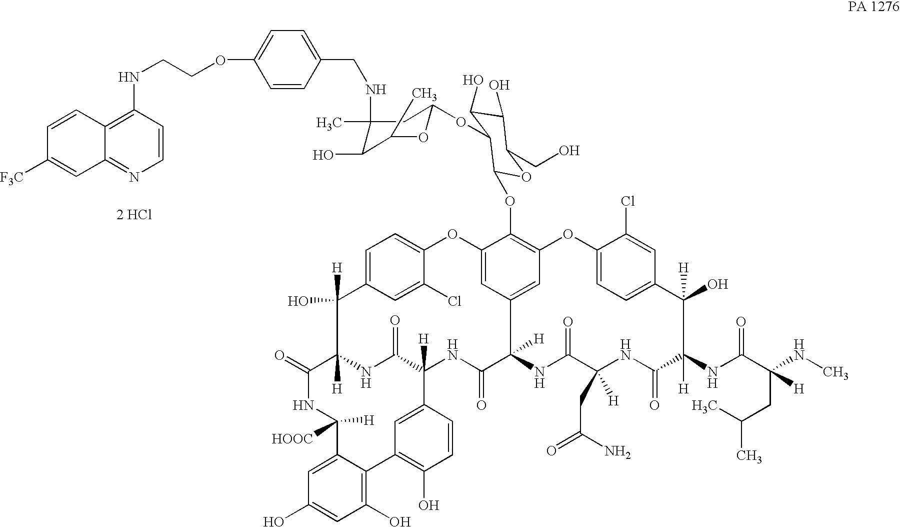

### 41.1. 4-[2-(7-Trifluoroethylquinolin-4-ylamino)-ethoxy]-benzaldehyde

This compound is prepared according to the procedure described in example 32.1 starting from (2-bromoethyl)-(7-trifluoromethylquinolin-4-yl)-amine (7.0 g, 21.8 mmol) (prepared according to the method described by B. Meunier et al., FR 2862304), 4-hydroxybenzaldehyde (3.2 g, 26.0 mmol) and potassium carbonate (9.0 g, 65.4 mmol).

The title compound is obtained as a beige powder after crystallisation in dichloromethane/n-hexane (3.4 g, 43%)

¹H NMR (250 MHz, DMSO) δ ppm: 3.75 (2H, q, J=5.2 Hz), 4.39 (1H, t, J=5.2 Hz), 6.73 (1H, d, J=5.4 Hz), 7.15 (2H, d, J=8.7 Hz), 7.68 (2H, m), 7.86 (2H, d, J=8.7 Hz), 8.09 (1H, s), 8.51 (2H, m), 9.86 (1H, s). MS (DCI/NH₃>0) m/z: 361 (M+H⁺).

### 41.2. N-4-{4-[2-(7-Trifluoromethylquinolin-4-ylamino)-ethoxy]-benzyl}-vancomycin dihydrochloride salt

This compound is prepared according to the procedure described in example 36 starting from vancomycin hydrochloride (10.7 g, 7.2 mmol), 4-[2-(7-trifluoromethyl-quinolin-4-ylamino)-ethoxy]-benzaldehyde (3.4 g, 9.4 mmol) (example 41.1), N,N-diisopropylethylamine (2.4 mL, 14.4 mmol) and sodium cyanoborohydride (1.8 g, 28.8 mmol). The dihydrochloride salt is prepared according to the procedure described in example 39.4 with 5.3 mL of a 0.1 N aqueous hydrochloric acid solution (0.5 mmol. PA 1276 is obtained as a white powder (0.5 g, 4%, purity>99%)

RMN ¹H (500 MHz, DMSO d6) δ ppm: 0.87 (3H, d, J=5.8 Hz), 0.93 (3H, d, J=5.8 Hz), 1.13 (3H, d, J=6.2 Hz), 1.51 (3H, s), 1.63-1.67 (3H, m), 1.85 (1H, broad d, J=12.6 Hz), 1.99-2.17 (2H, m), 2.51 (1H, m), 2.59 (3H, s), 3.29 (2H, m), 3.46-3.61 (4H, m), 3.70 (1H, m), 3.78 (2H, d, J=5.2 Hz), 3.78-3.91 (3H, m), 4.17-4.22 (2H, m), 4.29 (3H, t, J=5.3 Hz), 4.44 (2H, m), 4.67 (1H, m), 4.93 (1H, broad s), 5.12 (1H, d, J=6.1 Hz), 5.16 (1H, s), 5.19 (1H, s), 5.22 (1H, broad s), 5.30 (1H, broad s), 5.38 (2H, m), 5.62 (1H, s), 5.78 (2H, d, J=7.7 Hz), 5.98 (2H, m), 6.25 (1H, d, J=1.9 Hz), 6.43 (1H, d, J=1.9 Hz), 6.59 (1H, broad s), 6.69 (1H, s), 6.73 (2H, d, J=8.5 Hz), 6.78 (2H, m), 7.01 (3H, d, J=8.7 Hz), 7.18 (1H, s), 7.24 (1H, d, J=8.3 Hz), 7.34 (1H, d, J=8.3 Hz), 7.43 (2H, d, J=8.7 Hz), 7.48 (2H, d, J=7.8 Hz), 7.60 (1H, d, J=8.1 Hz), 7.75 (1H, d, J=8.1 Hz), 7.87 (1H, s), 7.98 (1H, broad s), 8.14 (1H, s), 8.57-8.59 (3H, m), 8.68 (1H, s), 8.87 (1H, broad s), 9.12 (1H, s), 9.19 (1H, s), 9.46 (1H,s). SM (IS>0) m/z: 1795.1 (M+H⁺), 897.8 (M+2H⁺). Elementary analysis: for C₈₅H₉₀ Cl₂F₃N₁₁O₂₅.2HCl.10.7H₂O: % theor. C 49.57, N 7.48; % exper. C 49.61, N 7.40.

Examples 42 Through 48 Below Exemplify the Creation of Hybrid Molecules in the Aminoquinoline-Oxazolidinone Family.

### Example 42

### Aminoquinoline-Oxazolidinone Hybrid Molecule, Ref PA 1183

### (5S)-[2-(7-Chloroquinolin-4-ylamino)-ethyl]-carbamic acid 3-(3-fluoro-4-morpholin-4-yl-phenyl)-2-oxo-oxazolidin-5-ylmethylester

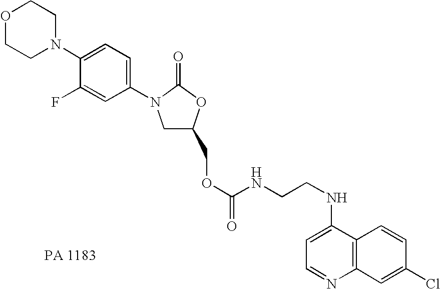

0.3 mL of triethylamine (2.0 mmol) is injected into a solution under argon of 3-(3-fluoro-4-morpholin-4-yl-phenyl)-5-hydroxymethyl-oxazolidin-2-one (prepared according to the method described by S. J. Brickner et al., J. Med. Chem. 1996, 39, 673-679) (0.6 g, 2.0 mmol) in 10 mL of dichloromethane. After this mixture is stirred for 5 min, a triphosgene solution (0.2 g, 0.8 mmol) in 2 mL of dichloromethane is added. The reaction mixture is stirred for 7 hr 30 at room temperature before the addition of a mixture of “N¹-(7-chloroquinolin-4-yl)-ethane-1,2-diamine” (prepared according to the method described by B. Meunier et al., ChemBioChem 2000, 1, 281-283) (0.5 g, 2.0 mmol) and triethylamine (0.3 mL, 2.0 mmol) in 15 mL of dichloromethane. Stirring is continued for 17 hr at room temperature. The reaction medium is then diluted with 20 mL of dichloromethane and washed with 10 mL of 1M aqueous NaOH followed by twice 50 mL of water. The organic layer is dried over sodium sulfate, filtered, and then concentrated in a rotary evaporator. The product is then purified by liquid chromatography on silica gel (SiO₂ 60 Å C.C 6-35 μm, eluent: 9:1 dichloromethane/methanol). After recrystallization from dichloromethane/n-hexane, PA 1183 is obtained as a light beige powder (0.5 g, 49%).

¹H NMR (250 MHz, CDCl₃) δ ppm: 3.00 (4H, m), 3.41 (2H, m), 3.59 (2H, m), 3.78 (1H, m), 3.85 (4H, m), 4.01 (1H, t, J=9.0 Hz), 4.40 (2H, m), 4.82 (1H, m), 5.71 (1H, t, J=6.0 Hz), 6.20 (1H, broad s), 6.30 (1H, d, J=5.3 Hz), 6.78 (1H, t, J=8.8 Hz), 6.92 (1H, dd, J=2.3 Hz, J=8.8 Hz), 7.32 (1H, dd, J=2.0 Hz, J=8.9 Hz), 7.42 (1H, dd, J=2.3 Hz, J=14.2 Hz), 7.72 (1H, d, J=8.9 Hz), 7.90 (1H, d, J=2.9 Hz), 8.47 (1H, d, J=5.3 Hz). MS (DCI/NH₃>0) m/z: 544 (M+H⁺). Elementary analysis: for C₂₆H₂₇ClFN₅O₅.0.4CH₂Cl₂0.15C₆H₁₂: % theor. C 55.52, N 11.86; % exper. C 55.43, N 11.86.

### Example 43

### Aminoquinolin-Oxazolidinone Hybrid Molecule, Ref PA 1185

### (5S)-3-(7-Chloroquinolin-4-ylamino)-N-[3-(3-fluoro-4-morpholin-4-yl-phenyl)-2-oxo-oxazolidin-5-ylmethyl]-propionamide

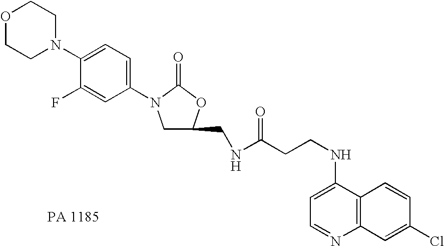

0.7 g of 3-(7-chloroquinolin-4-ylamino)propionic acid (example 4.1) (2.4 mmol), 1.3 g of PyBOP (2.4 mmol), and 1.3 mL of N-methylmorpholine (12.2 mmol) are added to a solution under argon of 5-aminomethyl-3-(3-fluoro-4-morpholin-4-yl-phenyl) -oxazolidin-2-one (prepared according to the method described by S. J. Brickner et al., J. Med. Chem. 1996, 39, 673-679) (0.7 g, 2.4 mmol) in 20 mL of DMF. After stirring for 24 hr at room temperature, the reaction medium is diluted with 100 mL of chloroform and washed with 3 times 100 mL of a saturated solution of bicarbonated water. The organic layer is dried over sodium sulfate, filtered, and then concentrated in a rotary evaporator. The product is then purified by liquid chromatography on silica gel (SiO₂ 60 Å C.C 6-35 μm, eluent: 85:15 dichloromethane/methanol). After recrystallization from chloroform/n-hexane, PA 1185 is obtained as a white powder (0.3 g, 24%).

¹H NMR (250 MHz, DMSO) δ ppm: 2.52 (2H, m), 2.93 (4H, m), 3.46 (4H, m), 3.68 (1H, m), 3.70 (4H, m), 4.01 (1H, t, J=9.0 Hz), 4.71 (1H, m), 6.48 (1H, d, J=5.6 Hz), 7.00 (1H, t, J=9.0 Hz), 7.08 (1H, dd, J=2.0 Hz, J=9.0 Hz), 7.44 (2H, m), 7.53 (1H, broad s), 7.77 (1H, d, J=2.4 Hz), 8.21 (1H, d, J=9.0 Hz), 8.39 (1H, m), 8.40 (1H, d, J=5.6 Hz). MS (IS>0) m/z: 528.50 (M+H⁺). Elementary analysis: for C₂₅H₂₇ClFN₅O₄—H₂O: % theor. C 57.19, N 12.83; % exper. C 57.02, N 12.66.

### Example 44

### Aminoquinoline-Oxazolidinone Hybrid Molecule, Ref PA 1193

### (5S)-2-(7-Chloroquinolin-4-ylamino)-N-[3-(3-fluoro-4-morpholin-4-yl-phenyl)-2-oxo-oxazolidin-5-ylmethyl]-acetamide

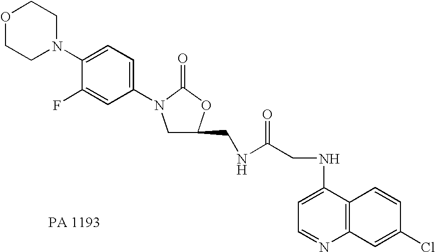

This compound is prepared according to the procedure described in example 43, from 0.7 g of 5-aminomethyl-3-(3-fluoro-4-morpholin-4-yl-phenyl)oxazolidin-2-one (2.2 mmol), 0.5 g of (7-chloroquinolin-4-yl-amino)acetic acid (2.2 mmol) (example 3.1), 1.2 g of PyBOP (2.2 mmol), and 1.2 mL de N-methylmorpholine (11.2 mmol) in 20 mL of dimethylformamide. PA 1193 is obtained as a white powder after purification by liquid chromatography on silica gel (SiO₂ 60 Å C.C 6-35 μm, eluent: 98:2 v/v chloroform/methanol), followed by recrystallization from chloroform/n-hexane (0.5 g, 48%).

¹H NMR (250 MHz, DMSO) δ ppm: 3.08 (4H, m), 3.57 (2H, m), 3.79 (1H, m), 3.86 (4H, m), 4.06 (1H, d, J=5.9 Hz), 4.16 (1H, t, J=9.0 Hz), 4.87 (1H, m), 6.31 (1H, d, J=5.4 Hz), 7.15 (1H, t, J=9.0 Hz), 7.24 (1H, dd, J=2.4 Hz, J=9.0 Hz), 7.59 (2H, m), 7.85 (1H, t, J=5.9 Hz), 7.91 (1H, d, J=2.2 Hz), 8.34 (2H, m), 8.39 (1H, t, 7=5.3 Hz). MS (IS>0) m/z: 514.30 (M+H⁺). Elementary analysis: for C₂₅H₂₅ClFN₅O₄.0.7H₂O: % theor. C 57.02, N 13.30; % exper. C 57.02, N 13.07.

### Example 45

### Aminoquinoline Oxazolidinone Hybrid Molecule, Ref PA 1196

### (5S-[2-(6-Chloroquinolin-2-ylamino)-ethyl]-carbamic acid 3-(3-fluoro-4-morpholin-4-yl-phenyl)-2-oxo-oxazolidin-5-ylmethyl ester

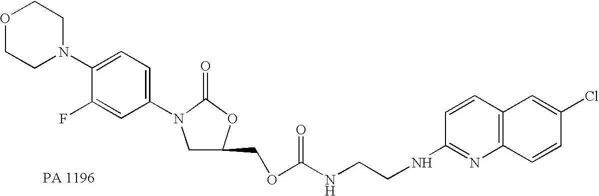

This compound is prepared according to the procedure described in example 42, from 0.6 g of 3-(3-fluoro-4-morpholin-4-yl-phenyl)-5-hydroxymethyl-oxazolidin-2-one (2.1 mmol), 0.3 mL of triethylamine (2.1 mmol), 0.2 g of triphosgene (0.8 mmol), 0.5 g of N′-(6-chloroquinolin-2-yl)-ethane-1.2-diamine (2.1 mmol) (prepared according to the method described by T. J. Egan et al., 3. Med. Chem. 2000, 43, 283-291), and 0.3 mL de triethylamine (2.1 mmol) in 10 mL of dichloromethane. PA 1196 is obtained as a white powder after purification by liquid chromatography on silica gel (SiO₂ 60 Å C.C 6-35 μm, eluent: 91.5:8.5 v/v chloroform/methanol), followed by recrystallization from chloroform/n-hexane (0.5 g, 48%).

¹H NMR (250 MHz, DMSO) δ ppm: 2.74 (4H, m), 3.23 (2H, m), 3.42 (2H, m), 3.72 (4H, m), 3.79 (1H, m), 4.16 (1H, t, J=9.1 Hz), 4.23 (2H, m), 4.88 (1H, m), 6.77 (1H, d, J=9.0 Hz), 7.04 (1H, t, J=9.1 Hz), 7.17 (1H, dd, J=2.2 Hz, J=9.1 Hz), 7.23 (1H, t, J=5.4 Hz), 7.39-7.59 (4H, m), 7.70 (1H, d, J=1.9 Hz), 7.82 (1H, d, J=9.0 Hz). MS (DCI/NH₃>0) m/z: 544 (M+H⁺).

### Example 46

### Preparation of an Aminoquinoline-Oxazolidinone, Ref PA 1205

### (5S)-N-[3-(4-{4-[2-(7-Chloroquinolin-4-ylamino)-acetyl]-piperazin-1-yl}-3-fluoro-phenyl)-2-oxo-oxazolidin-5-ylmethyl]-acetamide

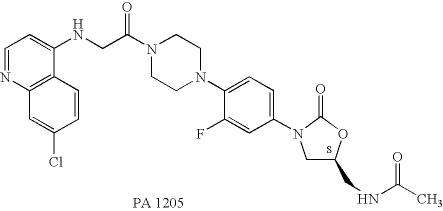

(5S)-N-[3-(3-Fluoro-4-piperazin-1-yl-phenyl)-2-oxo-oxazolidin-5-ylmethyl]-acetamide hydrochloride salt (prepared according to the method described by S. J. Brickner et al., J. Med. Chem. 1996, 39, 673-679) (0.5 g, 1.3 mmol) is dissolved in 20 mL of anhydrous dimethylformamide under argon. To this solution is added (7-chloroquinolin-4-ylamino)-acetic acid (example 3.1) (0.3 g, 1.3 mmol) followed by PyBOP® (0.7 g, 1.3 mmol) and finally N-methylmorpholine (0.9 mL, 8.0 mmol). The mixture is stirred at room temperature under argon for 23 hr. The mixture is then diluted with 100 mL of chloroform and washed three times with 150 mL of saturated aqueous NaHCO₃. The organic layer is dried over sodium sulfate, filtered and concentrated under vacuum. PA 1205 is obtained as a white powder after recrystallisation from hot methanol/n-hexane (0.3 g, 35%).

¹H NMR (250 MHz, DMSO) δ ppm: 1.83 (3H, s), 2.96-3.04 (4H, m), 3.40 (2H, t, J=5.5 Hz), 3.69 (5H, m), 4.08 (1H, t, J=8.8 Hz), 4.25 (2H, d, J=4.5 Hz), 4.70 (1H, m), 6.43 (1H, d, J=5.3 Hz), 7.08 (1H, t, J=9.4 Hz), 7.18 (1H, dd, J=2.2 Hz, J=9.4 Hz), 7.37 (1H, t, J=5.1 Hz), 7.50 (2H, m), 7.81 (1H, d, J=2.0 Hz), 8.21 (1H, d, J=9.0 Hz), 8.24 (1H, m), 8.41 (1H, d, J=5.4 Hz). MS (IS>0) m/z: 555.6 (M+H⁺). Elementary analysis: for C₂₇H₂₈ClFN₆O₄.0.5H₂O: % theor. C 57.49, N 14.90; % exper. C 57.59, N 14.64.

### Example 47

### Preparation of an Aminoquinoline-Oxazolidinone, Ref PA 1210

### (5S)-N-[3-(4-{4-[2-(7-Chloroquinolin-4-ylamino)-ethyl]-piperazin-1-yl}-3-fluoro-phenyl)-2-oxo-oxazolidin-5-ylmethyl]-acetamide

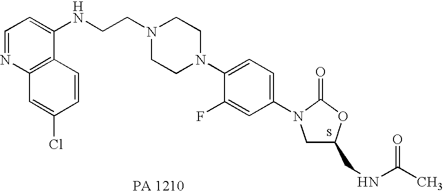 (2-Bromoethyl)-(7-chloroquinolin-4-yl)-amine (0.3 g, 1.0 mmol) is dissolved in 13 mL of anhydrous dimethylformamide under argon. To this solution is added (5S)-N-[3-(3-fluoro-4-piperazin-1-yl-phenyl)-2-oxo-oxazolidin-5-ylmethyl]-acetamide hydrochloride salt (prepared according to the method described by S. J. Brickner et al., 3. Med. Chem. 1996, 39, 673-679) (0.3 g, 1.0 mmol) followed by triethylamine (0.3 mL, 2.2 mmol). The mixture is stirred at room temperature under argon for 5 days. The mixture is then diluted with 100 mL of chloroform and washed three times with 150 mL of saturated aqueous NaHCO₃. The organic layer is dried over sodium sulfate, filtered and concentrated under vacuum. The residual oil is purified by chromatography on silica gel using a chloroform/ethanol 85/15 v/v eluent to afford PA 1210 as a white powder after recrystallisation from chloroform/n-hexane (0.2 g, 33%).

¹H NMR (250 MHz, DMSO) δ ppm: 1.94 (3H, s), 2.80 (6H, m), 3.11 (4H, broad s), 3.45-3.55 (4H, m), 3.80 (1H, m), 4.19 (1H, t, J=9.0 Hz), 4.81 (1H, m), 6.66 (1H, d, J=5.4 Hz), 7.17 (1H, t, J=9.4 Hz), 7.28 (1H, dd, J=2.3 Hz, J=9.4 Hz), 7.37 (1H, t, J=5.2 Hz), 7.59 (2H, m), 7.91 (1H, d, J=2.2 Hz), 8.24 (2H, m), 8.53 (1H, d, J=5.4 Hz). MS (IS>0) m/z: 541.5 (M+H⁺). Elementary analysis: for C₂₇H₃₀ClFN₆O₃.1.5H₂O: % theor. C 57.08, N 14.80; % exper. C 57.38, N 14.75.

### Example 48

### Preparation of an Aminoquinoline-Oxazolidinone, Ref PA 1215

### (5S)-N-[3-{4-[4-(7-Chloroquinolin-4-yl)-piperazin-1-yl]-3-fluoro-phenyl}-2-oxo-oxazolidin-5-ylmethyl]-acetamide

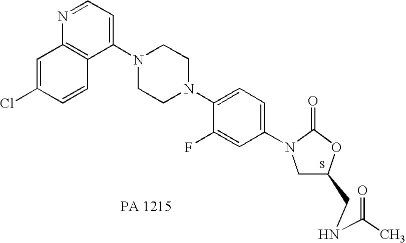

A mixture of (5S)-N-[3-(3-Fluoro-4-piperazin-1-yl-phenyl)-2-oxo-oxazolidin-5-ylmethyl]-acetamide hydrochloride salt (prepared according to the method described by S. J. Brickner et al., J. Med. Chem. 1996, 39, 673-679) (0.4 g, 1.1 mmol), 4,7-dichloroquinoline (0.2 g, 1.2 mmol) and N,N-diisopropylethylamine (0.5 mL, 3.1 mmol) in 20 mL of 2-ethoxyethanol is heated to reflux for 3 hr 40. The mixture is diluted with 60 mL of ethylacetate and 15 mL of chloroform and then washed three times with 150 mL of saturated aqueous NaHCO₃. The organic layer is dried over sodium sulfate, filtered and concentrated under vacuum. The residual oil is purified by chromatography on silica gel using a chloroform/methanol 9/1 v/v eluent to afford PA 1215 as a beige powder after recrystallisation from dichloromethane/n-hexane (0.3 g, 48%).

¹H NMR (250 MHz, CDCl₃) δ ppm: 2.03 (3H, s), 3.34-3.43 (8H, m), 3.57-3.81 (3H, m), 4.04 (1H, t, J=8.9 Hz), 4.79 (1H, m), 6.22 (1H, t, J=6.1 Hz), 6.90 (1H, d, J=5.0 Hz), 7.02 (1H, t, J=8.9 Hz), 7.12 (1H, dd, J=2.2 Hz, J=8.9 Hz), 7.46 (2H, m), 7.99 (1H, d, J=9.0 Hz), 8.09 (1H, d, J=1.8 Hz), 8.74 (1H, d, J=5.0 Hz). MS (IS>0) m/z: 498.2 (M+H⁺). Elementary analysis: for C₂₅H₂₅ClFN₅O₃.0.7H₂O: % theor. C 58.80, N 13.71; % exper. C 59.15, N 13.30.

### Example 49

### Stability Tests for the aminoquinoline-Cephalosporin Hybrid Molecules at Physiological pH and at Acidic pH

The stability of the aminoquinoline-cephalosporin_hybrid molecules given as examples was determined in solution at 37° C., at physiological pH (pH 7, phosphate buffer/acetonitrile, 75/25 v/v) and at acidic pH (pH 1, 0.1 M HCl/ethanol, 70/30 v/v) by high pressure liquid chromatography coupled to a UV-visible detector (Beckman Coulter ODS C18 column, 5 μm, 4.6×250 mm; eluents: A: 0.1% TFA, B: CH₃CN/H₂O 90/10 0.1% TFA, gradient: from 10% to 100% of B in 30 minutes, and then 100% of B for 10 minutes, flow rate 1 mL/minutes, λ=254 nm, volume injected: 10 μL).

The results of stability at pH 7 and pH 1 obtained with the various hybrid molecules of examples 6, 7 and 14 are listed in tables I and II below.

TABLE I
Stability at pH 7
Purity of the hybrid molecules (as a
percentage) as a function of time (hours)

| | Time (h) | | | | | | | |
|---|---|---|---|---|---|---|---|---|
| | 0 | 1 | 2 | 4 | 6 | 8 | 15 | 24 |
| PA 1089 | 100 | 100 | 100 | 100 | 98 | 97 | 92 | 83 |
| (example 6) | | | | | | | | |
| PA 1088 | 100 | 100 | 100 | 100 | — | — | — | 81 |
| (example 7) | | | | | | | | |
| PA 1074 | 100 | 100 | 100 | 100 | 100 | 97 | 88 | 87 |
| (example 14) | | | | | | | | |

TABLE II
Stability at pH 1
Purity of the hybrid molecules (as a
percentage) as a function of time (hours)

Time (h)

| | 0 | 1 | 2 | 4 | 6 | 24 |
|---|---|---|---|---|---|---|
| PA 1089 | 100 | 96 | 88 | 81 | — | 24 |
| (example 6) | | | | | | |
| Ceftriaxone | 100 | 67 | 46 | 21 | 4 | — |

The results in tables I and II demonstrate that the hybrid molecules obtained have excellent stability at the pH tested, particularly at pH 1 (pH of the stomach).

### Example 50

### Antibacterial Activity of the Hybrid Molecules

The antibacterial activity of the hybrid molecules given in the examples was evaluated by determination of the minimum inhibitory concentrations (MIC) in μg/mL by micromethod in liquid medium and minimum bactericidal concentrations (MBC) in μg/mL by subculture on an agar medium, on various Gram+ and Gram−, aerobic and anaerobic bacterial species: Staphylococcus aureus MSSA (methicillin-sensitive) CIP 4.83, Staphylococcus aureus MRSA (methicillin-resistant clinical isolate), Staphylococcus aureus NorA (quinolone-resistant by efflux) 1199B, Staphylococcus aureus MsrA (macrolide-resistant by efflux) PUL5054 (pMS97), Staphylococcus aureus VISA (intermediate sensitivity to vancomycin) CIP 106757, Staphylococcus epidermidis MSCNS (methicillin-sensitive coagulase negative Staphylococcus) D10, Staphylococcus epidermidis MRCNS (methicillin-resistant coagulase negative Staphylococcus) E93, Streptococcus pneumoniae PSSP (penicillin-sensitive) CQI 201 and CIP 69.2, Streptococcus pneumoniae PRSP (penicillin G resistant) CQR 162, a clinical isolate and CIP 104471, Streptococcus pneumoniae mefE (macrolide-resistant efflux) (clinical isolate), Streptococcus pyogenesCIP 56.41T, Enterococcus faecalis VRE (vancomycin-resistant) CIP 104 676, Enterococcus faecium VRE VanA (vancomycin-resistant) CIP 107387, Enterococcus faecalis VRE VanA (vancomycin-resistant) CIP 106996, Enterococcus faecalis VRE VanB (vancomycin-resistant) CIP 106998, Haemophilus influenzae (β-lactamase producer) CIP 102514, Moraxella catarrhalis CIP 7321T, Escherichia coli CIP 54127, Pseudomonas aeruginosa CIP 103467, Bacillus subtilis CIP 5262, Bacillus thuringiensis CIP 104676, Clostridium difficile CIP 104282, Bacteroides fragilis AIP 7716 (inoculation suspension: 10⁸ bacteria/mL, incubation at 37° C., under 5% CO₂ for Streptococcus, Haemophilus, and Enterococcus and under anaerobic conditions for Clostridium difficile).

The results obtained for the action of the hybrid molecules according to the invention on the various bacterial species indicated above are listed in tables III to XIII below.

Aminoquinoline-β-Lactam Hybrid Molecules

TABLE III
Antibacterial activity of an example of an aminoquinoline-penicillin
hybrid molecule on Staphylococcus aureus MSSA CIP 4.83
(MIC and MBC values in μg/mL)

| | MIC (μg/mL) | MBC (μg/mL) |
|---|---|---|
| PA 1007 (ex. 1) | 0.012 | 0.49 |
| Penicillin G | 0.008 | 0.06 |

TABLE IV
Antibacterial activity of examples of aminoquinoline-cephalosporin hybrid molecules (MIC and MBC values in μg/mL)

| | | | S. pneumoniae | S. pneumoniae | S. pneumoniae | | E. faecalis | | |
|---|---|---|---|---|---|---|---|---|---|
| | | S. aureus | PSSP | PRSP | PRSP | S. pyogenes | VRE | M. catarrhalis | H. influenzae |
| | | CIP 4.83 | CIP 6.92 | CIP 104471 | Clinical isolate | CIP 56.41T | CIP 104676 | CIP73.21T | CIP 102514 |
| PA 1046 | MIC | 0.20 | 0.012 | 0.006 | 0.05 | 0.003 | 6.25 | 0.78 | 3.12 |
| (ex. 5) | MBC | >50 | 0.012 | 0.025 | 0.1 | 0.003 | 50 | 0.78 | 25 |
| PA 1089 | MIC | 0.20 | nd | 0.39 | 0.39 | nd | 6.25 | nd | 3.12 |
| (ex. 6) | MBC | 0.39 | nd | 0.39 | 0.39 | nd | 12.5 | nd | >50 |
| PA 1092 | MIC | 12.5 | nd | 1.56 | 6.25 | nd | >50 | nd | 12.5 |
| (ex. 8) | MBC | >50 | nd | 3.12 | 12.5 | nd | >50 | nd | 50 |
| PA 1037 | MIC | 0.39 | nd | 1.56 | 3.12 | nd | 6.25 | nd | 6.25 |
| (ex. 9) | MBC | >50 | nd | 3.12 | 6.25 | nd | 25 | nd | 25 |
| PA 1053 | MIC | 0.78 | nd | 1.56 | 3.12 | nd | 25 | nd | 6.25 |
| (ex. 12) | MBC | >50 | nd | 3.12 | 6.25 | nd | >50 | nd | 25 |
| PA 1074 | MIC | 0.78 | nd | 25 | 25 | nd | 50 | nd | 50 |
| (ex. 14) | MBC | 1.56 | nd | 50 | 50 | nd | >50 | nd | >50 |
| PA 1100 | MIC | 0.20 | 0.006 | 0.20 | 0.39 | 0.003 | 12.5 | 0.78 | 0.78 |
| (ex. 15) | MBC | 25 | 0.006 | 0.78 | 0.39 | 0.003 | 25 | 1.56 | 25 |
| PA 1101 | MIC | 0.20 | 0.006 | 0.20 | 0.20 | 0.0015 | 25 | 0.78 | 1.56 |
| (ex. 16) | MBC | 25 | 0.006 | 0.78 | 0.39 | 0.0015 | 25 | 0.78 | 12.5 |
| Ceftriaxone | MIC | 0.20 | 0.006 | 0.05 | 0.39 | 0.006 | 1.56 | <0.01 | <0.001 |
| | MBC | >50 | 0.006 | 0.39 | 0.78 | 0.006 | 50 | 0.0015 | 1.56 |

The results in tables III and IV above clearly show that the anti-bacterial activity of the aminoquinoline-β-lactam hybrid molecules according to the invention is very significant which is quite unexpected for the person skilled in the art, in particular on the Gram+ bacteria such as S. pneumoniae and S. pyogenes.

TABLE V
Antibacterial activity of the constituent structures of an example
of a hybrid aminoquinoline-cephalosporin molecule, tested
separately and in a 1:1 (mol/mol) association (MIC in μg/mL).

| | | PA 1117 | 7-ACA + PA | PA 1046 |
|---|---|---|---|---|
| | 7-ACA | (ex. 3.1) | 1117 (1:1) | (ex. 5) |
| S. aureus | 50 | >50 | >50 | 0.20 |
| CIP 4.83 | | | | |
| S. pneumoniae PRSP | 50 | 50 | 50 | 0.006 |
| CIP 104471 | | | | |
| S. pneumoniae PRSP | 50 | 50 | 50 | 0.05 |
| clinical isolate | | | | |
| E. faecalis VRE | >50 | >50 | >50 | 6.25 |
| CIP 104676 | | | | |
| H. influenzae | 50 | 50 | 25 | 3.12 |
| CIP 102514 | | | | |

7-ACA: 7-aminocephalosporanic acid;
PA 1117: (7-chloro-quinolin-4-ylamino)-acetic acid;
PA 1046: coupling product of 7-ACA and PA 1117.
The results shown in Table V clearly demonstrate the amplification effect of the antibiotic activity when Q and A are linked by a covalent bond.

TABLE VI
Antibacterial activity of an example of a hybrid
aminoquinoline-cephalosporin molecule in the
presence of human serum (MIC in μg/mL).

| | Ceftriaxone | PA 1046 (ex. 5) |
|---|---|---|
| S. aureus CIP 4.83 | | |
| without serum | 0.20 | 0.2 |
| +50% human serum | 25 | 0.78 |
| S. pneumoniae PRSP clinical isolate | | |
| without serum | 0.78 | 0.20 |
| +50% human serum | 12.5 | 1.56 |

This table shows that unlike the reference molecule, the example of a hybrid aminoquinoline-cephalosporin molecule remains active in vitro in the presence of human serum.

Hybrid Aminoquinoline-Quinolone Molecules

TABLE VII
Antibacterial activity of examples of hybrid aminoquinoline-quinolone
molecules (MIC in μg/mL).

| | | PA | PA | PA | PA |
|---|---|---|---|---|---|
| | | 1126 | 1127 | 1284 | 1285 |
| | Ciprofloxacin | (ex. 24) | (ex. 25) | (ex. 26) | (ex. 27) |
| S. aureus | 0.312 | 0.156 | 1.25 | 0.156 | 0.02 |
| MSSA CIP 4.83 | | | | | |
| S. aureus | >50 | 0.18 | 3.0 | — | — |
| NorA 1199B | | | | | |
| S. pneumoniae | 1.25 | 0.078 | 2.5 | 0.039 | 0.039 |
| PRSP | | | | | |
| E. faecalis | 1.25 | 0.078 | 1.25 | 0.625 | 0.156 |
| VRE CIP 104676 | | | | | |
| B. subtilis | 0.04 | <0.001 | 0.312 | — | — |
| CIP 5262 | | | | | |
| B. thuringiensis | 0.156 | 0.156 | 0.625 | 0.625 | 0.156 |
| CIP 104676 | | | | | |
| E. coli | 0.01 | 0.612 | 0.156 | 1.25 | 0.625 |
| CIP 54127 | | | | | |
| H. influenzae | 0.1 | 0.312 | 0.156 | 0.625 | 0.312 |
| CIP 102514 | | | | | |
| P. aeruginosa | 0.156 | 5 | 5 | >5 | >5 |
| CIP 103467 | | | | | |

These results indicate the contribution of the aminoquinoline by a very marked gain in antibacterial activity when it is bound to an antibiotic in the quinolone family.

Hybrid Aminoquinoline-Nitroimidazole Molecules

TABLE VIII
Antibacterial activity of examples of hybrid
aminoquinoline-nitroimidazole molecules
(MIC in μg/mL).

| | | PA 1129 | PA 1130 |
|---|---|---|---|
| | Metronidazole | (ex. 28) | (ex. 29) |
| B. fragilis | 0.2 | 0.78 | 3.12 |
| API 7716 | | | |

Hybrid aminoquinoline-nitroimidazole molecules are active against a strain of an anaerobic bacterium.

Hybrid Aminoquinoline-Streptogramin Molecules

TABLE IX
Antibacterial activity of examples of hybrid aminoquinoline-
streptogramin molecules (MIC in μg/mL).

| | Pristinamycin I_{A} | PA 1182 (ex. 31) |
|---|---|---|
| S. aureus MSSA | 2.5 | 0.31 |
| CIP 4.83 | | |
| S. aureus MRSA | 5 | 1.25 |
| clinical isolate | | |
| S. pneumoniae PSSP | 0.31 | 0.31 |
| CQI 201 | | |
| S. pyogenes | 1.25 | 0.32 |
| CIP 56.41T | | |

The aminoquinoline noticeably improves the activity of the streptogramin, as shown in the example in the preceding table.

Hybrid Aminoquinoline-Macrolide Molecules

TABLE X
Antibacterial activity of an example of a hybrid aminoquinoline-
macrolide molecule (MIC in μg/mL).

| | Erythromycin | PA 1169 (ex. 35) |
|---|---|---|
| S. aureus MSSA | 0.156 | 0.156 |
| CIP 4.83 | | |
| S. aureus MRSA | 0.156 | 0.31 |
| clinical isolate | | |
| S. pneumoniae PSSP | 0.039 | 0.005 |
| CQI 201 | | |
| S. pneumoniae PRSP | >5 | >5 |
| clinical isolate | | |
| S. pyogenes | 0.039 | 0.078 |
| CIP 56.41T | | |
| S. pneumoniae mef_{E} | 5 | 1.25 |

In the example of a hybrid aminoquinoline-macrolide molecule, the aminoquinoline contributes a worthwhile gain in activity against penicillin-sensitive S. pneumoniae, and also against a strain that is macrolide resistant by efflux.

Hybrid Aminoquinoline-Glycopeptide Molecules

TABLE XI
Antibacterial activity of examples of hybrid aminoquinoline-
glycopeptide molecules (MIC in μg/mL).

| | | PA 1159 | PA 1158 | PA 1157 |
|---|---|---|---|---|
| | Vancomycin | (ex. 38) | (ex. 37) | (ex. 36) |
| S. aureus MSSA | 0.78 | 0.2 | 0.1 | 0.012 |
| CIP 4.83 | | | | |
| S. aureus MRSA | 0.78 | 0.1 | 0.2 | 0.2 |
| clinical isolate | | | | |
| S. aureus MsrA | 0.75 | <0.045 | <0.045 | <0.045 |
| PUL 5054 | | | | |
| S. aureus VISA | 1.56 | 0.2 | 0.39 | 0.1 |
| CIP 106757 | | | | |
| S. epidermidis | 1.56 | 0.2 | 0.1 | 0.05 |
| MSCNS (D10) | | | | |
| S. epidermidis | 0.78 | 0.05 | 0.05 | 0.003 |
| MRCNS (E93) | | | | |
| S. pneumoniae PSSP | 0.2 | 0.006 | 0.012 | 0.003 |
| (CQI 201) | | | | |
| S. pneumoniae PRSP | 0.39 | 0.025 | 0.025 | 0.003 |
| (CQR 162) | | | | |
| S. pyogenes | 0.2 | 0.125 | 0.062 | 0.125 |
| CIP 56.41T | | | | |
| E. faecium VRE VanA | >50 | 25 | 25 | 6.25 |
| CIP107387 | | | | |
| E. faecalis VRE VanA | >50 | 1.56 | 6.25 | 3.125 |
| CIP106996 | | | | |
| E. faecalis VRE VanB | 25 | 12.5 | 6.25 | 6.25 |
| CIP106998 | | | | |
| C. difficile CIP104282 | 0.31 | 0.16 | 0.078 | 0.078 |

The effect of the covalent binding of an aminoquinoline to an antibiotic residue in the glycopeptide family is particularly remarkable, with clearly improved bacterial activity against sensitive strains and also against resistant strains.

Hybrid Aminoquinoline Oxazolidinone Molecules

TABLE XII
Antibacterial activity of an example of a hybrid aminoquinoline-
oxazolidinone molecule (MIC in μg/mL).

| | Linezolid | PA 1185 (ex. 43) |
|---|---|---|
| S. pneumoniae PSSP | 1.25 | 1.25 |
| CQI 201 | | |
| C. difficile | 0.16 | 0.16 |
| CIP 104282 | | |

The hybrid aminoquinoline-oxazolidinone molecule tested in the example demonstrated an anti-bacterial activity that was equivalent to that of the reference molecule.

Example 51 Exemplifies the In Vivo Antibacterial Efficacy of the Hybrid Molecules.

Experimental septicaemia was induced in mice as follows. Briefly, male Swiss OF1 mice (IFFA CREDO-Charles River) weighing 18 to 20 grams were infected intraperitoneally with 0.5 mL of a bacterial suspension containing 8.5×10⁷ cfu of Staphylococcus aureus MSSA 53.154 (Institut Pasteur). The hybrid molecules were administered subcutaneously at 1 and 6 h post infection. Control and treatment groups at each dose were composed of seven mice. All the infected but untreated control mice died within 9 days. The 50% effective dose (ED₅₀) values were calculated by a computerized program (GraphPad) from the survival rates on day 9 after infection.

The in vivo efficacy of the hybrid molecules in a murine septicaemia model induced by Staphylococcus aureus MSSA 53.154 was summarized in tables XIII and XIV.

Hybrid Molecules of the Family of Aminoquinoline-Cephalosporin.

TABLE XIII
in vivo efficacy of hybrid molecules of the family of aminoquinoline-
cephalosporin in an experimental septicaemia.
The hybrid molecules of the family of aminoquinoline-
cephalosporin were formulated in water as their sodium salt.

| | ED₅₀ (mg/kg) |
|---|---|
| Ceftriaxone | 0.09 |
| PA 1046 (ex. 5) | 0.04 |
| PA 1100 (ex. 15) | 0.16 |
| PA 1101 (ex. 16) | 0.13 |

The good in vitro antibacterial activity of the hybrid molecules of the family of aminoquinoline-cephalosporin was confirmed by the results obtained in the in vivo experiment. Cephaloquines demonstrated efficacy against a S. aureus infection. The sodium salt of PA 1046 was even 2 times more effective than ceftriaxone.

Hybrid Molecules of the Family of Aminoquinoline-Glycopeptide.

TABLE XIV
in vivo efficacy of hybrid molecules of the family of aminoquinoline-
glycopeptide in an experimental septicaemia.
The hybrid molecules of the family of aminoquinoline-glycopeptide
were formulated in water as their bis(hydrochloride) salt.

| | ED₅₀ (mg/kg) |
|---|---|
| Vancomycin | 0.16 |
| PA 1157 (ex. 36) | 0.09 |
| PA 1158 (ex. 37) | 0.26 |
| PA 1159 (ex. 38) | 0.37 |

Vancomyquines were also efficacious in combating a S. aureus infection in a murine septicaemia model. Vancomycin was less potent than the bis(dihydrochloride) salt of the vancomyquine PA 1157 and required an higher dose to cure mice.

### Example 52

### Toxicity Tests

Oral Toxicity of an Example of the Aminoquinoline-β-Lactam Hybrid Molecules

The test compound: PA 1046 (example 5) was emulsified in a mixture of Tween 80/methylcellulose in water (0.5% of Tween 80, 0.6% of methylcellulose and 98.9% of water) at different concentrations. The emulsion was administered orally to 6-week-old female Swiss CD1 mice (Janvier) on day 0 and on day 2. The number of survival mice was counted on day 30.

TABLE XV
Oral toxicity of an example of the
aminoquinoline-β-lactam hybrid molecules.

| Doses (mg/kg) | Survival (%) |
|---|---|
| 100 | 5/5 (100%) |
| 200 | 5/5 (100%) |
| 400 | 5/5 (100%) |

After two oral administations of PA 1046 with doses as high as 400 mg/kg, all the mice survive 30 days later thus exemplifying the low toxicity of this hybrid molecule.

Cytoxicity (MTT) Assay of Examples of Hybrid Molecules of the Aminoquinoline-β-Lactam and Aminoquinoline-Glycopeptide Families.

Cellular toxicity was measured by a colorimetric assay that makes use of the tetrazolium salt, MTT. Human lung fibroblast cells (ATCC CCL-171) were plated in 96-well plates at 10⁴ cells per well and incubated under 5% CO₂ at 37° C. for 24 hr to allow adherence of cells to the plate. Test compounds were prepared as a stock solution at 1 mg/mL in 1% DMSO/water and diluted with water for the serial dilutions (500-7.8 μg/mL). Cells underwent a 18 hr treatment under 5% CO₂ at 37° C. with experimental drugs. Corresponding controls received either no drug treatment or treatment with SDS (sodium dodecyl sulfate). The optical density of each sample was read on a microplate spectrophotometer reader at 600 nm and cell survival was expressed as a fraction of that of untreated controls. A drug is said cytotoxic when this fraction is ≦−30%

TABLE XVI
Cytotoxicity assay of an example of the aminoquinoline-β-lactam hybrid
molecules (% of survival cells compared to the untreated controls)
PA 1046 was formulated as its sodium salt.

| Dilutions (μg/mL) | SDS | PA 1046 (ex. 5) |
|---|---|---|
| 7.8 | +2.1% | −5.6% |
| 15.6 | −7.0% | −8.3% |
| 31.2 | −16.4% | −5.6% |
| 62.5 | −77.9% | −9.0% |
| 125 | −81.2% | −14.1% |
| 250 | −81.1% | −17.8% |
| 500 | −83.0% | −18.1% |

The hybrid molecule PA 1046 proved to be non-cytotoxic until the last tested dilution (500 μg/mL) whereas SDS which is known as a cytoxic compound killed more than 80% of the human lung fibroblast cells above a concentration of 31.2 μg/mL.

TABLE XVII
Cytotoxicity assay of examples of the aminoquinoline-glycopeptide hybrid
molecules (% of survival cells compared to the untreated controls)
PA 1157 and PA 1158 were formulated as their bis(hydrochloride) salts.

| Dilutions (μg/mL) | SDS | PA 1158 (ex. 37) | PA 1157 (ex. 36) |
|---|---|---|---|
| 7.8 | +2.1% | −11.8% | −22.0% |
| 15.6 | −7.0% | −13.2% | −22.5% |
| 31.2 | −16.4% | −10.7% | −24.2% |
| 62.5 | −77.9% | −16.9% | −24.9% |
| 125 | −81.2% | −16.9% | −26.9% |
| 250 | −81.1% | −19.8% | −26.2% |
| 500 | −83.0% | −18.4% | −29.2% |

The hybrid molecules PA 11157 and PA 1158 stayed under the threshold of cytoxicity (fixed at less than −30% of survival compared to untreated controls) when SDS exceeded this treshold as soon as a concentration of 31.2 μg/mL.

What is claimed is:

1. A hybrid aminoquinoline-antibiotic compound of general formula (I):
Q—(Y₁)ₚ—(U)_{p′}—(Y₂)_{p″}-A (I)
wherein
Q represents an aminoquinoline-type molecule (IIa), (IIb), (IIIa), (IIIb), (IIIc) or (IIId) as follows: 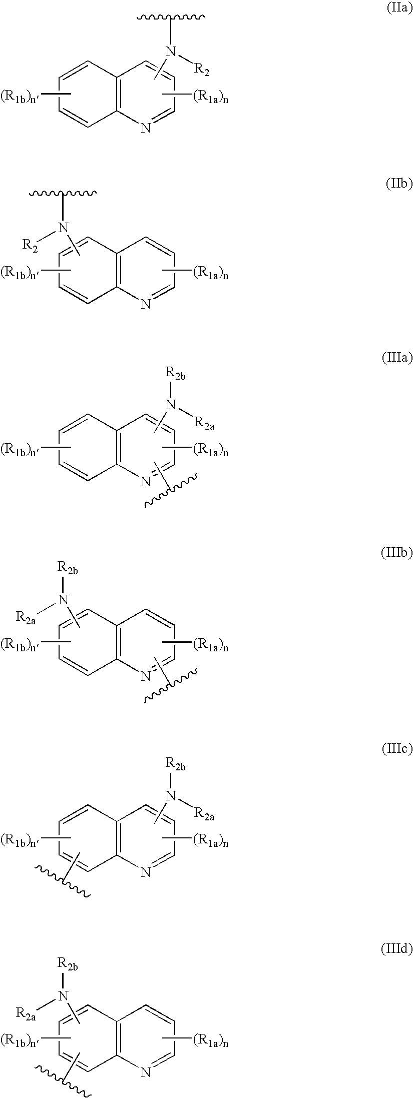
in the above formulae:
the sign [symbol] indicates the anchoring site of the other fragments Y₁, U, Y₂, or A;
n and n′ represent, independently of each other, 0, 1, 2 or 3;
R₁ₐ and R_{1b} (generally R₁) represent one or more substituents which are identical or different, occupying any position and representing a substituent which is selected from the group consisting of halogen, trifluoromethyl, trifluoromethoxy, amine, sulfate, sulfonate, phosphate, phosphonate, nitro, cyano, aryl or heteroaryl or alkyl, alkylamino, dialkylamino, alkoxy, alkylthio, alkylsulfonyl, alkylsulfamoyl, alkylsulfonylamino, alkylcarbamoyl, dialkylcarbamoyl, alkoxycarbonyl, alkylcarbonylamino, the said alkyl groups comprising 1, 2, 3, 4, 5 or 6 carbon atoms, which are linear, branched or cyclic, saturated or unsaturated, containing if need be one or more amine, amide, thioamide, sulfonyl, sulfonamide, carboxy, thiocarboxy, carbonyl, thiocarbonyl, hydroxyimine, ether or thioether functions and themselves being able to bear 1 to 4 substituents, which are identical or different, and which are selected from among halogen, hydroxy, trifluoromethyl, trifluoromethoxy, carboxy, carbonyl, amine, nitro, urea, aryl, or heteroaryl,
R₂ₐ and R_{2b} (generally R₂) being substituents which are identical or different, being able if need be to form a cyclic structure together or with Y₁, Y₂, U or A and representing a hydrogen atom or a linear, branched or cyclic C1, C2, C3, C4, C5 or C6 alkyl substituent containing if need be one or more amine, amide, thioamide, sulfonyl, urea, thiourea, carbamate, oxime, sulfonamide, carboxy, thiocarboxy, carbonyl, thiocarbonyl, ether or thioether functions and being able to bear 1 to 4 substituents, which are identical or different, and which are selected from halogen, hydroxy, trifluoromethyl, trifluoromethoxy, methoxy, carboxy, amine, nitro, aryl, or heteroaryl, R₂ₐ and R_{2b} being not simultaneously a hydrogen atom;
p, p′, p″ are, independently of each other, 0 or 1,
Y₁ and Y₂, which are identical or different, and can be linked by a single or multiple bond to Q, U or A, and represent a saturated or unsaturated, linear, branched or cyclic C1, C2, C3, C4, C5 or C6 alkyl chain, containing if need be one or more amine, amide, thioamide, sulfonyl, sulfonamide, oxo, carboxy, thiocarboxy, carbonyl, thiocarbonyl, urea, thiourea, carbamate, oxime, ether or thioether function, or an aryl or heteroaryl group, wherein the alkyl chain can additionally bear 1 to 4 substituents, which are identical or different, and which are preferably selected from the group consisting of halogen, hydroxy, trifluoromethyl, trifluoromethoxy, methoxy, carboxy, carbonyl, amine, nitro, oxime, aryl or heteroaryl such as defined herein after, or selected from among substituents of the type alkyl, alkylamino, dialkylamino, alkoxy, alkylthio, alkylsulfonyl, alkylsulfonamino, alkylsulfamoyl, alkylureido, alkylcarbamoyloxy, alkoxycarbonylamino, alkylcarbamoyl, dialkylcarbamoyl, alkylcarbonylamino, alkylcarbonyl, alkylcarbonyloxy, alkoxycarbonyl, alkoxyimine, the said alkyl groups comprising from 1 to 6 linear, branched or cyclic carbon atoms which can themselves contain one or more amine, amide, thioamide, sulfonyl, sulfonamide, carboxy, thiocarboxy, carbonyl, thiocarbonyl, oxime, ether or thioether function, or an aryl or heteroaryl group, such as those defined herein after, wherein the C1, C2, C3, C4, C5 or C6 chain may form a cyclic structure with R₂ including N from the aminoquinoline part Q and/or the function U and Y₁ and Y₂ may be linked together or to Q, U or A by a single or multiple bond,
U, which can be linked by a single or multiple bond to Q, Y₁, Y₂ or A, is an amine, amide, thioamide, sulfonyl, sulfonamide, carboxy, thiocarboxy, carbonyl, urea, thiourea, carbamate, ether, thioether, thiocarbonyl, sulfonate, oxime, oxyamine, alkylamine (NR), alkoxyimine (C═N—OR) or alkoxyiminocarbonyl (C(O)—C═N—OR) function with R representing a hydrogen atom or a C1, C2, C3, C4, C5 or C6 alkyl substituent, which is linear, branched or cyclic, containing if need be one or more amine, amide, thioamide, sulfonyl, sulfonamide, carboxy, thiocarboxy, carbonyl, thiocarbonyl, ether or thioether functions, with the proviso that U is not a carbonyl when antibacterial antibiotic residue A is an oxazolidinone of formula (XIVa) 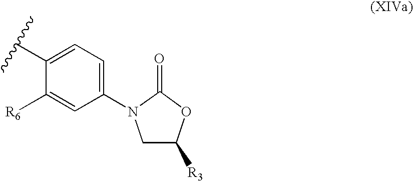
A represents an antibacterial antibiotic residue selected from the group consisting of β-lactams, quinolones, oxazolidinones, fosfomycin derivatives, nitroimidazoles, nitrofurans, sulfamides, streptogramins, synergistins, lincosamides, tetracyclins, chloramphenicol derivatives, fusidic acid derivatives, diaminopyrimidines, aminosides, polypeptides, and glycopeptides,
Q being not a 2-aminoquinoline when A is a carbapenem,
Q being not a 3-aminoquinoline when A is a nitroimidazole or an oxazolidinone,
Q being not a 6-aminoquinoline when A is a macrolide or a quinolone,
Q being not an aminoquinolinium when A is a cephalosporin of formula (VIIIb), (VIIId) or (IXb): 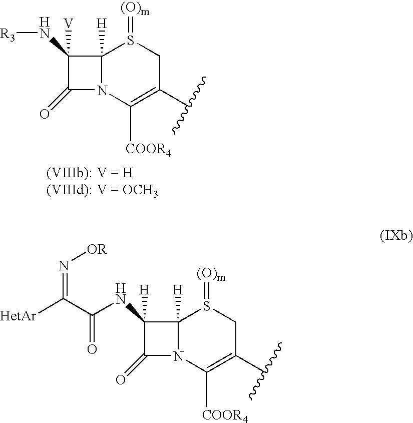
when A is oxazolidinone, the link —(Y₁)ₚ—(U)_{p′}—(Y₂)_{p″} is not a direct link (p=p′=p″=0), not a carbonyl (p=p″=0, p′=1), and not a C1-alkyl group (p′=p″=0, p=1), and:
1) When A is 1-cyclopropyl-6-fluoro-1,4-dihydro-quinoline-3-carboxylic acid or 1-cyclopropyl-6,8-difluoro-1,4-dihydro-quinoline-3-carboxylic acid, and when the link —(Y₁)ₚ—(U)_{p′}—(Y₂)_{p″} between A and Q is a piperazine, then Q is other than 7-chloro-4-aminoquinoline; i.e. compounds having the formulae: 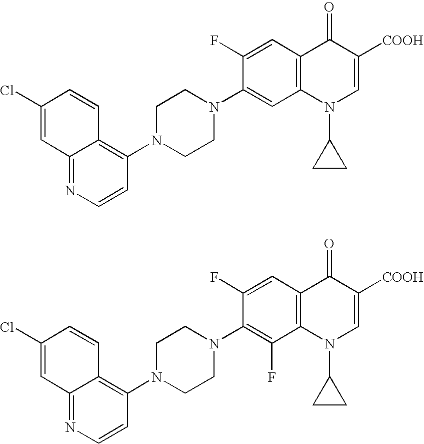
2) When A is a β-lactam having the formula 3-chloro-azetidin-2-one substituted at the 4 position, and when in the link —(Y₁)ₚ—(U)_{p′}—(Y₂)_{p″}—, p, p′, and p″ equal 0, thus forming a direct covalent bond between the nitrogen N1 of A and the extracyclic nitrogen of a 2-aminoquinoline, then Q is other than 2-amino-4-methylquinoline, i.e. for example, compounds having the formula: 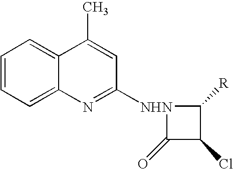
3) When A is (5S)-4-{5-acetylamino-methyl)-2-oxo-oxazolidin-3-yl}-2-fluoro-phenyl, and the link —(Y₁)ₚ—(U)_{p′}—(Y₂)_{p″} is a 4-piperazin-1-yl link including R₂ and N of the aminoquinoline then Q is other than quinolin-4-yl, i.e. the compound having the formula: 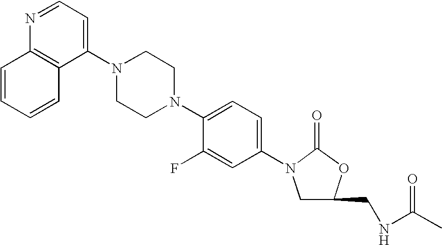
4) When A is a diaminopyrimidine and the link —(Y)ₚ—(U)_{p′}—(Y₂)_{p″} is a methylene link, then Q is other than the following quinolines: 2-morpholino-4-methylquinolin-7-yl, 4-methyl-8-aminoquinolin-6-yl, 4-methyl-5-aminoquinolin-6-yl, 2-dimethylamino methylquinolin-6-yl, 2-dimethylamino-4,8-dimethylquinolin-6-yl, 2-morpholino-4,8-dimethyl-quinolin-6-yl, 2-methyl-4-dimethylamino-8-methoxyquinolin-6-yl, i.e. for example compounds having the formula: 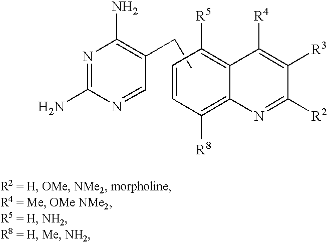
5) When A is 2-methyl-5-nitro-imidazol-1-yl linked directly to the extracyclic nitrogen atom of the aminoquinoline Q (p=p′=p″=0), then Q is other than the following quinolines: 7-chloroquinolin-4-ylamino, i.e. compounds having the formulae: 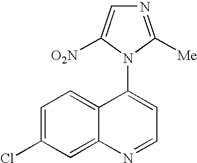
6) When A is 2-methyl-5-nitro-imidazol-1-yl, and the link —(Y₁)ₚ—(U)_{p′}—(Y₂)_{p″} is 2-ethyl(1-cyclohexan-4-yl)-amine, then Q is other than a 7-chloro-quinolin ylamino, i.e. the compound having the formula:

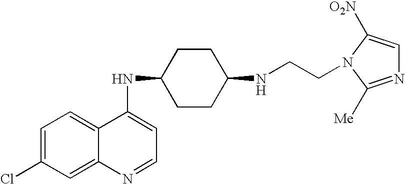

2. The compound according to claim 1, wherein the antibacterial antibiotic A is a β-lactams selected from the group consisting of: a penam (or penicillin) of formula (IV), an oxapenams of formula (V), a penem of formula (VI), a carbapenems of formula (VII), a cephem (or cephalosporin) of formula (VIIIa), (VIIIb), (IXa) or (IXb), a cephamycin of formula (VIIIc) or (VIIId), an oxacephems of formula (Xa) or (Xb), a carbacephem of formula (XIa) or (XIb), and a monobactam of formula (XII), as follows: 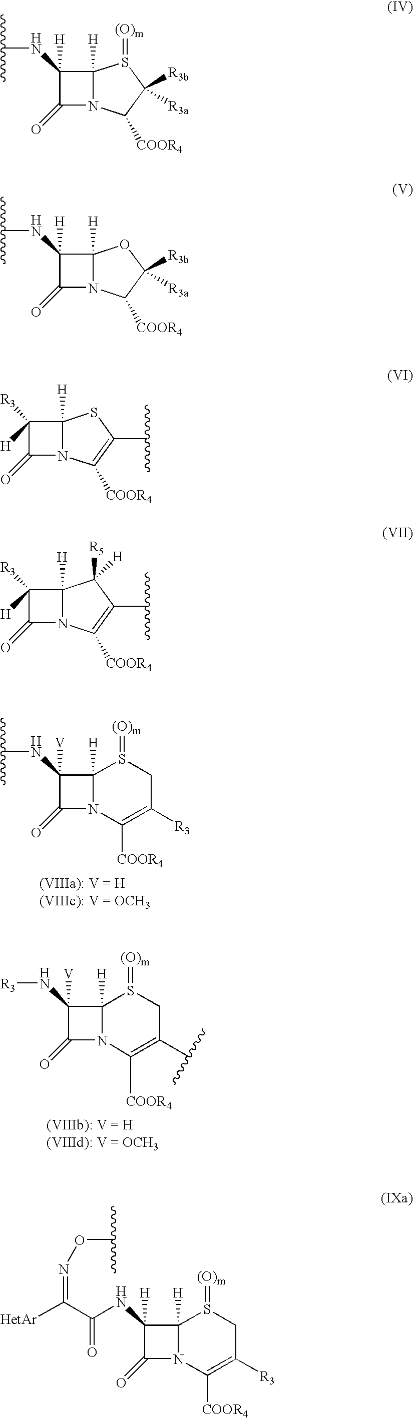 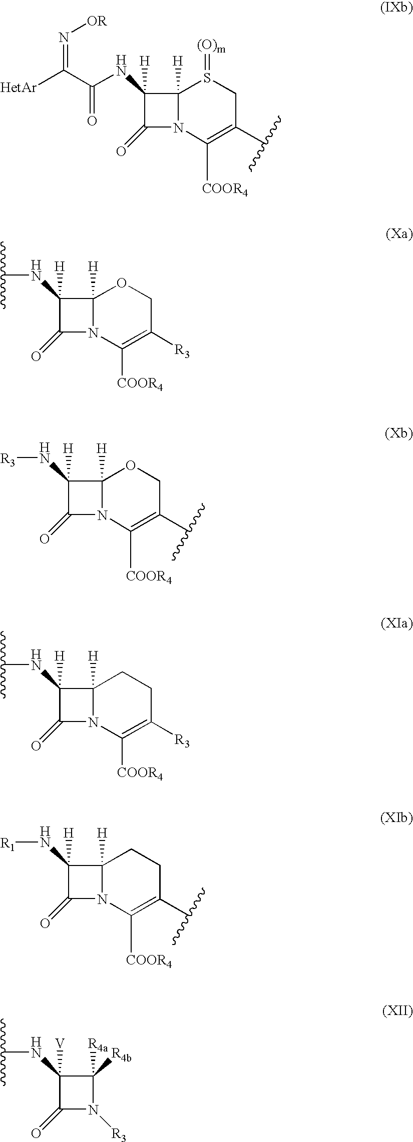
Wherein
R₃ₐ and R_{3b} (generally R₃) represent substituents which are identical or different and which are selected from the group consisting of halogen, hydroxy, trifluoromethyl, trifluoromethoxy, carboxy, aldehyde, amine, sulfate, sulfonate, phosphate, phosphonate, nitro, cyano, aryl or heteroaryl or alkyl, alkylamino, dialkylamino, alkoxy, alkylthio, alkylsulfonyl, alkylsulfonylamino, alkylsulfamoyl, alkylureido, alkylcarbamoyloxy, alkoxycarbonylamino, alkylcarbamoyl, dialkylcarbamoyl, alkylcarbonylamino, alkylcarbonyl, alkylcarbonyloxy, alkyloxycarbonyl, alkoxyimine, the said alkyl groups comprising 1, 2, 3, 4, 5 or 6 carbon atoms, which are saturated or unsaturated, linear, branched or cyclic, containing if need be one or more amine, amide, thioamide, sulfonyl, sulfonamide, oxo, carboxy, thiocarboxy, carbonyl, thiocarbonyl, urea, thiourea, carbamate, oxime, ether or thioether functions that can themselves bear 1 to 4 substituents, which are identical or different, and which are selected from halogen, hydroxy, trifluoromethyl, methyl, trifluoromethoxy, carboxy, carbonyl, amine, nitro, urea, aryl or heteroaryl or heterocycle,
R₄ₐ and R_{4b} (generally R₄), which are identical or different, being able if need be to form, together, a cyclic structure or a multiple bond, represent a hydrogen atom or a saturated or unsaturated, linear, branched or cyclic C1, C2, C3, C4, C5 or C6 alkyl substituent, containing if need be one or more amine, amide, thioamide, sulfonyl, sulfonamide, carboxy, thiocarboxy, carbonyl, thiocarbonyl, oxime, urea, carbamate, ether or thioether functions and being able to bear 1 to 4 substituents, which are identical or different, and which are selected from halogen, hydroxy, trifluoromethyl, trifluoromethoxy, methoxy, carboxy, amine, nitro, aryl, or heteroaryl,
R₅ is a hydrogen atom or a saturated or unsaturated, linear, branched or cyclic C1, C2, C3, C4, C5 or C6 alkyl substituent,
V represents a methoxy group or a hydrogen atom,
“HetAr” represents a heteroaryl group.

3. The compound according to claim 1, wherein the antibacterial antibiotic residue A represents a quinolone moiety represented by the following formula (XIIIa) or (XIIIb), 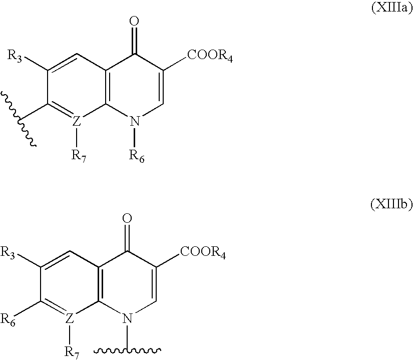
wherein
R₃ represents a substituent selected from the group consisting of halogen, hydroxy, trifluoromethyl, trifluoromethoxy, carboxy, aldehyde, amine, sulfate, sulfonate, phosphate, phosphonate, nitro, cyano, aryl or heteroaryl or alkyl, alkylamino, dialkylamino, alkoxy, alkylthio, alkylsulfonyl, alkylsulfonylamino, alkylsulfamoyl, alkylureido, alkylcarbamoyloxy, alkoxycarbonylamino, alkylcarbamoyl, dialkylcarbamoyl, alkylcarbonylamino, alkylcarbonyl, alkylcarbonyloxy, alkyloxycarbonyl, alkoxyimine, the said alkyl groups comprising 1, 2, 3, 4, 5 or 6 carbon atoms, which are saturated or unsaturated, linear, branched or cyclic, containing if need be one or more amine, amide, thioamide, sulfonyl, sulfonamide, oxo, carboxy, thiocarboxy, carbonyl, thiocarbonyl, urea, thiourea, carbamate, oxime, ether or thioether functions that can themselves bear 1 to 4 substituents, which are identical or different, and which are selected from halogen, hydroxy, trifluoromethyl, methyl, trifluoromethoxy, carboxy, carbonyl, amine, nitro, urea, aryl or heteroaryl or heterocycle,
R₄ represents a hydrogen atom or a saturated or unsaturated, linear, branched or cyclic C1, C2, C3, C4, C5 or C6 alkyl substituent, containing if need be one or more amine, amide, thioamide, sulfonyl, sulfonamide, carboxy, thiocarboxy, carbonyl, thiocarbonyl, oxime, urea, carbamate, ether or thioether functions and being able to bear 1 to 4 substituents, which are identical or different, and which are selected from halogen, hydroxy, trifluoromethyl, trifluoromethoxy, methoxy, carboxy, amine, nitro, aryl, or heteroaryl,
R₆ and R₇ are substituents which are identical or different, being able if need be able to form, together, a cyclic structure and representing a hydrogen atom or a substituent which is selected from the group consisting of halogen, hydroxy, heterocycle, aryl or heteroaryl, or an alkyl, alkoxy or alkylamine substituent, said alkyl groups comprising 1, 2, 3, 4, 5 or 6 carbon atoms, which are saturated or unsaturated, linear, branched or cyclic, containing if need be one or more amine, amide, thioamide, sulfonyl, sulfonamide, carboxy, thiocarboxy, carbonyl, thiocarbonyl, ether or thioether functions and being able to bear 1 to 4 substituents, which are identical or different, and which are selected from halogen, hydroxy, trifluoromethyl, trifluoromethoxy, carboxy, amine, nitro, aryl, or heteroaryl,
Z is a nitrogen or carbon atom.

4. The compound according to claim 1, wherein the antibacterial antibiotic residue A represents an oxazolidinone residue represented by the following formula (XIVa), (XVIb) or (XIVc), 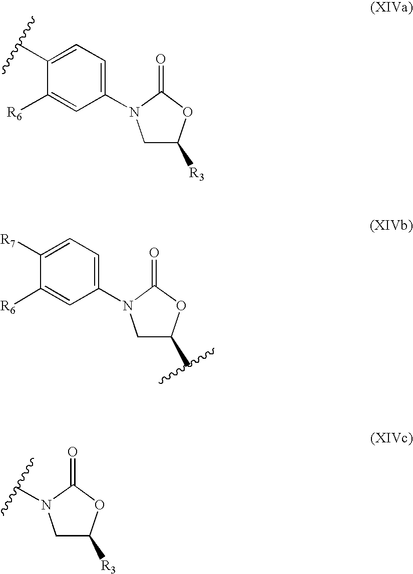
in which R₃, R₆ and R₇ are as follows:
R₃ is a substituent selected from the group consisting of halogen, hydroxy, trifluoromethyl, trifluoromethoxy, carboxy, aldehyde, amine, sulfate, sulfonate, phosphate, phosphonate, nitro, cyano, aryl or heteroaryl or alkyl, alkylamino, dialkylamino, alkoxy, alkylthio, alkylsulfonyl, alkylsulfonylamino, alkylsulfamoyl, alkylureido, alkylcarbamoyloxy, alkoxycarbonylamino, alkylcarbamoyl, dialkylcarbamoyl, alkylcarbonylamino, alkylcarbonyl, alkylcarbonyloxy, alkyloxycarbonyl, alkoxyimine, the said alkyl groups comprising 1, 2, 3, 4, 5 or 6 carbon atoms, which are saturated or unsaturated, linear, branched or cyclic, containing if need be one or more amine, amide, thioamide, sulfonyl, sulfonamide, oxo, carboxy, thiocarboxy, carbonyl, thiocarbonyl, urea, thiourea, carbamate, oxime, ether or thioether functions that can themselves bear 1 to 4 substituents, which are identical or different, and which are selected from halogen, hydroxy, trifluoromethyl, methyl, trifluoromethoxy, carboxy, carbonyl, amine, nitro, urea, aryl or heteroaryl or heterocycle,
R₆ and R₇ are substituents which are identical or different, being able if need be able to form, together, a cyclic structure and representing a hydrogen atom or a substituent which is selected from the group consisting of halogen, hydroxy, heterocycle, aryl or heteroaryl, or an alkyl, alkoxy or alkylamine substituent, said alkyl groups comprising 1, 2, 3, 4, 5 or 6 carbon atoms, which are saturated or unsaturated, linear, branched or cyclic, containing if need be one or more amine, amide, thioamide, sulfonyl, sulfonamide, carboxy, thiocarboxy, carbonyl, thiocarbonyl, ether or thioether functions and being able to bear 1 to 4 substituents, which are identical or different, and which are selected from halogen, hydroxy, trifluoromethyl, trifluoromethoxy, carboxy, amine, nitro, aryl, or heteroaryl.

5. The compound according to claim 1, wherein the antibacterial antibiotic residue A represents a fosfomycin derivative having the following formula (XV), 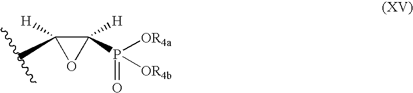
Wherein R₄ₐ and R_{4b} which are identical or different, being able if need be to form, together, a cyclic structure or a multiple bond, represent a hydrogen atom or a saturated or unsaturated, linear, branched or cyclic C1, C2, C3, C4, C5 or C6 alkyl substituent, containing if need be one or more amine, amide, thioamide, sulfonyl, sulfonamide, carboxy, thiocarboxy, carbonyl, thiocarbonyl, oxime, urea, carbamate, ether or thioether functions and being able to bear 1 to 4 substituents, which are identical or different, and which are selected from halogen, hydroxy, trifluoromethyl, trifluoromethoxy, methoxy, carboxy, amine, nitro, aryl, or heteroaryl,
R₅ is a hydrogen atom or a saturated or unsaturated, linear, branched or cyclic C1, C2, C3, C4, C5 or C6 alkyl substituent.

6. The compound according to claim 1, wherein the antibacterial antibiotic residue A represents a nitroimidazole having the following formula (XVIa) or (XVIb) or a nitrofuran residue having the following formula (XVII), 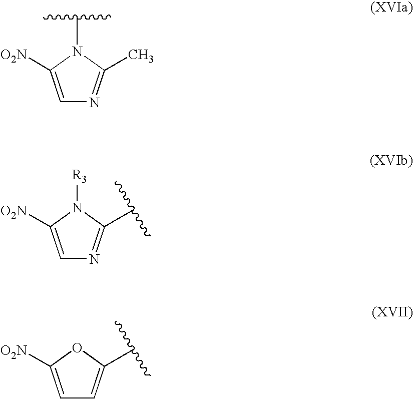
in which R₃ is selected from the group consisting of halogen, hydroxy, trifluoromethyl, trifluoromethoxy, carboxy, aldehyde, amine, sulfate, sulfonate, phosphate, phosphonate, nitro, cyano, aryl or heteroaryl or alkyl, alkylamino, dialkylamino, alkoxy, alkylthio, alkylsulfonyl, alkylsulfonylamino, alkylsulfamoyl, alkylureido, alkylcarbamoyloxy, alkoxycarbonylamino, alkylcarbamoyl, dialkylcarbamoyl, alkylcarbonylamino, alkylcarbonyl, alkylcarbonyloxy, alkyloxycarbonyl, alkoxyimine, the said alkyl groups comprising 1, 2, 3, 4, 5 or 6 carbon atoms, which are saturated or unsaturated, linear, branched or cyclic, containing if need be one or more amine, amide, thioamide, sulfonyl, sulfonamide, oxo, carboxy, thiocarboxy, carbonyl, thiocarbonyl, urea, thiourea, carbamate, oxime, ether or thioether functions that can themselves bear 1 to 4 substituents, which are identical or different, and which are selected from halogen, hydroxy, trifluoromethyl, methyl, trifluoromethoxy, carboxy, carbonyl, amine, nitro, urea, aryl or heteroaryl or heterocycle.

7. The compound according to claim 1, wherein the antibacterial antibiotic residue A represents a sulfamide having the following formula (XVIII),

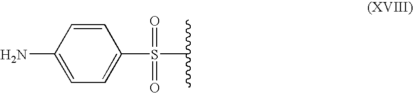

8. The compound according to claim 1, wherein the antibacterial antibiotic residue A represents a streptogramin residue or a synergistin residue having the following formula (XIXa), (XIXb) (XIXc), (XXa) or (XXb), 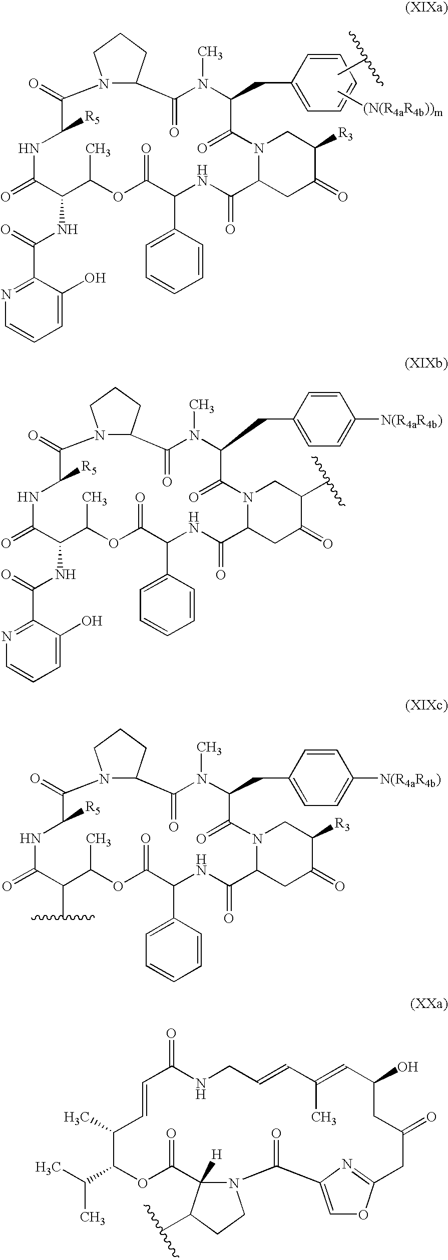 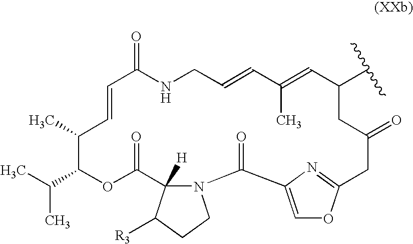
in which R₃, R₁, R_{1b}, R₅ and m are as follows:
R₃ is a substituent selected from the group consisting of halogen, hydroxy, trifluoromethyl, trifluoromethoxy, carboxy, aldehyde, amine, sulfate, sulfonate, phosphate, phosphonate, nitro, cyano, aryl or heteroaryl or alkyl, alkylamino, dialkylamino, alkoxy, alkylthio, alkylsulfonyl, alkylsulfonylamino, alkylsulfamoyl, alkylureido, alkylcarbamoyloxy, alkoxycarbonylamino, alkylcarbamoyl, dialkylcarbamoyl, alkylcarbonylamino, alkylcarbonyl, alkylcarbonyloxy, alkyloxycarbonyl, alkoxyimine, the said alkyl groups comprising 1, 2, 3, 4, 5 or 6 carbon atoms, which are saturated or unsaturated, linear, branched or cyclic, containing if need be one or more amine, amide, thioamide, sulfonyl, sulfonamide, oxo, carboxy, thiocarboxy, carbonyl, thiocarbonyl, urea, thiourea, carbamate, oxime, ether or thioether functions that can themselves bear 1 to 4 substituents, which are identical or different, and which are selected from halogen, hydroxy, trifluoromethyl, methyl, trifluoromethoxy, carboxy, carbonyl, amine, nitro, urea, aryl or heteroaryl or heterocycle,
R₄ₐ and R_{4b} which are identical or different, being able if need be to form, together, a cyclic structure or a multiple bond, represent a hydrogen atom or a saturated or unsaturated, linear, branched or cyclic C1, C2, C3, C4, C5 or C6 alkyl substituent, containing if need be one or more amine, amide, thioamide, sulfonyl, sulfonamide, carboxy, thiocarboxy, carbonyl, thiocarbonyl, oxime, urea, carbamate, ether or thioether functions and being able to bear 1 to 4 substituents, which are identical or different, and which are selected from halogen, hydroxy, trifluoromethyl, trifluoromethoxy, methoxy, carboxy, amine, nitro, aryl, or heteroaryl,
R₅ is a hydrogen atom or a saturated or unsaturated, linear, branched or cyclic C1, C2, C3, C4, C5 or C6 alkyl substituent.

9. The compound according to claim 1, wherein the antibacterial antibiotic residue A represents a lincosamide having the following formula (XXI),

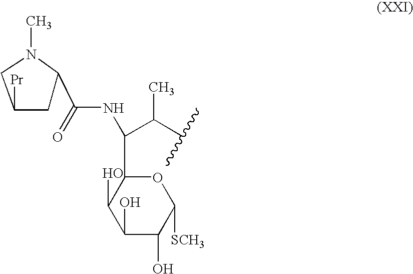

10. The compound according to claim 1, wherein the antibacterial antibiotic residue A represents a tetracyclin residue having the following formulae (XXIIa), (XXIIb) and (XXIIc), 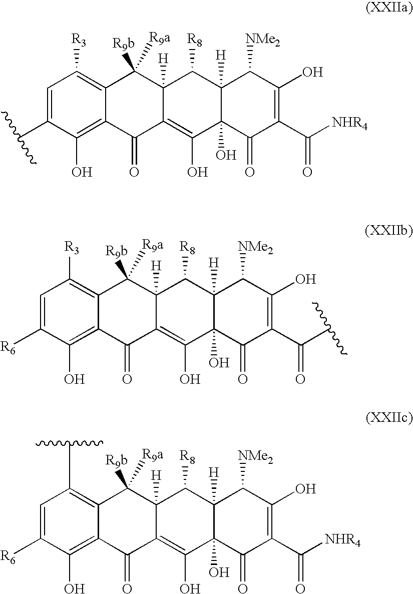
in which R₃, R₄ and R₆ are as follows:
R₃ is a substituent selected from the group consisting of halogen, hydroxy, trifluoromethyl, trifluoromethoxy, carboxy, aldehyde, amine, sulfate, sulfonate, phosphate, phosphonate, nitro, cyano, aryl or heteroaryl or alkyl, alkylamino, dialkylamino, alkoxy, alkylthio, alkylsulfonyl, alkylsulfonylamino, alkylsulfamoyl, alkylureido, alkylcarbamoyloxy, alkoxycarbonylamino, alkylcarbamoyl, dialkylcarbamoyl, alkylcarbonylamino, alkylcarbonyl, alkylcarbonyloxy, alkyloxycarbonyl, alkoxyimine, the said alkyl groups comprising 1, 2, 3, 4, 5 or 6 carbon atoms, which are saturated or unsaturated, linear, branched or cyclic, containing if need be one or more amine, amide, thioamide, sulfonyl, sulfonamide, oxo, carboxy, thiocarboxy, carbonyl, thiocarbonyl, urea, thiourea, carbamate, oxime, ether or thioether functions that can themselves bear 1 to 4 substituents, which are identical or different, and which are selected from halogen, hydroxy, trifluoromethyl, methyl, trifluoromethoxy, carboxy, carbonyl, amine, nitro, urea, aryl or heteroaryl or heterocycle,
R₄ represents a hydrogen atom or a saturated or unsaturated, linear, branched or cyclic C1, C2, C3, C4, C5 or C6 alkyl substituent, containing if need be one or more amine, amide, thioamide, sulfonyl, sulfonamide, carboxy, thiocarboxy, carbonyl, thiocarbonyl, oxime, urea, carbamate, ether or thioether functions and being able to bear 1 to 4 substituents, which are identical or different, and which are selected from halogen, hydroxy, trifluoromethyl, trifluoromethoxy, methoxy, carboxy, amine, nitro, aryl, or heteroaryl,
R₆ is a hydrogen atom or a substituent which is selected from the group consisting of halogen, hydroxy, heterocycle, aryl or heteroaryl, or an alkyl, alkoxy or alkylamine substituent, said alkyl groups comprising 1, 2, 3, 4, 5 or 6 carbon atoms, which are saturated or unsaturated, linear, branched or cyclic, containing if need be one or more amine, amide, thioamide, sulfonyl, sulfonamide, carboxy, thiocarboxy, carbonyl, thiocarbonyl, ether or thioether functions and being able to bear 1 to 4 substituents, which are identical or different, and which are selected from halogen, hydroxy, trifluoromethyl, trifluoromethoxy, carboxy, amine, nitro, aryl, or heteroaryl,
R₈ and R₉ₐ, R_{9b}, which are identical or different, represent a hydrogen atom or a substituent which is selected from the group consisting of hydroxy and methyl.

11. The compound according to claim 1, wherein the antibacterial antibiotic residue A represents a chloramphenicol having the following formula (XXIIIa) or (XXIIIb), 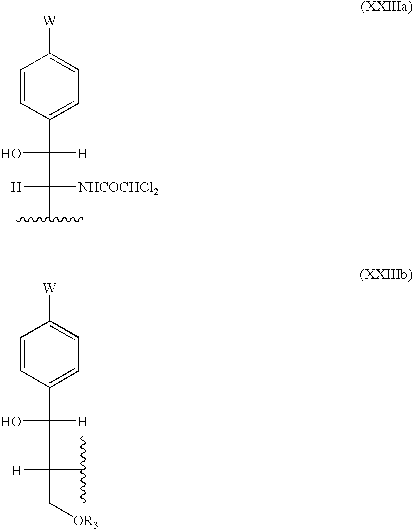
in which
R₃ is a substituent selected from the group consisting of halogen, hydroxy, trifluoromethyl, trifluoromethoxy, carboxy, aldehyde, amine, sulfate, sulfonate, phosphate, phosphonate, nitro, cyano, aryl or heteroaryl or alkyl, alkylamino, dialkylamino, alkoxy, alkylthio, alkylsulfonyl, alkylsulfonylamino, alkylsulfamoyl, alkylureido, alkylcarbamoyloxy, alkoxycarbonylamino, alkylcarbamoyl, dialkylcarbamoyl, alkylcarbonylamino, alkylcarbonyl, alkylcarbonyloxy, alkyloxycarbonyl, alkoxyimine, the said alkyl groups comprising 1, 2, 3, 4, 5 or 6 carbon atoms, which are saturated or unsaturated, linear, branched or cyclic, containing if need be one or more amine, amide, thioamide, sulfonyl, sulfonamide, oxo, carboxy, thiocarboxy, carbonyl, thiocarbonyl, urea, thiourea, carbamate, oxime, ether or thioether functions that can themselves bear 1 to 4 substituents, which are identical or different, and which are selected from halogen, hydroxy, trifluoromethyl, methyl, trifluoromethoxy, carboxy, carbonyl, amine, nitro, urea, aryl or heteroaryl or heterocycle,
W represents an NO₂ or SO₂R₅ substituent, wherein R₅ is a hydrogen atom or a saturated or unsaturated, linear, branched or cyclic C1, C2, C3, C4, C5 or C6 alkyl substituent.

12. The compound according to claim 1, wherein the antibacterial antibiotic residue A represents a derivative of fusidic acid of the following formula (XXIVa), (XXIVb) or (XXIVc),

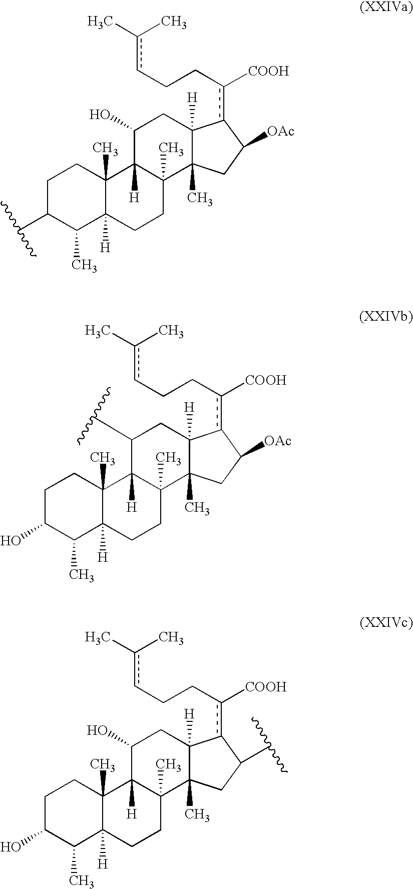

13. The compound according to claim 1, wherein the antibacterial antibiotic residue A represents a diaminopyrimidine of the following formula (XXV), 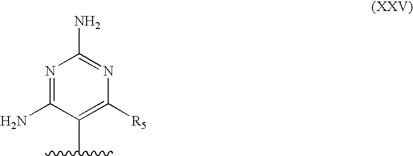
Wherein R₅ is a hydrogen atom or a saturated or unsaturated, linear, branched or cyclic C1, C2, C3, C4, C5 or C6 alkyl substituent.

14. The compound according to claim 1, wherein the antibiotic residue A represents an aminoside which is formed by the union of a genin moiety from the group of aminocyclitols, with one or more oses at least one of which is an aminosugar, which are linked together via glycosidic bridges.

15. A hybrid aminoquinoline-antibiotic compound of general formula (I):
Q—(Y₁)ₚ—(U)_{p′}—(Y₂)_{p″}-A (I)
wherein
Q represents an aminoquinoline-type molecule (IIa), (IIb), (IIIa), (IIIb), (IIIc) or (IIId) as follows: 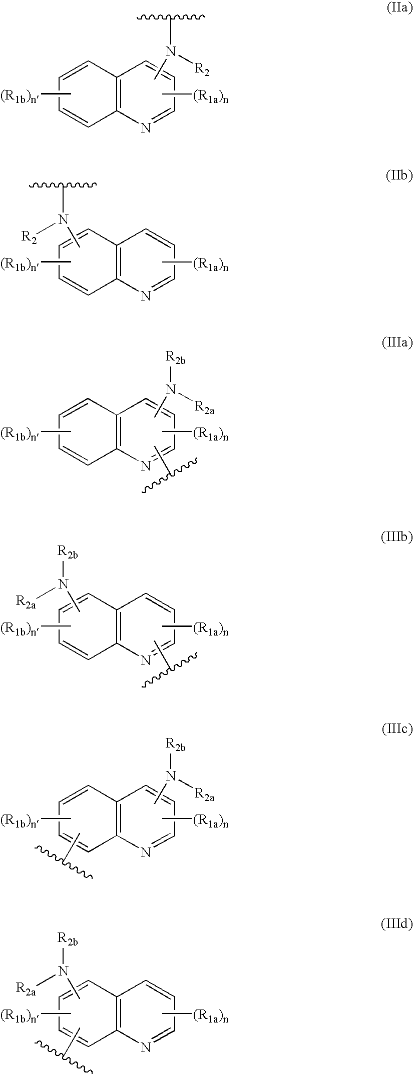
in the above formulae:
the sign [symbol] indicates the anchoring site of the other fragments Y₁, U, Y₂, or A;
n and n′ represent, independently of each other, 0, 1, 2 or 3;
R₁ₐ and R_{1b} (generally R₁) represent one or more substituents which are identical or different, occupying any position and representing a substituent which is selected from the group consisting of halogen, trifluoromethyl, trifluoromethoxy, carboxy, amine, sulfate, sulfonate, phosphate, phosphonate, nitro, cyano, aryl or heteroaryl or alkyl, alkylamino, dialkylamino, alkoxy, alkylthio, alkylsulfonyl, alkylsulfamoyl, alkylsulfonylamino, alkylcarbamoyl, dialkylcarbamoyl, alkylcarbonyloxy, alkoxycarbonyl, alkoxycarbonyloxy, alkylcarbonylamino, the said alkyl groups comprising 1, 2, 3, 4, 5 or 6 carbon atoms, which are linear, branched or cyclic, saturated or unsaturated, containing if need be one or more amine, amide, thioamide, sulfonyl, sulfonamide, carboxy, thiocarboxy, carbonyl, thiocarbonyl, hydroxyimine, ether or thioether functions and themselves being able to bear 1 to 4 substituents, which are identical or different, and which are selected from among halogen, hydroxy, trifluoromethyl, trifluoromethoxy, carboxy, carbonyl, amine, nitro, urea, aryl, or heteroaryl,
R₂ₐ and R_{2b} (generally R₂) being substituents which are identical or different, being able if need be to form a cyclic structure together or with Y₁, Y₂, U or A and representing a hydrogen atom or a linear, branched or cyclic C1, C2, C3, C4, C5 or C6 alkyl substituent containing if need be one or more amine, amide, thioamide, sulfonyl, urea, thiourea, carbamate, oxime, sulfonamide, carboxy, thiocarboxy, carbonyl, thiocarbonyl, ether or thioether functions and being able to bear 1 to 4 substituents, which are identical or different, and which are selected from halogen, hydroxy, trifluoromethyl, trifluoromethoxy, methoxy, carboxy, amine, nitro, aryl, or heteroaryl, R₂ₐ and R_{2b} being not simultaneously a hydrogen atom;
p, p′, p″ are, independently of each other, 0 or 1,
Y₁ and Y₂, which are identical or different, and can be linked by a single or multiple bond to Q, U or A, and represent a saturated or unsaturated, linear, branched or cyclic C1, C2, C3, C4, C5 or C6 alkyl chain, containing if need be one or more amine, amide, thioamide, sulfonyl, sulfonamide, oxo, carboxy, thiocarboxy, carbonyl, thiocarbonyl, urea, thiourea, carbamate, oxime, ether or thioether function, or an aryl or heteroaryl group, wherein the alkyl chain can additionally bear 1 to 4 substituents, which are identical or different, and which are preferably selected from the group consisting of halogen, hydroxy, trifluoromethyl, trifluoromethoxy, methoxy, carboxy, carbonyl, amine, nitro, oxime, aryl or heteroaryl such as defined herein after, or selected from among substituents of the type alkyl, alkylamino, dialkylamino, alkoxy, alkylthio, alkylsulfonyl, alkylsulfonamino, alkylsulfamoyl, alkylureido, alkylcarbamoyloxy, alkoxycarbonylamino, alkylcarbamoyl, dialkylcarbamoyl, alkylcarbonylamino, alkylcarbonyl, alkylcarbonyloxy, alkoxycarbonyl, alkoxyimine, the said alkyl groups comprising from 1 to 6 linear, branched or cyclic carbon atoms which can themselves contain one or more amine, amide, thioamide, sulfonyl, sulfonamide, carboxy, thiocarboxy, carbonyl, thiocarbonyl, oxime, ether or thioether function, or an aryl or heteroaryl group, such as those defined herein after, wherein the C1, C2, C3, C4, C5 or C6 chain may form a cyclic structure with R₂ including N from the aminoquinoline part Q and/or the function U and Y₁ and Y₂ may be linked together or to Q, U or A by a single or multiple bond,
U, which can be linked by a single or multiple bond to Q, Y₁, Y₂ or A, is an amine, amide, thioamide, sulfonyl, sulfonamide, carboxy, thiocarboxy, carbonyl, urea, thiourea, carbamate, ether, thioether, thiocarbonyl, sulfonate, oxime, oxyamine, alkylamine (NR), alkoxyimine (C═N—OR) or alkoxyiminocarbonyl (C(O)—C═N—OR) function with R representing a hydrogen atom or a C1, C2, C3, C4, C5 or C6 alkyl substituent, which is linear, branched or cyclic, containing if need be one or more amine, amide, thioamide, sulfonyl, sulfonamide, carboxy, thiocarboxy, carbonyl, thiocarbonyl, ether or thioether functions,
A represents an antibacterial antibiotic residue of the macrolide family selected from the group consisting of a compound:
having 14 atoms such as those described the formulae (XXVIa), (XXVIb), (XXVIc) and (XXVId), 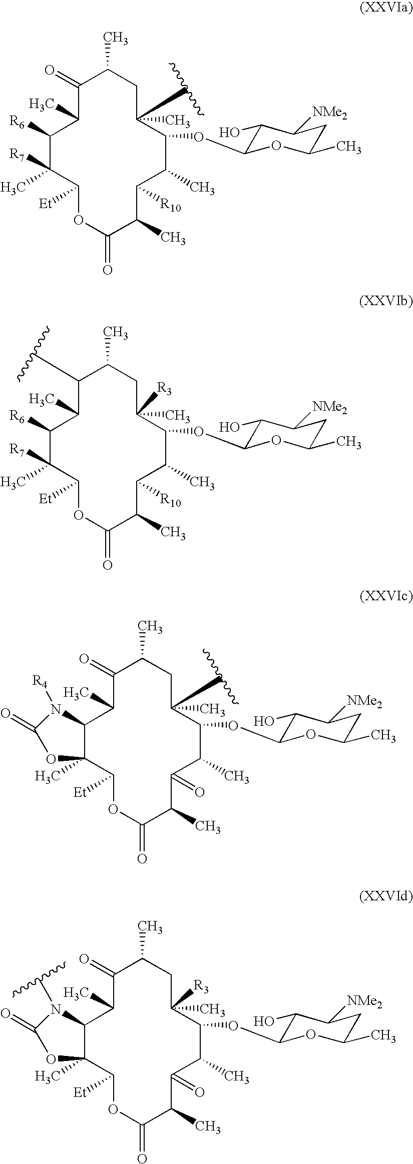
having 15 atoms such as those described the following formulae (XXVIIa), (XXVIIb), (XXVIIc) and (XXVIId), 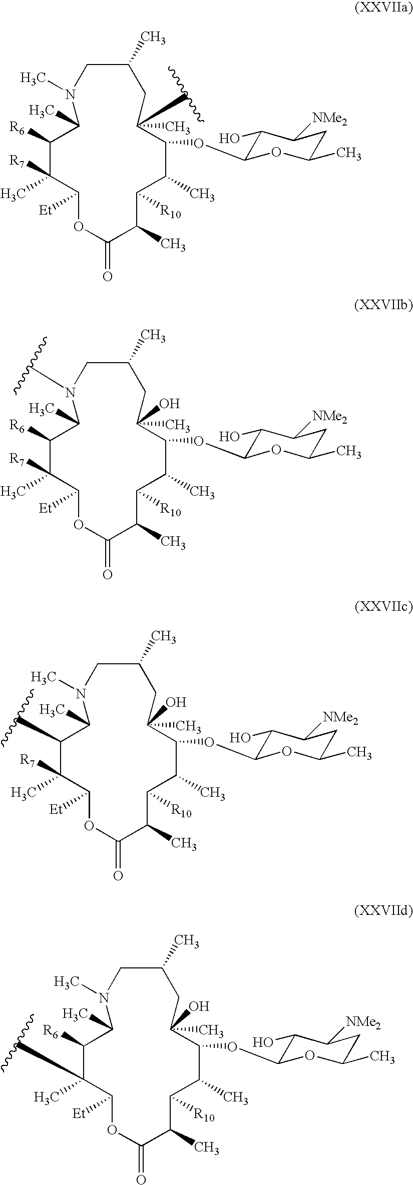
and
having 16 atoms such as those described the following formulae (XXVIIIa), (XXVIIIb), (XXVIIIc), (XXVIIId) and (XVIIIe), 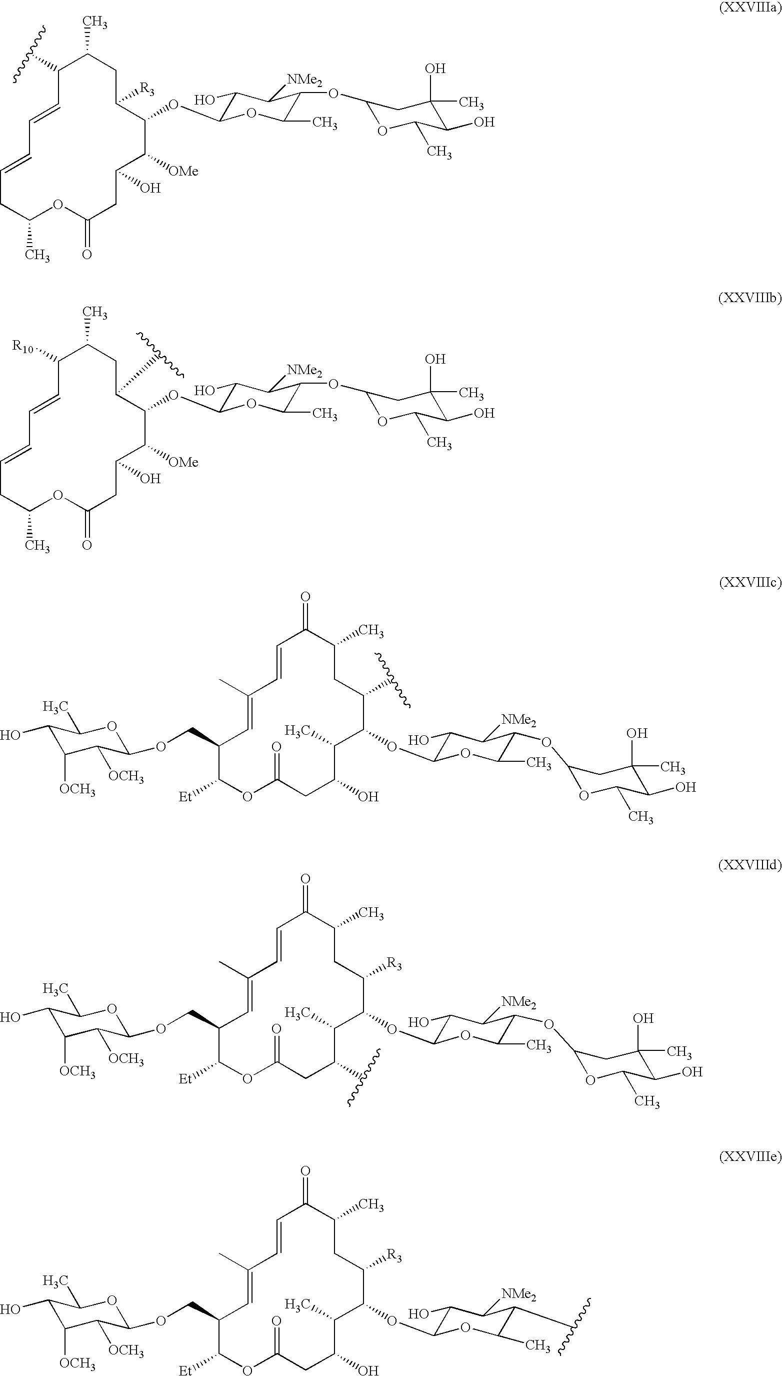
wherein
R₃, R₄, R₆ and R₇ are as follows:
R₃ represents a substituents selected from the group consisting of halogen, hydroxy, trifluoromethyl, trifluoromethoxy, carboxy, aldehyde, amine, sulfate, sulfonate, phosphate, phosphonate, nitro, cyano, aryl or heteroaryl or alkyl, alkylamino, dialkylamino, alkoxy, alkylthio, alkylsulfonyl, alkylsulfonylamino, alkylsulfamoyl, alkylureido, alkylcarbamoyloxy, alkoxycarbonylamino, alkylcarbamoyl, dialkylcarbamoyl, alkylcarbonylamino, alkylcarbonyl, alkylcarbonyloxy, alkyloxycarbonyl, alkoxyimine, the said alkyl groups comprising 1, 2, 3, 4, 5 or 6 carbon atoms, which are saturated or unsaturated, linear, branched or cyclic, containing if need be one or more amine, amide, thioamide, sulfonyl, sulfonamide, oxo, carboxy, thiocarboxy, carbonyl, thiocarbonyl, urea, thiourea, carbamate, oxime, ether or thioether functions that can themselves bear 1 to 4 substituents, which are identical or different, and which are selected from halogen, hydroxy, trifluoromethyl, methyl, trifluoromethoxy, carboxy, carbonyl, amine, nitro, urea, aryl or heteroaryl or heterocycle,
R₄ represents a hydrogen atom or a saturated or unsaturated, linear, branched or cyclic C1, C2, C3, C4, C5 or C6 alkyl substituent, containing if need be one or more amine, amide, thioamide, sulfonyl, sulfonamide, carboxy, thiocarboxy, carbonyl, thiocarbonyl, oxime, urea, carbamate, ether or thioether functions and being able to bear 1 to 4 substituents, which are identical or different, and which are selected from halogen, hydroxy, trifluoromethyl, trifluoromethoxy, methoxy, carboxy, amine, nitro, aryl, or heteroaryl,
R₆ and R₇ are substituents which are identical or different, being able if need be able to form, together, a cyclic structure and representing a hydrogen atom or a substituent which is selected from the group consisting of halogen, hydroxy, heterocycle, aryl or heteroaryl, or an alkyl, alkoxy or alkylamine substituent, said alkyl groups comprising 1, 2, 3, 4, 5 or 6 carbon atoms, which are saturated or unsaturated, linear, branched or cyclic, containing if need be one or more amine, amide, thioamide, sulfonyl, sulfonamide, carboxy, thiocarboxy, carbonyl, thiocarbonyl, ether or thioether functions and being able to bear 1 to 4 substituents, which are identical or different, and which are selected from halogen, hydroxy, trifluoromethyl, trifluoromethoxy, carboxy, amine, nitro, aryl, or heteroaryl,
R₁₀ is an oxygen atom linked via a double bond of carbonyl type to the macrocycle or a hydroxy group or an osidic derivative linked via a glycosidic bridge to the macrocycle and being able to bear 1 to 6 substituents, which are identical or different, and which are selected from hydroxy, alkyl, alkylamino, dialkylamino, or alkoxy, said alkyl groups including 1, 2, 3, 4, 5, or 6 carbon atoms which are linear or branched, saturated or unsaturated, and may bear a carboxy substituent.

16. The compound according to claim 1, wherein the antibiotic residue A represents a polypeptide residue such as derivatives of polymyxines or of bacitracin linking various peptidic structures.

17. The compound according to claim 1, wherein the antibiotic residue A represents a glycopeptide residue selected from among:
the derivatives of vancomycin described by the formulae (XXIXa), (XXIXb), (XXIXc), (XXIXd), (XXIXe) and (XXIXf) as follows, 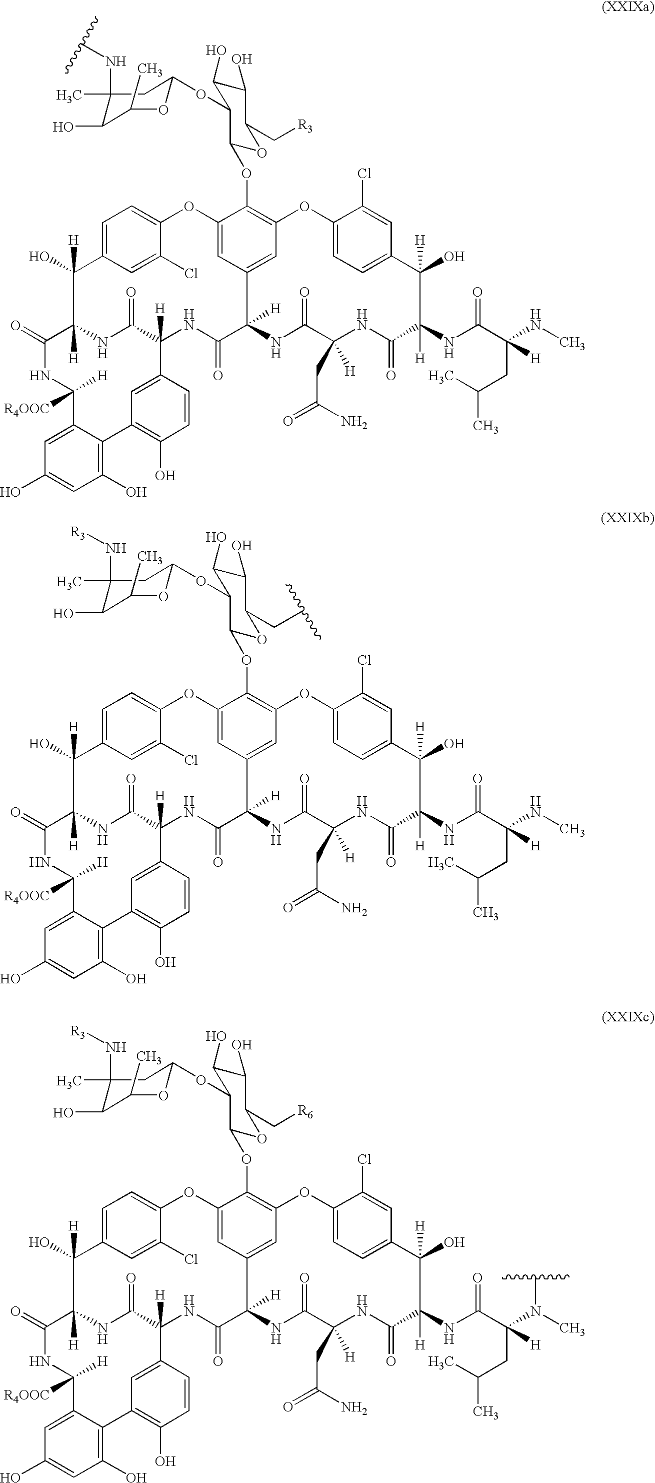 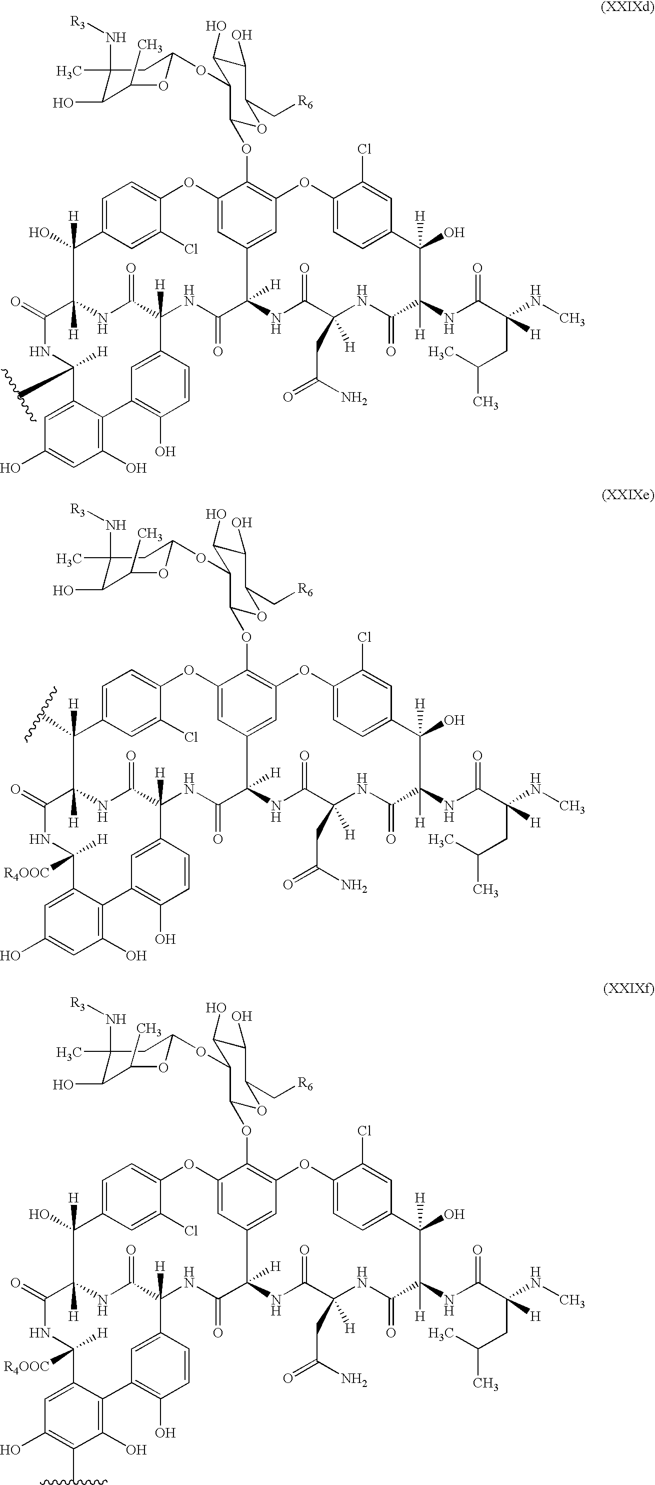
or the derivatives of teicoplanin described by the formula (XXXa) or (XXXb), as follows, 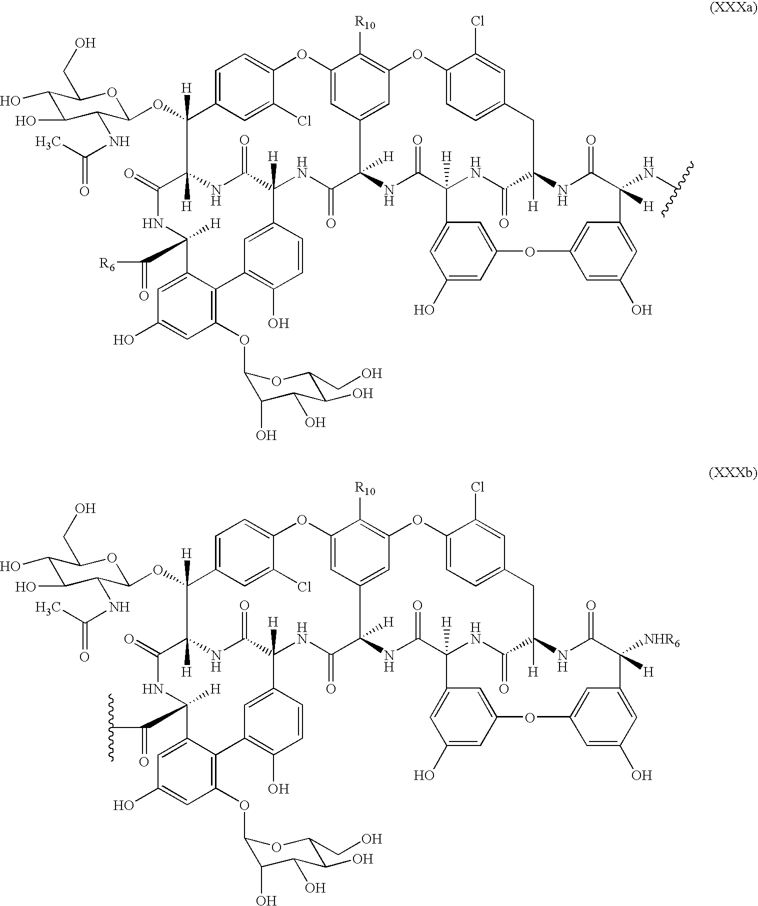
in which R₃, R₄ and R₆ are as follows:
R₃ is a substituent selected from the group consisting of halogen, hydroxy, trifluoromethyl, trifluoromethoxy, carboxy, aldehyde, amine, sulfate, sulfonate, phosphate, phosphonate, nitro, cyano, aryl or heteroaryl or alkyl, alkylamino, dialkylamino, alkoxy, alkylthio, alkylsulfonyl, alkylsulfonylamino, alkylsulfamoyl, alkylureido, alkylcarbamoyloxy, alkoxycarbonylamino, alkylcarbamoyl, dialkylcarbamoyl, alkylcarbonylamino, alkylcarbonyl, alkylcarbonyloxy, alkyloxycarbonyl, alkoxyimine, the said alkyl groups comprising 1, 2, 3, 4, 5 or 6 carbon atoms, which are saturated or unsaturated, linear, branched or cyclic, containing if need be one or more amine, amide, thioamide, sulfonyl, sulfonamide, oxo, carboxy, thiocarboxy, carbonyl, thiocarbonyl, urea, thiourea, carbamate, oxime, ether or thioether functions that can themselves bear 1 to 4 substituents, which are identical or different, and which are selected from halogen, hydroxy, trifluoromethyl, methyl, trifluoromethoxy, carboxy, carbonyl, amine, nitro, urea, aryl or heteroaryl or heterocycle,
R₄ represents a hydrogen atom or a saturated or unsaturated, linear, branched or cyclic C1, C2, C3, C4, C5 or C6 alkyl substituent, containing if need be one or more amine, amide, thioamide, sulfonyl, sulfonamide, carboxy, thiocarboxy, carbonyl, thiocarbonyl, oxime, urea, carbamate, ether or thioether functions and being able to bear 1 to 4 substituents, which are identical or different, and which are selected from halogen, hydroxy, trifluoromethyl, trifluoromethoxy, methoxy, carboxy, amine, nitro, aryl, or heteroaryl,
R₄ is a hydrogen atom or a substituent which is selected from the group consisting of halogen, hydroxy, heterocycle, aryl or heteroaryl, or an alkyl, alkoxy or alkylamine substituent, said alkyl groups comprising 1, 2, 3, 4, 5 or 6 carbon atoms, which are saturated or unsaturated, linear, branched or cyclic, containing if need be one or more amine, amide, thioamide, sulfonyl, sulfonamide, carboxy, thiocarboxy, carbonyl, thiocarbonyl, ether or thioether functions and being able to bear 1 to 4 substituents, which are identical or different, and which are selected from halogen, hydroxy, trifluoromethyl, trifluoromethoxy, carboxy, amine, nitro, aryl, or heteroaryl.

18. The compound according to claim 1 having the following general formula (I):
Q—(Y₁)ₚ—(U)_{p′}—(Y₂)_{p″}-A (I)
wherein Q is selected from among 4-aminoquinolines, 2-aminoquinolines and 8-aminoquinolines have the following formulae (XXXIIIa), (XXXIIIb), (XXXIIIc), (XXXIIId) and (XXXIIIe): 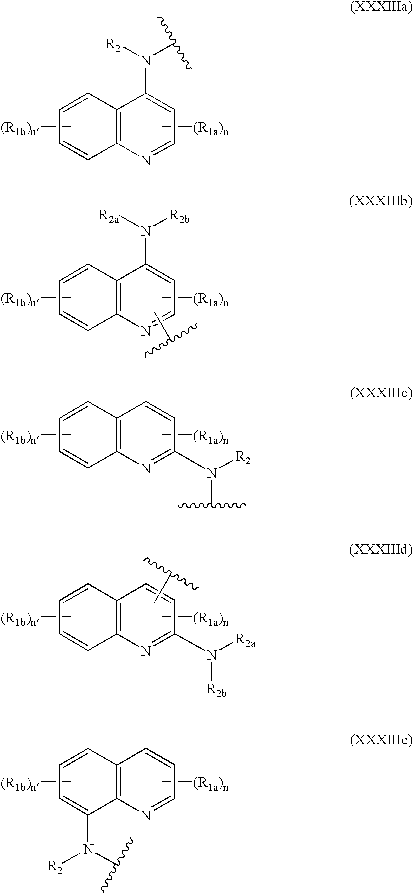
wherein R₁ₐ, R_{1b}, (generally R₁), R₂ₐ, R_{2b}, (generally R₂), n and n′ are as defined in claim 1.

19. The compound according to claim 18, wherein R₁ represents a halogen atom or a methyl, methoxy, trifluoromethyl, trifluoromethoxy, cyano, amine or nitro group occupying any position, in the formulae (XXXIIIa), (XXXIIIb) and (XXXIIIe) R₂ represents a hydrogen atom or a methyl group or forms a cyclic structure with Y₁ and eventually U, including the N of the aminoquinoline (preferably a piperidine or a piperazine) and in the formulae (XXXIIIb) and (XXXIIId) R₂ₐ and R_{2b} represent a hydrogen atom or a methyl, cyclopropyl, or 2-(diethylamino)ethyl group, or a heterocycle when R₂ₐ and R_{2b} form a cyclic structure together (preferably aziridin-1-yl, morpholin-4-yl, piperidin-1-yl, piperazin-1-yl, or 4-methylpiperazin-1-yl).

20. The compound according to claim 18, wherein the compounds described by formula (I) are those having the —(Y₁)ₚ—(U)_{p′}—(Y₂)_{p″} groups selected from among a group in which p=p′=p″=0, the link between Q and A being direct, a group in which p′=1 and p=p″=0, U being as defined previously and advantageously representing a carbonyl group, a group in which p′=1 and p=p″=0, U being as defined previously and advantageously representing a thioether group, a group in which p′=1 and p=p″=0, U being as defined previously and advantageously representing an alkoxyiminocarbonyl group (preferably hydroxyiminocarbonyl or methoxyiminocarbonyl), a group in which p=1 and p′=p″=0, Y₁ being as defined previously and advantageously representing a linear or branched C1, C2, C3, C4, C5 or C6 alkyl chain being able to form a cyclic structure with A or R₂ including the N of the aminoquinoline, a group in which p=1 and p′=p″=0, Y₁ being as defined previously and advantageously representing a C1, C2, C3, C4, C5 or C6 alkyl chain substituted by fluorine atoms, a group in which p=1 and p′=p″=0, Y₁ being as defined previously and advantageously representing a C1, C2, C3, C4, C5 or C6 alkyl chain containing an amine or ether function, a group in which p=p′=1 and p″=0, U being as defined previously and advantageously representing a carbonyl group and Y₁ being as defined previously and advantageously representing a linear or branched C1, C2, C3, C4, C5 or C6 alkyl chain and being able to form a cyclic structure with R₂ including the N of the aminoquinoline, a group in which p=p′=1 and p″=0, U being as defined previously and advantageously representing an amine group and Y₁ being as defined previously and advantageously representing a linear or branched C1, C2, C3, C4, C5, or C6 alkyl chain able to contain an amine, ether, amide, or urea function and being able to form a cyclic structure with U and/or R₂ including the N of the aminoquinoline, a group in which p=p′=1 and p″=0, U being as defined previously and advantageously representing a thioether function and Y₁ being as defined previously and advantageously representing a linear or branched C1, C2, C3, C4, C5, or C6 alkyl chain and being able to be substituted by fluorine atoms, a group in which p=p′=1 and p″=0, U being as defined previously and advantageously representing an ether function and Y₁ being as defined previously and advantageously representing a C1, C2, C3, C4, C5, or C6 alkyl chain, a group in which p=p′=1 and p″=0, U being as defined previously and advantageously representing a carbamate function and Y₁ being as defined previously and advantageously representing a linear or branched, saturated or unsaturated C1, C2, C3, C4, C5, or C6 alkyl chain and being able to contain an ether function and/or aryl group, a group in which p′=p″=1 and p=0, U being as defined previously and advantageously representing an amide function and Y₂ being as defined previously and advantageously representing a linear or branched C1, C2, C3, C4, C5, or C6 alkyl chain being able to contain an amine or thioether function, a group in which p=p′=1, U being as defined previously and advantageously representing an amine function and Y₁ and Y₂ being as defined previously and advantageously representing a linear or branched C1, C2, C3, C4, C5, or C6 alkyl chain able to be substituted by fluorine atoms or a hydroxy group and being able to form a cyclic structure with U and/or R₂ including the N of the aminoquinoline, a group in which p=p′=p″=1, U being as defined previously and advantageously representing an ether function and Y₁ and Y₂ being as defined previously and advantageously representing a linear or branched C1, C2, C3, C4, C5, or C6 alkyl chain able to contain an aryl group, a group in which p=p′=p″=1, U being as defined previously and advantageously representing a thioether function and Y₁ and Y₂ being as defined previously and advantageously representing a linear or branched C1, C2, C3, C4, C5, or C6 alkyl chain, a group in which p=p′=p″=1, U being as defined previously and advantageously representing an amide function and Y₁ and Y₂ being as defined previously and advantageously representing a linear or branched C1, C2, C3, C4, C5, or C6 alkyl chain and being able to be substituted by fluorine atoms, a group in which p=p′=p″=1, U being as defined previously and advantageously representing a carbamate function and Y₁ and Y₂ being as defined previously and advantageously representing a linear or branched C1, C2, C3, C4, C5, or C6 alkyl chain and being able to be substituted by fluorine atoms, a group in which p=p′=p″=1, U being as defined previously and advantageously representing a urea function and Y₁ and Y₂ being as defined previously and advantageously representing a linear or branched C1, C2, C3, C4, C5, or C6 alkyl chain and being able to be substituted by fluorine atoms.

21. A compound represented by the structure (XXXIVa), (XXXIVb), or 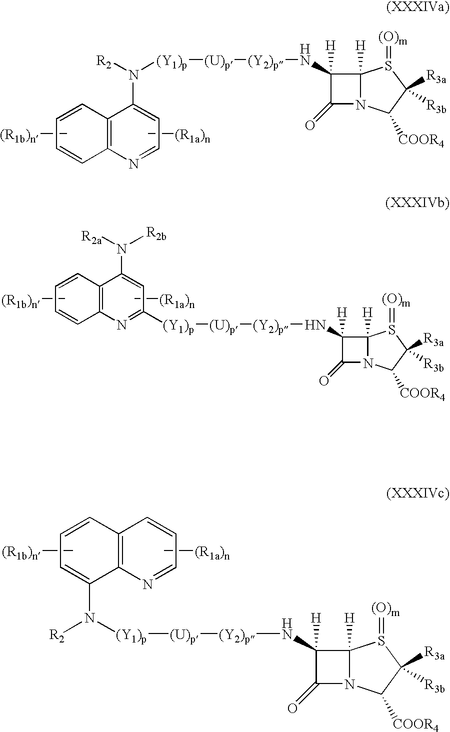
by the structure (XXXVa), (XXXVb), or (XXXVc), 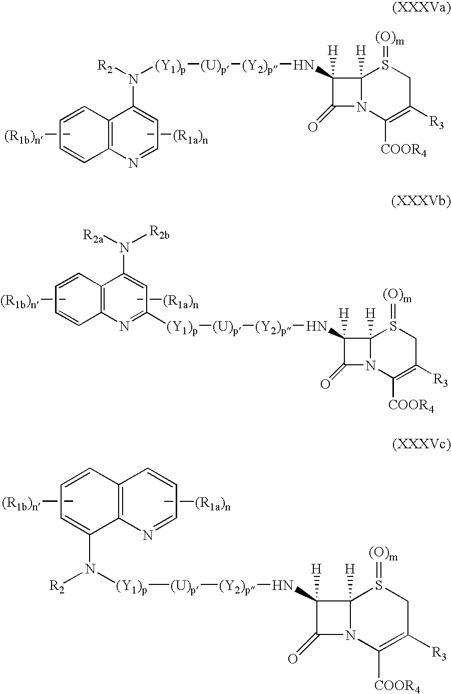
by the structure (XXXVd), (XXXVe), (XXXVf), or (XXXVg), 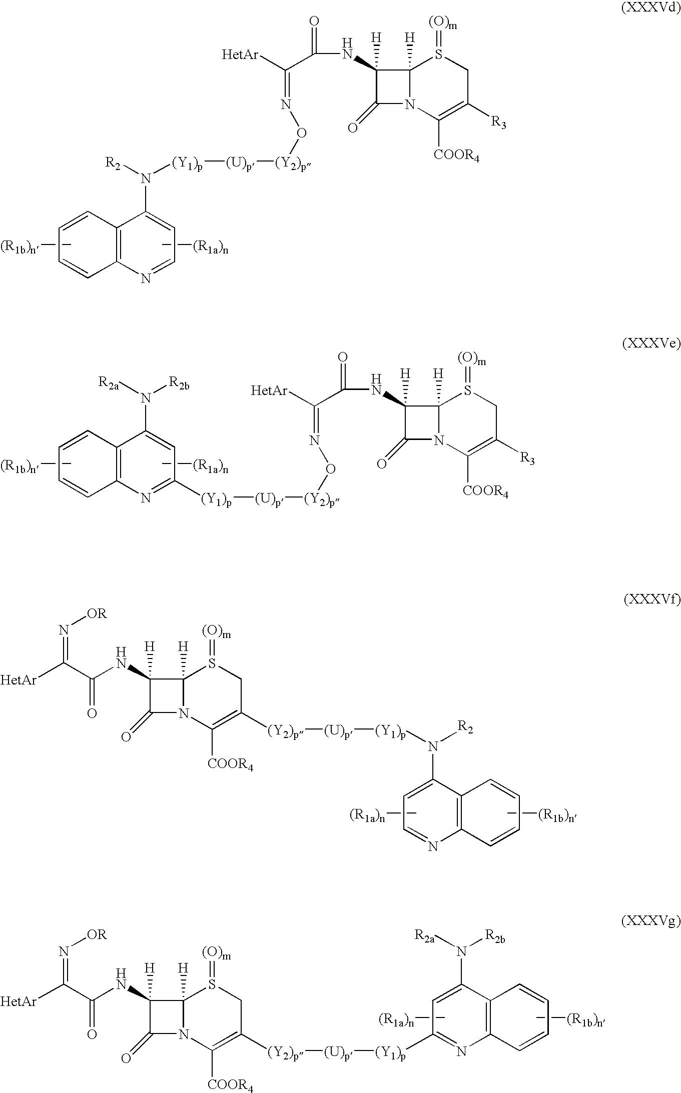
wherein R₁ₐ, R_{1b}, R₂, R₃ₐ, R_{3b}, R₄, Y₁, Y₂, U, p, p′, p″, m, n, and n′ are as follows:
n and n′ represent, independently of each other, 0, 1, 2 or 3;
R₁ₐ and R_{1b} (generally R₁) represent one or more substituents which are identical or different, occupying any position and representing a substituent which is selected from the group consisting of halogen, trifluoromethyl, trifluoromethoxy, carboxy, carboxy, amine, sulfate, sulfonate, phosphate, phosphonate, nitro, cyano, aryl or heteroaryl or alkyl, alkylamino, dialkylamino, alkoxy, alkylthio, alkylsulfonyl, alkylsulfamoyl, alkylsulfonylamino, alkylcarbamoyl, dialkylcarbamoyl, alkoxycarbonyl, alkylcarbonylamino, the said alkyl groups comprising 1, 2, 3, 4, 5 or 6 carbon atoms, which are linear, branched or cyclic, saturated or unsaturated, containing if need be one or more amine, amide, thioamide, sulfonyl, sulfonamide, carboxy, thiocarboxy, carbonyl, thiocarbonyl, hydroxyimine, ether or thioether functions and themselves being able to bear 1 to 4 substituents, which are identical or different, and which are selected from among halogen, hydroxy, trifluoromethyl, trifluoromethoxy, carboxy, carbonyl, amine, nitro, urea, aryl, or heteroaryl,
R₂ₐ and R_{2b} (generally R₂) being substituents which are identical or different, being able if need be to form a cyclic structure together or with Y₁, Y₂, U or A and representing a hydrogen atom or a linear, branched or cyclic C1, C2, C3, C4, C5 or C6 alkyl substituent containing if need be one or more amine, amide, thioamide, sulfonyl, urea, thiourea, carbamate, oxime, sulfonamide, carboxy, thiocarboxy, carbonyl, thiocarbonyl, ether or thioether functions and being able to bear 1 to 4 substituents, which are identical or different, and which are selected from halogen, hydroxy, trifluoromethyl, trifluoromethoxy, methoxy, carboxy, amine, nitro, aryl, or heteroaryl, R₂ₐ and R_{2b} being not simultaneously a hydrogen atom;
p, p′, p″ are, independently of each other, 0 or 1,
Y₁ and Y₂, which are identical or different, and can be linked by a single or multiple bond to Q, U or A, and represent a saturated or unsaturated, linear, branched or cyclic C1, C2, C3, C4, C5 or C6 alkyl chain, containing if need be one or more amine, amide, thioamide, sulfonyl, sulfonamide, oxo, carboxy, thiocarboxy, carbonyl, thiocarbonyl, urea, thiourea, carbamate, oxime, ether or thioether function, or an aryl or heteroaryl group, wherein the alkyl chain can additionally bear 1 to 4 substituents, which are identical or different, and which are preferably selected from the group consisting of halogen, hydroxy, trifluoromethyl, trifluoromethoxy, methoxy, carboxy, carbonyl, amine, nitro, oxime, aryl or heteroaryl such as defined herein after, or selected from among substituents of the type alkyl, alkylamino, dialkylamino, alkoxy, alkylthio, alkylsulfonyl, alkylsulfonamino, alkylsulfamoyl, alkylureido, alkylcarbamoyloxy, alkoxycarbonylamino, alkylcarbamoyl, dialkylcarbamoyl, alkylcarbonylamino, alkylcarbonyl, alkylcarbonyloxy, alkoxycarbonyl, alkoxyimine, the said alkyl groups comprising from 1 to 6 linear, branched or cyclic carbon atoms which can themselves contain one or more amine, amide, thioamide, sulfonyl, sulfonamide, carboxy, thiocarboxy, carbonyl, thiocarbonyl, oxime, ether or thioether function, or an aryl or heteroaryl group, such as those defined herein after, wherein the C1, C2, C3, C4, C5 or C6 chain may form a cyclic structure with R₂ including N from the aminoquinoline part Q and/or the function U and Y₁ and Y₂ may be linked together or to Q, U or A by a single or multiple bond,
U, which can be linked by a single or multiple bond to Q, Y₁, Y₂ or A, is an amine, amide, thioamide, sulfonyl, sulfonamide, carboxy, thiocarboxy, carbonyl, urea, thiourea, carbamate, ether, thioether, thiocarbonyl, sulfonate, oxime, oxyamine, alkylamine (NR), alkoxyimine (C═N—OR) or alkoxyiminocarbonyl (C(O)—C═N—OR) function with R representing a hydrogen atom or a C1, C2, C3, C4, C5 or C6 alkyl substituent, which is linear, branched or cyclic, containing if need be one or more amine, amide, thioamide, sulfonyl, sulfonamide, carboxy, thiocarboxy, carbonyl, thiocarbonyl, ether or thioether functions,
R₃ₐ and R_{3b} (generally R₃) represent substituents which are identical or different and which are selected from the group consisting of halogen, hydroxy, trifluoromethyl, trifluoromethoxy, carboxy, aldehyde, amine, sulfate, sulfonate, phosphate, phosphonate, nitro, cyano, aryl or heteroaryl or alkyl, alkylamino, dialkylamino, alkoxy, alkylthio, alkylsulfonyl, alkylsulfonylamino, alkylsulfamoyl, alkylureido, alkylcarbamoyloxy, alkoxycarbonylamino, alkylcarbamoyl, dialkylcarbamoyl, alkylcarbonylamino, alkylcarbonyl, alkylcarbonyloxy, alkyloxycarbonyl, alkoxyimine, the said alkyl groups comprising 1, 2, 3, 4, 5 or 6 carbon atoms, which are saturated or unsaturated, linear, branched or cyclic, containing if need be one or more amine, amide, thioamide, sulfonyl, sulfonamide, oxo, carboxy, thiocarboxy, carbonyl, thiocarbonyl, urea, thiourea, carbamate, oxime, ether or thioether functions that can themselves bear 1 to 4 substituents, which are identical or different, and which are selected from halogen, hydroxy, trifluoromethyl, methyl, trifluoromethoxy, carboxy, carbonyl, amine, nitro, urea, aryl or heteroaryl or heterocycle,
R₄ represents a hydrogen atom or a saturated or unsaturated, linear, branched or cyclic C1, C2, C3, C4, C5 or C6 alkyl substituent, containing if need be one or more amine, amide, thioamide, sulfonyl, sulfonamide, carboxy, thiocarboxy, carbonyl, thiocarbonyl, oxime, urea, carbamate, ether or thioether functions and being able to bear 1 to 4 substituents, which are identical or different, and which are selected from halogen, hydroxy, trifluoromethyl, trifluoromethoxy, methoxy, carboxy, amine, nitro, aryl, or heteroaryl,
“HetAr” represents a heteroaryl group.

22. The compound according to claim 21, wherein said hybrid molecule has a link (Y₁)ₚ—(U)_{p′}—(Y₂)_{p″} selected from the group consisting of a carbonyl (p′=1, p=p″=0), an alkoxyiminocarbonyle (p′=1, p=p″=0), and a (C1-C6)alkyl-carbonyle (p=p=1, p″=0).

23. The compound according to claim 22, wherein said alkoxyiminocarbonyle is a hydroxyiminocarbonyl or a methoxyiminocarbonyl.

24. The compound according to claim 22, wherein said (C₁-C₆)alkyl-carbonyl is selected from the group consisting of acetyl, 3-propionyl, 2-propionyl, 2-methyl-2-propionyl, 4-butyryl, 3-methyl-3-butyryl, and piperidine-4-carbonyl (by including R₂ and N of aminoquinoline group).

25. The compound according to claim 21, wherein:
in the hybrid aminoquinoline-β-lactam molecules defined by formulae (XXXIVa), (XXXIVb), (XXXIVc), (XXXVa), (XXXVb), (XXXVc), (XXXVd), (XXXVe), (XXXVf) and (XXXVg), R₁ represents a halogen atom or a methyl, methoxy, trifluoromethyl, trifluoromethoxy, carboxy, cyano, amine, or nitro group occupying any position, R₂ represents a hydrogen atom or a methyl group or forms a cyclic structure with Y₁ including the N of the aminoquinoline, R₄ is a hydrogen atom or a moiety that is easily hydrolyzable in vivo in the area of prodrug molecules (preferably 2,2-dimethyl-propionyloxymethyl);
in the aminoquinoline-penicillin hybrid molecules having the formula (XXXIVa) or (XXXIVb), R₃ₐ and R_{3b} are two identical substituents of alkyl type (preferably two methyl substituents);
in the aminoquinoline-cephalosporin type hybrid molecules having the formulae (XXXVa), (XXXVb), (XXXVc), (XXXVd) or (XXXVe), R₃ is a halogen or a saturated or unsaturated C1, C2, C3, C4, C5, or C6 alkyl chain possibly containing a carboxy or ether function (preferably methyl, vinyl, acetoxymethyl or methoxymethyl group) and being able to bear a heteroaryl or heterocycle substituent (preferably pyridinium-1-ylmethyl, 1-methyl-1H-tetrazol-5-ylsulfanylmethyl or 6-hydroxy-2-methyl-5-oxo-2,5-dihydro-[1,2,4]triazin-3-ylsulfanylmethyl);
in the aminoquinoline-cephalosporin type hybrid molecules having the formula (XXXVf), (XXXVg) or (XXXVh), R represents a hydrogen atom or a C1, C2, C3, C4, C5, or C6 alkyl substituent (preferably methyl) and “HetAr” represents a heteroaryl of 2-amino-thiazol-4-yl, 2-amino-5-chloro-thiazol-4-yl or 5-amino-[1,2,4]-thiadiazol-3-yl type;
in the aminoquinoline-penicillin or aminoquinoline-cephalosporin type hybrid molecules having the formulae (XXXIVa), (XXXIVb), (XXXVc), (XXXVa), (XXXVb), (XXXVc), the group (Y₁)ₚ—(U)_{p′}—(Y₂)_{p″} is a group which comprise as (Y₁)ₚ—(U)_{p′}(Y₂)_{p″} group a carbonyl moiety (p′=1, p=p″=0), alkoxyiminocarbonyl (p′=1, p=p″=0) (preferably hydroxyiminocarbonyl or methoxylaminocarbonyl), or alkylcarbonyl (p=p′=1, p″=0) the said alkyl group comprising 1, 2, 3, 4, 5 or 6 carbon atoms (preferably acetyl, 3-propionyl, 2-propionyl, 2-methyl-2-propionyl, 4-butyryl, 3-methyl-3-butyryl or piperidine-4-carbonyl (which include R₂ and the N of the aminoquinoline)),
in the compounds having the formula (XXXVd), the group (Y₁)ₚ—(U)_{p′}—(Y₂)_{p″} is a group which comprise as (Y₁)ₚ—(U)_{p′}—(Y₂)_{p″} group a C1, C2, C3, C4, C5, ou C6 alkyl moiety (p=1, p′=p″=0) (preferably 2-ethyl, 3-propyl, 2-propyl, 2-methyl-2-propyl, 2,2-difluoro-3-propyl, or 4-piperidin-1-yl),
in the compounds having the formula (XXXVe) the group (Y₁)ₚ—(U)_{p′}—(Y₂)_{p″} is a group which comprise as (Y₁)ₚ—(U)_{p′}—(Y₂)_{p″} group an alkylcarbamoyl moiety (p=0, p′=p″=1) (preferably 2-ethylcarbamoyl, 3-propylcarbamoyl, 2-propylcarbamoyl, 1-carbonylpiperidin-4-yl),
in the compounds having the formula (XXXVf) the group (Y₁)ₚ—(U)_{p′}—(Y₂)_{p″}—is a group which comprise as (Y₁)ₚ—(U)_{p′}—(Y₂)_{p″} group an alkylamine moiety (p=p′=1, p″=0) (preferably methylamino, 2-ethylamino, 3-propylamino, 2-propylamino, 2,2-difluoro-3-propylamino, 4-piperidin-1-yl, 4-piperazin-1-yl or piperidin-4-ylamino (which include R₂ and the N of the aminoquinoline)), dialkylamine (p=p′=p″=1) (preferably methylamino-2-ethyl, methylamino-3-propyl, methylamino-2-propyl, methylamino-2,2-difluoro-3-propyl, 4-piperidin-1-ylmethyl, 4-methylpiperazin-1-yl or 4-methylaminopiperidin-1-yl (which include R₂ and the N of the aminoquinoline)), alkylsulfanyl (p=p′=1, p″=0) (preferably methylsulfanyl, 2-ethylsulfanyl, 3-propylsulfanyl, 2-propylsulfanyl, 2,2-difluoro-3-propylsulfanyl, or piperidin-4-ylsulfanyl (which include R₂ and the N of the aminoquinoline)) or dialkylsulfanyl (p=p′=p″=1) (preferably methylsulfanyl-2-ethyl, methylsulfanyl-3-propyl, methylsulfanyl-2-propyl, methylsulfanyl-2,2-difluoro-3-propyl, 4-methylsulfanylpiperidin-1-yl (which include R₂ and the N of the aminoquinoline)),
in the compounds having the formula (XXXVg) the group (Y₁)ₚ—(U)_{p′}—(Y₂)_{p″} is a group which comprise as (Y₁)ₚ—(U)_{p′}—(Y₂)_{p″} group a thioether moiety (p′=1, p=p″=0), alkylsulfanyl (p′=p″=1, p=0) (preferably methylsulfanyl), alkylaminoalkylcarbamoyl (p=0, p′=p″=1) (preferably methylamino-2-ethylcarbamoyl, methylamino-3-propylcarbamoyl, methylamino-2-propylcarbamoyl, 4-methylpiperazine-1-carbonyl, 4-methylaminopiperidine-1-carbonyl, 1-methylpiperidin-4-ylcarbamoyl) or alkylsulfanylalkylcarbamoyl (p=0, p′=p″=1) (preferably methylsulfanyl-2-ethylcarbamoyl, methylsulfanyl-3-propylcarbamoyl, methylsulfanyl-2-propylcarbamoyl, 4-methylsulfanylpiperidine-1-carbonyl),

26. A compound represented by the structure (XXXVIa) or (XXXVIb): 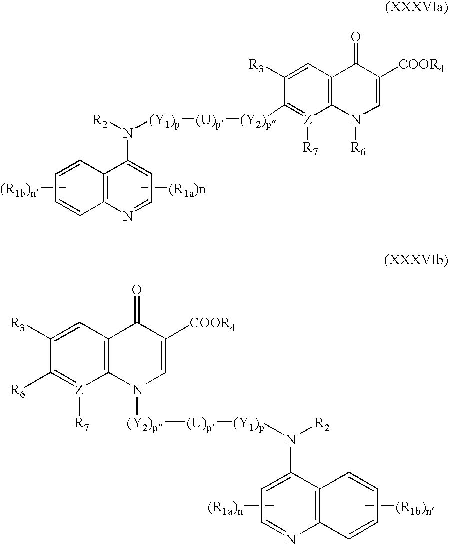
in which R₁, R₂, R₃, R₄, R₆, R₇, Y₁, Y₂, U, Z, p, p′, p″, n, and n′ are as follows:
n and n′ represent, independently of each other, 0, 1, 2 or 3;
R₁ₐ and R_{1b} (generally R₁) represent one or more substituents which are identical or different, occupying any position and representing a substituent which is selected from the group consisting of halogen, trifluoromethyl, trifluoromethoxy, carboxy, amine, sulfate, sulfonate, phosphate, phosphonate, nitro, cyano, aryl or heteroaryl or alkyl, alkylamino, dialkylamino, alkoxy, alkylthio, alkylsulfonyl, alkylsulfamoyl, alkylsulfonylamino, alkylcarbamoyl, dialkylcarbamoyl, alkylcarbonyloxy, alkoxycarbonyl, alkylcarbonylamino, the said alkyl groups comprising 1, 2, 3, 4, 5 or 6 carbon atoms, which are linear, branched or cyclic, saturated or unsaturated, containing if need be one or more amine, amide, thioamide, sulfonyl, sulfonamide, carboxy, thiocarboxy, carbonyl, thiocarbonyl, hydroxyimine, ether or thioether functions and themselves being able to bear 1 to 4 substituents, which are identical or different, and which are selected from among halogen, hydroxy, trifluoromethyl, trifluoromethoxy, carboxy, carbonyl, amine, nitro, urea, aryl, or heteroaryl,
R₂ₐ and R_{2b} (generally R₂) being substituents which are identical or different, being able if need be to form a cyclic structure together or with Y₁, Y₂, U or A and representing a hydrogen atom or a linear, branched or cyclic C1, C2, C3, C4, C5 or C6 alkyl substituent containing if need be one or more amine, amide, thioamide, sulfonyl, urea, thiourea, carbamate, oxime, sulfonamide, carboxy, thiocarboxy, carbonyl, thiocarbonyl, ether or thioether functions and being able to bear 1 to 4 substituents, which are identical or different, and which are selected from halogen, hydroxy, trifluoromethyl, trifluoromethoxy, methoxy, carboxy, amine, nitro, aryl, or heteroaryl, R₂ₐ and R_{2b} being not simultaneously a hydrogen atom;
p, p′, p″ are, independently of each other, 0 or 1,
Y₁ and Y₂, which are identical or different, and can be linked by a single or multiple bond to Q, U or A, and represent a saturated or unsaturated, linear, branched or cyclic C1, C2, C3, C4, C5 or C6 alkyl chain, containing if need be one or more amine, amide, thioamide, sulfonyl, sulfonamide, oxo, carboxy, thiocarboxy, carbonyl, thiocarbonyl, urea, thiourea, carbamate, oxime, ether or thioether function, or an aryl or heteroaryl group, wherein the alkyl chain can additionally bear 1 to 4 substituents, which are identical or different, and which are preferably selected from the group consisting of halogen, hydroxy, trifluoromethyl, trifluoromethoxy, methoxy, carboxy, carbonyl, amine, nitro, oxime, aryl or heteroaryl such as defined herein after, or selected from among substituents of the type alkyl, alkylamino, dialkylamino, alkoxy, alkylthio, alkylsulfonyl, alkylsulfonamino, alkylsulfamoyl, alkylureido, alkylcarbamoyloxy, alkoxycarbonylamino, alkylcarbamoyl, dialkylcarbamoyl, alkylcarbonylamino, alkylcarbonyl, alkylcarbonyloxy, alkoxycarbonyl, alkoxyimine, the said alkyl groups comprising from 1 to 6 linear, branched or cyclic carbon atoms which can themselves contain one or more amine, amide, thioamide, sulfonyl, sulfonamide, carboxy, thiocarboxy, carbonyl, thiocarbonyl, oxime, ether or thioether function, or an aryl or heteroaryl group, such as those defined herein after, wherein the C1, C2, C3, C4, C5 or C6 chain may form a cyclic structure with R₂ including N from the aminoquinoline part Q and/or the function U and Y₁ and Y₂ may be linked together or to Q, U or A by a single or multiple bond,
U, which can be linked by a single or multiple bond to Q, Y₁, Y₂ or A, is an amine, amide, thioamide, sulfonyl, sulfonamide, carboxy, thiocarboxy, carbonyl, urea, thiourea, carbamate, ether, thioether, thiocarbonyl, sulfonate, oxime, oxyamine, alkylamine (NR), alkoxyimine (C═N—OR) or alkoxyiminocarbonyl (C(O)—C═N—OR) function with R representing a hydrogen atom or a C1, C2, C3, C4, C5 or C6 alkyl substituent, which is linear, branched or cyclic, containing if need be one or more amine, amide, thioamide, sulfonyl, sulfonamide, carboxy, thiocarboxy, carbonyl, thiocarbonyl, ether or thioether functions,
R₃ₐ and R_{3b} (generally R₃) represent substituents which are identical or different and which are selected from the group consisting of halogen, hydroxy, trifluoromethyl, trifluoromethoxy, carboxy, aldehyde, amine, sulfate, sulfonate, phosphate, phosphonate, nitro, cyano, aryl or heteroaryl or alkyl, alkylamino, dialkylamino, alkoxy, alkylthio, alkylsulfonyl, alkylsulfonylamino, alkylsulfamoyl, alkylureido, alkylcarbamoyloxy, alkoxycarbonylamino, alkylcarbamoyl, dialkylcarbamoyl, alkylcarbonylamino, alkylcarbonyl, alkylcarbonyloxy, alkyloxycarbonyl, alkoxyimine, the said alkyl groups comprising 1, 2, 3, 4, 5 or 6 carbon atoms, which are saturated or unsaturated, linear, branched or cyclic, containing if need be one or more amine, amide, thioamide, sulfonyl, sulfonamide, oxo, carboxy, thiocarboxy, carbonyl, thiocarbonyl, urea, thiourea, carbamate, oxime, ether or thioether functions that can themselves bear 1 to 4 substituents, which are identical or different, and which are selected from halogen, hydroxy, trifluoromethyl, methyl, trifluoromethoxy, carboxy, carbonyl, amine, nitro, urea, aryl or heteroaryl or heterocycle,
R₄ represents a hydrogen atom or a saturated or unsaturated, linear, branched or cyclic C1, C2, C3, C4, C5 or C6 alkyl substituent, containing if need be one or more amine, amide, thioamide, sulfonyl, sulfonamide, carboxy, thiocarboxy, carbonyl, thiocarbonyl, oxime, urea, carbamate, ether or thioether functions and being able to bear 1 to 4 substituents, which are identical or different, and which are selected from halogen, hydroxy, trifluoromethyl, trifluoromethoxy, methoxy, carboxy, amine, nitro, aryl, or heteroaryl,
R₆ and R₇ are substituents which are identical or different, being able if need be able to form, together, a cyclic structure and representing a hydrogen atom or a substituent which is selected from the group consisting of halogen, hydroxy, heterocycle, aryl or heteroaryl, or an alkyl, alkoxy or alkylamine substituent, said alkyl groups comprising 1, 2, 3, 4, 5 or 6 carbon atoms, which are saturated or unsaturated, linear, branched or cyclic, containing if need be one or more amine, amide, thioamide, sulfonyl, sulfonamide, carboxy, thiocarboxy, carbonyl, thiocarbonyl, ether or thioether functions and being able to bear 1 to 4 substituents, which are identical or different, and which are selected from halogen, hydroxy, trifluoromethyl, trifluoromethoxy, carboxy, amine, nitro, aryl, or heteroaryl.

27. The compound according to claim 26, wherein,
in the hybrid molecules of aminoquinoline-quinolone type having the formulae (XXXVIa) and (XXXVIb), Z is a carbon atom, R₁ represents a halogen atom or a methyl, methoxy, trifluoromethyl, trifluoromethoxy, carboxy, cyano, amine, or nitro group occupying any position, R₂ represents a hydrogen atom or a methyl group or forms a cyclic structure with Y₁ including the N of the aminoquinoline, R₃ is a hydrogen or fluorine atom and R₄ is a hydrogen atom
in the hybrid aminoquinoline-quinolone molecules defined by formula (XXXVIa), R₆ is a linear, branched, or cyclic C1, C2, C3, C4, C5, or C6 alkyl chain or forms a cyclic structure with R₇, and R₇ is a hydrogen or halogen atom, a methoxy group, or forms a cyclic structure with R₆, such as a 3-methyl-3,4-dihydro-2S[1,4]-oxazine; the (Y₁)ₚ—(U)ₚ—(Y₂)ₚN group is a group in which p=p′=p″=0, Q being directly linked to A, or a group in which p=p′=1 and p″=0, U being as defined above and advantageously representing an amine function and Y₁ being as defined above and representing a C1, C2, C3, C4, C5, or C6 alkyl chain and that can form a cyclic structure with U or R₂ (including the N of the aminoquinoline) and possibly containing an amine function.
in the hybrid aminoquinoline-quinolone molecules defined by formula (XXXVIb), R₆ is a heterocycle containing 1 or 2 heteroatoms (preferably piperazin-1-yl, N-methylpiperazin-1-yl, 3-methylpiperazin-1-yl or 3-amino-pyrrolidin-1-yl); the (Y₁)ₚ—(U)_{p′}—(Y₂)_{p″} group is a group in which p=p′=p″=0, Q being directly linked to A, and the exocyclic nitrogen atom of the aminoquinoline corresponds to the endocyclic nitrogen atom of the quinolone, or a group in which p=1 and p′=p″=0, Y₁ being as defined above and advantageously representing a C1, C2, C3, C4, C5, or C6 alkyl chain and that can form a cyclic structure with R₂.

28. The compound according to claim 1, wherein it is represented by the formula (XXXVII), in which R₁, R₂, R₃, Y₁, Y₂, U, p, p′, p″, n, and n′ are as defined above:

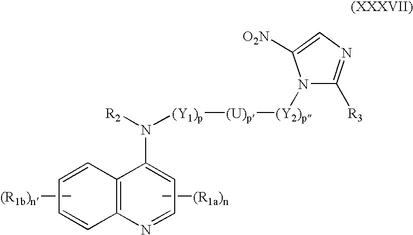

29. The compound according to claim 28, wherein, in the hybrid aminoquinoline-nitroimidazole molecules defined by formula (XXXVII), R₁ represents a halogen atom or a methyl, methoxy, trifluoromethyl, trifluoromethoxy, carboxy, cyano, amine, or nitro group occupying any position, and R₂ represents a hydrogen atom or a methyl group or forms a cyclic structure with Y₁ including the N of the aminoquinoline, R₃ is a methyl group and insofar as the (Y₁)ₚ—(U)_{p′}—(Y₂)_{p″} group is a group in which p=1 and p′=p″=0, Y₁ represents a C1, C2, C3, C4, C5, or C6 alkyl chain or a group in which p=p′=p″=1, U represents an amine function, Y₁ represents a C1, C2, C3, C4, C5, or C6 alkyl chain that can form a cyclic structure with R₂ including the N of the aminoquinoline, and Y₂ represents a C1, C2, C3, C4, C5, or C6 alkyl chain bearing a hydroxy function.

30. A compound represented by the formula (XXXVIII): 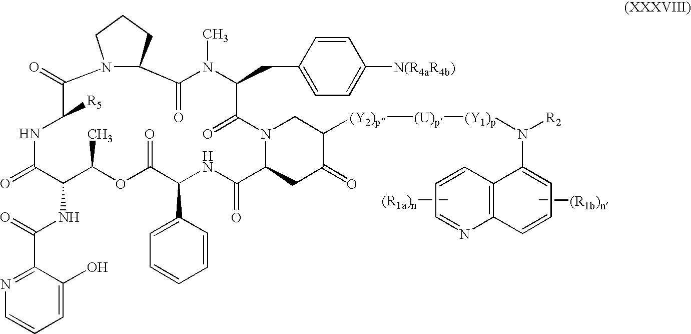
Wherein R₁, R₂, R₄ₐ, R_{4b}, R₅, Y₁, Y₂, U, p, p′, p″, n, and n′ are as defined as follows:
n and n′ represent, independently of each other, 0, 1, 2 or 3;
R₁ₐ and R_{1b} (generally R₁) represent one or more substituents which are identical or different, occupying any position and representing a substituent which is selected from the group consisting of halogen, trifluoromethyl, trifluoromethoxy, carboxy, amine, sulfate, sulfonate, phosphate, phosphonate, nitro, cyano, aryl or heteroaryl or alkyl, alkylamino, dialkylamino, alkoxy, alkylthio, alkylsulfonyl, alkylsulfamoyl, alkylsulfonylamino, alkylcarbamoyl, dialkylcarbamoyl, alkylcarbonyloxy, alkoxycarbonyl, alkylcarbonylamino, the said alkyl groups comprising 1, 2, 3, 4, 5 or 6 carbon atoms, which are linear, branched or cyclic, saturated or unsaturated, containing if need be one or more amine, amide, thioamide, sulfonyl, sulfonamide, carboxy, thiocarboxy, carbonyl, thiocarbonyl, hydroxyimine, ether or thioether functions and themselves being able to bear 1 to 4 substituents, which are identical or different, and which are selected from among halogen, hydroxy, trifluoromethyl, trifluoromethoxy, carboxy, carbonyl, amine, nitro, urea, aryl, or heteroaryl,
R₂ₐ and R_{2b} (generally R₂) being substituents which are identical or different, being able if need be to form a cyclic structure together or with Y₁, Y₂, U or A and representing a hydrogen atom or a linear, branched or cyclic C1, C2, C3, C4, C5 or C6 alkyl substituent containing if need be one or more amine, amide, thioamide, sulfonyl, urea, thiourea, carbamate, oxime, sulfonamide, carboxy, thiocarboxy, carbonyl, thiocarbonyl, ether or thioether functions and being able to bear 1 to 4 substituents, which are identical or different, and which are selected from halogen, hydroxy, trifluoromethyl, trifluoromethoxy, methoxy, carboxy, amine, nitro, aryl, or heteroaryl, R₂ₐ and R_{2b} being not simultaneously a hydrogen atom;
p, p′, p″ are, independently of each other, 0 or 1,
Y₁ and Y₂, which are identical or different, and can be linked by a single or multiple bond to Q, U or A, and represent a saturated or unsaturated, linear, branched or cyclic C1, C2, C3, C4, C5 or C6 alkyl chain, containing if need be one or more amine, amide, thioamide, sulfonyl, sulfonamide, oxo, carboxy, thiocarboxy, carbonyl, thiocarbonyl, urea, thiourea, carbamate, oxime, ether or thioether function, or an aryl or heteroaryl group, wherein the alkyl chain can additionally bear 1 to 4 substituents, which are identical or different, and which are preferably selected from the group consisting of halogen, hydroxy, trifluoromethyl, trifluoromethoxy, methoxy, carboxy, carbonyl, amine, nitro, oxime, aryl or heteroaryl such as defined herein after, or selected from among substituents of the type alkyl, alkylamino, dialkylamino, alkoxy, alkylthio, alkylsulfonyl, alkylsulfonamino, alkylsulfamoyl, alkylureido, alkylcarbamoyloxy, alkoxycarbonylamino, alkylcarbamoyl, dialkylcarbamoyl, alkylcarbonylamino, alkylcarbonyl, alkylcarbonyloxy, alkoxycarbonyl, alkoxyimine, the said alkyl groups comprising from 1 to 6 linear, branched or cyclic carbon atoms which can themselves contain one or more amine, amide, thioamide, sulfonyl, sulfonamide, carboxy, thiocarboxy, carbonyl, thiocarbonyl, oxime, ether or thioether function, or an aryl or heteroaryl group, such as those defined herein after, wherein the C1, C2, C3, C4, C5 or C6 chain may form a cyclic structure with R₂ including N from the aminoquinoline part Q and/or the function U and Y₁ and Y₂ may be linked together or to Q, U or A by a single or multiple bond,
U, which can be linked by a single or multiple bond to Q, Y₁, Y₂ or A, is an amine, amide, thioamide, sulfonyl, sulfonamide, carboxy, thiocarboxy, carbonyl, urea, thiourea, carbamate, ether, thioether, thiocarbonyl, sulfonate, oxime, oxyamine, alkylamine (NR), alkoxyimine (C═N—OR) or alkoxyiminocarbonyl (C(O)—C═N—OR) function with R representing a hydrogen atom or a C1, C2, C3, C4, C5 or C6 alkyl substituent, which is linear, branched or cyclic, containing if need be one or more amine, amide, thioamide, sulfonyl, sulfonamide, carboxy, thiocarboxy, carbonyl, thiocarbonyl, ether or thioether functions,
R₄ represents a hydrogen atom or a saturated or unsaturated, linear, branched or cyclic C1, C2, C3, C4, C5 or C6 alkyl substituent, containing if need be one or more amine, amide, thioamide, sulfonyl, sulfonamide, carboxy, thiocarboxy, carbonyl, thiocarbonyl, oxime, urea, carbamate, ether or thioether functions and being able to bear 1 to 4 substituents, which are identical or different, and which are selected from halogen, hydroxy, trifluoromethyl, trifluoromethoxy, methoxy, carboxy, amine, nitro, aryl, or heteroaryl,
R₅ is a hydrogen atom or a saturated or unsaturated, linear, branched or cyclic C1, C2, C3, C4, C5 or C6 alkyl substituent,

31. The compound according to claim 30, wherein, in the hybrid aminoquinoline-streptogramin molecules defined by formula (XXXVIII),
R₁ represents a halogen atom or a methyl, methoxy, trifluoromethyl, trifluoromethoxy, carboxy, cyano, amine, or nitro group occupying any position, and R₂ represents a hydrogen atom or a methyl group or forms a cyclic structure with Y₁ including the N of the aminoquinoline, and R₄ₐ, R_{4b} and R₅ are C1, C2, C3, C4, C5, or C6 alkyl chains (preferably R₄ₐ and R_{4b} are methyl substituent and R₅ an ethyl substituent);
the (Y₁)ₚ—(U)_{p′}—(Y₂)_{p″} group is a group in which p=p′=p″=1, U represents a thioether function, and Y₁ and Y₂ represent a C1, C2, C3, C4, C5, or C6 alkyl chain.

32. The compound according to claim 1, wherein it is represented by the formula (XXXIX), in which R₁, R₂, R₄, R₅, Y₁, Y₂, U, p, p′, p″, n, and n′ are as defined above:

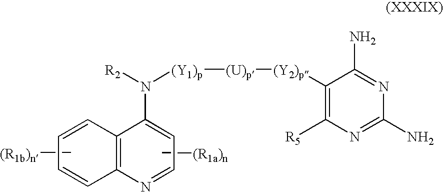

33. The compound according to claim 32, wherein, in the hybrid aminoquinoline-diaminopyrimidine molecules defined by formula (XXXIX), R₁ represents a halogen atom or a methyl, methoxy, trifluoromethyl, trifluoromethoxy, carboxy, cyano, amine, or nitro group occupying any position, and R₂ represents a hydrogen atom or a methyl group or forms a cyclic structure with Y₁ including the N of the aminoquinoline, R₅ is a hydrogen atom, and the (Y₁)ₚ—(U)_{p′}—(Y₂)_{p″} group is a group in which p=p′=p″=1, U advantageously represents an ether function, Y₁ represents a C1, C2, C3, C4, C5, or C6 alkyl chain, and Y₂ represents a C1, C2, C3, C4, C5, or C6 alkyl chain containing an aryl group which itself can bear 1 to 4 identical or different substituents.

34. The compound according to claim 15, wherein it is represented by the formula (XLa), (XLb), or (XLc), in which R₁, R₂, R₅, R₆, R₇, R₁₀, Y₁, Y₂, U, p, p′, p″, n, and n′ are as defined in claim 1 or as follows: 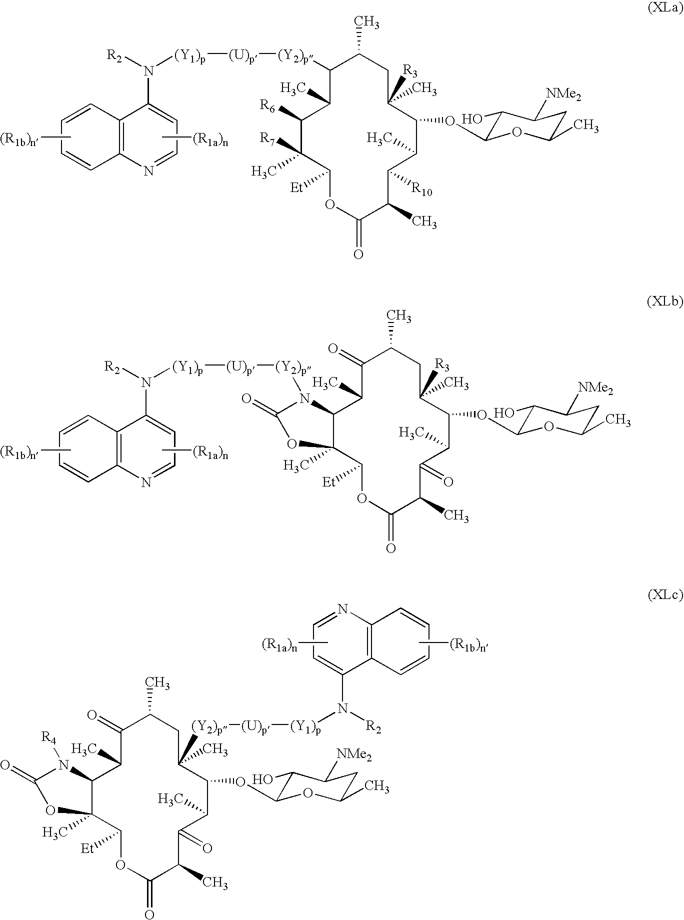
in which R₁, R₂, R₄ₐ, R_{4b}, R₅, Y₁, Y₂, U, p, p′, p″, n, and n′ are as defined in claim 1 or as follows:
n and n′ represent, independently of each other, 0, 1, 2 or 3;
R₁ₐ and R_{1b} (generally R₁) represent one or more substituents which are identical or different, occupying any position and representing a substituent which is selected from the group consisting of halogen, trifluoromethyl, trifluoromethoxy, carboxy, amine, sulfate, sulfonate, phosphate, phosphonate, nitro, cyano, aryl or heteroaryl or alkyl, alkylamino, dialkylamino, alkoxy, alkylthio, alkylsulfonyl, alkylsulfamoyl, alkylsulfonylamino, alkylcarbamoyl, dialkylcarbamoyl, alkylcarbonyloxy, alkoxycarbonyl, alkylcarbonylamino, the said alkyl groups comprising 1, 2, 3, 4, 5 or 6 carbon atoms, which are linear, branched or cyclic, saturated or unsaturated, containing if need be one or more amine, amide, thioamide, sulfonyl, sulfonamide, carboxy, thiocarboxy, carbonyl, thiocarbonyl, hydroxyimine, ether or thioether functions and themselves being able to bear 1 to 4 substituents, which are identical or different, and which are selected from among halogen, hydroxy, trifluoromethyl, trifluoromethoxy, carboxy, carbonyl, amine, nitro, urea, aryl, or heteroaryl,
R₂ₐ and R_{2b} (generally R₂) being substituents which are identical or different, being able if need be to form a cyclic structure together or with Y₁, Y₂, U or A and representing a hydrogen atom or a linear, branched or cyclic C1, C2, C3, C4, C5 or C6 alkyl substituent containing if need be one or more amine, amide, thioamide, sulfonyl, urea, thiourea, carbamate, oxime, sulfonamide, carboxy, thiocarboxy, carbonyl, thiocarbonyl, ether or thioether functions and being able to bear 1 to 4 substituents, which are identical or different, and which are selected from halogen, hydroxy, trifluoromethyl, trifluoromethoxy, methoxy, carboxy, amine, nitro, aryl, or heteroaryl, R₂ₐ and R_{2b} being not simultaneously a hydrogen atom;
p, p′, p″ are, independently of each other, 0 or 1,
Y₁ and Y₂, which are identical or different, and can be linked by a single or multiple bond to Q, U or A, and represent a saturated or unsaturated, linear, branched or cyclic C1, C2, C3, C4, C5 or C6 alkyl chain, containing if need be one or more amine, amide, thioamide, sulfonyl, sulfonamide, oxo, carboxy, thiocarboxy, carbonyl, thiocarbonyl, urea, thiourea, carbamate, oxime, ether or thioether function, or an aryl or heteroaryl group, wherein the alkyl chain can additionally bear 1 to 4 substituents, which are identical or different, and which are preferably selected from the group consisting of halogen, hydroxy, trifluoromethyl, trifluoromethoxy, methoxy, carboxy, carbonyl, amine, nitro, oxime, aryl or heteroaryl such as defined herein after, or selected from among substituents of the type alkyl, alkylamino, dialkylamino, alkoxy, alkylthio, alkylsulfonyl, alkylsulfonamino, alkylsulfamoyl, alkylureido, alkylcarbamoyloxy, alkoxycarbonylamino, alkylcarbamoyl, dialkylcarbamoyl, alkylcarbonylamino, alkylcarbonyl, alkylcarbonyloxy, alkoxycarbonyl, alkoxyimine, the said alkyl groups comprising from 1 to 6 linear, branched or cyclic carbon atoms which can themselves contain one or more amine, amide, thioamide, sulfonyl, sulfonamide, carboxy, thiocarboxy, carbonyl, thiocarbonyl, oxime, ether or thioether function, or an aryl or heteroaryl group, such as those defined herein after, wherein the C1, C2, C3, C4, C5 or C6 chain may form a cyclic structure with R₂ including N from the aminoquinoline part Q and/or the function U and Y₁ and Y₂ may be linked together or to Q, U or A by a single or multiple bond,
U, which can be linked by a single or multiple bond to Q, Y₁, Y₂ or A, is an amine, amide, thioamide, sulfonyl, sulfonamide, carboxy, thiocarboxy, carbonyl, urea, thiourea, carbamate, ether, thioether, thiocarbonyl, sulfonate, oxime, oxyamine, alkylamine (NR), alkoxyimine (C═N—OR) or alkoxyiminocarbonyl (C(O)C═N—OR) function with R representing a hydrogen atom or a C1, C2, C3, C4, C5 or C6 alkyl substituent, which is linear, branched or cyclic, containing if need be one or more amine, amide, thioamide, sulfonyl, sulfonamide, carboxy, thiocarboxy, carbonyl, thiocarbonyl, ether or thioether functions,
R₄ represents a hydrogen atom or a saturated or unsaturated, linear, branched or cyclic C1, C2, C3, C4, C5 or C6 alkyl substituent, containing if need be one or more amine, amide, thioamide, sulfonyl, sulfonamide, carboxy, thiocarboxy, carbonyl, thiocarbonyl, oxime, urea, carbamate, ether or thioether functions and being able to bear 1 to 4 substituents, which are identical or different, and which are selected from halogen, hydroxy, trifluoromethyl, trifluoromethoxy, methoxy, carboxy, amine, nitro, aryl, or heteroaryl,
R₅ is a hydrogen atom or a saturated or unsaturated, linear, branched or cyclic C1, C2, C3, C4, C5 or C6 alkyl substituent,
R₁₀ is an oxygen atom linked via a double bond of carbonyl type to the macrocycle or a hydroxy group or an osidic derivative linked via a glycosidic bridge to the macrocycle and being able to bear 1 to 6 substituents, which are identical or different, and which are selected from hydroxy, alkyl, alkylamino, dialkylamino, or alkoxy, said alkyl groups including 1, 2, 3, 4, 5, or 6 carbon atoms which are linear or branched, saturated or unsaturated, and may bear a carboxy substituent.

35. The compound according to claim 34, wherein:
in the hybrid aminoquinoline-macrolide molecules having the formulae (XLa), (XLb) and (XLc), R₁ represents a halogen atom or a methyl, methoxy, trifluoromethyl, trifluoromethoxy, carboxy, cyano, amine, or nitro group occupying any position, and R₂ represents a hydrogen atom or a methyl group or forms a cyclic structure with Y₁ including the N of the aminoquinoline, R₃ is a hydroxy or methoxy group, R₄ is a hydrogen atom, Rᵣ and R₇ are hydroxy groups, R₁₀ is an oxygen atom linked via a double bond of carbonyl type to the macrocycle or an osidic derivative linked via a glycosidic bridge to the macrocycle and being able to bear 1 to 6 substituents;
in the hybrid aminoquinoline-macrolide molecules having the formula (XLa), the (Y₁)ₚ—(U)_{p′}—(Y₂)_{p″} group is a group in which p=p′=1 and p″=0, U represents an oxyamine function linked via a double bond to A (thus forming an oxime function), and Y₁ represents a C1, C2, C3, C4, C5, or C6 alkyl chain that may contain an ether function;
in the hybrid aminoquinoline-macrolide molecules having the formula (XLb), the (Y₁)ₚ—(U)_{p′}—Y₂)_{p″} group is a group in which p=1 and p′=p″=0, Y1 represents a C1, C2, C3, C4, C5, or C6 alkyl chain that may contain an ether function;
in the hybrid aminoquinoline-macrolide molecules having the formula (XLc), the (Y₁)ₚ—(U)_{p′}—(Y₂)_{p″} group is a group in which p=p′=1 and p″=0, U represents an ether or carbamate function, and Y₁ represents a saturated or unsaturated C1, C2, C3, C4, C5, or C6 alkyl chain that may contain an ether function and/or an aryl group.

36. A compound represented by the formula (XLIa) or (XLIb): 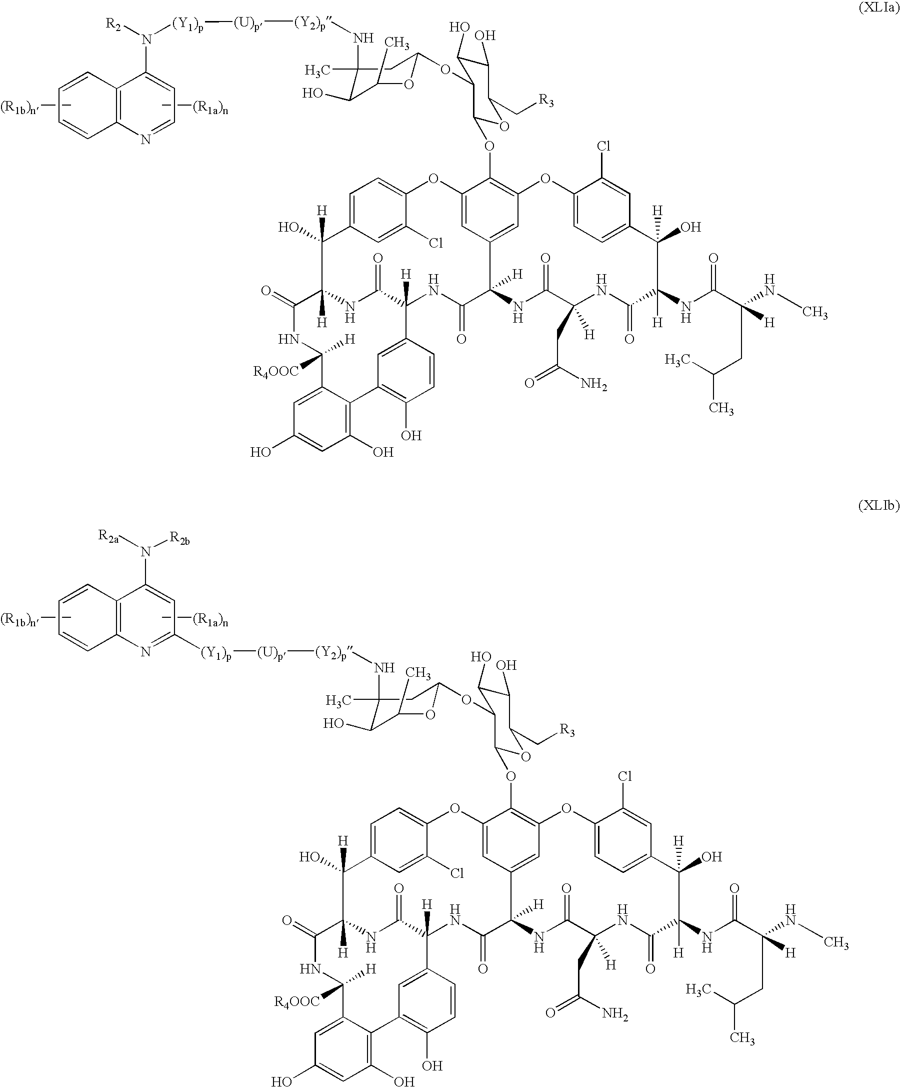
wherein R₁, R₂, Y₁, Y₂, U, p, p′, p″, n, and n′ are as follows:
n and n′ represent, independently of each other, 0, 1, 2 or 3;
R₁ₐ and R_{1b} (generally R₁) represent one or more substituents which are identical or different, occupying any position and representing a substituent which is selected from the group consisting of halogen, trifluoromethyl, trifluoromethoxy, carboxy, amine, sulfate, sulfonate, phosphate, phosphonate, nitro, cyano, aryl or heteroaryl or alkyl, alkylamino, dialkylamino, alkoxy, alkylthio, alkylsulfonyl, alkylsulfamoyl, alkylsulfonylamino, alkylcarbamoyl, dialkylcarbamoyl, alkylcarbonyloxy, alkoxycarbonyl, alkylcarbonylamino, the said alkyl groups comprising 1, 2, 3, 4, 5 or 6 carbon atoms, which are linear, branched or cyclic, saturated or unsaturated, containing if need be one or more amine, amide, thioamide, sulfonyl, sulfonamide, carboxy, thiocarboxy, carbonyl, thiocarbonyl, hydroxyimine, ether or thioether functions and themselves being able to bear 1 to 4 substituents, which are identical or different, and which are selected from among halogen, hydroxy, trifluoromethyl, trifluoromethoxy, carboxy, carbonyl, amine, nitro, urea, aryl, or heteroaryl,
R₂, and R_{2b} (generally R₂) being substituents which are identical or different, being able if need be to form a cyclic structure together or with Y₁, Y₂, U or A and representing a hydrogen atom or a linear, branched or cyclic C1, C2, C3, C4, C5 or C6 alkyl substituent containing if need be one or more amine, amide, thioamide, sulfonyl, urea, thiourea, carbamate, oxime, sulfonamide, carboxy, thiocarboxy, carbonyl, thiocarbonyl, ether or thioether functions and being able to bear 1 to 4 substituents, which are identical or different, and which are selected from halogen, hydroxy, trifluoromethyl, trifluoromethoxy, methoxy, carboxy, amine, nitro, aryl, or heteroaryl, R₂ₐ and R_{2b} being not simultaneously a hydrogen atom;
p, p′, p″ are, independently of each other, 0 or 1,
Y₁ and Y₂, which are identical or different, and can be linked by a single or multiple bond to Q, U or A, and represent a saturated or unsaturated, linear, branched or cyclic C1, C2, C3, C4, C5 or C6 alkyl chain, containing if need be one or more amine, amide, thioamide, sulfonyl, sulfonamide, oxo, carboxy, thiocarboxy, carbonyl, thiocarbonyl, urea, thiourea, carbamate, oxime, ether or thioether function, or an aryl or heteroaryl group, wherein the alkyl chain can additionally bear 1 to 4 substituents, which are identical or different, and which are preferably selected from the group consisting of halogen, hydroxy, trifluoromethyl, trifluoromethoxy, methoxy, carboxy, carbonyl, amine, nitro, oxime, aryl or heteroaryl such as defined herein after, or selected from among substituents of the type alkyl, alkylamino, dialkylamino, alkoxy, alkylthio, alkylsulfonyl, alkylsulfonamino, alkylsulfamoyl, alkylureido, alkylcarbamoyloxy, alkoxycarbonylamino, alkylcarbamoyl, dialkylcarbamoyl, alkylcarbonylamino, alkylcarbonyl, alkylcarbonyloxy, alkoxycarbonyl, alkoxyimine, the said alkyl groups comprising from 1 to 6 linear, branched or cyclic carbon atoms which can themselves contain one or more amine, amide, thioamide, sulfonyl, sulfonamide, carboxy, thiocarboxy, carbonyl, thiocarbonyl, oxime, ether or thioether function, or an aryl or heteroaryl group, such as those defined herein after, wherein the C1, C2, C3, C4, C5 or C6 chain may form a cyclic structure with R₂ including N from the aminoquinoline part Q and/or the function U and Y₁ and Y₂ may be linked together or to Q, U or A by a single or multiple bond,
U, which can be linked by a single or multiple bond to Q, Y₁, Y₂ or A, is an amine, amide, thioamide, sulfonyl, sulfonamide, carboxy, thiocarboxy, carbonyl, urea, thiourea, carbamate, ether, thioether, thiocarbonyl, sulfonate, oxime, oxyamine, alkylamine (NR), alkoxyimine (C═N—OR) or alkoxyiminocarbonyl (C(O)—C═N—OR) function with R representing a hydrogen atom or a C1, C2, C3, C4, C5 or C6 alkyl substituent, which is linear, branched or cyclic, containing if need be one or more amine, amide, thioamide, sulfonyl, sulfonamide, carboxy, thiocarboxy, carbonyl, thiocarbonyl, ether or thioether functions.

37. The compound according to claim 36, wherein, in the hybrid aminoquinoline-glycopeptide molecules having the formula (XLIa) or (XLIb), R₁ represents a halogen atom or a methyl, methoxy, trifluoromethyl, trifluoromethoxy, carboxy, cyano, amine, or nitro group occupying any position, and R₂ represents a hydrogen atom, a methyl, cyclopropyl, or 2-(diethylamino)ethyl group, or forms a cyclic structure with Y₁ including the N of the aminoquinoline, or a heterocycle when R₂ₐ and R_{2b} together form a cyclic structure, R₄ is a hydrogen atom, and R₃ is a hydroxy group; in the hybrid aminoquinoline-glycopeptide molecules having the formula (XLIa), the (Y₁)ₚ—(U)ₚ—(Y₂)_{p″} group is a group in which p=1 and p′=p″=0, Y₁ represents a C1, C2, C3, C4, C5, or C6 alkyl chain that may form a cyclic structure with the nitrogen atom of the A residue and R₂ (including the N of the aminoquinoline) and is able to be substituted by fluorine atoms, or a group in which p=p′=p″=1, U represents an ether or amine function, Y₁ represents a C1, C2, C3, C4, C5, or C6 alkyl chain that may form a cyclic structure with U and R₂ (including the N of the aminoquinoline), and Y₂ represents a C1, C2, C3, C4, C5, or C6 alkyl chain that may contain an aryl group as defined above and which itself may bear 1 to 4 identical or different substituents; in the hybrid aminoquinoline-glycopeptide molecules having the formula (XLIb), the (Y₁)ₚ—(U)_{p′}—(Y₂)_{p″} group is a group in which p=1 and p′=p″=0, Y₁ represents a C1, C2, C3, C4, C5, or C6 alkyl chain, or a group in which p=0 and p′=p″=1, U represents an amide function, and Y₂ represents a C1, C2, C3, C4, C5, or C6 alkyl chain.

38. A compound represented by the formula (XLIIa), (XLIIb), or (XLIIc): 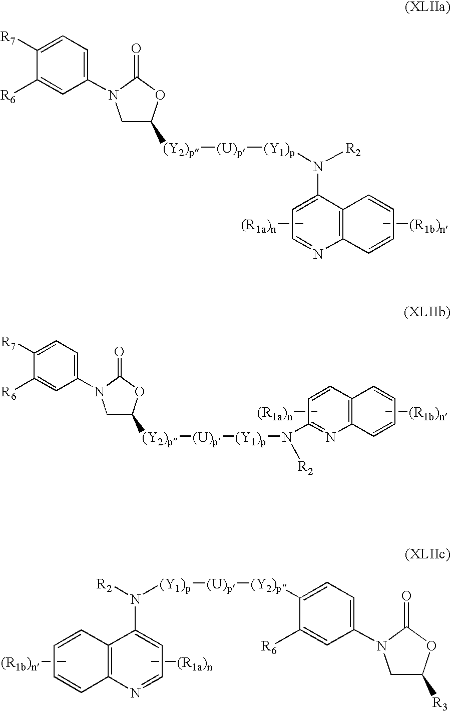
in which R₁, R₂, R₆, R₇, Y₁, Y₂, U, p, p′, p″, n, and n′ are as follows:
n and n′ represent, independently of each other, 0, 1, 2 or 3;
R₁ₐ and R_{1b} (generally R₁) represent one or more substituents which are identical or different, occupying any position and representing a substituent which is selected from the group consisting of halogen, trifluoromethyl, trifluoromethoxy, carboxy, amine, sulfate, sulfonate, phosphate, phosphonate, nitro, cyano, aryl or heteroaryl or alkyl, alkylamino, dialkylamino, alkoxy, alkylthio, alkylsulfonyl, alkylsulfamoyl, alkylsulfonylamino, alkylcarbamoyl, dialkylcarbamoyl, alkylcarbonyloxy, alkoxycarbonyl, alkylcarbonylamino, the said alkyl groups comprising 1, 2, 3, 4, 5 or 6 carbon atoms, which are linear, branched or cyclic, saturated or unsaturated, containing if need be one or more amine, amide, thioamide, sulfonyl, sulfonamide, carboxy, thiocarboxy, carbonyl, thiocarbonyl, hydroxyimine, ether or thioether functions and themselves being able to bear 1 to 4 substituents, which are identical or different, and which are selected from among halogen, hydroxy, trifluoromethyl, trifluoromethoxy, carboxy, carbonyl, amine, nitro, urea, aryl, or heteroaryl,
R₂ₐ and R_{2b} (generally R₂) being substituents which are identical or different, being able if need be to form a cyclic structure together or with Y₁, Y₂, U or A and representing a hydrogen atom or a linear, branched or cyclic C1, C2, C3, C4, C5 or C6 alkyl substituent containing if need be one or more amine, amide, thioamide, sulfonyl, urea, thiourea, carbamate, oxime, sulfonamide, carboxy, thiocarboxy, carbonyl, thiocarbonyl, ether or thioether functions and being able to bear 1 to 4 substituents, which are identical or different, and which are selected from halogen, hydroxy, trifluoromethyl, trifluoromethoxy, methoxy, carboxy, amine, nitro, aryl, or heteroaryl, R₂ₐ and R_{2b} being not simultaneously a hydrogen atom;
p, p′, p″ are, independently of each other, 0 or 1,
Y₁ and Y₂, which are identical or different, and can be linked by a single or multiple bond to Q, U or A, and represent a saturated or unsaturated, linear, branched or cyclic C1, C2, C3, C4, C5 or C6 alkyl chain, containing if need be one or more amine, amide, thioamide, sulfonyl, sulfonamide, oxo, carboxy, thiocarboxy, carbonyl, thiocarbonyl, urea, thiourea, carbamate, oxime, ether or thioether function, or an aryl or heteroaryl group, wherein the alkyl chain can additionally bear 1 to 4 substituents, which are identical or different, and which are preferably selected from the group consisting of halogen, hydroxy, trifluoromethyl, trifluoromethoxy, methoxy, carboxy, carbonyl, amine, nitro, oxime, aryl or heteroaryl such as defined herein after, or selected from among substituents of the type alkyl, alkylamino, dialkylamino, alkoxy, alkylthio, alkylsulfonyl, alkylsulfonamino, alkylsulfamoyl, alkylureido, alkylcarbamoyloxy, alkoxycarbonylamino, alkylcarbamoyl, dialkylcarbamoyl, alkylcarbonylamino, alkylcarbonyl, alkylcarbonyloxy, alkoxycarbonyl, alkoxyimine, the said alkyl groups comprising from 1 to 6 linear, branched or cyclic carbon atoms which can themselves contain one or more amine, amide, thioamide, sulfonyl, sulfonamide, carboxy, thiocarboxy, carbonyl, thiocarbonyl, oxime, ether or thioether function, or an aryl or heteroaryl group, such as those defined herein after, wherein the C1, C2, C3, C4, C5 or C6 chain may form a cyclic structure with R₂ including N from the aminoquinoline part Q and/or the function U and Y₁ and Y₂ may be linked together or to Q, U or A by a single or multiple bond,
U, which can be linked by a single or multiple bond to Q, Y₁, Y₂ or A, is an amine, amide, thioamide, sulfonyl, sulfonamide, carboxy, thiocarboxy, carbonyl, urea, thiourea, carbamate, ether, thioether, thiocarbonyl, sulfonate, oxime, oxyamine, alkylamine (NR), alkoxyimine (C═N—OR) or alkoxyiminocarbonyl (C(O)—C═N—OR) function with R representing a hydrogen atom or a C1, C2, C3, C4, C5 or C6 alkyl substituent, which is linear, branched or cyclic, containing if need be one or more amine, amide, thioamide, sulfonyl, sulfonamide, carboxy, thiocarboxy, carbonyl, thiocarbonyl, ether or thioether functions
R₆ and R₇ are substituents which are identical or different, being able if need be able to form, together, a cyclic structure and representing a hydrogen atom or a substituent which is selected from the group consisting of halogen, hydroxy, heterocycle, aryl or heteroaryl, or an alkyl, alkoxy or alkylamine substituent, said alkyl groups comprising 1, 2, 3, 4, 5 or 6 carbon atoms, which are saturated or unsaturated, linear, branched or cyclic, containing if need be one or more amine, amide, thioamide, sulfonyl, sulfonamide, carboxy, thiocarboxy, carbonyl, thiocarbonyl, ether or thioether functions and being able to bear 1 to 4 substituents, which are identical or different, and which are selected from halogen, hydroxy, trifluoromethyl, trifluoromethoxy, carboxy, amine, nitro, aryl, or heteroaryl.

39. The compound according to claim 38, wherein, in the hybrid aminoquinoline-oxazolidinone molecules having the formula (XLIIa), (XLIIb), or (XLIIc), in a preferred embodiment, R₁ represents a halogen atom or a methyl, methoxy, trifluoromethyl, trifluoromethoxy, carboxy, cyano, amine, or nitro group occupying any position, and R₂ represents a hydrogen atom or a methyl, cyclopropyl, or 2-(diethylamino)ethyl group, or forms a cyclic structure with Y₁ including the N of the aminoquinoline, R₆ is a hydrogen or fluorine atom, R₇ is a heterocycle containing 5 to 6 members and containing 1 to 4 heteroatoms chosen from among nitrogen, sulfur, and oxygen (preferably morpholin-4-yl or piperazin-1-yl), and R₃ is a C1, C2, C3, C4, C5, or C6 alkyl chain that may contain an amide, carbamate, or ether function and that can be substituted by a heterocycle; in the hybrid aminoquinoline-oxazolidinone molecules having the formula (XLIIa), the (Y₁)ₚ—(U)_{p′}—(Y₂)_{p″} group is a group in which p=p′=p″=1, U represents an amide or carbamate function, and Y₁ and Y₂ represent a C1, C2, C3, C4, C5, or C6 alkyl chain that can form a cyclic structure with U and/or R₂ including the N of the aminoquinoline; in the hybrid aminoquinoline-oxazolidinone molecules having the formula (XLIIb), the (Y₁)ₚ—(U)_{p′}—(Y₂)_{p″} group is a group in which p=p′=p″=1, U represents a carbamate function, and Y₁ and Y₂ represent a C1, C2, C3, C4, C5, or C6 alkyl chain that can form a cyclic structure with U and/or R₂ including the N of the aminoquinoline, in the hybrid aminoquinoline-oxazolidinone molecules having the formula (XLIIc), the (Y₁)ₚ—(U)_{p′}—Y₂)_{p″} group is a group in which p=p′=p″=0, the link between Q and A being direct or a group in which p=p′=1 and p″=0, U represents an amine function, and Y₁ represents a C1, C2, C3, C4, C5, or C6 alkyl chain that can form a cyclic structure with U and/or R₂ including the N of the aminoquinoline and optionally containing an amine, amide, urea, or carbamate function.

40. The compound according to claim 1, selected from the group consisting of:
(2S,5R,6R)-6-{[1-(7-Chloroquinolin-4-yl)-piperidin-4-carbonyl]-amino}-3,3-dimethyl-7-oxo-4-thia-1-aza-bicyclo[3.2.0]heptane-2-carboxylic acid 2,2-dimethyl-propionyloxymethyl ester;
(2S,5R,6R)-3,3-Dimethyl-7-oxo-6-[3-(quinolin-8-ylamino)-propionylamino]-4-thia-1-aza-bicyclo[3.2.0]heptane-2-carboxylic acid 2,2-dimethyl-propionyloxymethyl ester;
(2S,5R,6R)-6-[2-(7-Chloroquinolin-4-ylamino)-acetylamino]-3,3-dimethyl-7-oxo-4-thia-1-aza-bicyclo[3.2.0]heptane-2-carboxylic acid 2,2-dimethyl-propionyloxymethyl ester;
(2S,5R,6R)-6-[3-(7-Chloroquinolin-4-ylamino)-propionylamino]-3,3-dimethyl-7-oxo-4-thia-1-aza-bicyclo[3.2.0]heptane-2-carboxylic acid 2,2-dimethyl-propionyloxymethyl ester;
(6R,7R-3-Acetoxymethyl-7-[2-(7-chloroquinolin-4-ylamino)-acetylamino]-8-oxo-5-thia-1-aza-bicyclo[4.2.0]oct-2-ene-2-carboxylic acid;
(6R,7R)-3-Acetoxymethyl-7-[2-(7-chloroquinolin-4-ylamino)-acetylamino]-8-oxo-5-thia-1-aza-bicyclo[4.2.0]oct-2-ene-2-carboxylic acid hydrochloride;
(6R,7R)-3-Acetoxymethyl-7-[2-(7-chloroquinolin-4-ylamino)-acetylamino]-5,8-dioxo-5λ⁴-thia-1-aza-bicyclo[4.2.0]oct-2-ene-2-carboxylic acid hydrochloride;
(6R,7R-3-Acetoxymethyl-7-[2-(7-chloroquinolin-4-ylamino)-acetylamino]-5,8-dioxo-5λ⁴-thia-1-aza-bicyclo[4.2.0]oct-2-ene-2-carboxylic acid;
(6R,7R-3-Acetoxymethyl-7-[3-(7-chloroquinolin-4-ylamino)-propionylamino]-8-oxo-5-thia-1-aza-bicyclo[4.2.0]oct-2-ene-2-carboxylic acid;
(6R,7R-3-Acetoxymethyl-7-[3-(7-chloroquinolin-4-ylamino)-propionylamino]-5,8-dioxo-5×4-thia-1-aza-bicyclo[4.2.0]oct-2-ene-2-carboxylic acid;
(6R,7R-3-Acetoxymethyl-7-[3-(7-chloroquinolin-4-ylamino)-propionylamino]-5,8-dioxo-5λ⁴-thia-1-aza-bicyclo[4.2.0]oct-2-ene-2-carboxylic acid hydrochloride;
(6R,7R-3-Acetoxymethyl-7-[4-7-chloroquinolin-4-ylamino)-butyrylamino]-8-oxo-5-thia-1-aza-bicyclo[4.2.0]oct-2-ene-2-carboxylic acid;
(6R,7R)-3-Acetoxymethyl-7-{[1-(7-chloroquinolin-4-yl)-piperidin-4-carbonyl]-amino}-8-oxo-5-thia-1-aza-bicyclo[4.2.0]oct-2-ene-2-carboxylic acid;
(6R,7R-3-Acetoxymethyl-7-{[1-(7-chloroquinolin-4-yl)-piperidine-4-carbonyl]-amino}-8-oxo-5-thia-1-aza-bicyclo[4.2.0]oct-2-ene-2-carboxylic acid hydrochloride;
(6R,7R-3-Acetoxymethyl-7-[2-(7-trifluoromethyl-quinolin-4-ylamino)-acetylamino]-8-oxo-5-thia-1-aza-bicyclo[4.2.0]oct-2-ene-2-carboxylic acid;
(6R,7R-3-Acetoxymethyl-7-[2-(2-methylquinolin-4-ylamino)-acetylamino]-8-oxo-5-thia-1-aza-bicyclo[4.2.0]oct-2-ene-2-carboxylic acid;
(6R,7R)-3-Acetoxymethyl-7-[4-morpholin-4-yl-quinoline-2-carbonyl)-amino]-8-oxo-5-thia-1-aza-bicyclo[4.2.0]oct-2-ene-2-carboxylic acid;
(6R,7R)-3-Acetoxymethyl-7-{[(4-(2-diethylamino-ethylamino)-quinoline-2-carbonyl]-amino}-8-oxo-5-thia-1-aza-bicyclo[4.2.0]oct-2-ene-2-carboxylic acid;
(6R,7R)-7-[2-(2-Aminothiazol-4-yl)-2-methoxyimino-acetylamino]-3-[2-(7-chloro-quinolin-4-ylamino)-ethylsulfanylmethyI]-8-oxo-5-thia-1-aza-bicyclo[4.2.0]oct-2-ene-2-carboxylic acid;
(6R,7R)-7-[2-(7-Chloroquinolin-4-ylamino)-acetylamino]-3-methyl-8-oxo-5-thia-1-aza-bicyclo[4.2.0]oct-2-ene-2-carboxylic acid
(6R,7R,11R/11S)-3-Acetoxymethyl-7-[2-(7-chloroquinolin-4-ylamino)-propionylamino]-8-oxo-5-thia-1-aza-bicyclo[4.2.0]oct-2-ene-2-carboxylic acid
(6R,7R)-3-acetoxymethyl-7-{2-(2-aminothiazol-4-yl)-2-[2(Z)-(7-chloroquinolin-4-ylamino)-ethoxyimino]-acetylamino}-8-oxo-5-thia-1-aza-bicyclo[4.2.0]oct-2-ene-2-carboxylic acid
7-[4-(7-Chloroquinolin-4-yl)-piperazin-1-yl]-1-cyclopropyl-6-fluoro-1,4-dihydro-quinoline-3-carboxylic acid hydrochloride;
7-{4-[2-(7-Chloroquinolin-4-ylamino)-ethyl]-piperazin-1-yl}-cyclopropyl-6-fluoro-4-oxo-1,4-dihydro-quinoline-3-carboxylic acid hydrochloride;
(7-Chloro-quinolin-4-yl)[2-(2-methyl-5-nitro-imidazol-1-yl)-ethyl]-amine;
1-Cyclopropopyl-6-fluoro-4-oxo-7-[4-(7-trifluoromethyl-quinolin-4-yl)-piperazin-1-yl]-1,4-dihydroquinoline-3-carboxylic acid
7-[4-(8-Chloroquinolin-4-yl)-piperazin-1-yl]-1-cyclopropopyl-6-fluoro-4-oxo-1,4-dihydroquinoline-3-carboxylic acid
[2-(2-Methyl-5-nitroimidazol-1-yl)-ethyl]-(7-trifluoromethyl-quinolin-4-yl)-amine;
1-[2-7-Chloroquinolin-4-ylamino)-ethylamino]-3-2-methyl-5-nitro-imidazo-1-yl) -propan-2-ol;
5δ-{4-[2-(7-Chloroquinolin-4-ylamino)-ethylamino]-methylsulfanyl}pristinamycin I_{A};
5-{4-[2-(7-Chloroquinolin-4-ylamino)-ethoxy]-benzyl}pyrimidine-2,4-diamine;
5-{4-[2-(7-Chloroquinolin-4-ylamino)-ethoxy]-3-methoxy-benzyl}pyrimidine-2,4-diamine;
5-{3-[2-(7-Chloroquinolin-4-ylamino)-ethoxy]-4,5-dimethoxy-benzyl}pyrimidine-2,4-diamine;
10{O-[3-(7-Chloroquinolin-4-ylamino)-propyl]-oxime}-erythromycin;
N-4-{4-[2-7-Chloroquinolin-4-ylamino)-ethoxy]-benzyl}-vancomycin;
N-4-[4-(7-Chloroquinolin-4-ylamino)-butyl]-vancomycin;
N-4-[4-(7-Chloroquinolin-4-ylamino)-ethyl]-vancomycin;
N-4-{4-[2-(2-Methylquinolin-4-ylamino)-ethoxy]-benzyl}-vancomycin
N-4-{4-[2-(2-Trifluoromethylquinolin-4-ylamino)-ethoxy]-benzyl}-vancomycin
N-4-{4-[2-(7-Trifluoromethylquinolin-4-ylamino)-ethoxy]-benzyl}-vancomycin
(5S)-[2-(7-Chloro-quinolin-4-ylamino)-ethyl]-carbamic acid 3-(3-fluoro-4-morpholin-4-yl-phenyl)-2-oxo-oxazolidin-5-ylmethylester;
(5S)-3-(7-Chloroquinolin-4-ylamino)-N-[3-(3-fluoro-4-morpholin-4-yl-phenyl)-2-oxo-oxazolidin-5-ylmethyl]-propionamide;
(5S)-2-(7-Chloroquinolin-4-ylamino)-N-[3-(3-fluoro-4-morpholin-4-yl-phenyl)-2-oxo-oxazolidin-5-ylmethyl]-acetamide;
(5S)-[2-(6-Chloroquinolin-2-ylamino)-ethyl]-carbamic acid 3-(3-fluoro-4-morpholin-4-yl-phenyl)-2-oxo-oxazolidin-5-ylmethyl ester
(5S)-N-[3-(4-{4-[2-(7-Chloroquinolin-4-ylamino)-acetyl]-piperazin-1-yl}-3-fluoro-phenyl)-2-oxo-oxazolidin-5-ylmethyl]-acetamide
(5S)-N-[3-(4-{4-[2-(7-Chloroquinolin-4-ylamino)-ethyl]-piperazin-1-yl}-3-fluoro-phenyl)-2-oxo-oxazolidin-5-ylmethyl]-acetamide
(5S)-N-[3-{4-[4-(7-Chloroquinolin-4-yl)-piperazin-1-yl]-3-fluoro-phenyl}-2-oxo-oxazolidin-5-ylmethyl]-acetamide,
and salts, or mixture thereof.

41. The compound according to claim 1, wherein it is in the form of a salt, for example such as salts of alkali metals, or of alkaline-earth metals, of ammonium salt or of salts of nitrogen-containing bases.

42. The compound according to claim 1, wherein it is used as an antibacterial agent.

43. A pharmaceutical composition, wherein it comprises, as active ingredient, at least one compound according to claim 1, mixed with a pharmaceutically acceptable excipient.

44. The pharmaceutical composition according to claim 43, wherein it is in a form which is capable of being administered in an injectable, pulverizable or ingestable form, for example via the intramuscular, intravenous, subcutaneous, intradermal, oral, topical, rectal, vaginal, ophthalmic, nasal, transdermal or parenteral route.

45. A method for carrying out a treatment of disinfection of a medical material comprising using a pharmaceutical composition comprising a compound as defined according to claim 1.

46. A method for treating a bacterial infection of an animal, or of a human being, said method comprising administering a compound according to claim 1 in an amount effective to said animal or human being.

47. A method for treating an infection or a bacterial contamination comprising administering a pharmaceutically effective amount of a compound according to claim 1, said infection or a bacterial contamination being selected from the group consisting of an infection or a bacterial contamination due to Staphylococcus aureus, Staphylococcus aureus MSSA (methicillin-sensitive), Staphylococcus aureus MSRA (methicillin-resistant), Staphylococcus aureus NorA (quinolone resistant by efflux), Staphylococcus aureus MsrA (macrolide-resistant by efflux) or Staphylococcus aureusVISA (or GISA) (vancomycin-resistant), Staphylococcus epidermidis, Staphylococcus epidermidis MSCNS (methicillin-sensitive coagulase negative) or Staphylococcus epidermidis MRCNS (methicillin-resistant coagulase negative), Streptococcus pneumoniae, Streptococcus pneumoniae PSSP (penicillin-sensitive), Streptococcus pneumoniae PRSP (penicillin resistant) or Streptococcus pneumoniae mefE (macrolide-resistant by efflux), Streptococcus pyogenes, Enterococcus faecalis, Enterococcus faecalisVSE (vancomycin-sensible), Enterococcus faecalis VRE (vancomycin-resistant), Enterococcus faecium, Enterococcus faecium VSE (vancomycin-sensible), Enterococcus faecium VRE (vancomycin-resistant), Haemophilus influenzae, Moraxella catarrhalis, Escherichia coli, Pseudomonas aeruginosa, Bacillus subtilis, Bacillus thuringiensis, Clostridium difficile or Bacteroides fragilis.

48. The method according to claim 46, wherein said infection is a condition caused by a gram positive bacteria.

49. The method according to claim 46, wherein said infection is a condition caused by gram positive bacteria and gram negative bacteria.

50. The method according to claim 46, wherein said infection is a human bacterial infection selected from the group consisting of pneumonia, meningitis, otitis, sinusitis, skin or mucosal infections, nosocomial infections, osteomyelitis, iatrogenic infections, urinary infections, genital infections, biliary infections, dental infections, otitis, sinusitis, endocarditis, bacterial angina, ORL conditions, scarlet fever, erysipela, impetigo, subcutaneous gangrene, complications of influenza, abdominal infections, infantile diarrhea, alimentary intoxications and food poisoning, bacteremia, abscesses or lesions, peritonitis, and wound infections.

51. The method according to claim 46, wherein said infection is wherein said compound is selected from the group consisting of:
a hybrid aminoquinoline-β-lactam molecule of a formulae selected from the group consisting of (XXXIVa), (XXXIVc), (XXXVa), (XXXVb), (XXXVd) and (XXXVf),
a hybrid aminoquinoline-quinolone molecules of formula (XXXVIa),
a hybrid aminoquinoline-nitroimidazole molecules of formula (XXXVII),
a hybrid aminoquinoline-streptogramin molecules of formula (XXXVIII),
a hybrid aminoquinoline-diaminopyrimidine molecules of formula (XXXIX),
a hybrid molecules of formula (XLa),
a hybrid aminoquinoline-glycopeptide molecules of formula (XLIa), and
a hybrid aminoquinoline-oxazolidinone molecules of a formulae selected from the group consisting of (XLIIa), (XLIIb) and (XLIIc).

52. The pharmaceutical composition according to claim 43, said composition containing an effective quantity of at least one compound having formula (I), as defined according to claim 1, in combination with other pharmacologically active substances.

53. The pharmaceutical compositions according to claim 52, wherein at least one compound having formula (I), as defined according to any one of claims 1 to 42, is combined with a resistance enzyme inhibitor.

54. The pharmaceutical compositions according to claim 53, wherein the resistance enzyme inhibitor is a β-lactamase inhibitor, preferably chosen from among clavulanic acid (3-(2-hydroxyethylidene)-7-oxo-4-oxa-1-azabicyclo[3.2.0]heptane-2-carboxylic acid), sulbactam sodium (sodium 4,4 dioxide [2S-(2 alpha, 5 alpha)]-3,3-dimethyl-4,4,7-trioxo-4Δ⁶-thia-1-azabicyclo[3,2,0]heptane-2-carboxylate), and tazobactam sodium (sodium [2S-(2 alpha,3,bêta,5 alpha)]-3-methyl-4,4,7-trioxo-3-(1H-[1,2,3]—triazol-1-ylmethyl)-4λ⁶-thia-1-azabicyclo[3,2,0]heptane-2-carboxylate).

55. A method for agri-food industry comprising the use of a compound according to claim 1.

56. A method for treating a bacterial infection of an animal or of a human being other than an infection caused by mycoplasma sp., said method comprising administering a pharmaceutically effective amount of a compound according to claim 1, wherein A is 1-cyclopropyl-6-fluoro-1,4-dihydroquinoline-3-carboxylic acid or 1-cyclopropyl-6,8-difluoro-1,4-dihydroquinoline-3-carboxylic acid, the link —(Y₁)ₚ—(U)_{p′}—(Y₂)_{p″}— between A and Q is a piperazine, and Q is 7-chloro-4-aminoquinoline.

57. A method for treating a bacterial infection of an animal or of a human being, said method comprising administering a pharmaceutically effective amount of a compound of formula (I):
Q—(Y₁)ₚ—(U)_{p′}—(Y₂)_{p″}-A (I)
wherein A is 2-methyl-5-nitro-imidazol-1-yl, linked directly to the extracyclic nitrogen atom of the aminoquinoline Q (p=p′=p″=0), wherein Q is chosen from the group consisting of 7-chloroquinolin-4-ylamino, 2-methyl-8-hydroxyquinolin-4-ylamino, 2-methyl-3-n-propyl-8-hydroxy-quinolin-4-ylamino, and 2-methyl-5-nitro-8-hydroxyquinolin-4-ylamino,
or wherein A is 2-methyl-5-nitro-imidazol-1-yl, the link —(Y₁)ₚ—(U)_{p′}—(Y₂)_{p″} is a 2-ethyl-(1-cyclohexan-4-yl)-amine link, and Q is 7chloro-quinolin-4-ylamino.

58. A method of preparing a compound Q—(Y₁)ₚ—(U)_{p′}—(Y₂)_{p″}-A, as defined in claim 1, wherein said method comprises the reaction sequence selected from the group consisting of:
a) binding the (Y₁)ₚ—(U)_{p′}—(Y₂)_{p″} group to an aminoquinoline Q, then the reaction of this intermediate compound with an antibiotic A;
b) binding the (Y₁)ₚ—(U)_{p′}—(Y₂)_{p″} group to an antibiotic A, then coupling this intermediate with an aminoquinoline Q; and
c) binding an amino-(Y₁)ₚ—(U)_{p′}—(Y₂)_{p″} group to a corresponding quinoline, enabling an intermediate compound Q-(Y₁)ₚ—(U)_{p′}—(Y₂)_{p″} to be obtained, and then grafting this intermediate compound onto an antibiotic A.
